(19) United States
(12) Patent Application Publication
(10) Pub. No.: US 2023/0151061 A1
(43) Pub. Date: May 18, 2023

(54) T CELL EPITOPE CLUSTERS AND RELATED COMPOSITIONS USEFUL IN THE PREVENTION, DIAGNOSIS, AND TREATMENT OF COVID-19

(71) Applicant: EPIVAX, INC., Providence, RI (US)

(72) Inventors: Anne De Groot, Providence, RI (US); William Martin, Providence, RI (US)

(21) Appl. No.: 17/795,282

(22) PCT Filed: Feb. 12, 2021
(86) PCT No.: PCT/US2021/017748

§ 371 Date: Jul. 26, 2022

Related U.S. Application Data
(60) Provisional application No. 62/976,715, filed on Feb. 14, 2020.
(60) Provisional application No. 62/983,012, filed on Feb. 28, 2020.
(60) Provisional application No. 62/991,790, filed on Mar. 19, 2020.
(60) Provisional application No. 62/991,814, filed on Mar. 19, 2020.
(60) Provisional application No. 63/001,632, filed on Mar. 30, 2020.
(60) Provisional application No. 63/001,624, filed on Mar. 30, 2020.
(60) Provisional application No. 63/004,729, filed on Apr. 3, 2020.
(60) Provisional application No. 63/006,962, filed on Apr. 8, 2020.
(60) Provisional application No. 63/065,135, filed on Aug. 13, 2020.
(60) Provisional application No. 63/065,161, filed on Aug. 13, 2020.
(60) Provisional application No. 63/065,152, filed on Aug. 13, 2020.
(60) Provisional application No. 63/065,129, filed on Aug. 13, 2020.
(60) Provisional application No. 63/073,161, filed on Sep. 1, 2020.
(60) Provisional application No. 63/073,156, filed on Sep. 1, 2020.
(60) Provisional application No. 63/081,062, filed on Sep. 21, 2020.
(60) Provisional application No. 63/065,163, filed on Aug. 13, 2020.
(60) Provisional application No. 63/081,055, filed on Sep. 21, 2020.
(60) Provisional application No. 63/083,389, filed on Sep. 25, 2020.
(60) Provisional application No. 63/092,229, filed on Oct. 15, 2020.

(51) Int. Cl. C07K 14/005; A61K 39/215; A61P 31/14; G01N 33/50; G01N 33/569

(52) U.S. Cl. CPC C07K 14/005; A61K 39/215; A61P 31/14; G01N 33/5091; G01N 33/505; G01N 33/56983; C12N 2770/20022; C12N 2770/20034; C07K 2319/00; A61K 2039/5254

(57) ABSTRACT

The present disclosure relates to novel epitope-based compositions, including vaccines, against severe acute respiratory syndrome coronavirus 2 (SARS-CoV-2) and diseases caused by SARS-CoV-2, including the highly contagious coronavirus disease 2019. The disclosure relates to immunogenic polypeptides (including concatemeric polypeptides, hybrid Ii-key constructs, and chimeric or fusion polypeptides as disclosed herein) and the uses thereof, particularly in vaccine compositions. The disclosure also relates to nucleic acids, vectors, and cells which express the polypeptides and the uses thereof. The polypeptides of the invention more specifically comprise an agretope predicted to be a ligand of HLA class I and/or HLA class II MHC molecules, as well as an epitope that is predicted to be recognized by T-cells in the context of MHC class I and/or class II molecules. The compositions are particularly suited to produce vaccines, particularly for vaccinating against SARS-CoV-2 infection and related diseases caused by SARS-CoV-2, including COVID-19.

### CROSS-REFERENCE TO RELATED APPLICATION

This application depends from and claims priority to U.S. Provisional Application No. 62/976,715 filed Feb. 14, 2020, U.S. Provisional Application No. 62/991,790 filed Mar. 19, 2020, U.S. Provisional Application No. 63/001,632 filed Mar. 30, 2020, U.S. Provisional Application No. 63/065,129 filed Aug. 13, 2020, U.S. Provisional Application No. 63/073,161 filed Sep. 1, 2020, U.S. Provisional Application No. 63/083,389 filed Sep. 25, 2020, U.S. Provisional Application No. 63/092,229 filed Oct. 15, 2020, U.S. Provisional Application No. 62/983,012 filed Feb. 28, 2020, U.S. Provisional Application No. 62/991,814 filed Mar. 19, 2020, U.S. Provisional Application No. 63/065,161 filed Aug. 13, 2020, U.S. Provisional Application No. 63/081,062 filed Sep. 21, 2020, U.S. Provisional Application No. 63/001,624 filed Mar. 30, 2020, U.S. Provisional Application No. 63/065,135 filed Aug. 13, 2020, U.S. Provisional Application No. 63/004,729 filed Apr. 3, 2020, U.S. Provisional Application No. 63/065,152 filed Aug. 13, 2020, U.S. Provisional Application No. 63/006,962 filed Apr. 8, 2020, U.S. Provisional Application No. 63/065,163 filed Aug. 13, 2020, U.S. Provisional Application No. 63/073,156 filed Sep. 1, 2020, and U.S. Provisional Application No. 63/081,055 filed Sep. 21, 2020, the entire contents of each of which are incorporated herein by reference.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created Feb. 10, 2021, is named “EPV0038WO Sequence Listing 1_ST25.txt” and is 148 KB bytes in size.

### FIELD

The present disclosure generally relates to novel T-cell epitope-based compounds and compositions, including vaccines, effective against severe acute respiratory syndrome coronavirus 2 (SARS-CoV-2) infection (or a closely related virus such as Severe Acute Respiratory Syndrome (SARS) or Middle East respiratory syndrome coronavirus (MERS-CoV)) and/or related diseases caused by SARS-CoV-2, including COVID-19 in a subject in need thereof. Such T-cell epitope compounds and compositions include immunogenic T-cell epitope polypeptides (including concatemeric polypeptides, hybrid Ii-key constructs, and chimeric or fusion polypeptides), as well as nucleic acids, plasmids, vectors (including expression vectors), and cells which express the polypeptides, pharmaceutical compositions, and vaccines. The present disclosure also generally relates to methods, assays, and kits for detecting a cell-mediated immune response, including a T-cell response (e.g., CD8+ and/or CD4+ T-cell response), against severe acute respiratory syndrome coronavirus 2 (SARS-CoV-2) or a related coronavirus, as well as methods, assays, and kits for the diagnosis of a SARS-CoV-2 infection or related coronaviruses infection.

### BACKGROUND

Severe acute respiratory syndrome coronavirus 2 (SARS-CoV-2) is a positive-sense single-stranded ribonucleic (RNA) virus belonging to the Coronaviridae family. SARS-CoV-2 (which may also be referred to herein as “COVID-19 virus”) was first identified in Wuhan, China in late 2019 and is the cause of the highly contagious coronavirus disease 2019 (which has been termed and may be referred to herein as “COVID-19”, “2019 novel coronavirus”, or “2019-nCoV”). SARS-CoV-2 infection causes a broad range of disease, known as coronavirus disease 2019 (COVID-19), from mild or no symptoms to serious complications that may be rapidly fatal, often in adults over 65 years old and individuals with underlying medical conditions including cardiovascular disease, type 2 diabetes, and obesity. The global spread of COVID-19 was declared a pandemic by the World Health Organization (WHO) on Mar. 11, 2020. As of Dec. 25, 2020, the global spread of severe acute respiratory syndrome coronavirus 2 (SARS-CoV-2) has resulted in over 79 million cases of COVID-19, 1.7 million deaths and global economic disruption in less than 10 months since the first case appeared in Wuhan, China. Recovery from natural infection in non-severe disease, and resistance to severe disease in younger individuals suggests that the immune system can be harnessed to help bring an end to the COVID-19 pandemic by vaccination strategies that recapitulate protective immune responses.

While immune correlates of COVID-19 protection are not yet defined, several studies show cellular adaptive immune mechanisms contribute to SARS-CoV-2 control. Humoral immune responses also contribute to protection and have been the focus of current vaccine development efforts. Virus-specific IgM and IgG antibodies are found in nearly all infections. Seroconversion is observed 7 to 14 days after onset of symptoms and persists for weeks after virus clearance. Antibody levels wane four to five months after infection, but durable memory B cell immunity has been described in mild and severe disease. Antibodies are found against the surface spike glycoprotein and the internal nucleocapsid protein. Neutralizing antibodies target the receptor binding domain of spike, preventing cell entry via the angiotensin-converting enzyme 2 (ACE2) host receptor. Neutralizing antibodies are found in more than 90% of persons who seroconvert. In a prospective study of exposed healthcare workers, anti-COVID-19 IgG titers were correlated with protection from subsequent PCR test positivity, suggesting that either antibodies, or T cell response (responsible for driving higher Ab titers) or both were correlates of protection from subsequent infection. In other studies, spike-specific follicular helper CD4 T cells (Tfh) frequencies correlate with neutralizing antibody responses. Although much of the current COVID-19 vaccine focus has been on generating antibody responses, this latter finding identifies a critical role for T cells in generating immunity.

More recently, correlations between a wide range of T cell responses and protection from infection have begun to emerge. A large prospective study showed numbers of SARS-CoV-2-specific T cells indirectly correlate with disease risk. Individuals with low T cell responses to spike, membrane and nucleocapsid proteins develop COVID-19 while high responders do not, even if seronegative. T cell breadth is another key feature of the protected response, as patients with mild disease have higher TCR clonality in blood and bronchoalveolar lavage in comparison with severe disease. T cell phenotype and function may also help to predict mild versus severe cases.

Poor outcomes are associated with multiple signs of T cell impairment including enhanced expression of PD-1 and TIM-3 exhaustion markers, higher inhibitory molecule levels including CTLA-4 and TIGIT, and low frequencies of polyfunctional CD4 and CD8 T cells, as well as low GzmB-producing CD8 T cells. In contrast, non-severe patients present with lower levels of inhibitory molecules and higher GzmA, GzmB, and perforin effectors. Moreover, in recovered patients, Tfh are found in the periphery at the time of viral clearance and persist into convalescence in contrast with an absence of lymph node Tfh found in patients who died of COVID-19. These findings underscore the importance of defining T cell epitope specificities to better understand COVID-19 immunity and to develop antibody- and T cell-directed vaccines that exploit T cell immunity.

There is an urgent need for the identification of CD4+ and CD8+ effector T cell epitopes contained in SARS-CoV-2 and for their use in the development of effective pharmaceuticals and vaccines. There is also an urgent need for methods, assays, and kits for detecting an immune response, including a cell-mediated immune response, such as a T-cell response (e.g., CD8+ and/or CD4+ T-cell response), against the SARS CoV-2 or related coronaviruses, as well as methods, assays, and kits for the diagnosis of a SARS-CoV-2 infection or related coronaviruses infection.

### SUMMARY

Accordingly, the present disclosure provides novel, therapeutic T cell epitope compounds and compositions (including one or more of e.g., peptides or polypeptides as disclosed herein, including polypeptides having an amino acid sequence comprising, consisting of, or consisting essentially of one or more of SEQ ID NOS. 4-224 and 454-456 and/or fragments and variants thereof, and optionally 1 to 12 additional amino acids distributed in any ratio on the N terminus and/or C-terminus of the polypeptide of SEQ ID NOS. 4-224 and 454-456; concatemeric peptides as disclosed herein; hybrid Ii-key constructs as disclosed herein, including hybrid Ii-key constructs comprising, consisting of, or consisting essentially of one or more of SEQ ID NOS: 232-452 and 457-459 and variants and fragments thereof; chimeric or fusion polypeptide compositions as disclosed herein (which in aspects may be isolated, synthetic, or recombinant); nucleic acids as disclosed herein, expression cassettes, plasmids, expression vectors, recombinant viruses, or cells as disclosed herein; vaccine compositions or formulations as disclosed herein, and/or pharmaceutical compositions as disclosed herein), and use of the same, e.g., in methods of stimulating, inducing, and/or expanding an immune response e.g., against coronavirus infection, including SARS-CoV-2 infection and related diseases caused by SARS-CoV-2, including COVID-19, and methods of treating and/or preventing against SARS-CoV-2 infection and related diseases caused by SARS-CoV-2, including COVID-19 in a subject.

In aspects, a T-cell epitope compound or composition of the present disclosure includes one or more peptides or polypeptides a disclosed herein. In aspects, the present disclosure is directed to a peptide or polypeptide having an amino acid sequence comprising, consisting of, or consisting essentially of one or more of SEQ ID NOS: 4-224 and 454-456, or fragments or variants thereof. The phrase “consisting essentially of” is intended to mean that a peptide or polypeptide according to the present disclosure, in addition to the sequence according to any of SEQ ID NOS: 4-224 and 454-456 or a fragment or variant thereof, contains additional amino acids or residues that may be present at either terminus of the peptide and/or on a side chain that are not necessarily forming part of the peptide or polypeptide that functions as an WIC ligand and provided they do not substantially impair the activity of the peptide to function as a T-cell epitope. The polypeptides of the present disclosure may be isolated, synthetic, and/or recombinant, and may comprise post-transcriptional modifications such as glycosylation, added chemical groups, etc. In aspects, the peptides or polypeptides can be either in neutral (uncharged) or salt forms, and may be either free of or include modifications such as glycosylation, side chain oxidation, or phosphorylation. In aspects, the peptides or polypeptides of the instant disclosure can be capped with an n-terminal acetyl and/or c-terminal amino group. In aspects, peptides or polypeptides of the instant disclosure having SEQ ID NOS: 9, 34, 49, 54, 59, 94, 99, 104, 109, 142, 147, 181, 191, 196, 210-213, 215-218, 220, and 223 are capped with an n-terminal acetyl and a c-terminal amino group. In aspects, peptides or polypeptides of the instant disclosure having SEQ ID NOS: 89, 114, 186, 214, 119, 221-222, and 224 are capped with an n-terminal acetyl and are not capped at the c-terminus.

In aspects, the instant disclosure is directed to a peptide or polypeptide comprising, consisting, or consisting essentially of an amino acid sequence of SEQ ID NOS: 4-224 and 454-456 (and/or fragments or variants thereof). In aspects, the instant disclosure is directed to a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 4-224 and 454-456. In aspects, the instant disclosure is directed to a peptide or polypeptide have a core amino acid sequence comprising, consisting of, or consisting essentially of one or more peptides or polypeptides having an amino acid sequence of SEQ ID NOS: 4-224 and 454-456, and optionally having extensions of 1 to 12 amino acids on the C-terminal and/or the N-terminal of the core amino acid sequence, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio). In aspects, the instant disclosure is directed to a peptide or polypeptide have a core sequence comprising, consisting of, or consisting essentially of one or more peptides or polypeptides having an amino acid sequence of SEQ ID NOS. 4-224 and 454-456 (and/or fragments and variants thereof), optionally with extensions of 1 to 12 amino acids on the C-terminal and/or the N-terminal, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio), provided that the polypeptide with the flanking amino acids is still able to bind to a same HLA molecule (i.e., retain MHC binding propensity) as said polypeptide core sequence without said flanking amino acids. In aspects, said polypeptide with the flanking amino acids is still able to bind to a same HLA molecule (i.e., retain MHC binding propensity) and/or retain the same TCR specificity as said polypeptide core sequence without said flanking amino acids. In aspects, said polypeptide with the flanking amino acids is still able to bind to a same HLA molecule (i.e., retain MHC binding propensity) and/or retain the same TCR specificity, and/or retain anti-viral activity, including anti-SARS-CoV-2 activity, as said polypeptide core sequence without said flanking amino acids. In aspects, said flanking amino acid sequences are those that also flank the peptides or polypeptides included therein in the naturally occurring protein. For example, for a peptide or polypeptide have a core sequence comprising, consisting of, or consisting essentially of one or more peptides or polypeptides having an amino acid sequence of SEQ ID NOS: 4-8 and 210 (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS: 4-8 and 210 in the amino acid sequence of the envelope (SEQ ID NO: 1) of 2019-nCoV. For a peptide or polypeptide have a core sequence comprising, consisting of, or consisting essentially of one or more peptides or polypeptides having an amino acid sequence of SEQ ID NOS: 9-63 and 211-213 (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS. 9-63 and 213 in the amino acid sequence of the membrane (SEQ ID NO: 2) of 2019-nCoV. For a peptide or polypeptide have a core sequence comprising, consisting of, or consisting essentially of one or more peptides or polypeptides having an amino acid sequence of SEQ ID NOS. 64-209 and 214-224 and 454-456 (and/or fragments and variants thereof), optionally with extensions of 1 to 12 amino acids on the C-terminal and/or the N-terminal, the extensions of 1 to 12 amino acids are those found flanking the amino acid sequence of 64-209 and 214-224 and 454-456 in the amino acid sequence of the spike (SEQ ID NO: 3) of 2019-nCoV. Additional examples are found in the specification, below. In aspects, said flanking amino acid sequences as described herein may serve as a MHC stabilizing region. In aspects, the use of a longer peptide may allow endogenous processing by patient cells and may lead to more effective antigen presentation and induction of T cell responses. In aspects, the extension(s) may serve to improve the biochemical properties of the peptides or polypeptides (e.g., but not limited to, solubility or stability) or to improve the likelihood for efficient proteasomal processing of the peptide. In aspects, the polypeptides of the present disclosure may be isolated, synthetic, and/or recombinant, and may comprise post-transcriptional modifications such as glycosylation, added chemical groups, etc. In aspects, the peptides or polypeptides can be either in neutral (uncharged) or salt forms, and may be either free of or include modifications such as glycosylation, side chain oxidation, or phosphorylation. In certain aspects, the peptides or polypeptides of the instant disclosure can be capped with an n-terminal acetyl and/or c-terminal amino group.

In aspects, the present disclosure is directed to a concatemeric polypeptide or peptide that comprises at one or more of the instantly-disclosed polypeptides or peptides (e.g., but not limited to, a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 4-224 and 454-456) linked, fused, or joined together (e.g., fused in-frame, chemically-linked, or otherwise bound) to an additional peptide or polypeptide. Such additional peptide or polypeptide may be one or more of the instantly instantly-disclosed polypeptides or peptides, or may be an additional peptide or polypeptide of interest. In aspects, a concatemeric peptide is composed of 3 or more, 4 or more, 5 or more 6 or more 7 or more, 8 or more, 9 or more of the instantly-disclosed peptides or polypeptides. In other aspects, the concatemeric peptides or polypeptides include 1000 or more, 1000 or less, 900 or less, 500 or less, 100 or less, 75 or less, 50 or less, 40 or less, 30 or less, 20 or less or 100 or less peptide epitopes. In yet other embodiments, a concatemeric peptide has 3-100, 5-100, 10-100, 15-100, 20-100, 25-100, 30-100, 35-100, 40-100, 45-100, 50-100, 55-100, 60-100, 65-100, 70-100, 75-100, 80-100, 90-100, 5-50, 10-50, 15-50, 20-50, 25-50, 30-50, 35-50, 40-50, 45-50, 100-150, 100-200, 100-300, 100-400, 100-500, 50-500, 50-800, 50-1,000, or 100-1,000 of the instantly-disclosed peptides or polypeptides linked, fused, or joined together. Each peptide or polypeptide of the concatemeric polypeptide may optionally have one or more linkers, which may optionally be cleavage sensitive sites, adjacent to their N and/or C terminal end. In such a concatemeric peptide, two or more of the peptide epitopes may have a cleavage sensitive site between them. Alternatively two or more of the peptide epitopes may be connected directly to one another or through a linker that is not a cleavage sensitive site. In aspects, the instantly-disclosed concatemeric polypeptide or peptide sequences do not correspond to a naturally occurring sequence, i.e., each of the one or more of the instantly-disclosed polypeptides or peptides (e.g., but not limited to, a peptide or polypeptide comprising, consisting, or consisting essentially of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 4-224 and 454-456) are linked, fused, or joined together (e.g., fused in-frame, chemically-linked, or otherwise bound) to an additional peptide or polypeptide (which may be one or more of the instantly-disclosed peptides) in such a fashion such that the overall concatemeric polypeptide does not correspond to a naturally occurring coronavirus sequence. In aspects, the concatemeric polypeptides of the present disclosure may be isolated, synthetic, and/or recombinant, and may comprise post-transcriptional modifications such as glycosylation, added chemical groups, etc. In aspects, the concatemeric polypeptides can be either in neutral (uncharged) or salt forms, and may be either free of or include modifications such as glycosylation, side chain oxidation, or phosphorylation. In certain aspects, the concatemeric polypeptides of the instant disclosure can be capped with an N-terminal acetyl and/or C-terminal amino group.

In aspects, one or more peptides or polypeptides or concatemeric polypeptides of the instant disclosure (e.g., but not limited to, a peptide or polypeptide comprising, consisting, or consisting essentially of an amino acid sequence of SEQ ID NOS: SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: SEQ ID NOS: 2-224, or a concatemeric polypeptide, or a hybrid Ii-key compound (e.g., but not limited to, a peptide or polypeptide comprising, consisting of, or consisting essentially of one or more of SEQ ID NOS: 232-452 and 457-459 and variants and fragments thereof)), is joined to, linked to (e.g., fused in-frame, chemically-linked, or otherwise bound), and/or inserted into a heterologous polypeptide. In aspects, the one or more peptides or polypeptides or concatemeric polypeptides of the instant disclosure may be joined to, linked to (e.g., fused in-frame, chemically-linked, or otherwise bound), and/or inserted into a heterologous polypeptide as a whole, although it may be made up from a joined to, linked to (e.g., fused in-frame, chemically-linked, or otherwise bound), and/or inserted amino acid sequence, together with flanking amino acids of the heterologous polypeptide.

In aspects, the present disclosure is directed to hybrid Ii-key constructs that are comprised of 3 elements, similar to the '813 hybrid. The 3 elements are: 1) an N-terminal element comprising, consisting or, or consisting essentially of 4-16 residues of the mammalian Ii-Key peptide LRMKLPKPPKPVSKMR (SEQ ID NO: 225) and modifications of SEQ ID No: 225 that retain antigen presentation enhancing activity (which in aspects may comprise, consist of, or consist essentially of the residues LRMK (amino acids 1-4) of SEQ ID NO.: 225 and 0-12 additional sequential residues of SEQ ID NO.: 225, and modifications of SEQ ID No: 225 that retain antigen presentation enhancing activity; or in other aspects may comprise, consist or, or consist essentially of LRMK (SEQ ID NO: 226)); 2) a C-terminal element comprising an WIC Class II and/or WIC Class I-presented epitope in the form of a polypeptide or peptidomimetic structure which binds to the antigenic peptide binding site of an WIC Class II and/or Class I molecule(s) (which in aspects comprises one or more of the instantly disclosed peptides or polypeptides (e.g., but not limited to, a peptide or polypeptide comprising, consisting, or consisting essentially of an amino acid sequence of SEQ ID NOS. 4-224 and 454-456 (and/or fragments or variants thereof), and optionally 1 to 12 additional amino acids distributed in any ratio on the N terminus and/or C-terminus of the polypeptide of SEQ ID NOS. 4-224 and 454-456)); and 3) an optional intervening chemical structure covalently linking the N-terminal and C-terminal elements of the hybrid, which in aspects the chemical structure being a covalently joined group of atoms which when arranged in a linear fashion forms a flexible chain which extends up to the length of 20 amino acids likewise arranged in a linear fashion. In aspects, the intervening chemical structure comprises, consists of, or consists essentially of a delta-aminovaleric acid, which is 5-aminopentanoic acid (and may be referred to as “ava”). In aspects, the intervening chemical structure may comprise, consist of, or consist essentially of ava, ala-ala-ala, gly-gly, or other biologically accepted functional equivalent of ava, ala-ala-ala, and gly-gly. Specific Ii-key hybrid constructs are disclosed in Table 2, and include SEQ ID NOS: 232-452 and 457-459. In certain aspects, a C-terminal element comprising an MHC Class II and/or MHC Class I-presented epitope in the form of a polypeptide or peptidomimetic structure which binds to the antigenic peptide binding site of an MHC Class II and/or Class I molecule(s) (which in aspects comprises one or more of the instantly disclosed peptides or polypeptides (e.g., but not limited to, a peptide or polypeptide comprising, consisting, or consisting essentially of an amino acid sequence of SEQ ID NOS. 4-224 and 454-456 (and/or fragments or variants thereof), and optionally 1 to 12 additional amino acids distributed in any ratio on the N terminus and/or C-terminus of the polypeptide of SEQ ID NOS. 4-224 and 454-456)) of the hybrid Ii-key constructs of instant disclosure can be capped with an n-terminal acetyl and/or c-terminal amino group.

In aspects of the instantly-disclosed hybrid Ii-key constructs comprising a peptide or polypeptide of the instant disclosure having SEQ ID NOS: 9, 34, 49, 54, 59, 94, 99, 104, 109, 142, 147, 181, 191, 196, 210-213, 215-218, 220, and 223 as the C-terminal element, such peptides are capped with an n-terminal acetyl and a c-terminal amino group. In aspects of the instantly-disclosed hybrid Ii-key constructs comprising a peptide or polypeptide of the instant disclosure having SEQ ID NOS: 89, 114, 186, 214, 119, 221-222, and 224 as the C-terminal element, such peptides are capped with an n-terminal acetyl and are not capped at the c-terminus. In aspects of the hybrid Ii-key constructs of SEQ ID NOS: 237, 262, 277, 283, 287, 322, 327, 332, 337, 370, 375, 409, 419, 424, 438-441, 443-446, 448, and 451, the disclosed peptide sequence of the C-terminal element are capped with an n-terminal acetyl and a c-terminal amino group. In aspects of the hybrid Ii-key constructs of SEQ ID NOS: 317, 342, 414, 442, 447 449, 450, and 452, the disclosed peptide sequence of the C-terminal element are capped with an n-terminal acetyl and are not capped at the c-terminus.

In aspects, the present disclosure is directed to a chimeric or fusion polypeptide composition (which in aspects may be isolated, synthetic, or recombinant) comprising one or more peptides, polypeptides, concatemeric peptides, or hybrid Ii-Key constructs of the present disclosure. In aspects, a chimeric or fusion polypeptide composition of the present disclosure comprises one or more peptides, polypeptides, concatemeric peptides, and/or hydrid Ii-Key constructs of the present disclosure (e.g., a peptide or polypeptide comprising, consisting of, or consisting essentially of one or more peptides or polypeptides having an amino acid sequence of SEQ ID NOS: 4-224 and 454-456 (and/or fragments or variants thereof), and optionally 1 to 12 additional amino acids distributed in any ratio on the N-terminus and/or C-terminus of the polypeptide of SEQ ID NOS: 2-224) joined to, linked to (e.g., fused in-frame, chemically-linked, or otherwise bound), and/or inserted into a heterologous polypeptide. In aspects, the one or more peptides, polypeptides, concatemeric peptides, and/or hydrid Ii-Key constructs of the present disclosure may be inserted into the heterologous polypeptide, may be added to the C-terminus (with or without the use of linkers, as is known in the art), and/or added to the N-terminus (with or without the use of linkers, as is known in the art) of the heterologous polypeptide. In aspects of the above chimeric or fusion polypeptide compositions, the one or more peptide, polypeptides, concatemeric peptides, and/or hydrid Ii-Key constructs may be joined to, linked to (e.g., fused in-frame, chemically-linked, or otherwise bound), and/or inserted into a heterologous polypeptide as a whole, although it may be made up from a joined to, linked to (e.g., fused in-frame, chemically-linked, or otherwise bound), and/or inserted amino acid sequence, together with flanking amino acids of the heterologous polypeptide. In aspects, a chimeric or fusion polypeptide composition of the present disclosure comprises a peptide, polypeptide, concatemeric peptides, and/or hydrid Ii-Key constructs of the instant disclosure, said peptide, polypeptide, concatemeric peptides, and/or hydrid Ii-Key constructs having a sequence that is not naturally included in the heterologous polypeptide and/or is not located at its natural position in the heterologous polypeptide. For example, in aspects, the one or more peptides, polypeptides, and/or concatemeric peptides of the present disclosure may be inserted into a SARS-CoV-2 sequence in which the SARS-CoV-2 sequence does not include the one or more peptides, polypeptides, and/or concatemeric peptides of the present disclosure (e.g., the SARS-CoV-2 sequence is mutated to not include the one or more peptides, polypeptides, and/or concatemeric peptides of the present disclosure) or the one or more peptides, polypeptides, and/or concatemeric peptides of the present disclosure is inserted into a SARS-CoV-2 sequence but not at its natural position. In aspects, the one or more of peptide, polypeptide, concatemeric peptides, and/or hydrid Ii-Key constructs of the present disclosure can be joined, linked to (e.g., fused in-frame, chemically-linked, or otherwise bound), and/or inserted into the heterologous polypeptide. In aspects of above-described chimeric or fusion polypeptide compositions, the chimeric or fusion polypeptides may be isolated, synthetic, or recombinant.

In aspects, the instant disclosure is directed to a nucleic acid (e.g., DNA or RNA, including mRNA) encoding one or more peptides, polypeptides, concatemeric peptides, hybrid Ii-key constructs and/or chimeric or fusion polypeptides as described herein. For example, in aspects, the instant disclosure is directed to a nucleic acid encoding a peptide or polypeptide comprising, consisting of, or consisting essentially of one or more peptides or polypeptides having an amino acid sequence of SEQ ID NOS. 4-224 and 454-456 (and/or fragments or variants thereof), and optionally 1 to 12 additional amino acids distributed in any ratio on the N terminus and/or C-terminus of the polypeptide of SEQ ID NOS. 4-224 and 454-456. Additionally, the instant disclosure is directed to a nucleic acid encoding an Ii-key construct as disclosed herein, including the Ii-key constructs of Table 2, include SEQ ID NOS: 232-452 and 457-459. In aspects, the present disclosure is directed to a vector, such as an expression vector, comprising such a nucleic acid as described. In aspects, the present disclosure is directed to expression cassettes, plasmids, expression vectors, recombinant viruses, or cells comprising a nucleic acid as described herein. In aspects, the present disclosure is directed to a cell or vaccine comprising such a vector as described. In aspects, the present disclosure is directed to a cell comprising a vector of the present disclosure.

In aspects, the instant disclosure is directed to a pharmaceutical composition, the pharmaceutical composition comprising a T-cell epitope compound or composition of the instant disclosure (e.g., one or more of polypeptides as disclosed herein; concatemeric peptides as disclosed herein; hybrid Ii-key constructs as disclosed herein; chimeric or fusion polypeptide compositions as disclosed herein; nucleic acids as disclosed herein, including nucleic acids encoding such peptides, polypeptides, concatemeric peptides, hybrid Ii-key constructs, or chimeric or fusion polypeptide compositions as disclosed herein; expression cassettes, plasmids, expression vectors, recombinant viruses, cells as disclosed herein) and a pharmaceutically acceptable carrier, excipient, and/or adjuvant. In aspects, the one or more nucleic acids encoding said peptides or polypeptides are DNA, RNA, or mRNA.

In aspects of the above-described pharmaceutical compositions, the composition comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 40, at least 50, at least 100, at least 200, at least 300, at least 400, at least 500, at least 600, at least 700, at least 800, at least 900, or at least 1000, peptides, polypeptides, concatemeric peptides, and/or Ii-key constructs as disclosed herein, including every value or range therebetween.

In aspects, the instant disclosure is directed to a vaccine comprising a T-cell epitope compound or composition of the instant disclosure (e.g., one or more of: polypeptides as disclosed herein; concatemeric peptides as disclosed herein; hybrid Ii-key constructs as disclosed herein; chimeric or fusion polypeptide compositions as disclosed herein; nucleic acids as disclosed herein, including nucleic acids encoding such peptides, polypeptides, concatemeric peptides, hybrid Ii-key constructs, or chimeric of fusion polypeptide compositions as disclosed herein; expression cassettes, plasmids, expression vectors, recombinant viruses, cells as disclosed herein; pharmaceutical compositions as disclosed herein; or vaccines as described herein) and, optionally, a carrier, excipient, and/or an adjuvant.

The present disclosure also relates to methods of immunizing or inducing an immune response in a subject, said method comprising administering to said subject one more peptides, polypeptides, concatemeric peptides, hybrid Ii-key constructs, chimeric or fusion polypeptides, nucleic acids, expression cassettes, plasmids, expression vectors, recombinant viruses, cells, pharmaceutical compositions, or vaccines as described herein. In aspects, the subject is a human. In aspects, the present disclosure is directed to methods of immunizing or inducing an immune response in a subject, comprising administering to said subject a T-cell epitope compound or composition of the instant disclosure (e.g., one or more of: polypeptides as disclosed herein; concatemeric peptides as disclosed herein; chimeric or fusion polypeptide compositions as disclosed herein; hybrid Ii-key constructs as disclosed herein, nucleic acids as disclosed herein, including nucleic acids encoding such peptides, polypeptides, concatemeric peptides, or chimeric or fusion polypeptide compositions as disclosed herein; expression cassettes, plasmids, expression vectors, recombinant viruses, cells as disclosed herein; pharmaceutical compositions as disclosed herein; or vaccines as described herein). In aspects, the subject is a human. In aspects, the present disclosure is directed to a method of stimulating, inducing, and/or expanding an immune response to a SARS-CoV-2 infection and/or related diseases caused by SARS-CoV-2, including COVID-19, in a subject, comprising administering to said subject a T-cell epitope compound or composition of the instant disclosure.

The present disclosure also relates to methods of treating and/or preventing a SARS-CoV-2 infection and/or related diseases caused by SARS-CoV-2, including COVID-19, in a subject, such as a human, comprising administering to said subject a T-cell epitope compound or composition of the instant disclosure (e.g., one or more of: polypeptides as disclosed herein; concatemeric peptides as disclosed herein; hybrid Ii-key constructs as disclosed herein; chimeric or fusion polypeptide compositions as disclosed herein; nucleic acids as disclosed herein, including nucleic acids encoding such peptides, polypeptides, concatemeric peptides, hybrid Ii-key constructs, or chimeric or fusion polypeptide compositions as disclosed herein; expression cassettes, plasmids, expression vectors, recombinant viruses, cells as disclosed herein; pharmaceutical compositions as disclosed herein; or vaccines as described herein).

As should be understood, the T-cell epitope compounds or compositions of the instant disclosure as described herein may be used to induce an immune response and/or to vaccinate a subject. It is particularly useful to vaccinate against SARS-CoV-2 infection and/or related diseases caused by SARS-CoV-2, including COVID-19.

In aspects, the instant disclosure provide novel methods, assays, and kits for detecting an immune response, and in aspects a cell-mediated immune (“CMI”) response, including a T cell response (e.g., CD8+ and/or CD4+ T cell response) against SARS-CoV-2 or a related coronavirus, as well as methods, assays, and kits for the diagnosis of a SARS-CoV-2 infection or related coronaviruses infection, as well as diseases caused by SARS-CoV-2, including the highly contagious coronavirus disease 2019. The instantly-disclosed assays, methods, and kits use or include one or more T-cell epitope compounds and compositions (including peptides or polypeptides (including Ii-Key constructs and concatemers)) a disclosed herein.

In aspects, the present disclosure is directed to methods of measuring a CMI response against COVID-19 or a related coronavirus infection in a subject by incubating a sample from the subject which comprises T-cells or other cells of the immune system with one or more peptides or polypeptides of the instant disclosure. In aspects, production of IFN-γ or other cytokine or immune effector molecule(s) is then detected. The presence or level of immune effector is then indicative of the level of cell mediated responsiveness of the subject. In aspects, preferably, the sample is whole blood which is collected in a suitable container comprising the antigen. Optionally, a simple sugar such as dextrose is added to the incubation mixture. Accordingly, one aspect of the present disclosure relates to a method for measuring a CMI response in a subject, said method comprising collecting a sample from said subject wherein said sample comprises cells of the immune system which are capable of producing immune effector molecules following stimulation by an antigen, incubating said sample with one or more peptides or polypeptides of the instant disclosure and then measuring the presence of or elevation in the level of an immune effector molecule wherein the presence or level of said immune effector molecule is indicative of the capacity of said subject to mount a cell-mediated immune response against SARS-CoV-2 or a related coronavirus infection. In aspects, the presence of or elevation in the level of an immune effector molecule wherein the presence or level of said immune effector molecule is indicative of the capacity of said subject to mount a cell-mediated immune response against SARS-CoV-2 or a related coronavirus infection.

In aspects, the present disclosure is directed to methods of assaying for SARS-CoV-2 or related coronavirus peptide-specific T-cells, the method comprising providing a fluid containing T-cells, adding one or more peptides or polypeptides of the instant disclosure to the fluid, incubating the fluid to cause cytokine release, and detecting the released cytokine. Preferably the method comprises providing the fluid containing T-cells in contact with a surface carrying an immobilized first antibody to the cytokine, adding the peptide or polypeptide to the fluid, incubating the resulting fluid mixture under conditions to cause any peptide or polypeptide-specific T-cells that have been pre-sensitized in vivo to the peptide or polypeptide to secrete the cytokine, and detecting any secreted cytokine bound to the immobilized first antibody. In aspects, the cells are preferably peripheral blood mononuclear cells (PMBC). They may suitably be taken from a patient known to be suffering, or to have suffered, from COVID-19 infection or a related coronavirus infection. In aspects, the cells used are fresh. In aspects, the assay is used to identify or quantitate peptide or polypeptide-specific T-cells e.g. CD8+ or CD4+ cells that have been activated or pre-sensitized in vivo to a particular peptide or polypeptide. In aspects, these are unrestimulated T-cells, i.e. cells capable of immediate effector function without the need to effect division/differentiation by in vitro culture. When a peptide or polypeptide in question is presented to such cells, the cells secrete various cytokines, of which any one may be selected for the purposes of this assay. In aspects, the cytokine selected is interferon-γ (IFN γ).

In aspects, the present disclosure provides a method of detecting an anti-SARS-CoV-2 (or related coronavirus) T cell response (which in aspects can included CD4+ and/or CD8+ T cell response) comprising contacting a population of T cells of an individual with a peptide or polypeptide of the instant disclosure, wherein one or more of said peptides or polypeptides may be substituted by an analogue which binds a T cell receptor that recognizes the peptide, and determining whether T cells of the T cell population recognize the peptide(s).

In aspects, the present disclosure provides a method of diagnosing a SARS-CoV-2 or related coronavirus infection in a host, or exposure of a host, to SARS-CoV-2 or related coronavirus comprising: (i) contacting a population of T cells from the host with one or more peptides or analogues as disclosed here, and analogues thereof which can bind a T cell receptor which recognizes any of the said peptides; and (ii) determining whether the T cells of said T cell population recognize the peptide(s) and/or analogue(s).

### BRIEF DESCRIPTION OF THE FIGURES

The present disclosure may be better understood with reference to the following figures.

FIG. 1 depicts the amino acid sequences of the envelope (SEQ ID NO: 1), membrane (SEQ ID NO: 2), and the spike (SEQ ID NO: 3) of 2019-nCoV.

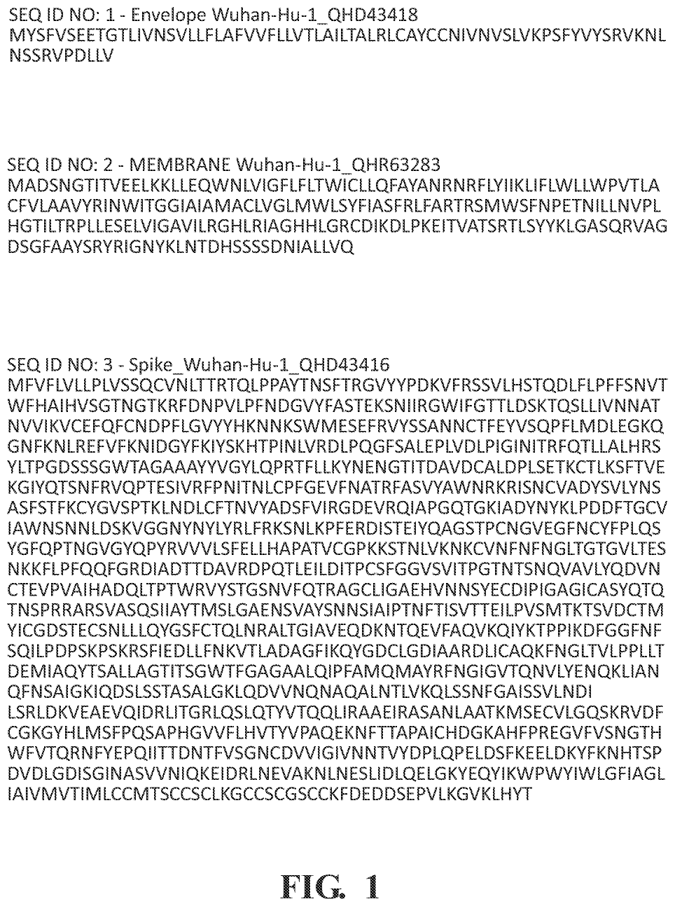

FIG. 2A and FIG. 2B, ex vivo immune recall responses differentiate SARS-CoV-2 naïve and experienced individuals and exhibit different COVID-19 immunotypes.

FIG. 3A and FIG. 3B shows that strong ex vivo immune recall responses are found or may be found in SARS-CoV-2 experienced individuals using polypeptides of the instant disclosure.

FIG. 4 shows polypeptides of the instant disclosure stimulate ex vivo immune recall response in natural SARS-CoV-2 infection.

FIG. 5A and FIG. 5B show polypeptides of the instant disclosure stimulate or may stimulate higher IFN-γ responses in naïve and COVID-19 convalescent donors following expansion in culture.

FIG. 6A and FIG. 6B show polypeptides of the instant disclosure stimulate or may stimulate low frequency epitope-specific T cells following expansion in culture in naïve and COVID-19 convalescent donors.

FIG. 7 shows polypeptides of the instant disclosure stimulate low frequency epitope-specific T cells following expansion in culture in naïve and COVID-19 convalescent donors.

FIGS. 8A-D show predicted SARS-CoV-2 T cell epitopes are antigenic ex vivo in COVID-19 convalescent donors but not healthy donors. (FIG. 8A) Convalescent and pre-SARS-CoV-2 donors (naïve) were stimulated with the total peptide pool consisting of our 32 predicted epitopes and IFNγ producing cells were measured by Fluorospot assay. Open circles identify responses to low-dose restimulation. Horizontal line indicates positivity criteria at SFC/10⁶ spenocytes=25. (FIG. 8B) IFNγ responses to individual peptides were also assessed, identifying the breadth of response in individual donors, (FIG. 8C) the frequency of responses to unique peptides within the cohort (vertical lines denote 20% of each cohort), and (FIG. 8D) the depth of response indicated by the frequency of IFNγ-producing, epitope-specific cells, vertical lines indicate positivity criteria at SFC/10⁶ spenocytes=25). *=p<0.05.

FIGS. 9A-B show SARS-CoV-2 experienced individuals exhibit variable immune recall responses ex vivo. (FIG. 9A) Significant responses to individual peptides (combined per source antigen) identify three distinct immunotype cohorts within covalescent donors (Donor notations; *=pneumonia, **=hospitalized, non-ICU). (FIG. 9B) Correlation of cumulative T cell responses and age according to gender.

FIGS. 10A-D show antigen-specific T cell expansion increases responses in COVID-19 convalescents and uncovers pre-existing SARS-CoV-2 immunity in healthy donors. (FIG. 10A) PBMCs of convalescent and naïve donors were restimulated with the total peptide pool following 8-day expansion culture and IFNγ producing cells were measured by Fluorospot assay. Horizontal line indicates positivity criteria at SFC/10⁶ spenocytes=25. (FIG. 10B) IFNγ responses to individual peptides were also assessed, identifying the breadth of response in individual donors, (FIG. 10C) the frequency of responses to unique peptides within the cohort (vertical lines denote 20% of each cohort), (FIG. 10D) and the depth of response indicated by the frequency of IFNγ-producing, epitope-specific cells (vertical lines indicate positivity criteria at SFC/10⁶ spenocytes=25).

FIGS. 11A-D show EPV-CoV-19 immunization stimulates strong type 1-skewed T cell responses in HLA-DR3 transgenic mice. Eight days post-boost, murine splenocytes were isolated and assayed for epitope-specific recall responses. Cells were plated in dual IFNγ/IL-4 fluorospot plates, and restimulated with peptide pools for 48 h. (FIG. 11A) Representative images and spot counts are shown for both. (FIG. 11B) IFNγ SFC counts were normalized to 1×10⁶ cells and adjusted by background subtraction, and (FIG. 11C) IFNγ SI index was determined by calculating the fold change of individual restimulation replicates over background. (FIG. 11C) IL-4 SFC and (FIG. 11D) SI were similarly calculated. Horizontal lines denote positivity criteria of SFC>25 and SI>5, respectively. (FIG. 11D) From the reported IFNγ and IL-4 stimulation indexes, we calculated the IFNγ:IL-4 ratio of each restimulation replicate to model the overall skewing of the immune response, identifying a sharply type 1 skewed phenotype in all vaccinated animals. Horizontal lines identify 40, 100, and 1000-fold skewing of type 1/type 2 response. (n=17, *=p<0.05, **=p<0.01, ***=p<0.001).

FIGS. 12A-E show EPV-CoV-19 immunization stimulates type 1-skewed Memory CD4 and CD8 T cells in HLA-DR3 transgenic mice. Splenocytes were restimulated with a vaccine-matched peptide pool 6 hours in the presence of brefeldin A and monensin. Following incubation, cells were stained for surface markers, fixed and permeabilized, stained for intracellular markers, and expression of markers was recorded by flow cytometry. Memory CD4⁺ T cells and CD8⁺ T cells were assessed for IFNγ, IL-4, or IL-5-production (both frequency in parent T cell population and mean fluorescence intensity (MFI) of cytokines). (FIG. 12A) Representative images of type 1 and type 2 skewed, epitope-specific memory T cell populations are shown. (FIG. 12B) The fold increase of epitope-specific responses (over CD28 stimulated controls) identify vaccine-specific induction of IFNγ, but not (FIG. 12C) IL-4, or (FIG. 12D) IL-5. (FIG. 12E) From ICS generated data, we calculated the fold increase of IFNγ or IL-4 and/or IL-5 producing cells with peptide restimulation and used the ratio of type 1-to-type 2 responses to model Th-skewing and Tc-skewing in vaccinated animals. (n=17, *=p<0.05, *=p<0.01, ***=p<0.001)

### DETAILED DESCRIPTION OF THE INVENTION

The present disclosure generally relates to T cell epitope-based compounds and compositions, including vaccines, for use against SARS-CoV-2 infection and related diseases caused by SARS-CoV-2, including COVID-19. The disclosure relates to immunogenic peptides, polypeptides, concatemeric peptides, hybrid Ii-key constructs, and/or chimeric or fusion polypeptides and the uses thereof, particularly in pharmaceutical and vaccine compositions. The present disclosure also relates to nucleic acids, vectors (including expression vectors), and cells which express the peptides, polypeptides, concatemeric peptides, hybrid Ii-key constructs, and/or chimeric or fusion polypeptides and the uses thereof. The peptides, polypeptides, concatemeric peptides, hybrid Ii-key constructs, and/or chimeric or fusion polypeptides of the present disclosure more specifically comprise an agretope predicted to be a ligand of HLA class I and/or HLA class II MHC molecules, as well as an epitope that is predicted to be recognized by T-cells (including CD8+ and/or CD4+ T-cells) in the context of MHC class I and/or class II molecules. The instant disclosure is particularly suited to produce vaccines for humans, particularly for vaccinating against coronavirus infection, including SARS-CoV-2 infection and related diseases caused by SARS-CoV-2, including COVID-19.

It is possible to exploit epitope-specific T-cells to induce immunity against specific antigens. This discovery has implications for the design of therapeutic regimens and antigen-specific therapies against particular pathogens and infections. The instant disclosure and data relates to identified SARS-CoV-2 T-cell epitopes that are recognized in natural infection and stimulate pre-existing immunity to SARS-CoV-2. These epitopes are excellent candidates for T-cell-directed vaccine development. A T-cell targeting vaccine composed of conserved epitopes may provide rapid, effective, and long-term immunity at sites of infection with production of tissue resident memory CD8+ T-cells, as well as memory CD4+ T-cells that support antibody responses. Influenza vaccination during the 2009 H1N1 pandemic demonstrated that memory CD4+ T-cells are able to support naïve B-cell response to a novel hemagglutinin. A T-cell-directed SARS-CoV-2 vaccine could generate robust CD4+ T-cell memory that would provide early control of acute infection with a novel SARS-CoV-2 virus in the absence of pre-existing cross-protective antibodies. Thus, administration of T-cell epitopes, including a T-cell epitope compound or composition of the present disclosure (including one or more of peptides or polypeptides having a sequence comprising, consisting of, or consisting essentially of one or more of SEQ ID NOS. 4-224 and 454-456 and/or fragments and variants thereof, and optionally 1 to 12 additional amino acids distributed in any ratio on the N terminus and/or C-terminus of the polypeptide of SEQ ID NOS. 4-224 and 454-456 (in aspects, the polypeptides may be isolated, synthetic, or recombinant) as disclosed herein; concatemeric peptides as disclosed herein; hybrid Ii-key constructs as disclosed herein, including the specific Ii-key hybrid constructs are disclosed in Table 2 (SEQ ID NOS: 232-452 and 457-459); chimeric of fusion polypeptide compositions as disclosed herein; nucleic acids encoding such peptides, polypeptides, concatemeric peptides, hybrid Ii-key constructs, or chimeric of fusion polypeptide compositions as disclosed herein; expression cassettes, plasmids, expression vectors, recombinant viruses, or cells which express such peptides, polypeptides, concatemeric peptides, hybrid Ii-key constructs, or chimeric of fusion polypeptide compositions as disclosed herein; vaccine compositions or formulations as disclosed herein; and/or pharmaceutical compositions or formulations as disclosed herein), optionally in conjunction with a drug (such as an antiviral drug), a protein, or a inactivated or live attenuated virus, can induce an immune response, e.g., against a pathogen, including SARS-CoV-2 and related diseases caused by SARS-CoV-2, including COVID-19. T-cell epitopes, including T-cell epitope compounds and compositions of the present disclosure, can be used to deliberately manipulate the immune system toward immunity.

For example, the T-cell epitope compounds and compositions of the present disclosure are useful in the selective engagement and activation of the immunogenic T-cells. It is demonstrated herein that certain naturally occurring T-cells (in aspects, including CD4+ and CD8+ T-cells), can be engaged, activated, and/or applied to induce immunity or induce an immune response against pathogens such as SARS-CoV-2 and related diseases caused by SARS-CoV-2, including COVID-19. By using the T-cell epitope compounds and compositions of the present disclosure to selectively activate naturally occurring T-cells, it is herein shown that such T-cell epitope compounds and compositions can be used to stimulate, induce, and/or expand an immune response to a coronavirus, including SARS-CoV-2 and related diseases caused by SARS-CoV-2, including COVID-19 in a subject, and thus can be used in methods of treating and/or preventing SARS-CoV-2 and related diseases caused by SARS-CoV-2, including COVID-19 in a subject.

### Definitions

To further facilitate an understanding of the present disclosure, a number of terms and phrases are defined below. Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Ranges provided herein are understood to be shorthand for all of the values within the range. For example, a range of 1 to 25 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, or 25, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 25 may comprise 1 to 5, 1 to 10, 1 to 15, and 1 to 20 in one direction, or 25 to 20, 25 to 15, 25 to 10, and 25 to 5 in the other direction.

As used herein, the term “biological sample” as refers to any sample of tissue, cells, or secretions from an organism.

As used herein, the term “medical condition” includes, but is not limited to, any condition or disease manifested as one or more physical and/or psychological symptoms for which treatment and/or prevention is desirable, and includes previously and newly identified diseases and other disorders.

As used herein, the term “immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells, metastatic tumor cells, malignant melanoma, invading pathogens (including a virus), cells or tissues infected with pathogens, or, in cases of autoimmunity or pathological inflammation, normal human cells or tissues. In aspects, an immune response includes a measurable cytotoxic T lymphocyte (CTL) response (e.g., against a virus expressing an immunogenic polypeptide) or a measurable B cell response, such as the production of antibodies, (e.g., against an immunogenic polypeptide). One of ordinary skill would know various assays to determine whether an immune response against a peptide, polypeptide, or related composition was generated, including use of the experiments and assays as disclosed in the Examples herein. Various B lymphocyte and T lymphocyte assays are well known, such as ELISAs, EliSpot assays, cytotoxic T lymphocyte (CTL) assays, such as chromium release assays, proliferation assays using peripheral blood lymphocytes (PBL), tetramer assays, and other cytokine production assays. See Benjamini et al. (1991), hereby incorporated by reference.

As used herein, the term “effective amount”, “therapeutically effective amount”, or the like of a composition, including a T-cell epitope compound or composition of the present disclosure (including one or more of peptides or polypeptides having a sequence comprising, consisting of, or consisting essentially of one or more of SEQ ID NOS. 4-224 and 454-456 and/or fragments and variants thereof, and optionally 1 to 12 additional amino acids distributed in any ratio on the N terminus and/or C-terminus of the polypeptide of SEQ ID NOS. 4-224 and 454-456 as disclosed herein; concatemeric peptides as disclosed herein; hybrid Ii-key constructs as disclosed herein, including the specific Ii-key hybrid constructs are disclosed in Table 2 (SEQ ID NOS: 232-452 and 457-459); chimeric of fusion polypeptide compositions as disclosed herein; nucleic acids encoding such peptides, polypeptides, concatemeric peptides, hybrid Ii-key constructs, or chimeric of fusion polypeptide compositions as disclosed herein; expression cassettes, plasmids, expression vectors, recombinant viruses, or cells which express such peptides, polypeptides, concatemeric peptides or chimeric of fusion polypeptide compositions as disclosed herein; vaccine compositions or formulations, and/or pharmaceutical compositions or formulations as disclosed herein) is a quantity sufficient to achieve a desired therapeutic and/or prophylactic effect, e.g., an amount that results in the prevention of, or a decrease in, the symptoms and/or underlying causes associated with a disease that is being treated, such as SARS-CoV-2 infection and related diseases caused by SARS-CoV-2, including COVID-19, or an amount to measurably to inhibit virus (for example, SARS-CoV-2) replication or infectivity. The amount of a composition of the present disclosure administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The T-cell epitope compounds and compositions of the present disclosure can also be administered in combination with each other or with one or more additional therapeutic compounds.

As used herein, “anti-SARS-CoV-2 activity”, “anti-SARS-CoV-2 polypeptides”, “anti-SARS-CoV-2 compounds and compositions”, and the like are intended to mean that the T-cell epitope compounds and compositions of the of the present disclosure (including polypeptides, concatemeric polypeptides, hybrid Ii-key constructs, chimeric or fusion proteins, nucleic acids, plasmids, vectors, pharmaceutical compositions, vaccines, and other compositions of the instant disclosure) have anti-SARS-CoV-2 activity and thus are capable of suppressing, controlling, and/or killing an invading SARS-CoV-2 virus. For example, anti-SARS-CoV-2 activity means that the instantly-disclosed therapeutic T-cell epitope compounds and compositions are, in aspects: capable of stimulating, inducing, and/or expanding an immune response to SARS-CoV-2 (e.g., a cellular (CD4+ and/or CD8+ T-cell response) or humoral immune response to SARS-CoV-2) and/or associated diseases in a subject; capable of stimulating, inducing, and/or expanding a SARS-CoV-2-specific IFNγ response (e.g., by lymphocytes such as PMBC, or effector CD4+ and/or CD8+ T-cells), capable of inhibiting SARS-CoV-2 viral replication or infectivity, and/or capable of inducing immunity against SARS-CoV-2. In aspects, a T-cell epitope compound or composition of the present disclosure having anti-SARS-CoV-2 activity will reduce the disease symptoms resulting from SARS-CoV-2 challenge by at least about 5% to about 50%, at least about 10% to about 60%, at least about 30% to about 70%, at least about 40% to about 80%, or at least about 50% to about 90% or greater, including any value or range therebetween. Anti-SARS-CoV-2 activity can be determined by various experiments and assays as known to those of skill in the art, including methods such as by antibody titrations of sera, e.g., by ELISA and/or seroneutralization assay analysis and/or by vaccination challenge evaluation, including use of experiments and assays as disclosed in the Examples herein.

As used herein, the term “T-cell epitope” means an MHC ligand or protein determinant, 7 to 30 amino acids in length, and capable of specific binding to human leukocyte antigen (HLA) molecules and interacting with specific T cell receptors (TCRs). As used herein, in the context of a T cell epitope that is known or determined (e.g. predicted) to engage a T cell, the terms “engage”, “engagement” or the like means that when bound to a MHC molecule (e.g. human leukocyte antigen (HLA) molecules), the T cell epitope is capable of interacting with the TCR of the T cell and activating the T cell. Generally, T-cell epitopes are linear and do not express specific three-dimensional characteristics. T-cell epitopes are not affected by the presence of denaturing solvents. The ability to interact with T-cell epitopes can be predicted by in silico methods (De Groot A S et al., (1997), AIDS Res Hum Retroviruses, 13(7):539-41; Schafer J R et al., (1998), Vaccine, 16(19):1880-4; De Groot A S et al., (2001), Vaccine, 19(31):4385-95; De Groot A R et al., (2003), Vaccine, 21(27-30):4486-504, all of which are herein incorporated by reference in their entirety

As used herein, the term “T-cell epitope cluster” refers to polypeptide that contains between about 4 to about 40 MHC binding motifs. In particular embodiments, the T-cell epitope cluster contains between about 5 to about 35 MHC binding motifs, between about 8 and about 30 MHC binding motifs; and between about 10 and 20 MHC binding motifs.

As used herein, the term “immune-stimulating T-cell epitope polypeptide” refers to a molecule capable of inducing an immune response, e.g., a humoral, T cell-based, or innate immune response.

As used herein, the term “regulatory T cell”, “Treg” or the like, means a subpopulation of T cells that suppress immune effector function, including the suppression or down regulation of CD4+ and/or CD8+ effector T cell (Teff) induction, proliferation, and/or cytokine production, through a variety of different mechanisms including cell-cell contact and suppressive cytokine production. In aspects, CD4+ Tregs are characterized by the presence of certain cell surface markers including but not limited to CD4, CD25, and FoxP3. In aspects, upon activation, CD4+ regulatory T cells secrete immune suppressive cytokines and chemokines including but not limited to IL-10 and/or TGFβ. CD4+ Tregs may also exert immune suppressive effects through direct killing of target cells, characterized by the expression upon activation of effector molecules including but not limited to granzyme B and perforin. In aspects, CD8+ Tregs are characterized by the presence of certain cell surface markers including but not limited to CD8, CD25, and, upon activation, FoxP3. In aspects, upon activation, regulatory CD8+ T cells secrete immune suppressive cytokines and chemokines including but not limited to IFNγ, IL-10, and/or TGFβ. In aspects, CD8⁺ Tregs may also exert immune suppressive effects through direct killing of target cells, characterized by the expression upon activation of effector molecules including but not limited to granzyme B and/or perforin.

As used herein, the term “B-cell epitope” means a protein determinant capable of specific binding to an antibody. B-cell epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

As used herein, the terms “the major histocompatibility complex (MHC)”, “MHC molecules”, “MHC proteins” or “HLA proteins” are to be understood as meaning, in particular, proteins capable of binding peptides resulting from the proteolytic cleavage of protein antigens and representing potential T-cell epitopes, transporting them to the cell surface and presenting them there to specific cells, in particular cytotoxic T-lymphocytes or T-helper cells. The major histocompatibility complex in the genome comprises the genetic region whose gene products expressed on the cell surface are important for binding and presenting endogenous and/or foreign antigens and thus for regulating immunological processes. The major histocompatibility complex is classified into two gene groups coding for different proteins, namely molecules of MHC class I and molecules of MHC class II. The molecules of the two MHC classes are specialized for different antigen sources. The molecules of MHC class I present endogenously synthesized antigens, for example viral proteins and tumor antigens. The molecules of MHC class II present protein antigens originating from exogenous sources, for example bacterial products. The cellular biology and the expression patterns of the two MHC classes are adapted to these different roles. MHC molecules of class I consist of a heavy chain and a light chain and are capable of binding a peptide of about 8 to 11 amino acids, but usually 9 or 10 amino acids, if this peptide has suitable binding motifs, and presenting it to cytotoxic T-lymphocytes. The peptide bound by the MHC molecules of class I originates from an endogenous protein antigen. The heavy chain of the MHC molecules of class I is preferably an HLA-A, HLA-B or HLA-C monomer, and the light chain is β-2-microglobulin. MHC molecules of class II consist of an α-chain and a β-chain and are capable of binding a peptide of about 12 to 25 amino acids if this peptide has suitable binding motifs, and presenting it to T-helper cells. The peptide bound by the MHC molecules of class II usually originates from an extracellular of exogenous protein antigen. The α-chain and the β-chain are in particular HLA-DR, HLA-DQ and HLA-DP monomers.

As used herein, the term “MHC complex” refers to a protein complex capable of binding with a specific repertoire of polypeptides known as HLA ligands and transporting said ligands to the cell surface.

As used herein, the term “MHC Ligand” means a polypeptide capable of binding to one or more specific MHC alleles. The term “HLA ligand” is interchangeable with the term “MHC Ligand.” Cells expressing MHC/Ligand complexes on their surface are referred to as “Antigen Presenting Cells” (APCs). Similarly, as used herein, the term “MHC binding peptide” relates to a peptide which binds to an MHC class I and/or an MHC class II molecule. In the case of MHC class I/peptide complexes, the binding peptides are typically 8-10 amino acids long although longer or shorter peptides may be effective. In the case of MHC class II/peptide complexes, the binding peptides are typically 10-25 amino acids long and are in particular 13-18 amino acids long, whereas longer and shorter peptides may also be effective.

As used herein, the term “T Cell Receptor” or “TCR” refers to a protein complex expressed by T cells that is capable of engaging a specific repertoire of MHC/Ligand complexes as presented on the surface of cells, such as antigen presenting cells (APCs).

As used herein, the term “MHC Binding Motif” refers to a pattern of amino acids in a protein sequence that predicts binding to a particular MHC allele.

As used herein, the term “AAY cleavage motif” refers to the short amino acid motif consisting of the sequence “alanine-alanine-tyrosine” capable of promoting proteasome-mediated cleavage of a peptide or protein, promoting the binding of the transporter associated with antigen processing to a peptide or protein, and/or increasing proteasome degradation at specific sites within a peptide or protein.

As used herein, the term “immune synapse” means the protein complex formed by the simultaneous engagement of a given T cell epitope to both a cell surface MHC complex and TCR.

The term “polypeptide” refers to a polymer of amino acids, and not to a specific length; thus, peptides, oligopeptides and proteins are included within the definition of a polypeptide. As used herein, a polypeptide is said to be “isolated” or “purified” when it is substantially free of cellular material when it is isolated from recombinant and non-recombinant cells, or free of chemical precursors or other chemicals when it is chemically synthesized. A peptide or polypeptide (e.g., a polypeptide comprising, consisting of, or consisting essentially of one or more of SEQ ID NOS: 4-224 and 454-456 or variants and fragments thereof, which in aspects may be isolated, synthetic, or recombinant) of the present disclosure, however, can be joined to, linked to, or inserted into another polypeptide (e.g., a heterologous polypeptide) with which it is not normally associated in a cell and still be “isolated” or “purified.” Additionally, one or more T-cell epitopes of the present disclosure can be joined to, linked to, or inserted into another polypeptide wherein said one or more T-cell epitopes of the present disclosure is not naturally included in the polypeptide and/or said one or more T-cell epitopes of the present disclosure is not located at its natural position in the polypeptide. When a polypeptide is recombinantly produced, it can also be substantially free of culture medium, for example, culture medium represents less than about 20%, less than about 10%, or less than about 5% of the volume of the polypeptide preparation.

As used herein, a “concatemeric” peptide or polypeptide refers to a series of at least two peptides or polypeptides linked together. Such linkages may form of string-of-beads design. In aspects, each of the peptides or polypeptides of concatemeric polypeptide may optionally be spaced by one or more linkers, and in further aspects neutral linkers. The term “linker” refers to a peptide added between two peptide domains such as epitopes or vaccine sequences to connect said peptide domains. In aspects, a linker sequence is used to reduce steric hindrance between each one or more identified peptides of the instant disclosure, is well translated, and supports or allows processing of the each one or more identified polypeptides of the instant disclosure. In aspects, the linker should have little or no immunogenic sequence elements. In aspects, each peptide or polypeptide of the concatemeric polypeptide may optionally have one or more linkers, which may optionally be cleavage sensitive sites, adjacent to their N- and/or C-terminal end. In such a concatemeric peptide, two or more of the peptides may have a cleavage sensitive site between them. Alternatively two or more of the peptides may be connected directly to one another or through a linker that is not a cleavage sensitive site.

As used herein, the term “pharmaceutically acceptable” refers to approved or approvable by a regulatory agency of the Federal or a state government or listed in the U.S. Pharmacopeia or other generally recognized pharmacopeia for use in animals, including humans.

As used herein, the term “pharmaceutically acceptable excipient, carrier, or diluent” or the like refer to an excipient, carrier, or diluent that can be administered to a subject, together with an agent, and which does not destroy the pharmacological activity thereof and is nontoxic when administered in doses sufficient to deliver a therapeutic amount of the agent.

As used herein, the term “purpose built computer program” refers to a computer program designed to fulfill a specific purpose; typically to analyze a specific set of raw data and answer a specific scientific question.

As used herein, the term “z-score” indicates how many standard deviations an element is from the mean. A z-score can be calculated from the following formula: z=(X−μ)/σ; where z is the z-score, X is the value of the element, μ is the population mean, and σ is the standard deviation.

As used herein, the singular forms “a,” “an,” and “the” are intended to include the plural forms, including “at least one,” unless the content clearly indicates otherwise. “Or” means “and/or.” As used herein, the term “and/or” and “one or more” includes any and all combinations of the associated listed items. For example, the term “one or more” with respect to the “one or more of SEQ ID NOS: 4-224 and 454-456 of the present disclosure” includes any and all combinations of SEQ ID NOS: 4-224 and 454-456. The term “or a combination thereof” means a combination including at least one of the foregoing elements.

The following abbreviations and/or acronyms are used throughout this application:

| | |
|---|---|
| AAY | Alanine-Alanine-Tyrosine cleavage motif |
| MHC | major histocompatibility complex |
| 2019-nCoV | 2019 Novel Coronavirus |

A “variant” peptide or polypeptide (including a variant T-cell epitope) can differ in amino acid sequence by one or more substitutions, deletions, insertions, inversions, fusions, and truncations or a combination of any of these. In aspects, a variant peptide or polypeptide (including a variant T-cell epitope) can differ in amino acid sequence by one or more substitutions, deletions, insertions, inversions, fusions, and truncations or a combination of any of these provided said variants retain MHC binding propensity and/or TCR specificity, and/or SARS-CoV-2 activity.

The present disclosure also includes fragments of the peptide or polypeptides of the invention. The disclosure also encompasses fragments of the variants of the T-cell epitopes described herein, provided said fragments and/or variants at least in part retain MHC binding propensity and/or TCR specificity, and/or retain anti-SARS-CoV-2 activity.

The present disclosure also provides chimeric or fusion polypeptides (which in aspects may be isolated, synthetic, or recombinant) wherein one or more of the instantly-disclosed peptides, polypeptides, concatemeric peptides, and/or Ii-key constructs is a part thereof. In aspects, a chimeric or fusion polypeptide composition comprises one or more peptides, polypeptides, concatemeric peptides, and/or Ii-key constructs of the instant disclosure linked to a heterologous polypeptide. As previously stated, the term “heterologous polypeptide” is intended to mean that the one or more T-cell epitopes (e.g., one or more of SEQ ID NOS: 4-224 and 454-456) are heterologous to, or not included naturally, in the heterologous polypeptide. In aspects, the one or more peptides, polypeptides, concatemeric peptides, and/or Ii-key constructs of the instant disclosure may be inserted into the heterologous polypeptide (e.g., through mutagenesis or other known means in the art), may be added to the C-terminus (with or without the use of linkers, as is known in the art), and/or added to the N-terminus (with or without the use of linkers, as is known in the art) of the heterologous polypeptide. For example, protein engineering by mutagenesis can be performed using site-directed mutagenesis techniques, or other mutagenesis techniques known in the art (see e.g., James A. Brannigan and Anthony J. Wilkinson., 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety).

In aspects, chimeric or fusion polypeptides comprise one or more peptides, polypeptides, concatemeric peptides, and/or Ii-key constructs of the instant disclosure operatively linked to a heterologous polypeptide. “Operatively linked” indicates that the polypeptide (e.g., the one or more T-cell epitope polypeptides of the present disclosure) and the heterologous protein are fused in-frame or chemically-linked or otherwise bound. In aspects, the instantly-disclosed chimeric or fusion polypeptides may be isolated, synthetic, or recombinant

An “isolated” peptide, polypeptide, concatemeric peptide (e.g., an isolated T-cell activating T-cell epitope or T-cell epitope polypeptide), hybrid Ii-key construct, or chimeric or fusion polypeptide can be purified from cells that naturally express it, purified from cells that have been altered to express it (recombinant), or synthesized using known protein synthesis methods. In one embodiment, a peptide, polypeptide, concatemeric peptide, hybrid Ii-key construct, or chimeric or fusion polypeptide is produced by recombinant DNA or RNA techniques. For example, a nucleic acid molecule encoding the peptide, polypeptide, concatemeric peptide, hybrid Ii-key construct, or chimeric or fusion polypeptide is cloned into an expression vector, the expression vector introduced into a host cell and the peptide, polypeptide, concatemeric peptide, or chimeric or fusion polypeptide is expressed in the host cell. The peptide, polypeptide, concatemeric peptide, hybrid Ii-key construct, or chimeric or fusion polypeptide can then be isolated from the cells by an appropriate purification scheme using standard protein purification techniques.

For the purposes of the present disclosure, peptides, polypeptides, concatemeric peptides, hybrid Ii-key constructs, or chimeric or fusion polypeptides of the instant disclosure can include, for example, modified forms of naturally occurring amino acids such as D-stereoisomers, non-naturally occurring amino acids; amino acid analogs; and mimetics. Further, in aspects, peptides, polypeptides, concatemeric peptides, hybrid Ii-key constructs, or chimeric or fusion polypeptides of the instant disclosure can include retro-inverso peptides of the instantly disclosed peptides, polypeptides, concatemeric peptides, hybrid Ii-key constructs, or chimeric or fusion polypeptides of the instant disclosure, provided said peptides, polypeptides, concatemeric peptides, hybrid Ii-key constructs, chimeric or fusion polypeptides of the instant disclosure at least in part retain MHC binding propensity and/or TCR specificity, and/or retain anti-SARS-CoV-2 activity.

Although any methods and materials similar or equivalent to those described herein can be used in the practice or testing of the present disclosure, the preferred methods and materials are described. Other features, objects, and advantages of the present disclosure will be apparent from the description and the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All references cited herein are incorporated herein by reference in their entirety and for all purposes to the same extent as if each individual publication, patent, or patent application was specifically and individually indicated to be incorporated by reference in its entirety for all purposes.

### Peptides, Polypeptides, Concatemeric Polypeptides, Hybrid II-Key CONSTRUCTS, and CHIMERIC or FUSION POLYPEPTIDES

In aspects, the present disclosure provides a novel class of T-cell epitopes (which may be isolated, synthetic, or recombinant) and Ii-key contructs, which comprise a peptide or polypeptide chain derived from SARS-CoV-2 proteins (e.g., encoded proteins from a SARS-CoV-2 genome), including the envelope, membrane, spike, nucleocapsid, ORF3a, ORF6, ORF7a, ORFS, ORF10, ORF1ab non-structural protein 2 (NSP2), ORF1ab non-structural protein 3 (NSP3), ORF1ab non-structural protein 4 (NSP4), ORF1ab 3C-like proteinase, ORF1 ab non-structural protein 6 (NSP6), ORF1ab non-structural protein 7 (NSP7), ORF1ab non-structural protein 8 (NSP8), ORF1ab non-structural protein 9 (NSP9), ORF1ab non-structural protein 10 (NSP10), ORF1ab RNA-dependent RNA polymerase, ORF1ab helicase, ORF1ab 3′-5′ exonuclease, ORF1ab endoRNase, and ORF1ab 2′O-ribose methyltransferase proteins of SARS-CoV-2. T-cell epitopes of the present disclosure are highly conserved among known variants of their source proteins (e.g., present in more than 10% of known variants). As explained in more detail in the Examples, T-cell epitopes of the present disclosure are highly conserved among known variants of their source proteins, and SARS-CoV-2 (taxid: 2697049), SARS-CoV-1 (taxid: 694009), MERS-CoV (taxid: 1335626), and human CoV (taxids: 11137, 443239, 277944 and 31631) antigen sequences isolated from human hosts were obtained from GenBank at the National Center for Biotechnology Information. SARS-CoV-2 epitopes were compared across sequences obtained from isolates with fully sequenced genomes isolated from December 2019 to December 2020 for T cell epitope mapping. SARS-CoV-2 Wuhan-Hu-1 (GenBank id: MN908947) was selected as the reference strain.

As further described in the Examples, T-cell epitopes of the present disclosure comprise at least one putative T cell epitope as identified by EpiMatrix™ analysis. EpiMatrix™ is a proprietary computer algorithm developed by EpiVax (Providence, R.I.), which is used to screen protein sequences for the presence of putative T cell epitopes. The algorithm uses matrices for prediction of 9- and 10-mer peptides binding to MHC molecules. Each matrix is based on position-specific coefficients related to amino acid binding affinities that are elucidated by a method similar to, but not identical to, the pocket profile method (Sturniolo, T. et al., Nat. Biotechnol., 17:555-561, 1999). Input sequences are, for example, parsed into overlapping 9-mer frames or 10-mer where each frame overlaps the last by 8 or 9 amino acids, respectively. Each of the resulting frames form the mutated peptide and the non-mutated peptide are then scored for predicted binding affinity with respect to MHC class I alleles (e.g., but not limited to, HLA-A and HLA-B alleles) and MHC class II alleles (e.g., but not limited to HLA-DRB1 alleles). Raw scores are normalized against the scores of a large sample of randomly generated peptides. The resulting “Z” scores are normally distributed and directly comparable across alleles. The resulting “Z” score is reported. In aspects, any 9-mer or 10-mer peptide with an allele-specific EpiMatrix™ Z-score in excess of 1.64, theoretically the top 5% of any given sample, is considered a putative T cell epitope.

As also further described in the Examples, Peptides containing clusters of putative T cell epitopes are more likely to test positive in validating in vitro and in vivo assays. In aspects, the results of the initial EpiMatrix™ analysis is further screened for the presence of putative T cell epitope “clusters” using a second proprietary algorithm known as Clustimer™ algorithm. The Clustimer™ algorithm identifies sub-regions contained within any given amino acid sequence that contains a statistically unusually high number of putative T cell epitopes. Typical T-cell epitope “clusters” range from about 9 to roughly 30 amino acids in length and, considering their affinity to multiple alleles and across multiple 9-mer frames, can contain anywhere from about 4 to about 40 putative T cell epitopes. Table I discloses: an overview of MHC class II cluster selection from the envelope (SEQ ID NO: 1) of SARS-CoV-2, including the instantly-disclosed T cell epitopes of SEQ ID NOS: 4-8 and 210; an overview of MHC class II cluster selection from the membrane (SEQ ID NO: 2) of SARS-CoV-2, including the instantly-disclosed T cell epitopes of SEQ ID NOS: 9-63 and 211-213; and an overview of MHC class II cluster selection from the spike (SEQ ID NO: 3) of SARS-CoV-2, including the instantly-disclosed T cell epitopes of SEQ ID NOS: 64-209 and 214-224 and 454-456. Table 1 discloses the cluster sequence (bolded) of each the instantly-disclosed T cell epitopes of SEQ ID NOS: 4-224 and 454-456, the cluster score (without flanks), the number of EpiMatrix hits for class I MHC (which is the number of hits is the number of EpiMatrix Z-scores above 1.64 or top 5% found within the sequence), and the number of EpiMatrix hits for class II MHC (which is the number of hits is the number of EpiMatrix Z-scores above 1.64 or top 5% found within the sequence). Each epitope cluster identified an aggregate EpiMatrix™ score is calculated by summing the scores of the putative T cell epitopes and subtracting a correcting factor based on the length of the candidate epitope cluster and the expected score of a randomly generated cluster of the same length. EpiMatrix™ cluster scores in excess of +10 are considered significant. In aspects, the T-cell epitopes of the instant disclosure contain several putative T-cell epitopes forming a pattern known as a T-cell epitope cluster.

TABLE 1

| | | | Sequence | | | | | |
|---|---|---|---|---|---|---|---|---|
| | | | (cluster sequence | | | | | |
| | | | is bolded, flanks | | | | | |
| | | | are not bolded, | | EpiMatrix | | | |
| | | | and mutations | | Cluster | | | |
| SEQ | | | from WT sequence | EpiMatrix | Score | Number | Janus | EpiMatrix |
| ID | Input | | are | Hits | (w/o | of Human | Homology | Hits |
| NO: | Sequence | Flanks) | italicized) | (Class II) | Flanks) | Sequences | Score | (Class 1) |
| 4 | ENVELOPE | 0054- | PSFYVYSRVKNLNSSR | 16 | 32.67 | 13 | 0.9 | 7 |
| | (SEQ ID | 0069 | | | | | | |
| | NO: 1) | | | | | | | |
| 5 | ENVELOPE | 0055- | SFYVYSRVKNLNSSR | | | | | |
| | (SEQ ID | 0069 | | | | | | |
| | NO: 1) | | | | | | | |
| 6 | ENVELOPE | 0056- | FYVYSRVKNLNSSR | | | | | |
| | (SEQ ID | 0069 | | | | | | |
| | NO: 1) | | | | | | | |
| 7 | ENVELOPE | 0057- | YVYSRVKNLNSSR | | | | | |
| | (SEQ ID | 0069 | | | | | | |
| | NO: 1) | | | | | | | |
| 8 | ENVELOPE | 0057- | YVYSRVKNLNS | | | | | |
| | (SEQ ID | 0067 | | | | | | |
| | NO: 1) | | | | | | | |
| 9 | MEMBRANE | 0013- | LKKLLEQWNLVIGFL | 4 | 7.03 | 6 | 1.4 | 9 |
| | (SEQ ID | 0027 | | | | | | |
| | NO: 2) | | | | | | | |
| 10 | MEMBRANE | 0014- | KKLLEQWNLVIGFL | | | | | |
| | (SEQ ID | 0027 | | | | | | |
| | NO: 2) | | | | | | | |
| 11 | MEMBRANE | 0015- | KLLEQWNLVIGFL | | | | | |
| | (SEQ ID | 0027 | | | | | | |
| | NO: 2) | | | | | | | |
| 12 | MEMBRANE | 0016- | LLEQWNLVIGFL | | | | | |
| | (SEQ ID | 0027 | | | | | | |
| | NO: 2) | | | | | | | |
| 13 | MEMBRANE | 0016- | LLEQWNLVI | | | | | |
| | (SEQ ID | 0024 | | | | | | |
| | NO: 2) | | | | | | | |
| 14 | MEMBRANE | 0017- | LEQWNLVIGFLFLTW | 4 | 8.76 | 3 | 1 | 17 |
| | (SEQ ID | 0031 | | | | | | |
| | NO: 2) | | | | | | | |
| 15 | MEMBRANE | 0018- | EQWNLVIGFLFLTW | | | | | |
| | (SEQ ID | 0031 | | | | | | |
| | NO: 2) | | | | | | | |
| 16 | MEMBRANE | 0019- | QWNLVIGFLFLTW | | | | | |
| | (SEQ ID | 0031 | | | | | | |
| | NO: 2) | | | | | | | |
| 17 | MEMBRANE | 0020- | WNLVIGFLFLTW | | | | | |
| | (SEQ ID | 0031 | | | | | | |
| | NO: 2) | | | | | | | |
| 18 | MEMBRANE | 0020- | WNLVIGFLF | | | | | |
| | (SEQ ID | 0028 | | | | | | |
| | NO: 2) | | | | | | | |
| 19 | MEMBRANE | 0032- | ICLLQFAYANRNRFLYI | 14 | 26.89 | 2 | 0.73 | 9 |
| | (SEQ ID | 0048 | | | | | | |
| | NO: 2) | | | | | | | |
| 20 | MEMBRANE | 0033- | CLLQFAYANRNRFLY1 | | | | | |
| | (SEQ ID | 0048 | | | | | | |
| | NO: 2) | | | | | | | |
| 21 | MEMBRANE | 0034- | LLQFAYANRNRFLYI | | | | | |
| | (SEQ ID | 0048 | | | | | | |
| | NO: 2) | | | | | | | |
| 22 | MEMBRANE | 0035- | LQFAYANRNRFLYI | | | | | |
| | (SEQ ID | 0048 | | | | | | |
| | NO: 2) | | | | | | | |
| 23 | MEMBRANE | 0035- | LQFAYANRNRF | | | | | |
| | (SEQ ID | 0045 | | | | | | |
| | NO: 2) | | | | | | | |
| 24 | MEMBRANE | 0042- | RNRFLYIIKLIFLWLLWPV | 15 | 27.53 | 8 | 1.56 | 28 |
| | (SEQ ID | 0060 | | | | | | |
| | NO: 2) | | | | | | | |
| 25 | MEMBRANE | 0043- | NRFLYIIKLIFLWLLWPV | | | | | |
| | (SEQ ID | 0060 | | | | | | |
| | NO: 2) | | | | | | | |
| 26 | MEMBRANE | 0044- | RFLYIIKLIFLWLLWPV | | | | | |
| | (SEQ ID | 0060 | | | | | | |
| | NO: 2) | | | | | | | |
| 27 | MEMBRANE | 0045- | FLYIIKLIFLWLLWPV | | | | | |
| | (SEQ ID | 0060 | | | | | | |
| | NO: 2) | | | | | | | |
| 28 | MEMBRANE | 0045- | FLYIIKLIFLWLL | | | | | |
| | (SEQ ID | 0057 | | | | | | |
| | NO: 2) | | | | | | | |
| 29 | MEMBRANE | 0062- | LACFVLAAVYRINWI | 9 | 19.44 | 3 | 0.8 | 4 |
| | (SEQ ID | 0076 | | | | | | |
| | NO: 2) | | | | | | | |
| 30 | MEMBRANE | 0063- | ACFVLAAVYRINWI | | | | | |
| | (SEQ ID | 0076 | | | | | | |
| | NO: 2) | | | | | | | |
| 31 | MEMBRANE | 0064- | CFVLAAVYRINWI | | | | | |
| | (SEQ ID | 0076 | | | | | | |
| | NO: 2) | | | | | | | |
| 32 | MEMBRANE | 0065- | FVLAAVYRINWI | | | | | |
| | (SEQ ID | 0076 | | | | | | |
| | NO: 2) | | | | | | | |
| 33 | MEMBRANE | 0065- | FVLAAVYRI | | | | | |
| | (SEQ ID | 0073 | | | | | | |
| | NO: 2) | | | | | | | |
| 34 | MEMBRANE | 0093- | LSYFIASFRLFARTR | 8 | 18.15 | 5 | 0.56 | 10 |
| | (SEQ ID | 0107 | | | | | | |
| | NO: 2) | | | | | | | |
| 35 | MEMBRANE | 0094- | SYFIASFRLFARTR | | | | | |
| | (SEQ ID | 0107 | | | | | | |
| | NO: 2) | | | | | | | |
| | | | | | Flanks) | | | |
| 36 | MEMBRANE | 0095- | YFIASFRLFARTR | | | | | |
| | (SEQ ID | 0107 | | | | | | |
| | NO: 2) | | | | | | | |
| 37 | MEMBRANE | 0096- | FIASFRLFARTR | | | | | |
| | (SEQ ID | 0107 | | | | | | |
| | NO: 2) | | | | | | | |
| 38 | MEMBRANE | 0096- | FIASFRLFA | | | | | |
| | (SEQ ID | 0104 | | | | | | |
| | NO: 2) | | | | | | | |
| 39 | MEMBRANE | 0097- | IASFRLFARTRSMWSFNP | 13 | 23.84 | 5 | 0.8 | 12 |
| | (SEQ ID | 0114 | | | | | | |
| | NO: 2) | | | | | | | |
| 40 | MEMBRANE | 0098- | ASFRLFARTRSMWSFNP | | | | | |
| | (SEQ ID | 0114 | | | | | | |
| | NO: 2) | | | | | | | |
| 41 | MEMBRANE | 0099- | SFRLFARTRSMWSFNP | | | | | |
| | (SEQ ID | 0114 | | | | | | |
| | NO: 2) | | | | | | | |
| 42 | MEMBRANE | 0100- | FRLFARTRSMWSFNP | | | | | |
| | (SEQ ID | 0114 | | | | | | |
| | NO: 2) | | | | | | | |
| 43 | MEMBRANE | 0100- | FRLFARTRSMWS | | | | | |
| | (SEQ ID | 0111 | | | | | | |
| | NO: 2) | | | | | | | |
| 44 | MEMBRANE | 0146- | RGHLRIAGHHLGRCD | 6 | 10.96 | 1 | 0.14 | 2 |
| | (SEQ ID | 0160 | | | | | | |
| | NO: 2) | | | | | | | |
| 45 | MEMBRANE | 0147- | GHLRIAGHHLGRCD | | | | | |
| | (SEQ ID | 0160 | | | | | | |
| | NO: 2) | | | | | | | |
| 46 | MEMBRANE | 0148- | HLRIAGHHLGRCD | | | | | |
| | (SEQ ID | 0160 | | | | | | |
| | NO: 2) | | | | | | | |
| 47 | MEMBRANE | 0149- | LRIAGHHLGRCD | | | | | |
| | (SEQ ID | 0160 | | | | | | |
| | NO: 2) | | | | | | | |
| 48 | MEMBRANE | 0149- | LRIAGHHLG | | | | | |
| | (SEQ ID | 0157 | | | | | | |
| | NO: 2) | | | | | | | |
| 49 | MEMBRANE | 0165- | PKEITVATSRTLSYY | 5 | 9.81 | 1 | 0.14 | 11 |
| | (SEQ ID | 0179 | | | | | | |
| | NO: 2) | | | | | | | |
| 50 | MEMBRANE | 0166- | KEITVATSRTLSYY | | | | | |
| | (SEQ ID | 0179 | | | | | | |
| | NO: 2) | | | | | | | |
| 51 | MEMBRANE | 0167- | EITVATSRTLSYY | | | | | |
| | (SEQ ID | 0179 | | | | | | |
| | NO: 2) | | | | | | | |
| 52 | MEMBRANE | 0168- | ITVATSRTLSYY | | | | | |
| | (SEQ ID | 0179 | | | | | | |
| | NO: 2) | | | | | | | |
| 53 | MEMBRANE | 0168- | ITVATSRTL | | | | | |
| | (SEQ ID | 0176 | | | | | | |
| | NO: 2) | | | | | | | |
| 54 | MEMBRANE | 0175- | TLSYYKLGASQRVAGD | 9 | 17.64 | 8 | 1.83 | 4 |
| | (SEQ ID | 0190 | | | | | | |
| | NO: 2) | | | | | | | |
| 55 | MEMBRANE | 0176- | LSYYKLGASQRVAGD | | | | | |
| | (SEQ ID | 0190 | | | | | | |
| | NO: 2) | | | | | | | |
| 56 | MEMBRANE | 0176- | SYYKLGASQRVAGD | | | | | |
| | (SEQ ID | 0190 | | | | | | |
| | NO: 2) | | | | | | | |
| 57 | MEMBRANE | 0178- | YYKLGASQRVAGD | | | | | |
| | (SEQ ID | 0190 | | | | | | |
| | NO: 2) | | | | | | | |
| 58 | MEMBRANE | 0178- | YYKLGASQRV | | | | | |
| | (SEQ ID | 0187 | | | | | | |
| | NO: 2) | | | | | | | |
| 59 | MEMBRANE | 0201- | IGNYKLNTDHSSSSDN1 | 8 | 14.38 | 4 | 0.78 | 5 |
| | (SEQ ID | 0217 | | | | | | |
| | NO: 2) | | | | | | | |
| 60 | MEMBRANE | 0202- | GNYKLNTDHSSSSDN1 | | | | | |
| | (SEQ ID | 0217 | | | | | | |
| | NO: 2) | | | | | | | |
| 61 | MEMBRANE | 0203- | NYKLNTDHSSSSDNI | | | | | |
| | (SEQ ID | 0217 | | | | | | |
| | NO: 2) | | | | | | | |
| 62 | MEMBRANE | 0204- | YKLNTDHSSSSDNI | | | | | |
| | (SEQ ID | 0217 | | | | | | |
| | NO: 2) | | | | | | | |
| 63 | MEMBRANE | 0204- | YKLNTDHSSSS | | | | | |
| | (SEQ ID | 0214 | | | | | | |
| | NO: 2) | | | | | | | |
| 64 | SPIKE (SEQ | 0025- | PPAYTNSFTRGVYYP | 6 | 12.8 | 1 | 0.17 | 10 |
| | ID NO: 3) | 0039 | | | | | | |
| 65 | SPIKE (SEQ | 0026- | PAYTNSFTRGVYYP | | | | | |
| | ID NO: 3) | 0039 | | | | | | |
| 66 | SPIKE (SEQ | 0027- | AYTNSFTRGVYYP | | | | | |
| | ID NO: 3) | 0039 | | | | | | |
| 67 | SPIKE (SEQ | 0028- | YTNSFTRGVYYP | | | | | |
| | ID NO: 3) | 0039 | | | | | | |
| 68 | SPIKE (SEQ | 0028- | YTNSFTRGV | | | | | |
| | ID NO: 3) | 0036 | | | | | | |
| 69 | SPIKE (SEQ | 0052- | QDLFLPFFSNVTWFH | 7 | 14.42 | 2 | 0.22 | 14 |
| | ID NO: 3) | 0066 | | | | | | |
| 70 | SPIKE (SEQ | 0053- | DLFLPFFSNVTWFH | | | | | |
| | ID NO: 3) | 0066 | | | | | | |
| 71 | SPIKE (SEQ | 0054- | LFLPFFSNVTWFH | | | | | |
| | ID NO: 3) | 0066 | | | | | | |
| 72 | SPIKE (SEQ | 0055- | FLPFFSNVTWFH | | | | | |
| | ID NO: 3) | 0066 | | | | | | |
| 73 | SPIKE (SEQ | 0055- | FLPFFSNVT | | | | | |
| | ID NO: 3) | 0063 | | | | | | |
| 74 | SPIKE (SEQ | 0061- | NVTWFHAIHVSGTNGT | 9 | 17.97 | 2 | 0.54 | 2 |
| | ID NO: 3) | 0076 | | | | | | |
| 75 | SPIKE (SEQ | 0062- | VTWFHAIHVSGTNGT | | | | | |
| | ID NO: 3) | 0076 | | | | | | |
| 76 | SPIKE (SEQ | 0063- | TWFHAIHVSGTNGT | | | | | |
| | ID NO: 3) | 0076 | | | | | | |
| 77 | SPIKE (SEQ | 0064- | WFHAIHVSGTNGT | | | | | |
| | ID NO: 3) | 0076 | | | | | | |
| 78 | SPIKE (SEQ | 0064- | WFHAIHVSGT | | | | | |
| | ID NO: 3) | 0073 | | | | | | |
| 79 | SPIKE (SEQ | 0087- | NDGVYFASTEKSNIIRG | 7 | 10.83 | 2 | 0.29 | 6 |
| | ID NO: 3) | 0103 | | | | | | |
| 80 | SPIKE (SEQ | 0088- | DGVYFASTEKSNIIRG | | | | | |
| | ID NO: 3) | 0103 | | | | | | |
| 81 | SPIKE (SEQ | 0089- | GVYFASTEKSNIIRG | | | | | |
| | ID NO: 3) | 0103 | | | | | | |
| 82 | SPIKE (SEQ | 0090- | VYFASTEKSNIIRG | | | | | |
| | ID NO: 3) | 0103 | | | | | | |
| 83 | SPIKE (SEQ | 0090- | VYFASTEKSNI | | | | | |
| | ID NO: 3) | 0100 | | | | | | |
| 84 | SPIKE (SEQ | 0114- | TQSLLIVNNATNVVIKV | 20 | 36.68 | 9 | 0.52 | 5 |
| | ID NO: 3) | 0130 | | | | | | |
| 85 | SPIKE (SEQ | 0115- | QSLLIVNNATNVVIKV | | | | | |
| | ID NO: 3) | 0130 | | | | | | |
| 86 | SPIKE (SEQ | 0116- | SLLIVNNATNVVIKV | | | | | |
| | ID NO: 3) | 0130 | | | | | | |
| 87 | SPIKE (SEQ | 0117- | LLIVNNATNVVIKV | | | | | |
| | ID NO: 3) | 0130 | | | | | | |
| 88 | SPIKE (SEQ | 0117- | LLIVNNATNVVI | | | | | |
| | ID NO: 3) | 0130 | | | | | | |
| 89 | SPIKE (SEQ | 0140- | FLGVYYHKNNKSWMESE | 11 | 20.72 | 0 | 0 | 6 |
| | ID NO: 3) | 0156 | | | | | | |
| 90 | SPIKE (SEQ | 0141- | LGVYYHKNNKSWMESE | | | | | |
| | ID NO: 3) | 0156 | | | | | | |
| 91 | SPIKE (SEQ | 0142- | GVYYHKNNKSWMESE | | | | | |
| | ID NO: 3) | 0156 | | | | | | |
| 92 | SPIKE (SEQ | 0143- | VYYHKNNKSWMESE | | | | | |
| | ID NO: 3) | 0156 | | | | | | |
| 93 | SPIKE (SEQ | 0143- | VYYHKNNKSWM | | | | | |
| | ID NO: 3) | 0153 | | | | | | |
| 94 | SPIKE (SEQ | 0154- | ESEFRVYSSANNCTFEYV | 11 | 18.25 | 1 | 0.09 | 17 |
| | ID NO: 3) | 0171 | | | | | | |
| 95 | SPIKE (SEQ | 0155- | SEFRVYSSANNCTFEYV | | | | | |
| | ID NO: 3) | 0171 | | | | | | |
| 96 | SPIKE (SEQ | 0156- | EFRVYSSANNCTFEYV | | | | | |
| | ID NO: 3) | 0171 | | | | | | |
| 97 | SPIKE (SEQ | 0157- | FRVYSSANNCTFEYV | | | | | |
| | ID NO: 3) | 0171 | | | | | | |
| 98 | SPIKE (SEQ | 0157- | FRVYSSANNCTF | | | | | |
| | ID NO: 3) | 0168 | | | | | | |
| 99 | SPIKE (SEQ | 0198- | DGYFKIYSKHTPINL | 3 | 5.48 | 1 | 0.08 | 8 |
| | ID NO: 3) | 0212 | | | | | | |
| 100 | SPIKE (SEQ | 0199- | GYFKIYSKHTPINL | | | | | |
| | ID NO: 3) | 0212 | | | | | | |
| 101 | SPIKE (SEQ | 0200- | YFKIYSKHTPINL | | | | | |
| | ID NO: 3) | 0212 | | | | | | |
| 102 | SPIKE (SEQ | 0201- | FKIYSKHTPINL | | | | | |
| | ID NO: 3) | 0212 | | | | | | |
| 103 | SPIKE (SEQ | 0201- | FKIYSKHTP | | | | | |
| | ID NO: 3) | 0209 | | | | | | |
| 104 | SPIKE (SEQ | 0272- | PRTFLLKYNENGTITDA | 11 | 18.68 | 3 | 0.31 | 2 |
| | ID NO: 3) | 0288 | | | | | | |
| 105 | SPIKE (SEQ | 0273- | RTFLLKYNENGTITDA | | | | | |
| | ID NO: 3) | 0288 | | | | | | |
| 106 | SPIKE (SEQ | 0274- | TFLLKYNENGTITDA | | | | | |
| | ID NO: 3) | 0288 | | | | | | |
| 107 | SPIKE (SEQ | 0275- | FLLKYNENGTITDA | | | | | |
| | ID NO: 3) | 0288 | | | | | | |
| 108 | SPIKE (SEQ | 0275- | FLLKYNENGTI | | | | | |
| | ID NO: 3) | 0285 | | | | | | |
| 109 | SPIKE (SEQ | 0315- | TSNFRVQPTESIVRF | 6 | 10.62 | 0 | 0 | 1 |
| | ID NO: 3) | 0329 | | | | | | |
| 110 | SPIKE (SEQ | 0316- | SNFRVQPTESIVRF | | | | | |
| | ID NO: 3) | 0329 | | | | | | |
| 111 | SPIKE (SEQ | 0317- | NFRVQPTESIVRF | | | | | |
| | ID NO: 3) | 0329 | | | | | | |
| 112 | SPIKE (SEQ | 0318- | FRVQPTESIVRF | | | | | |
| | ID NO: 3) | 0329 | | | | | | |
| 113 | SPIKE (SEQ | 0318- | FRVQPTESI | | | | | |
| | ID NO: 3) | 0326 | | | | | | |
| 114 | SPIKE (SEQ | 0338- | FGEVFNATRFASVYA | 4 | 6.48 | 3 | 0.38 | 6 |
| | ID NO: 3) | 0352 | | | | | | |
| 115 | SPIKE (SEQ | 0339- | GEVFNATRFASVYA | | | | | |
| | ID NO: 3) | 0352 | | | | | | |
| 116 | SPIKE (SEQ | 0340- | EVFNATRFASVYA | | | | | |
| | ID NO: 3) | 0352 | | | | | | |
| 117 | SPIKE (SEQ | 0341- | VFNATRFASVYA | | | | | |
| | ID NO: 3) | 0352 | | | | | | |
| 118 | SPIKE (SEQ | 0341- | VFNATRFAS | | | | | |
| | ID NO: 3) | 0349 | | | | | | |
| 119 | SPIKE (SEQ | 0347- | FASVYAWNRKRISN | 5 | 9.2 | 2 | 0.8 | 5 |
| | ID NO: 3) | 0360 | | | | | | |
| 120 | SPIKE (SEQ | 0348- | ASVYAWNRKRISN | | | | | |
| | ID NO: 3) | 0360 | | | | | | |
| 121 | SPIKE (SEQ | 0349- | SVYAWNRKRISN | | | | | |
| | ID NO: 3) | 0360 | | | | | | |
| 122 | SPIKE (SEQ | 0350- | VYAWNRKRISN | | | | | |
| | ID NO: 3) | 0360 | | | | | | |
| 123 | SPIKE (SEQ | 0350- | VYAWNRKRIS | | | | | |
| | ID NO: 3) | 0359 | | | | | | |
| 124 | SPIKE (SEQ | 0362- | VADYSVLYNSASFST | 18 | 33.15 | 6 | 0.61 | 9 |
| | ID NO: 3) | 0376 | | | | | | |
| 125 | SPIKE (SEQ | 0363- | ADYSVLYNSASFST | | | | | |
| | ID NO: 3) | 0376 | | | | | | |
| 126 | SPIKE (SEQ | 0364- | DYSVLYNSASFST | | | | | |
| | ID NO: 3) | 0376 | | | | | | |
| 127 | SPIKE (SEQ | 0365- | YSVLYNSASFST | | | | | |
| | ID NO: 3) | 0376 | | | | | | |
| 128 | SPIKE (SEQ | 0446- | GGNYNYLYRLFRKSN | 6 | 12.55 | 2 | 0.27 | 9 |
| | ID NO: 3) | 0460 | | | | | | |
| 129 | SPIKE (SEQ | 0447- | GNYNYLYRLFRKSN | | | | | |
| | ID NO: 3) | 0460 | | | | | | |
| 130 | SPIKE (SEQ | 0448- | NYNYLYRLFRKSN | | | | | |
| | ID NO: 3) | 0460 | | | | | | |
| 131 | SPIKE (SEQ | 0449- | YNYLYRLFRKSN | | | | | |
| | ID NO: 3) | 0460 | | | | | | |
| 132 | SPIKE (SEQ | 0449- | YNYLYRLFR | | | | | |
| | ID NO: 3) | 0457 | | | | | | |
| 133 | SPIKE (SEQ | 0450- | NYLYRLFRKSNLKPF | 8 | 17.64 | 8 | 0.91 | 5 |
| | ID NO: 3) | 0464 | | | | | | |
| 134 | SPIKE (SEQ | 0451- | YLYRLFRKSNLKPF | | | | | |
| | ID NO: 3) | 0464 | | | | | | |
| 135 | SPIKE (SEQ | 0452- | LYRLFRKSNLKPF | | | | | |
| | ID NO: 3) | 0464 | | | | | | |
| 136 | SPIKE (SEQ | 0453- | YRLFRKSNLKPF | | | | | |
| | ID NO: 3) | 0464 | | | | | | |
| 137 | SPIKE (SEQ | 0453- | YRLFRKSNL | | | | | |
| | ID NO: 3) | 0461 | | | | | | |
| 138 | SPIKE (SEQ | 0454- | RLFRKSNLKPFERD | 6 | 12.72 | 12 | 1.55 | 4 |
| | ID NO: 3) | 0467 | | | | | | |
| 139 | SPIKE (SEQ | 0455- | LFRKSNLKPFERD | | | | | |
| | ID NO: 3) | 0467 | | | | | | |
| 140 | SPIKE (SEQ | 0456- | FRKSNLKPFERD | | | | | |
| | ID NO: 3) | 0467 | | | | | | |
| 141 | SPIKE (SEQ | 0456- | FRKSNLKPF | | | | | |
| | ID NO: 3) | 0464 | | | | | | |
| 142 | SPIKE (SEQ | 0483- | VEGFNCYFPLQSYGFQPT | 11 | 17.96 | 0 | 0 | 10 |
| | ID NO: 3) | 0500 | | | | | | |
| 143 | SPIKE (SEQ | 0484- | EGFNCYFPLQSYGFQPT | | | | | |
| | ID NO: 3) | 0500 | | | | | | |
| 144 | SPIKE (SEQ | 0485- | GFNCYFPLQSYGFQPT | | | | | |
| | ID NO: 3) | 0500 | | | | | | |
| 145 | SPIKE (SEQ | 0486- | FNCYFPLQSYGFQPT | | | | | |
| | ID NO: 3) | 0500 | | | | | | |
| 146 | SPIKE (SEQ | 0486- | FNCYFPLQSYGF | | | | | |
| | ID NO: 3) | 0497 | | | | | | |
| 147 | SPIKE (SEQ | 0492- | LQSYGFQPTNGVGYQPY | 7 | 10.08 | 1 | 0.14 | 3 |
| | ID NO: 3) | 0508 | | | | | | |
| 148 | SPIKE (SEQ | 0493- | QSYGFQPTNGVGYQPY | | | | | |
| | ID NO: 3) | 0508 | | | | | | |
| 149 | SPIKE (SEQ | 0494- | SYGFQPTNGVGYQPY | | | | | |
| | ID NO: 3) | 0508 | | | | | | |
| 150 | SPIKE (SEQ | 0495- | YGFQPTNGVGYQPY | | | | | |
| | ID NO: 3) | 0508 | | | | | | |
| 151 | SPIKE (SEQ | 0495- | YGFQPTNGVGY | | | | | |
| | ID NO: 3) | 0503 | | | | | | |
| 152 | SPIKE (SEQ | 0512- | VLSFELLHAPATVCG | 7 | 14.88 | 4 | 0.75 | 4 |
| | ID NO: 3) | 0526 | | | | | | |
| 153 | SPIKE (SEQ | 0513- | LSFELLHAPATVCG | | | | | |
| | ID NO: 3) | 0526 | | | | | | |
| 154 | SPIKE (SEQ | 0514- | SFELLHAPATVCG | | | | | |
| | ID NO: 3) | 0526 | | | | | | |
| 155 | SPIKE (SEQ | 0515- | FELLHAPATVCG | | | | | |
| | ID NO: 3) | 0526 | | | | | | |
| 156 | SPIKE (SEQ | 0515- | FELLHAPAT | | | | | |
| | ID NO: 3) | 0523 | | | | | | |
| 157 | SPIKE (SEQ | 0536- | NKCVNFNFNGLTGTG | 7 | 12.54 | 0 | 0 | 2 |
| | ID NO: 3) | 0550 | | | | | | |
| 158 | SPIKE (SEQ | 0537- | KCVNFNFNGLTGTG | | | | | |
| | ID NO: 3) | 0550 | | | | | | |
| 159 | SPIKE (SEQ | 0538- | CVNFNFNGLTGTG | | | | | |
| | ID NO: 3) | 0550 | | | | | | |
| 160 | SPIKE (SEQ | 0539- | VNFNFNGLTGTG | | | | | |
| | ID NO: 3) | 0550 | | | | | | |
| 161 | SPIKE (SEQ | 0539- | VNFNFNGLT | | | | | |
| | ID NO: 3) | 0547 | | | | | | |
| 162 | SPIKE (SEQ | 0540- | NFNFNGLTGTGVLTE | 5 | 9.07 | 0 | 0 | 0 |
| | ID NO: 3) | 0554 | | | | | | |
| 163 | SPIKE (SEQ | 0541- | FNFNGLTGTGVLTE | | | | | |
| | ID NO: 3) | 0554 | | | | | | |
| 164 | SPIKE (SEQ | 0542- | NFNGLTGTGVLTE | | | | | |
| | ID NO: 3) | 0554 | | | | | | |
| 165 | SPIKE (SEQ | 0543- | FNGLTGTGVLTE | | | | | |
| | ID NO: 3) | 0554 | | | | | | |
| 166 | SPIKE (SEQ | 0543- | FNGLTGTGV | | | | | |
| | ID NO: 3) | 0551 | | | | | | |
| 167 | SPIKE (SEQ | 0761- | TQLNRALTGIAVEQ | 8 | 15.4 | 7 | 0.82 | 3 |
| | ID NO: 3) | 0774 | | | | | | |
| 168 | SPIKE (SEQ | 0762- | QLNRALTGIAVEQ | | | | | |
| | ID NO: 3) | 0774 | | | | | | |
| 169 | SPIKE (SEQ | 0763- | LNRALTGIAVEQ | | | | | |
| | ID NO: 3) | 0774 | | | | | | |
| 170 | SPIKE (SEQ | 0763- | LNRALTGIA | | | | | |
| | ID NO: 3) | 0771 | | | | | | |
| 171 | SPIKE (SEQ | 0866- | TDEMIAQYTSALLAG | 10 | 17.71 | 3 | 0.3 | 8 |
| | ID NO: 3) | 0880 | | | | | | |
| 172 | SPIKE (SEQ | 0867- | DEMIAQYTSALLAG | | | | | |
| | ID NO: 3) | 0880 | | | | | | |
| 173 | SPIKE (SEQ | 0868- | EMIAQYTSALLAG | | | | | |
| | ID NO: 3) | 0880 | | | | | | |
| 174 | SPIKE (SEQ | 0869- | MIAQYTSALLAG | | | | | |
| | ID NO: 3) | 0880 | | | | | | |
| 175 | SPIKE (SEQ | 0866- | MIAQYTSALL | | | | | |
| | ID NO: 3) | 0878 | | | | | | |
| 176 | SPIKE (SEQ | 0895- | QIPFAMQMAYRFNGIGV | 7 | 10.2 | 0 | 0 | 15 |
| | ID NO: 3) | 0911 | | | | | | |
| 177 | SPIKE (SEQ | 0896- | IPFAMQMAYRFNGIGV | | | | | |
| | ID NO: 3) | 0911 | | | | | | |
| 178 | SPIKE (SEQ | 0897- | PFAMQMAYRFNGIGV | | | | | |
| | ID NO: 3) | 0911 | | | | | | |
| 179 | SPIKE (SEQ | 0898- | FAMQMAYRFNGIGV | | | | | |
| | ID NO: 3) | 0911 | | | | | | |
| 180 | SPIKE (SEQ | 0899- | FAMQMAYRFNG | | | | | |
| | ID NO: 3) | 0908 | | | | | | |
| 181 | SPIKE (SEQ | 0920- | QKLIANQFNSAIGKI | 5 | 8.58 | 2 | 0.29 | 7 |
| | ID NO: 3) | 0934 | | | | | | |
| 182 | SPIKE (SEQ | 0921- | KLIANQFNSAIGKI | | | | | |
| | ID NO: 3) | 0934 | | | | | | |
| 183 | SPIKE (SEQ | 0922- | LIANQFNSAIGKI | | | | | |
| | ID NO: 3) | 0934 | | | | | | |
| 184 | SPIKE (SEQ | 0923- | IANQFNSAIGKI | | | | | |
| | ID NO: 3) | 0934 | | | | | | |
| 185 | SPIKE (SEQ | 0923- | IANQFNSAI | | | | | |
| | ID NO: 3) | 0931 | | | | | | |
| 186 | SPIKE (SEQ | 0924- | ANQFNSAIGKIQDSL | 7 | 14.98 | 5 | 2 | 2 |
| | ID NO: 3) | 0938 | | | | | | |
| 187 | SPIKE (SEQ | 0925- | NQFNSAIGKIQDSL | | | | | |
| | ID NO: 3) | 0938 | | | | | | |
| 188 | SPIKE (SEQ | 0926- | QFNSAIGKIQDSL | | | | | |
| | ID NO: 3) | 0938 | | | | | | |
| 189 | SPIKE (SEQ | 0927- | FNSAIGKIQDSL | | | | | |
| | ID NO: 3) | 0938 | | | | | | |
| 190 | SPIKE (SEQ | 0924- | FNSAIGKIQ | | | | | |
| | ID NO: 3) | 0935 | | | | | | |
| 191 | SPIKE (SEQ | 0998- | TGRLQSLQTYVTQQL | 6 | 12.24 | 1 | 0.14 | 3 |
| | ID NO: 3) | 1012 | | | | | | |
| 192 | SPIKE (SEQ | 0999- | GRLQSLQTYVTQQL | | | | | |
| | ID NO: 3) | 1012 | | | | | | |
| 193 | SPIKE (SEQ | 1000- | RLQSLQTYVTQQL | | | | | |
| | ID NO: 3) | 1012 | | | | | | |
| 194 | SPIKE (SEQ | 1001- | LQSLQTYVTQQL | | | | | |
| | ID NO: 3) | 1012 | | | | | | |
| 195 | SPIKE (SEQ | 1001- | LQSLQTYVT | | | | | |
| | ID NO: 3) | 1009 | | | | | | |
| 196 | SPIKE (SEQ | 1044- | GKGYHLMSFPQSAPH | 4 | 7.89 | 3 | 0.67 | 4 |
| | ID NO: 3) | 1058 | | | | | | |
| 197 | SPIKE (SEQ | 1045- | KGYHLMSFPQSAPH | | | | | |
| | ID NO: 3) | 1058 | | | | | | |
| 198 | SPIKE (SEQ | 1046- | GYHLMSFPQSAPH | | | | | |
| | ID NO: 3) | 1058 | | | | | | |
| 199 | SPIKE (SEQ | 1047- | YHLMSFPQSAPH | | | | | |
| | ID NO: 3) | 1058 | | | | | | |
| 200 | SPIKE (SEQ | 1047- | YHLMSFPQS | | | | | |
| | ID NO: 3) | 1055 | | | | | | |
| 201 | SPIKE (SEQ | 1127- | DVVIGIVNNTVYDPL | 9 | 16.52 | 1 | 0.44 | 5 |
| | ID NO: 3) | 1141 | | | | | | |
| 202 | SPIKE (SEQ | 1128- | VVIGIVNNTVYDPL | | | | | |
| | ID NO: 3) | 1141 | | | | | | |
| 203 | SPIKE (SEQ | 1129- | VIGIVNNTVYDPL | | | | | |
| | ID NO: 3) | 1141 | | | | | | |
| 204 | SPIKE (SEQ | 1129- | VIGIVNNTVY | | | | | |
| | ID NO: 3) | 1138 | | | | | | |
| 205 | SPIKE (SEQ | 1152- | LDKYFKNHTSPDVDLG | 7 | 12.63 | 0 | 0 | 3 |
| | ID NO: 3) | 1167 | | | | | | |
| 206 | SPIKE (SEQ | 1153- | DKYFKNHTSPDVDLG | | | | | |
| | ID NO: 3) | 1167 | | | | | | |
| 207 | SPIKE (SEQ | 1154- | KYFKNHTSPDVDLG | | | | | |
| | ID NO: 3) | 1167 | | | | | | |
| 208 | SPIKE (SEQ | 1155- | YFKNHTSPDVDLG | | | | | |
| | ID NO: 3) | 1167 | | | | | | |
| 209 | SPIKE (SEQ | 1155- | YFKNHTSPDV | | | | | |
| | ID NO: 3) | 1164 | | | | | | |
| 210 | ENVELOPE | 0001- | MYSFVSEETGTLIVN | 5 | 9.73 | 2 | 1.5 | 13 |
| | (SEQ ID | 0015 | | | | | | |
| | NO: 1) | | | | | | | |
| 211 | MEMBRANE | 0032- | ISLLQFAYANRNRFLYI | 14 | 26.89 | 2 | .73 | 9 |
| | (SEQ ID | 0048 | | | | | | |
| | NO: 2) | | | | | | | |
| 212 | MEMBRANE | 0097- | IASFRLFARTRSMWSFN | 13 | 23.84 | 5 | .80 | 11 |
| | (SEQ ID | 0113 | | | | | | |
| | NO: 2) | | | | | | | |
| 213 | MEMBRANE | 0146- | RGHLRIAGHHLGRSD | 6 | 10.96 | 1 | 0.14 | 2 |
| | (SEQ ID | 0160 | | | | | | |
| | NO: 2) | | | | | | | |
| 214 | SPIKE (SEQ | 0025- | PPAYTNSFTRGVYY | 6 | 12.8 | 1 | 0.17 | 10 |
| | ID NO: 3) | 0038 | | | | | | |
| 215 | SPIKE (SEQ | 0087- | NEGVYFASTEKSNIIR | 7 | 10.83 | 2 | 0.29 | 4 |
| | ID NO: 3) | 0102 | | | | | | |
| 216 | SPIKE (SEQ | 0239- | QTLLALHRSYLTPGDSs | 14 | 25.33 | 7 | 0.53 | 10 |
| | ID NO: 3) | 0255 | | | | | | |
| 217 | SPIKE (SEQ | 0347- | FASVYAWNRKRISN [symbol] VAD | 7 | 11.13 | 3 | 0.63 | 5 |
| | ID NO: 3) | 0364 | | | | | | |
| 218 | SPIKE (SEQ | 0446- | GGNYNYLYRLFRKSNLKPFE | 31 | 58.68 | 14 | 1.10 | 14 |
| | ID NO: 3) | 0468 | RDI | | | | | |
| 219 | SPIKE (SEQ | 0536- | NKSVNFNFNGLTGT | 7 | 12.54 | 0 | 0.00 | 2 |
| | ID NO: 3) | 0549 | | | | | | |
| 220 | SPIKE (SEQ | 0797- | FGGFNFSQILPDPSKPSKRS | 8 | 10.25 | 1 | 0.13 | 4 |
| | ID NO: 3) | 0816 | | | | | | |
| 221 | SPIKE (SEQ | 0866- | TDEMIAQYTSALLA | 10 | 17.71 | 3 | 0.30 | 8 |
| | ID NO: 3) | 0879 | | | | | | |
| 222 | SPIKE (SEQ | 0895- | LQIPFAMQMAYRFNGIGV | 7 | 10.2 | 0 | 0.00 | 15 |
| | ID NO: 3) | 0911 | | | | | | |
| 223 | SPIKE (SEQ | 1152- | LDKYFKNHTSPDVDL | 9 | 16.52 | 1 | 0.44 | 2 |
| | ID NO: 3) | 1167 | | | | | | |
| 224 | SPIKE (SEQ | 1169- | ISGINASVVNIQKEI | 9 | 12.24 | 5 | 1.00 | 4 |
| | ID NO: 3) | 1183 | | | | | | |
| 454 | SPIKE (SEQ | 0087- | NDGVYFASTEKSNIIR | 7 | 10.83 | 2 | 0.29 | 4 |
| | ID NO: 3) | 0102 | | | | | | |
| 455 | SPIKE (SEQ | 0347- | FASVYAWNRKRISNCVAD | 7 | 11.13 | 3 | 0.63 | 5 |
| | ID NO: 3) | 0364 | | | | | | |
| 456 | SPIKE (SEQ | 0536- | NKCVNFNFNGLTGT | 7 | 12.54 | 0 | 0.00 | 2 |
| | ID NO: 3) | 0549 | | | | | | |

Putative T-cell epitopes were also screened for cross-conservation with the human proteome using JanusMatrix, as further described in more detail in the Examples. The JanusMatrix system (EpiVax, Providence, R.I.) useful for screening peptide sequences for cross-conservation with a host proteome. JanusMatrix is an algorithm that predicts the potential for cross-reactivity between peptide clusters and the host genome or proteome, based on conservation of TCR-facing residues in their putative MEW ligands. The JanusMatrix algorithm first considers all the predicted epitopes contained within a given protein sequence and divides each predicted epitope into its constituent agretope and epitope. Each sequence is then screened against a database of host proteins. Peptides with a compatible MHC-facing agretope (i.e., the agretopes of both the input peptide and its host counterparty are predicted to bind the same MHC allele) and exactly the same TCR-facing epitope are returned. The JanusMatrix Homology Score suggests a bias towards immune tolerance. In the case of a therapeutic protein, cross-conservation between autologous human epitopes and epitopes in the therapeutic may increase the likelihood that such a candidate will be tolerated by the human immune system. In the case of a vaccine, cross-conservation between human epitopes and the antigenic epitopes may indicate that such a candidate utilizes immune camouflage, thereby evading the immune response and making for an ineffective vaccine. When the host is, for example, a human, the peptide clusters are screened against human genomes and proteomes, based on conservation of TCR-facing residues in their putative HLA ligands. The peptides are then scored using the JanusMatrix Homology Score. In aspects, peptides with a JanusMatrix Homology Score below 2.5 or below 3.0 (and even below 2.0 in certain aspects) indicate low tolerogenicity potential and may be useful for vaccines. In aspects, peptides with a JanusMatrix Homology Score above 3.0 indicate high tolerogenicity potential and may not be useful for vaccines, and in aspects may be excluded from the T cell epitope compounds and compositions, methods, and assays/kits of the present disclosure. Table I discloses the JanusMatrix Homology score of the instantly-disclosed T cell epitopes of SEQ ID NOS: 4-224 and 454-456, all of which are well below 2.5.

In aspects, and as also described in further detail in the Examples, T-cell epitopes of the present disclosure are highly conserved among related coronaviruses that infect humans including highly pathogenic SARS-CoV and MERS-CoV and low pathogenicity common cold coronaviruses (CCCs) OC43, HKU1, NL63, and 229E. Prior exposure to these viruses may have established T cell memory that can be recalled upon SARS-CoV-2 infection and vaccination. As well, SARS-CoV-2 infection and vaccination establishes T cell memory that can influence responses in future infections to these viruses or yet-to-emerge coronaviruses. To identify potentially cross-reactive sequences, we screened the TCR-face of select SARS-CoV-2 epitopes for homology with coronaviruses that infect humans, using the JanusMatrix algorithm (e.g., Table 58A and Table 58B). All of the selected membrane and envelope sequences shared identical TCR-face patterns with SARS-CoV. Half the selected spike clusters were unique to SARS-CoV-2 and the other half are conserved with SARS-CoV. Only three selected clusters were cross-conserved outside SARS viruses. Given reports of pre-existing T cell immunity in people with no SARS-CoV-2 experience, the requirement for 100% identity at every TCR-face position was relaxed. Fixing two positions shown to be extensively involved in TCR interactions (positions 5 and 8), JanusMatrix predicted an expanded cross-conservation landscape for selected SARS-CoV-2 spike and membrane clusters. Most selected spike clusters were conserved, by these criteria, in the subset of coronaviruses that infect humans. The remainder of the selected sequences with cross-reactivity potential are cross-conserved among highly pathogenic beta-coronaviruses or among high and low pathogenicity beta-coronaviruses. Only three clusters were unique to SARS-CoV-2 by the criteria described above, and none are solely conserved with SARS-CoV. The single membrane selected clusters was cross-conserved in the highly pathogenic beta-coronaviruses and coronaviruses that infect humans subsets. As the vast majority of people were not exposed to SARS-CoV and MERS-CoV, we also explored cross-conservation between SARS-CoV-2 and CCCs only. Of the 32 selected peptides, only 2 are SARS-CoV-2-specific. Eighteen clusters (56.6%) are cross-conserved across OC43, HKU1, NL63, and 229E and 12 (37.5%) are cross-conserved in at least one of these four CCCs.

In aspects, T-cell epitopes of the present disclosure bind to at least one and preferably two or more common HLA class I and/or class II alleles with at least a moderate affinity (e.g., in aspects, <1000 μM IC₅₀, <500 μM IC₅₀, <400 μM IC₅₀, <300 μM IC₅₀, or <200 μM IC₅₀ in HLA binding assays based on soluble HLA molecules). In aspects, T-cell epitopes of the present disclosure are capable of being presented at the cell surface by cells in the context of at least one and, in other aspects, two or more alleles of the HLA. In this context, the epitope-HLA complex can be recognized by CD4+ and/or CD8+ T-cells having TCRs that are specific for the epitope-HLA complex and circulating in subjects. In aspects, the recognition of the epitope-HLA complex can cause the matching T-cell to be activated and to secrete activating cytokines (e.g., effector cytokines such as IFNγ) and chemokines.

In aspects, T-cell epitope compounds or compositions of the present disclosure include one or more peptides or polypeptides a disclosed herein. In aspects, the present disclosure is directed to a peptide or polypeptide having an amino acid sequence comprising, consisting of, or consisting essentially of one or more of SEQ ID NOS: 4-224 and 454-456, or fragments or variants thereof. The phrase “consisting essentially of” is intended to mean that a peptide or polypeptide according to the present disclosure, in addition to the sequence according to any of SEQ ID NOS: 4-224 and 454-456 or a fragment or variant thereof, contains additional amino acids or residues that may be present at either terminus of the peptide and/or on a side chain that are not necessarily forming part of the peptide or polypeptide that functions as an MHC ligand and provided they do not substantially impair the activity of the peptide to function as a T-cell epitope. In aspects, the peptides or polypeptides of the instant disclosure can be either in neutral (uncharged) or salt forms, and may be either free of or include modifications such as glycosylation, side chain oxidation, or phosphorylation. In aspects, the peptides or polypeptides of the instant disclosure can be capped with an n-terminal acetyl and/or c-terminal amino group. In aspects, peptides or polypeptides of the instant disclosure having SEQ ID NOS: 9, 34, 49, 54, 59, 94, 99, 104, 109, 142, 147, 181, 191, 196, 210-213, 215-218, 220, and 223 are capped with an n-terminal acetyl and a c-terminal amino group. In aspects, peptides or polypeptides of the instant disclosure having SEQ ID NOS: 89, 114, 186, 214, 119, 221-222, and 224 are capped with an n-terminal acetyl and are not capped at the c-terminus. Again, Table 1 discloses the cluster sequence (bolded) of each the instantly-disclosed T cell epitopes of SEQ ID NOS: 4-224 and 454-456, the cluster score (without flanks), the number of EpiMatrix hits for class I MHC (which is the number of hits is the number of EpiMatrix Z-scores above 1.64 or top 5% found within the sequence), and the number of EpiMatrix hits for class II MHC (which is the number of hits is the number of EpiMatrix Z-scores above 1.64 or top 5% found within the sequence).

In aspects, the instant disclosure is directed to a peptide or polypeptide comprising, consisting, or consisting essentially of an amino acid sequence of SEQ ID NOS. 4-224 and 454-456 (and/or fragments or variants thereof), and optionally 1 to 12 additional amino acids distributed in any ratio on the N terminus and/or C-terminus of the polypeptide of SEQ ID NOS. 4-244. In aspects, the instant disclosure is directed to a peptide or polypeptide have a core amino acid sequence comprising, consisting of, or consisting essentially of one or more peptides or polypeptides having an amino acid sequence of SEQ ID NOS. 4-224 and 454-456 (and/or fragments or variants thereof), and optionally having extensions of 1 to 12 amino acids on the C-terminal and/or the N-terminal of the core amino acid sequence, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio). In aspects, the instant disclosure is directed to a peptide or polypeptide have a core sequence comprising, consisting of, or consisting essentially of one or more peptides or polypeptides having an amino acid sequence of SEQ ID NOS. 4-224 and 454-456 (and/or fragments and variants thereof), optionally with extensions of 1 to 12 amino acids on the C-terminal and/or the N-terminal, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio), provided that the polypeptide with the flanking amino acids is still able to bind to the same HLA molecule (i.e., retain MEW binding propensity) as said polypeptide core sequence without said flanking amino acids. In aspects, said polypeptide with the flanking amino acids is still able to bind to the same HLA molecule (i.e., retain MEW binding propensity) and/or retain the same TCR specificity, and/or retain anti-SARS-CoV-2 activity, as said polypeptide core sequence without said flanking amino acids. In aspects, the extension(s) may serve and be designed to improve the biochemical properties of the peptides or polypeptides (e.g., but not limited to, solubility or stability) or to improve the likelihood for efficient proteasomal processing of the peptide. In aspects, the polypeptides of the present disclosure may be isolated, synthetic, and/or recombinant, and may comprise post-transcriptional modifications such as glycosylation, added chemical groups, etc. In aspects, said flanking amino acid sequences are those that also flank the peptides or polypeptides included therein in the naturally occurring protein (for example, as found in SARS-CoV-2 Wuhan-Hu-1 (GenBank id: MN908947), which was selected as the reference strain). In aspects, said flanking amino acids sequences are those that also flank the peptides or polypeptides included therein in the naturally occurring protein, for example, as described below:
For a peptide or polypeptide have a core sequence comprising, consisting of, or consisting essentially of one or more peptides or polypeptides having an amino acid sequence of SEQ ID NOS. 4-8 and 210 (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS. 4-8 and 210 in the amino acid sequence of the envelope (SEQ ID NO: 1) of 2019-nCoV.
For a peptide or polypeptide have a core sequence comprising, consisting of, or consisting essentially of one or more peptides or polypeptides having an amino acid sequence of SEQ ID NOS. 9-63 and 211-213 (and/or fragments and variants thereof), optionally with extensions of 1 to 12 amino acids on the C-terminal and/or the N-terminal, the extensions of 1 to 12 amino acids are those found flanking the amino acid sequence of SEQ ID NOS. 9-63 and 211-213 in the amino acid sequence of the membrane (SEQ ID NO: 2) of 2019-nCoV.
For a peptide or polypeptide have a core sequence comprising, consisting of, or consisting essentially of one or more peptides or polypeptides having an amino acid sequence of SEQ ID NOS. 64-209 and 214-224 and 454-456 (and/or fragments and variants thereof), optionally with extensions of 1 to 12 amino acids on the C-terminal and/or the N-terminal, the extensions of 1 to 12 amino acids are those found flanking the amino acid sequence of 64-209 and 214-224 and 454-456 in the amino acid sequence of the spike (SEQ ID NO: 3) of 2019-nCoV.

In aspects, said flanking amino acid sequences as described herein may serve as a WIC stabilizing region. The use of a longer peptide may allow endogenous processing by patient cells and may lead to more effective antigen presentation and induction of T-cell responses. In aspects, the peptides or polypeptides of the instant disclosure can be isolated, recombinant, and/or synthetic. In aspects, the peptides or polypeptides can be either in neutral (uncharged) or salt forms, and may be either free of or include modifications such as glycosylation, side chain oxidation, or phosphorylation. In aspects, the peptides or polypeptides of the instant disclosure can be capped with an n-terminal acetyl and/or c-terminal amino group.

In aspects, the instant disclosure is directed to one or more Class II polypeptides (“clusters”) of Table 1, (and/or fragments or variants thereof), and optionally 1 to 12 additional amino acids distributed in any ratio on the N terminus and/or C-terminus of a polypeptide of Table 1.

In aspects, the instant disclosure is directed to one or more Class II polypeptides (“clusters”) comprising, consisting, or consisting essentially of an amino acid sequence of SEQ ID NOS: 210, 9, 211, 34, 212, 213, 49, 54, 59, 214, 215, 89, 94, 99, 216, 104, 109, 114, 217, 218, 142, 147, 219, 220, 221, 222, 181, 186, 191, 196, 223, and 224 (and/or fragments or variants thereof), and optionally 1 to 12 additional amino acids distributed in any ratio on the N-terminus and/or C-terminus of the polypeptide of SEQ ID NOS: 210, 9, 211, 34, 212, 213, 49, 54, 59, 214, 215, 89, 94, 99, 216, 104, 109, 114, 217, 218, 142, 147, 219, 220, 221, 222, 181, 186, 191, 196, 223, and 224.

In aspects, the instant disclosure is directed to one or more Class II polypeptides (“clusters”) comprising, consisting, or consisting essentially of an amino acid sequence of SEQ ID NOS: 218, 19, 216, 212, 89, 94, 34, 142, 221, 54, 186, 59, 214, 223, 219, 191, 224, 222, 210, 49, 104, 217, 213, 215, 109, 220, 147, 181, 196, 9, 114, and 99 (and/or fragments or variants thereof), and optionally 1 to 12 additional amino acids distributed in any ratio on the N-terminus and/or C-terminus of the polypeptide of SEQ ID NOS: 218, 19, 216, 212, 89, 94, 34, 142, 221, 54, 186, 59, 214, 223, 219, 191, 224, 222, 210, 49, 104, 217, 213, 215, 109, 220, 147, 181, 196, 9, 114, and 99.

In aspects, the instant disclosure is directed to one or more Class II polypeptides (“clusters”) comprising, consisting, or consisting essentially of an amino acid sequence of SEQ ID NOS: 210, 9, 19, 34, 212, 44, 49, 54, 59, 214, 89, 94, 99, 216, 104, 109, 110, 114, 218, 142, 147, 220, 221, 176, 181, 186, 191, 196, 223, 224, 454, 455, and 456 (and/or fragments or variants thereof), and optionally 1 to 12 additional amino acids distributed in any ratio on the N-terminus and/or C-terminus of the polypeptide of SEQ ID NOS: 210, 9, 19, 34, 212, 44, 49, 54, 59, 214, 89, 94, 99, 216, 104, 109, 110, 114, 218, 142, 147, 220, 221, 176, 181, 186, 191, 196, 223, 224, 454, 455, and 456.

In aspects, the instant disclosure is directed to a polypeptide comprising an amino acid sequence having at least 75%, 80%, 85%, 90%, or 95% homology to any one of SEQ ID NOS: 4-224 and 454-456 (and/or fragments thereof), wherein said polypeptide is still able to bind to a same HLA molecule (i.e., retain MEW binding propensity) and/or retain the same TCR specificity, and/or retain anti-SARS-CoV-2 activity.

In aspects, the present disclosure is directed to a concatemeric polypeptide or peptide that comprises at one or more of the instantly-disclosed polypeptides or peptides (e.g., but not limited to, a peptide or polypeptide comprising, consisting, or consisting essentially of an amino acid sequence of SEQ ID NOS. 4-224 and 454-456 (and/or fragments or variants thereof), and optionally 1 to 12 additional amino acids distributed in any ratio on the N terminus and/or C-terminus of the polypeptide of SEQ ID NOS. 4-224 and 454-456) linked, fused, or joined together (e.g., fused in-frame, chemically-linked, or otherwise bound) to an additional peptide or polypeptide. Such additional peptide or polypeptide may be one or more of the instantly instantly-disclosed polypeptides or peptides, or may be an additional peptide or polypeptide of interest. In aspects, a concatemeric peptide is composed of 3 or more, 4 or more, 5 or more 6 or more 7 or more, 8 or more, 9 or more of the instantly-disclosed peptides or polypeptides. In other aspects, the concatemeric peptides or polypeptides include 1000 or more, 1000 or less, 900 or less, 500 or less, 100 or less, 75 or less, 50 or less, 40 or less, 30 or less, 20 or less or 100 or less peptide epitopes. In yet other embodiments, a concatemeric peptide has 3-100, 5-100, 10-100, 15-100, 20-100, 25-100, 30-100, 35-100, 40-100, 45-100, 50-100, 55-100, 60-100, 65-100, 70-100, 75-100, 80-100, 90-100, 5-50, 10-50, 15-50, 20-50, 25-50, 30-50, 35-50, 40-50, 45-50, 100-150, 100-200, 100-300, 100-400, 100-500, 50-500, 50-800, 50-1,000, or 100-1,000 of the instantly-disclosed peptides or polypeptides linked, fused, or joined together. Each peptide or polypeptide of the concatemeric polypeptide may optionally have one or more linkers, which may optionally be cleavage sensitive sites, adjacent to their N and/or C terminal end. Such suitable linkers and cleavage sensitive sites, including AAY cleavage motifs or a poly GS linker which may be included on the N-terminus of the C-terminal element, are known in the art. In such a concatemeric peptide, two or more of the peptide epitopes may have a linker, which may act as a cleavage sensitive site, between them. Alternatively two or more of the peptide epitopes may be connected directly to one another or through a linker that is not a cleavage sensitive site. Additional suitable linkers are also discussed below in the context of hybrid Ii-key constructs, but may be used in the instantly-disclosed concatemeric peptides. In aspects, the concatemeric peptides or polypeptides can be either in neutral (uncharged) or salt forms, and may be either free of or include modifications such as glycosylation, side chain oxidation, or phosphorylation. In aspects, the concatemeric peptides or polypeptides of the instant disclosure can be capped with an n-terminal acetyl and/or c-terminal amino group. In aspects, such linker is antigenically neutral, and the linker is preferably less than the length of a peptidyl backbone of 9 amino acids linearly arranged. In aspects, linker length is the length of a peptidyl backbone of between 2 and 8 amino acids, linearly arranged. In aspects, the spacer is unable to hydrogen bond in any spatially distinct manner to other distinct elements of the enhancing hybrid peptide.

In aspects, and with respect to antigenically neutral linker elements, various chemical groups may be incorporated as linkers instead of amino acids. Examples are described in U.S. Pat. No. 5,910,300, the contents of which are incorporated herein by reference. In aspects, a linker may be comprised of an aliphatic chain optimally interrupted by heteroatoms, for example a C2-C6 alkylene, or ═N—(CH2)2-6-N═. Alternatively, a spacer may be composed of alternating units, for example of hydrophobic, lipophilic, aliphatic and aryl-aliphatic sequences, optionally interrupted by heteroatoms such as 0, N, or S. Such components of a spacer are preferably chosen from the following classes of compounds: sterols, alkyl alcohols, polyglycerides with varying alkyl functions, alkyl-phenols, alkyl-amines, amides, hydroxyphobic polyoxyalkylenes, and the like. Other examples are hydrophobic polyanhydrides, polyorthoesters, polyphosphazenes, polyhydroxy acids, polycaprolactones, polylactic, polyglycolic polyhydroxy-butyric acids. A linker may also contain repeating short aliphatic chains, such as polypropylene, isopropylene, butylene, isobutylene, pentamethlyene, and the like, separated by oxygen atoms.

Additional peptidyl sequences which can be used in as possible linkers are described in U.S. Pat. No. 5,856,456, the contents of which are incorporated herein by reference. In one embodiment, a linker has a chemical group incorporated within which is subject to cleavage. Without limitation, such a chemical group may be designed for cleavage catalyzed by a protease, by a chemical group, or by a catalytic monoclonal antibody. In the case of a protease-sensitive chemical group, tryptic targets (two amino acids with cationic side chains), chymotryptic targets (with a hydrophobic side chain), and cathepsin sensitivity (B, D or S) are favored. The term ‘tryptic target’ is used herein to describe sequences of amino acids which are recognized by trypsin and trypsin-like enzymes. The term ‘chymotryptic target’ is used herein to describe sequences of amino acids which are recognized by chymotrypsin and chymotrypsin-like enzymes. In addition, chemical targets of catalytic monoclonal antibodies, and other chemically cleaved groups are well known to persons skilled in the art of peptide synthesis, enzymatic catalysis, and organic chemistry in general, and can be designed into the hybrid structure and synthesized, using routine experimental methods.

In aspects, a concatemeric polypeptide of the instant disclosure is produced using the EpiAssembler System (EpiVax). The EpiAssembler system is useful for assembling overlapping epitopes to Immunogenic Consensus Sequences (ICS). EpiAssembler is an algorithm that optimizes the balance between pathogen and population coverage. EpiAssembler uses the information from the sequences produced by Conservatrix and EpiMatrix to form highly immunogenic consensus sequences. In aspects, the present disclosure provides a concatemeric polypeptide having anti-SARS-CoV-2 activity. As previously described, anti-SARS-CoV-2 activity means that the instantly-disclosed therapeutic T-cell epitope compounds and compositions are, in aspects: capable of stimulating, inducing, and/or expanding an immune response to SARS-CoV-2 (e.g., a cellular (CD4+ and/or CD8+ T-cell response) or humoral immune response to SARS-CoV-2) and/or associated diseases in a subject; capable of stimulating, inducing, and/or expanding a SARS-CoV-2-specific IFNγ response (e.g., by lymphocytes such as PMBC, or effector CD4+ and/or CD8+ T-cells), capable of inhibiting SARS-CoV-2 viral replication or infectivity, and/or capable of inducing immunity against SARS-CoV-2. In aspects, a T-cell epitope compound or composition of the present disclosure having anti-SARS-CoV-2 activity will reduce the disease symptoms resulting from SARS-CoV-2 challenge by at least about 5% to about 50%, at least about 10% to about 60%, at least about 30% to about 70%, at least about 40% to about 80%, or at least about 50% to about 90% or greater, including any value or range therebetween. Again, anti-SARS-CoV-2 activity can be determined by various experiments and assays as known to those of skill in the art, including methods such as by antibody titrations of sera, e.g., by ELISA and/or seroneutralization assay analysis and/or by vaccination challenge evaluation, including use of experiments and assays as disclosed in the Examples herein.

In aspects, the concatemeric polypeptides of the instant disclosure can be isolated, recombinant, and/or synthetic. In aspects, the concatemeric peptides or polypeptides can be either in neutral (uncharged) or salt forms, and may be either free of or include modifications such as glycosylation, side chain oxidation, or phosphorylation. In aspects, the concatemeric peptides or polypeptides of the instant disclosure can be capped with an n-terminal acetyl and/or c-terminal amino group.

In aspects, one or more peptides or polypeptides of the instant disclosure (e.g., but not limited to, a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N-terminus and/or C-terminus of the polypeptide of SEQ ID NOS: 4-224 and 454-456; as well as the concatemeric polypeptides disclosed herein, and the hybrid Ii-key constructs) joined to, linked to (e.g., fused in-frame, chemically-linked, or otherwise bound), and/or inserted into a heterologous polypeptide. As previously described, with respect to the one or more T-cell epitopes of the instant disclosure, the term “heterologous polypeptide” is intended to mean that the one or more T-cell epitopes of the instant disclosure are heterologous to, or not included naturally, in the heterologous polypeptide. In aspects, a heterologous polypeptide may include, but are not limited to, e.g. monoclonal antibody, polyclonal antibody, mouse antibody, human antibody, humanized antibody, mono specific antibody, bispecific antibody, glycosylated antibody, Fc-modified antibody, or antibody-drug conjugates; an antibody of different class or subclass (e.g., IgG (e.g., IgG1, IgG2, IgG3, IgG4), IgM, IgA, IgD or IgE molecules) or antigen-specific antibody fragments thereof (including, but not limited to, a Fab, F(ab′)2, Fv, disulfide linked Fv, scFv, single domain antibody, closed conformation multispecific antibody, disulfide-linked scFv, diabody)). In aspects, one or more of the instantly-disclosed polypeptides may be inserted into the heterologous polypeptide (e.g., through recombinant techniques, mutagenesis, or other known means in the art), may be added to the C-terminus (with or without the use of linkers, as is known in the art), and/or added to the N-terminus (with or without the use of linkers, as is known in the art) of the heterologous polypeptide. In aspects, one or more of the instantly-disclosed polypeptides may be inserted into or replace amino acids in a Fc domain as disclosed in U.S. Pat. Nos. 7,442,778, 7,645,861, 7,655,764, 7,655,765, and/or 7,750,128 (each of which are herein incorporated by reference in their entirety). For example, protein engineering by mutagenesis can be performed using site-directed mutagenesis techniques, or other mutagenesis techniques known in the art (see e.g., James A. Brannigan and Anthony J. Wilkinson., 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety). In aspects, chimeric or fusion polypeptides comprise one or more of the instantly-disclosed polypeptides of the present disclosure operatively linked to a heterologous polypeptide. “Operatively linked” indicates that the one or more of the instantly-disclosed polypeptides and the heterologous protein are fused in-frame or chemically linked or otherwise bound. For example, in aspects, the one or more of the instantly-disclosed polypeptides may be covalently bound to one or more internal conjugation site(s) in an Fc domain as disclosed in U.S. Pat. Nos. 8,008,453, 9,114,175, and/or 10,188,740 (each of which are herein incorporated by reference in their entirety). In aspects, the one or more peptides or polypeptides of the instant disclosure (e.g., but not limited to, a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 4-224 and 454-456) may be joined to, linked to (e.g., fused in-frame, chemically-linked, or otherwise bound), and/or inserted into a heterologous polypeptide as a whole, although it may be made up from a joined to, linked to (e.g., fused in-frame, chemically-linked, or otherwise bound), and/or inserted amino acid sequence, together with flanking amino acids of the heterologous polypeptide. In aspects, the present disclosure is directed to polypeptide (which, in aspects, may be an isolated, synthetic, or recombinant) having a sequence comprising one or more of SEQ ID NOS: 4-224 and 454-456 (and/or fragments or variants thereof, and optionally 1 to 12 additional amino acids distributed in any ratio on the N terminus and/or C-terminus of the polypeptide of SEQ ID NOS: 4-224 and 454-456), wherein said one or more of SEQ ID NOS: 4-224 and 454-456 is not naturally included in the polypeptide and/or said one or more of SEQ ID NOS: 4-224 and 454-456 is not located at its natural position in the polypeptide. For example, in aspects, the one or more peptides, polypeptides, concatemeric peptides, and/or hybrid Ii-key constructs of the present disclosure may be inserted into a SARS-CoV-2 sequence in which the SARS-CoV-2 sequence does not include the one or more peptides, polypeptides, concatemeric peptides, and/or hybrid Ii-key constructs of the present disclosure (e.g., the SARS-CoV-2 sequence is mutated to not include the one or more peptides, polypeptides, concatemeric peptides, and/or hybrid Ii-key constructs of the present disclosure) or the one or more peptides, polypeptides, concatemeric peptides, and/or hybrid Ii-key constructs of the present disclosure is inserted into a SARS-CoV-2 sequence but not at its natural position. In aspects, the one or more peptides or polypeptides of the instant disclosure can be joined or linked to (e.g., fused in-frame, chemically-linked, or otherwise bound) to a small molecule (e.g., albumin or other known carriers and proteins), drug, or drag fragment, for example but not limited to, a drug or drug fragment that is binds with high affinity to defined HLAs.

In aspects, the present disclosure is directed to hybrid Ii-key constructs (as originally disclosed in U.S. Pat. No. 6,432,409, hereby incorporated by reference in its entirety), with such hybrid Ii-key constructs comprising one or more of one or more of SEQ ID NOS. 4-224 and 454-456 (and/or fragments and variants thereof, and optionally 1 to 12 additional amino acids distributed in any ratio on the N terminus and/or C-terminus of the polypeptide of SEQ ID NOS. 4-224 and 454-456). Such hybrid Ii-key constructs are based on the initial discovery that that an MHC Class II-restricted antigenic epitope that is covalently linked to a mammalian Ii-key peptide by an appropriate intervening chemical structure, to form a hybrid construct (and in aspects, a hybrid polypeptide), is presented to T lymphocytes by antigen presenting cells with significantly higher efficacy than is the precursor antigenic epitope. In aspects, the instantly-disclosed Ii-key hybrid constructs have an N-terminus comprised of a mammalian Ii-key peptide, or a modification thereof, which retains antigen presentation enhancing activity, and covalently, but indirectly, linked to the Ii-key peptide is the specific antigenic epitope(s) to be presented (which in aspects comprises one or more of the instantly disclosed peptides or polypeptides (e.g., but not limited to, a peptide or polypeptide comprising, consisting, or consisting essentially of an amino acid sequence of SEQ ID NOS. 4-224 and 454-456 (and/or fragments or variants thereof), and optionally 1 to 12 additional amino acids distributed in any ratio on the N terminus and/or C-terminus of the polypeptide of SEQ ID NOS. 4-224 and 454-456)). The intervening chemical structure may be referred to as a “spacer” or “linker”, and will be described in more detail below.

In aspects, the hybrid Ii-key constructs of the present disclosure are comprised of 3 elements, similar to the '813 hybrid. The 3 elements are: 1) an N-terminal element comprising, consisting or, or consisting essentially of 4-16 residues of the mammalian Ii-Key peptide LRMKLPKPPKPVSKMR (SEQ ID NO: 225) and modifications of SEQ ID No: 225 that retain antigen presentation enhancing activity (which in aspects may comprise, consist of, or consist essentially of the residues LRMK (amino acids 1-4) of SEQ ID NO.: 225 and 0-12 additional sequential residues of SEQ ID NO.: 225, and modifications of SEQ ID No: 225 that retain antigen presentation enhancing activity; or in other aspects may comprise, consist or, or consist essentially of LRMK (SEQ ID NO: 226)); 2) a C-terminal element comprising an MHC Class II and/or Class I-presented epitope in the form of a polypeptide or peptidomimetic structure which binds to the antigenic peptide binding site of an MHC Class II and/or Class I molecule(s) (which in aspects comprises one or more of the instantly disclosed peptides or polypeptides (e.g., but not limited to, a peptide or polypeptide comprising, consisting, or consisting essentially of an amino acid sequence of SEQ ID NOS. 4-224 and 454-456 (and/or fragments or variants thereof), and optionally 1 to 12 additional amino acids distributed in any ratio on the N terminus and/or C-terminus of the polypeptide of SEQ ID NOS. 4-224 and 454-456)); and 3) an optional intervening chemical structure covalently linking the N-terminal and C-terminal elements of the hybrid, which in aspects the chemical structure being a covalently joined group of atoms which when arranged in a linear fashion forms a flexible chain which extends up to the length of 20 amino acids likewise arranged in a linear fashion. In aspects, the intervening chemical structure comprises, consists of, or consists essentially of a delta-aminovaleric acid, which is 5-aminopentanoic acid (and may be referred to as “ava”). In aspects, the intervening chemical structure may comprise, consist of, or consist essentially of ava, ala-ala-ala, gly-gly, or other biologically accepted functional equivalent of ava, ala-ala-ala, and gly-gly. Specific Ii-key hybrid constructs are disclosed in Table 2, and include SEQ ID NOS: 232-452 and 457-459. For determining the junctional EpiMatrix hits (Class II) and junctional human hits of LRMK (SEQ ID NO: 226) in the Ii-key hybrid constructs of Table 2, Gly-Gly was used as a proxy for ava. In certain aspects, the C-terminal element comprising an MHC Class II and/or MHC Class I-presented epitope in the form of a polypeptide or peptidomimetic structure which binds to the antigenic peptide binding site of an MHC Class II and/or Class I molecule(s) (which in aspects comprises one or more of the instantly disclosed peptides or polypeptides (e.g., but not limited to, a peptide or polypeptide comprising, consisting, or consisting essentially of an amino acid sequence of SEQ ID NOS. 4-224 and 454-456 (and/or fragments or variants thereof), and optionally 1 to 12 additional amino acids distributed in any ratio on the N terminus and/or C-terminus of the polypeptide of SEQ ID NOS. 4-224 and 454-456) of the hybrid Ii-key constructs of instant disclosure can be capped with an n-terminal acetyl and/or c-terminal amino group. In aspects of the instantly-disclosed hybrid Ii-key constructs comprising a peptide or polypeptide of the instant disclosure having SEQ ID NOS: 9, 34, 49, 54, 59, 94, 99, 104, 109, 142, 147, 181, 191, 196, 210-213, 215-218, 220, and 223 as the C-terminal element, such peptides are capped with an n-terminal acetyl and a c-terminal amino group. In aspects of the instantly-disclosed hybrid Ii-key constructs comprising a peptide or polypeptide of the instant disclosure having SEQ ID NOS: 89, 114, 186, 214, 119, 221-222, and 224 as the C-terminal element, such peptides are capped with an n-terminal acetyl and are not capped at the c-terminus. In aspects of the hybrid Ii-key constructs of SEQ ID NOS: 237, 262, 277, 283, 287, 322, 327, 332, 337, 370, 375, 409, 419, 424, 438-441, 443-446, 448, and 451, the disclosed peptide sequence of the C-terminal element are capped with an n-terminal acetyl and a c-terminal amino group. In aspects of the hybrid Ii-key constructs of SEQ ID NOS: 317, 342, 414, 442, 447 449, 450, and 452, the disclosed peptide sequence of the C-terminal element are capped with an n-terminal acetyl and are not capped at the c-terminus.

TABLE 2

| | | Sequence | | | |
|---|---|---|---|---|---|
| | | Address | | | |
| | Input | (w/Flanks, | Sequence | | |
| | Sequence | not | (cluster sequence | LRMK (SEQ | |
| | (not | including | is bolded, | ID NO: 226) | LRMK (SEQ |
| | including | LRMK-ava | mutations | Junctional | ID NO: 226) |
| SEQ | LRMK-ava | (SEQ | from | EpiMatrix | Junctional |
| ID | (SEQ ID | ID NO | WT are | Hits | Human Hits |
| NO: | NO: 453)) | 453)) | italicized) | (Class II) | (Class II) |
| 232 | ENVELOPE (SEQ | 0054- | LRMK-ava-PSFYVYSRVKNLNSSR | 3 | 2 |
| | ID NO: 1) | 0069 | | | |
| 233 | ENVELOPE (SEQ | 0055- | LRMK-ava-SFYVYSRVKNLNSSR | | |
| | ID NO: 1) | 0069 | | | |
| 234 | ENVELOPE (SEQ | 0056- | LRMK-ava-FYVYSRVKNLNSSR | | |
| | ID NO: 1) | 0069 | | | |
| 235 | ENVELOPE (SEQ | 0057- | LRMK-ava-YVYSRVKNLNSSR | | |
| | ID NO: 1) | 0069 | | | |
| 236 | ENVELOPE (SEQ | 0057- | LRMK-ava-YVYSRVKNLNS | | |
| | ID NO: 1) | 0067 | | | |
| 237 | MEMBRANE | 0013- | LRMK-ava-LKKLLEQWNLVIGFL | 5 | 4 |
| | (SEQ ID NO: 2) | 0027 | | | |
| 238 | MEMBRANE | 0014- | LRMK-ava-KKLLEQWNLVIGFL | | |
| | (SEQ ID NO: 2) | 0027 | | | |
| 239 | MEMBRANE | 0015- | LRMK-ava-KLLEQWNLVIGFL | | |
| | (SEQ ID NO: 2) | 0027 | | | |
| 240 | MEMBRANE | 0016- | LRMK-ava-LLEQWNLVIGFL | | |
| | (SEQ ID NO: 2) | 0027 | | | |
| 241 | MEMBRANE | 0016- | LRMK-ava-LLEQWNLVI | | |
| | (SEQ ID NO: 2) | 0024 | | | |
| 242 | MEMBRANE | 0017- | LRMK-ava-LEQWNLVIGFLFLTW | 0 | 0 |
| | (SEQ ID NO: 2) | 0031 | | | |
| 243 | MEMBRANE | 0018- | LRMK-ava-EQWNLVIGFLFLTW | | |
| | (SEQ ID NO: 2) | 0031 | | | |
| 244 | MEMBRANE | 0019- | LRMK-ava-QWNLVIGFLFLTW | | |
| | (SEQ ID NO: 2) | 0031 | | | |
| 245 | MEMBRANE | 0020- | LRMK-ava-WNLVIGFLFLTW | | |
| | (SEQ ID NO: 2) | 0031 | | | |
| 246 | MEMBRANE | 0020- | LRMK-ava-WNLVIGFLF | | |
| | (SEQ ID NO: 2) | 0028 | | | |
| 247 | MEMBRANE | 0032- | LRMK-ava-ICLLQFAYANRNRFLYI | 2 | 1 |
| | (SEQ ID NO: 2) | 0048 | | | |
| 248 | MEMBRANE | 0033- | LRMK-ava-CLLQFAYANRNRFLYI | | |
| | (SEQ ID NO: 2) | 0048 | | | |
| 249 | MEMBRANE | 0034- | LRMK-ava-LLQFAYANRNRFLYI | | |
| | (SEQ ID NO: 2) | 0048 | | | |
| 250 | MEMBRANE | 0035- | LRMK-ava-LQFAYANRNRFLYI | | |
| | (SEQ ID NO: 2) | 0048 | | | |
| 251 | MEMBRANE | 0035- | LRMK-ava-LQFAYANRNRF | | |
| | (SEQ ID NO: 2) | 0045 | | | |
| 252 | MEMBRANE | 0042- | LRMK-ava- | 4 | 0 |
| | (SEQ ID NO: 2) | 0060 | RNRFLYIIKLIFLWLLWPV | | |
| 253 | MEMBRANE | 0043- | LRMK-ava-NRFLYIIKLI | | |
| | (SEQ ID NO: 2) | 0060 | FLWLLWPV | | |
| 254 | MEMBRANE | 0044- | LRMK-ava-RFLYIIKLIFLWLLWPV | | |
| | (SEQ ID NO: 2) | 0060 | | | |
| 255 | MEMBRANE | 0045- | LRMK-ava-FLYIIKLIFLWLLWPV | | |
| | (SEQ ID NO: 2) | 0060 | | | |
| 256 | MEMBRANE | 0045- | LRMK-ava-FLYIIKLIFLWLL | | |
| | (SEQ ID NO: 2) | 0057 | | | |
| 257 | MEMBRANE | 0062- | LRMK-ava-LACFVLAAVYRINWI | 1 | 1 |
| | (SEQ ID NO: 2) | 0076 | | | |
| 258 | MEMBRANE | 0063- | LRMK-ava-ACFVLAAVYRINWI | | |
| | (SEQ ID NO: 2) | 0076 | | | |
| 259 | MEMBRANE | 0064- | LRMK-ava-CFVLAAVYRINWI | | |
| | (SEQ ID NO: 2) | 0076 | | | |
| 260 | MEMBRANE | 0065- | LRMK-ava-FVLAAVYRINWI | | |
| | (SEQ ID NO: 2) | 0076 | | | |
| 261 | MEMBRANE | 0065- | LRMK-ava-FVLAAVYRI | | |
| | (SEQ ID NO: 2) | 0073 | | | |
| 262 | MEMBRANE | 0093- | LRMK-ava-LSYFIASFRLFARTR | 5 | 0 |
| | (SEQ ID NO: 2) | 0107 | | | |
| 263 | MEMBRANE | 0094- | LRMK-ava-SYFIASFRLFARTR | | |
| | (SEQ ID NO: 2) | 0107 | | | |
| 264 | MEMBRANE | 0095- | LRMK-ava-YFIASFRLFARTR | | |
| | (SEQ ID NO: 2) | 0107 | | | |
| 265 | MEMBRANE | 0096- | LRMK-ava-FIASFRLFARTR | | |
| | (SEQ ID NO: 2) | 0107 | | | |
| 266 | MEMBRANE | 0096- | LRMK-ava-FIASFRLFA | | |
| | (SEQ ID NO: 2) | 0104 | | | |
| 267 | MEMBRANE | 0097- | LRMK-ava-IASFRLFARTRS | 3 | 0 |
| | (SEQ ID NO: 2) | 0114 | MWSFNP | | |
| 268 | MEMBRANE | 0098- | LRMK-ava-ASFRLFARTRSMWSFNP | | |
| | (SEQ ID NO: 2) | 0114 | | | |
| 269 | MEMBRANE | 0099- | LRMK-ava-SFRLFARTRSMWSFNP | | |
| | (SEQ ID NO: 2) | 0114 | | | |
| 270 | MEMBRANE | 0100- | LRMK-ava-FRLFARTRSMWSFNP | | |
| | (SEQ ID NO: 2) | 0114 | | | |
| 271 | MEMBRANE | 0100- | LRMK-ava-FRLFARTRSMWS | | |
| | (SEQ ID NO: 2) | 0111 | | | |
| 272 | MEMBRANE | 0146- | LRMK-ava-RGHLRIAGHHLGRCD | 2 | 1 |
| | (SEQ ID NO: 2) | 0160 | | | |
| 273 | MEMBRANE | 0147- | LRMK-ava-GHLRIAGHHLGRCD | | |
| | (SEQ ID NO: 2) | 0160 | | | |
| 274 | MEMBRANE | 0148- | LRMK-ava-HLRIAGHHLGRCD | | |
| | (SEQ ID NO: 2) | 0160 | | | |
| 275 | MEMBRANE | 0149- | LRMK-ava-LRIAGHHLGRCD | | |
| | (SEQ ID NO: 2) | 0160 | | | |
| 276 | MEMBRANE | 0149- | LRMK-ava-LRIAGHHLG | | |
| | (SEQ ID NO: 2) | 0157 | | | |
| 277 | MEMBRANE | 0165- | LRMK-ava-PKEITVATSRTLSYY | 1 | 0 |
| | (SEQ ID NO: 2) | 0179 | | | |
| 278 | MEMBRANE | 0166- | LRMK-ava-KEITVATSRTLSYY | | |
| | (SEQ ID NO: 2) | 0179 | | | |
| 279 | MEMBRANE | 0167- | LRMK-ava-EITVATSRTLSYY | | |
| | (SEQ ID NO: 2) | 0179 | | | |
| 280 | MEMBRANE | 0168- | LRMK-ava-ITVATSRTLSYY | | |
| | (SEQ ID NO: 2) | 0179 | | | |
| 281 | MEMBRANE | 0168- | LRMK-ava-ITVATSRTL | | |
| | (SEQ ID NO: 2) | 0176 | | | |
| 282 | MEMBRANE | 0175- | LRMK-ava-TLSYYKLGASQRVAGD | 3 | 3 |
| | (SEQ ID NO: 2) | 0190 | | | |
| 283 | MEMBRANE | 0176- | LRMK-ava-LSYYKLGASQRVAGD | | |
| | (SEQ ID NO: 2) | 0190 | | | |
| 284 | MEMBRANE | 0177- | LRMK-ava-SYYKLGASQRVAGD | | |
| | (SEQ ID NO: 2) | 0190 | | | |
| 285 | MEMBRANE | 0178- | LRMK-ava-YYKLGASQRVAGD | | |
| | (SEQ ID NO: 2) | 0190 | | | |
| 286 | MEMBRANE | 0178- | LRMK-ava-YYKLGASQRV | | |
| | (SEQ ID NO: 2) | 0187 | | | |
| 287 | MEMBRANE | 0201- | LRMK-ava-IGNYKLNTDHSSSSDNI | 0 | 0 |
| | (SEQ ID NO: 2) | 0217 | | | |
| 288 | MEMBRANE | 0202- | LRMK-ava-GNYKLNTDHSSSSDNI | | |
| | (SEQ ID NO: 2) | 0217 | | | |
| 289 | MEMBRANE | 0203- | LRMK-ava-NYKLNTDHSSSSDNI | | |
| | (SEQ ID NO: 2) | 0217 | | | |
| 290 | MEMBRANE | 0204- | LRMK-ava-YKLNTDHSSSSDNI | | |
| | (SEQ ID NO: 2) | 0217 | | | |
| 291 | MEMBRANE | 0204- | LRMK-ava-YKLNTDHSSSS | | |
| | (SEQ ID NO: 2) | 0214 | | | |
| 292 | SPIKE (SEQ ID | 0025- | LRMK-ava-PPAYTNSFTRGVYYP | 0 | 0 |
| | NO: 3) | 0039 | | | |
| 293 | SPIKE (SEQ ID | 0026- | LRMK-ava-PAYTNSFTRGVYYP | | |
| | NO: 3) | 0039 | | | |
| 294 | SPIKE (SEQ ID | 0027- | LRMK-ava-AYTNSFTRGVYYP | | |
| | NO: 3) | 0039 | | | |
| 295 | SPIKE (SEQ ID | 0028- | LRMK-ava-YTNSFTRGVYYP | | |
| | NO: 3) | 0039 | | | |
| 296 | SPIKE (SEQ ID | 0028- | LRMK-ava-YTNSFTRGV | | |
| | NO: 3) | 0036 | | | |
| 297 | SPIKE (SEQ ID | 0052- | LRMK-ava-QDLFLPFFSNVTWFH | 1 | 0 |
| | NO: 3) | 0066 | | | |
| 298 | SPIKE (SEQ ID | 0053- | LRMK-ava-DLFLPFFSNVTWFH | | |
| | NO: 3) | 0066 | | | |
| 299 | SPIKE (SEQ ID | 0054- | LRMK-ava-LFLPFFSNVTWFH | | |
| | NO: 3) | 0066 | | | |
| 300 | SPIKE (SEQ ID | 0055- | LRMK-ava-FLPFFSNVTWFH | | |
| | NO: 3) | 0066 | | | |
| 301 | SPIKE (SEQ ID | 0055- | LRMK-ava-FLPFFSNVT | | |
| | NO: 3) | 0063 | | | |
| 302 | SPIKE (SEQ ID | 0061- | NVTWFHAIHVSGTNGT | 1 | 0 |
| | NO: 3) | 0076 | | | |
| 303 | SPIKE (SEQ ID | 0062- | LRMK-ava-VTWFHAIHVSGTNGT | | |
| | NO: 3) | 0076 | | | |
| 304 | SPIKE (SEQ ID | 0063- | LRMK-ava-TWFHAIHVSGTNGT | | |
| | NO: 3) | 0076 | | | |
| 305 | SPIKE (SEQ ID | 0064- | LRMK-ava-WFHAIHVSGTNGT | | |
| | NO: 3) | 0076 | | | |
| 306 | SPIKE (SEQ ID | 0064- | LRMK-ava-WFHAIHVSGT | | |
| | NO: 3) | 0073 | | | |
| 307 | SPIKE (SEQ ID | 0087- | LRMK-ava-NDGVYFASTEKSNIIRG | 1 | 0 |
| | NO: 3) | 0103 | | | |
| 308 | SPIKE (SEQ ID | 0088- | LRMK-ava-DGVYFASTEKSNIIRG | | |
| | NO: 3) | 0103 | | | |
| 309 | SPIKE (SEQ ID | 0089- | LRMK-ava-GVYFASTEKSNIIRG | | |
| | NO: 3) | 0103 | | | |
| 310 | SPIKE (SEQ ID | 0090- | LRMK-ava-VYFASTEKSNIIRG | | |
| | NO: 3) | 0103 | | | |
| 311 | SPIKE (SEQ ID | 0090- | LRMK-ava-VYFASTEKSNI | | |
| | NO: 3) | 0100 | | | |
| 312 | SPIKE (SEQ ID | 0114- | LRMK-ava-TQSLLIVNNATNVVIKV | 1 | 1 |
| | NO: 3) | 0130 | | | |
| 313 | SPIKE (SEQ ID | 0115- | LRMK-ava-QSLLIVNNATNVVIKV | | |
| | NO: 3) | 0130 | | | |
| 314 | SPIKE (SEQ ID | 0116- | LRMK-ava-SLLIVNNATNVVIKV | | |
| | NO: 3) | 0130 | | | |
| 315 | SPIKE (SEQ ID | 0117- | LRMK-ava-LLIVNNATNVVIKV | | |
| | NO: 3) | 0130 | | | |
| 316 | SPIKE (SEQ ID | 0117- | LRMK-ava-LLIVNNATNVVI | | |
| | NO: 3) | 0130 | | | |
| 317 | SPIKE (SEQ ID | 0140- | LRMK-ava-FLGVYYHKNNKSWMESE | 2 | 0 |
| | NO: 3) | 0156 | | | |
| 318 | SPIKE (SEQ ID | 0141- | LRMK-ava-LGVYYHKNNKSWMESE | | |
| | NO: 3) | 0156 | | | |
| 319 | SPIKE (SEQ ID | 0142- | LRMK-ava-GVYYHKNNKSWMESE | | |
| | NO: 3) | 0156 | | | |
| 320 | SPIKE (SEQ ID | 0143- | LRMK-ava-VYYHKNNKSWMESE | | |
| | NO: 3) | 0156 | | | |
| 321 | SPIKE (SEQ ID | 0143- | LRMK-ava-VYYHKNNKSWM | | |
| | NO: 3) | 0153 | | | |
| 322 | SPIKE (SEQ ID | 0154- | LRMK-ava-ESEFRVYSSANNCTFEYV | 2 | 2 |
| | NO: 3) | 0171 | | | |
| 323 | SPIKE (SEQ ID | 0155- | LRMK-ava-SEFRVYSSANNCTFEYV | | |
| | NO: 3) | 0171 | | | |
| 324 | SPIKE (SEQ ID | 0156- | LRMK-ava-EFRVYSSANNCTFEYV | | |
| | NO: 3) | 0171 | | | |
| 325 | SPIKE (SEQ ID | 0157- | LRMK-ava-FRVYSSANNCTFEYV | | |
| | NO: 3) | 0171 | | | |
| 326 | SPIKE (SEQ ID | 0157- | LRMK-ava-FRVYSSANNCTF | | |
| | NO: 3) | 0168 | | | |
| 327 | SPIKE (SEQ ID | 0198- | LRMK-ava-DGYFKIYSKHTPINL | 0 | 0 |
| | NO: 3) | 0212 | | | |
| 328 | SPIKE (SEQ ID | 0199- | LRMK-ava-GYFKIYSKHTPINL | | |
| | NO: 3) | 0212 | | | |
| 329 | SPIKE (SEQ ID | 0200- | LRMK-ava-YFKIYSKHTPINL | | |
| | NO: 3) | 0212 | | | |
| 330 | SPIKE (SEQ ID | 0201- | LRMK-ava-FKIYSKHTPINL | | |
| | NO: 3) | 0212 | | | |
| 331 | SPIKE (SEQ ID | 0201- | LRMK-ava-FKIYSKHTP | | |
| | NO: 3) | 0209 | | | |
| 332 | SPIKE (SEQ ID | 0272- | LRMK-ava-PRTFLLKYNENGTITDA | 2 | 0 |
| | NO: 3) | 0288 | | | |
| 333 | SPIKE (SEQ ID | 0273- | LRMK-ava-RTFLLKYNENGTITDA | | |
| | NO: 3) | 0288 | | | |
| 334 | SPIKE (SEQ ID | 0274- | LRMK-ava-TFLLKYNENGTITDA | | |
| | NO: 3) | 0288 | | | |
| 335 | SPIKE (SEQ ID | 0275- | LRMK-ava-FLLKYNENGTITDA | | |
| | NO: 3) | 0288 | | | |
| 336 | SPIKE (SEQ ID | 0275- | LRMK-ava-FLLKYNENGTI | | |
| | NO: 3) | 0285 | | | |
| 337 | SPIKE (SEQ ID | 0315- | LRMK-ava-TSNFRVQPTESIVRF | 2 | 0 |
| | NO: 3) | 0329 | | | |
| 338 | SPIKE (SEQ ID | 0316- | LRMK-ava-SNFRVQPTESIVRF | | |
| | NO: 3) | 0329 | | | |
| 339 | SPIKE (SEQ ID | 0317- | LRMK-ava-NFRVQPTESIVRF | | |
| | NO: 3) | 0329 | | | |
| 340 | SPIKE (SEQ ID | 0318- | LRMK-ava-FRVQPTESIVRF | | |
| | NO: 3) | 0329 | | | |
| 341 | SPIKE (SEQ ID | 0318- | LRMK-ava-FRVQPTESI | | |
| | NO: 3) | 0326 | | | |
| 342 | SPIKE (SEQ ID | 0338- | LRMK-ava-FGEVFNATRFASVYA | 0 | 0 |
| | NO: 3) | 0352 | | | |
| 343 | SPIKE (SEQ ID | 0339- | LRMK-ava-GEVFNATRFASVYA | | |
| | NO: 3) | 0352 | | | |
| 344 | SPIKE (SEQ ID | 0340- | LRMK-ava-EVFNATRFASVYA | | |
| | NO: 3) | 0352 | | | |
| 345 | SPIKE (SEQ ID | 0341- | LRMK-ava-VFNATRFASVYA | | |
| | NO: 3) | 0352 | | | |
| 346 | SPIKE (SEQ ID | 0341- | LRMK-ava-VFNATRFAS | | |
| | NO: 3) | 0349 | | | |
| 347 | SPIKE (SEQ ID | 0347- | LRMK-ava-FASVYAWNRKRISN | 1 | 0 |
| | NO: 3) | 0360 | | | |
| 348 | SPIKE (SEQ ID | 0348- | LRMK-ava-ASVYAWNRKRISN | | |
| | NO: 3) | 0360 | | | |
| 349 | SPIKE (SEQ ID | 0349- | LRMK-ava-SVYAWNRKRISN | | |
| | NO: 3) | 0360 | | | |
| 350 | SPIKE (SEQ ID | 0350- | LRMK-ava-VYAWNRKRISN | | |
| | NO: 3) | 0360 | | | |
| 351 | SPIKE (SEQ ID | 0350- | LRMK-ava-VYAWNRKRIS | | |
| | NO: 3) | 0359 | | | |
| 352 | SPIKE (SEQ ID | 0362- | LRMK-ava-VADYSVLYNSASFST | 2 | 1 |
| | NO: 3) | 0376 | | | |
| 353 | SPIKE (SEQ ID | 0363- | LRMK-ava-ADYSVLYNSASFST | | |
| | NO: 3) | 0376 | | | |
| 354 | SPIKE (SEQ ID | 0364- | LRMK-ava-DYSVLYNSASFST | | |
| | NO: 3) | 0376 | | | |
| 355 | SPIKE (SEQ ID | 0365- | LRMK-ava-YSVLYNSASFST | | |
| | NO: 3) | 0376 | | | |
| 356 | SPIKE (SEQ ID | 0446- | LRMK-ava-GGNYNYLYRLFRKSN | 0 | 0 |
| | NO: 3) | 0460 | | | |
| 357 | SPIKE (SEQ ID | 0447- | LRMK-ava-GNYNYLYRLFRKSN | | |
| | NO: 3) | 0460 | | | |
| 358 | SPIKE (SEQ ID | 0448- | LRMK-ava-NYNYLYRLFRKSN | | |
| | NO: 3) | 0460 | | | |
| 359 | SPIKE (SEQ ID | 0449- | LRMK-ava-YNYLYRLFRKSN | | |
| | NO: 3) | 0460 | | | |
| 360 | SPIKE (SEQ ID | 0449- | LRMK-ava-YNYLYRLFR | | |
| | NO: 3) | 0457 | | | |
| 361 | SPIKE (SEQ ID | 0450- | LRMK-ava-NYLYRLFRKSNLKPF | 1 | 0 |
| | NO: 3) | 0464 | | | |
| 362 | SPIKE (SEQ ID | 0451- | LRMK-ava-YLYRLFRKSNLKPF | | |
| | NO: 3) | 0464 | | | |
| 363 | SPIKE (SEQ ID | 0452- | LRMK-ava-LYRLFRKSNLKPF | | |
| | NO: 3) | 0464 | | | |
| 364 | SPIKE (SEQ ID | 0453- | LRMK-ava-YRLFRKSNLKPF | | |
| | NO: 3) | 0464 | | | |
| 365 | SPIKE (SEQ ID | 0453- | LRMK-ava-YRLFRKSNL | | |
| | NO: 3) | 0461 | | | |
| 366 | SPIKE (SEQ ID | 0454- | LRMK-ava-RLFRKSNLKPFERD | 2 | 2 |
| | NO: 3) | 0467 | | | |
| 367 | SPIKE (SEQ ID | 0455- | LRMK-ava-LFRKSNLKPFERD | | |
| | NO: 3) | 0467 | | | |
| 368 | SPIKE (SEQ ID | 0456- | LRMK-ava-FRKSNLKPFERD | | |
| | NO: 3) | 0467 | | | |
| 369 | SPIKE (SEQ ID | 0456- | LRMK-ava-FRKSNLKPF | | |
| | NO: 3) | 0464 | | | |
| 370 | SPIKE (SEQ ID | 0483- | LRMK-ava-VEGFNCYFPLQSYGFQPT | 1 | 0 |
| | NO: 3) | 0500 | | | |
| 371 | SPIKE (SEQ ID | 0484- | LRMK-ava-EGFNCYFPLQSYGFQPT | | |
| | NO: 3) | 0500 | | | |
| 372 | SPIKE (SEQ ID | 0485- | LRMK-ava-GFNCYFPLQSYGFQPT | | |
| | NO: 3) | 0500 | | | |
| 373 | SPIKE (SEQ ID | 0486- | LRMK-ava-FNCYFPLQSYGFQPT | | |
| | NO: 3) | 0500 | | | |
| 374 | SPIKE (SEQ ID | 0486- | LRMK-ava-FNCYFPLQSYGF | | |
| | NO: 3) | 0497 | | | |
| 375 | SPIKE (SEQ ID | 0492- | LRMK-ava-LQSYGFQPTNGVGYQPY | 1 | 0 |
| | NO: 3) | 0508 | | | |
| 376 | SPIKE (SEQ ID | 0493- | LRMK-ava-QSYGFQPTNGVGYQPY | | |
| | NO: 3) | 0508 | | | |
| 377 | SPIKE (SEQ ID | 0494- | LRMK-ava-SYGFQPTNGVGYQPY | | |
| | NO: 3) | 0508 | | | |
| 378 | SPIKE (SEQ ID | 0495- | LRMK-ava-YGFQPTNGVGYQPY | | |
| | NO: 3) | 0508 | | | |
| 379 | SPIKE (SEQ ID | 0495- | LRMK-ava-YGFQPTNGVGY | | |
| | NO: 3) | 0503 | | | |
| 380 | SPIKE (SEQ ID | 0512- | LRMK-ava-VLSFELLHAPATVCG | 4 | 4 |
| | NO: 3) | 0526 | | | |
| 381 | SPIKE (SEQ ID | 0513- | LRMK-ava-LSFELLHAPATVCG | | |
| | NO: 3) | 0526 | | | |
| 382 | SPIKE (SEQ ID | 0514- | LRMK-ava-SFELLHAPATVCG | | |
| | NO: 3) | 0526 | | | |
| 383 | SPIKE (SEQ ID | 0515- | LRMK-ava-FELLHAPATVCG | | |
| | NO: 3) | 0526 | | | |
| 384 | SPIKE (SEQ ID | 0515- | LRMK-ava-FELLHAPAT | | |
| | NO: 3) | 0523 | | | |
| 385 | SPIKE (SEQ ID | 0536- | LRMK-ava-NKCVNFNFNGLTGTG | 0 | 0 |
| | NO: 3) | 0550 | | | |
| 386 | SPIKE (SEQ ID | 0537- | LRMK-ava-KCVNFNFNGLTGTG | | |
| | NO: 3) | 0550 | | | |
| 387 | SPIKE (SEQ ID | 0538- | LRMK-ava-CVNFNFNGLTGTG | | |
| | NO: 3) | 0550 | | | |
| 388 | SPIKE (SEQ ID | 0539- | LRMK-ava-VNFNFNGLTGTG | | |
| | NO: 3) | 0550 | | | |
| 389 | SPIKE (SEQ ID | 0539- | LRMK-ava-VNFNFNGLT | | |
| | NO: 3) | 0547 | | | |
| 390 | SPIKE (SEQ ID | 0540- | LRMK-ava-NFNFNGLTGTGVLTE | 1 | 1 |
| | NO: 3) | 0554 | | | |
| 391 | SPIKE (SEQ ID | 0541- | LRMK-ava-FNFNGLTGTGVLTE | | |
| | NO: 3) | 0554 | | | |
| 392 | SPIKE (SEQ ID | 0542- | LRMK-ava-NFNGLTGTGVLTE | | |
| | NO: 3) | 0554 | | | |
| 393 | SPIKE (SEQ ID | 0543- | LRMK-ava-FNGLTGTGVLTE | | |
| | NO: 3) | 0554 | | | |
| 394 | SPIKE (SEQ ID | 0543- | LRMK-ava-FNGLTGTGV | | |
| | NO: 3) | 0551 | | | |
| 395 | SPIKE (SEQ ID | 0761- | LRMK-ava-TQLNRALTGIAVEQ | 0 | 0 |
| | NO: 3) | 0774 | | | |
| 396 | SPIKE (SEQ ID | 0762- | LRMK-ava-QLNRALTGIAVEQ | | |
| | NO: 3) | 0774 | | | |
| 397 | SPIKE (SEQ ID | 0763- | LRMK-ava-LNRALTGIAVEQ | | |
| | NO: 3) | 0774 | | | |
| 398 | SPIKE (SEQ ID | 0763- | LRMK-ava-LNRALTGIA | | |
| | NO: 3) | 0771 | | | |
| 399 | SPIKE (SEQ ID | 0866- | LRMK-ava-TDEMIAQYTSALLAG | 0 | 0 |
| | NO: 3) | 0880 | | | |
| 400 | SPIKE (SEQ ID | 0867- | LRMK-ava-DEMIAQYTSALLAG | | |
| | NO: 3) | 0880 | | | |
| 401 | SPIKE (SEQ ID | 0868- | LRMK-ava-EMIAQYTSALLAG | | |
| | NO: 3) | 0880 | | | |
| 402 | SPIKE (SEQ ID | 0869- | LRMK-ava-MIAQYTSALLAG | | |
| | NO: 3) | 0880 | | | |
| 403 | SPIKE (SEQ ID | 0866- | LRMK-ava-MIAQYTSALL | | |
| | NO: 3) | 0878 | | | |
| 404 | SPIKE (SEQ ID | 0895- | LRMK-ava-QIPFAMQMAYRFNGIGV | 0 | 0 |
| | NO: 3) | 0911 | | | |
| 405 | SPIKE (SEQ ID | 0896- | LRMK-ava-IPFAMQMAYRFNGIGV | | |
| | NO: 3) | 0911 | | | |
| 406 | SPIKE (SEQ ID | 0897- | LRMK-ava-PFAMQMAYRFNGIGV | | |
| | NO: 3) | 0911 | | | |
| 407 | SPIKE (SEQ ID | 0898- | LRMK-ava-FAMQMAYRFNGIGV | | |
| | NO: 3) | 0911 | | | |
| 408 | SPIKE (SEQ ID | 0899- | LRMK-ava-FAMQMAYRFNG | | |
| | NO: 3) | 0908 | | | |
| 409 | SPIKE (SEQ ID | 0920- | LRMK-ava-QKLIANQFNSAIGKI | 6 | 6 |
| | NO: 3) | 0934 | | | |
| 410 | SPIKE (SEQ ID | 0921- | LRMK-ava-KLIANQFNSAIGKI | | |
| | NO: 3) | 0934 | | | |
| 411 | SPIKE (SEQ ID | 0922- | LRMK-ava-LIANQFNSAIGKI | | |
| | NO: 3) | 0934 | | | |
| 412 | SPIKE (SEQ ID | 0923- | LRMK-ava-IANQFNSAIGKI | | |
| | NO: 3) | 0934 | | | |
| 413 | SPIKE (SEQ ID | 0923- | LRMK-ava-IANQFNSAI | | |
| | NO: 3) | 0931 | | | |
| 414 | SPIKE (SEQ ID | 0924- | LRMK-ava-ANQFNSAIGKIQDSL | 1 | 0 |
| | NO: 3) | 0938 | | | |
| 415 | SPIKE (SEQ ID | 0925- | LRMK-ava-NQFNSAIGKIQDSL | | |
| | NO: 3) | 0938 | | | |
| 416 | SPIKE (SEQ ID | 0926- | LRMK-ava-QFNSAIGKIQDSL | | |
| | NO: 3) | 0938 | | | |
| 417 | SPIKE (SEQ ID | 0927- | LRMK-ava-FNSAIGKIQDSL | | |
| | NO: 3) | 0938 | | | |
| 418 | SPIKE (SEQ ID | 0924- | LRMK-ava-FNSAIGKIQ | | |
| | NO: 3) | 0935 | | | |
| 419 | SPIKE (SEQ ID | 0998- | LRMK-ava-TGRLQSLQTYVTQQL | 1 | 0 |
| | NO: 3) | 1012 | | | |
| 420 | SPIKE (SEQ ID | 0999- | LRMK-ava-GRLQSLQTYVTQQL | | |
| | NO: 3) | 1012 | | | |
| 421 | SPIKE (SEQ ID | 1000- | LRMK-ava-RLQSLQTYVTQQL | | |
| | NO: 3) | 1012 | | | |
| 422 | SPIKE (SEQ ID | 1001- | LRMK-ava-LQSLQTYVTQQL | | |
| | NO: 3) | 1012 | | | |
| 423 | SPIKE (SEQ ID | 1001- | LRMK-ava-LQSLQTYVT | | |
| | NO: 3) | 1009 | | | |
| 424 | SPIKE (SEQ ID | 1044- | LRMK-ava-GKGYHLMSFPQSAPH | 1 | 0 |
| | NO: 3) | 1058 | | | |
| 425 | SPIKE (SEQ ID | 1045- | LRMK-ava-KGYHLMSFPQSAPH | | |
| | NO: 3) | 1058 | | | |
| 426 | SPIKE (SEQ ID | 1046- | LRMK-ava-GYHLMSFPQSAPH | | |
| | NO: 3) | 1058 | | | |
| 427 | SPIKE (SEQ ID | 1047- | LRMK-ava-YHLMSFPQSAPH | | |
| | NO: 3) | 1058 | | | |
| 428 | SPIKE (SEQ ID | 1047- | LRMK-ava-YHLMSFPQS | | |
| | NO: 3) | 1055 | | | |
| 429 | SPIKE (SEQ ID | 1127- | LRMK-ava-DVVIGIVNNTVYDPL | 0 | 0 |
| | NO: 3) | 1141 | | | |
| 430 | SPIKE (SEQ ID | 1128- | LRMK-ava-VVIGIVNNTVYDPL | | |
| | NO: 3) | 1141 | | | |
| 431 | SPIKE (SEQ ID | 1129- | LRMK-ava-VIGIVNNTVYDPL | | |
| | NO: 3) | 1141 | | | |
| 432 | SPIKE (SEQ ID | 1129- | LRMK-ava-VIGIVNNTVY | | |
| | NO: 3) | 1138 | | | |
| 433 | SPIKE (SEQ ID | 1152- | LRMK-ava-LDKYFKNHTSPDVDLG | 0 | 0 |
| | NO: 3) | 1167 | | | |
| 434 | SPIKE (SEQ ID | 1153- | LRMK-ava-DKYFKNHTSPDVDLG | | |
| | NO: 3) | 1167 | | | |
| 435 | SPIKE (SEQ ID | 1154- | LRMK-ava-KYFKNHTSPDVDLG | | |
| | NO: 3) | 1167 | | | |
| 436 | SPIKE (SEQ ID | 1155- | LRMK-ava-YFKNHTSPDVDLG | | |
| | NO: 3) | 1167 | | | |
| 437 | SPIKE (SEQ ID | 1155- | LRMK-ava-YFKNHTSPDV | | |
| | NO: 3) | 1164 | | | |
| 438 | ENVELOPE (SEQ | 0001- | LRMK-ava-MYSFVSEETGTLIVN | 1 | 0 |
| | ID NO: 1) | 0015 | | | |
| 439 | MEMBRANE | 0032- | LRMK-ava-ISLLQFAYANRNRFLYI | 7 | 7 |
| | (SEQ ID NO: 2) | 0048 | | | |
| 440 | MEMBRANE | 0097- | LRMK-ava-IASFRLFARTRSMWSFN | 3 | 0 |
| | (SEQ ID NO: 2) | 0113 | | | |
| 441 | MEMBRANE | 0146- | LRMK-ava-RGHLRIAGHHLGRSD | 2 | 1 |
| | (SEQ ID NO: 2) | 0160 | | | |
| 442 | SPIKE (SEQ ID | 0025- | LRMK-ava-PPAYTNSFTRGVYY | 0 | 0 |
| | NO: 3) | 0038 | | | |
| 443 | SPIKE (SEQ ID | 0087- | LRMK-ava-NEGVYFASTEKSNIIR | 1 | 1 |
| | NO: 3) | 0102 | | | |
| 444 | SPIKE (SEQ ID | 0239- | LRMK-ava-QTLLALHRSYLTPGDSS | 4 | 3 |
| | NO: 3) | 0255 | | | |
| 445 | SPIKE (SEQ ID | 0347- | LRMK-ava-FASVYAWNRKRI [symbol] | 1 | 0 |
| | NO: 3) | 0364 | NSVAD | | |
| 446 | SPIKE (SEQ ID | 0446- | LRMK-ava- | 0 | 0 |
| | NO: 3) | 0468 | GGNYNYLYRLFRKSNLKPFERDI | | |
| 447 | SPIKE (SEQ ID | 0536- | LRMK-ava-NKSVNFNFNGLTGT | 3 | 0 |
| | NO: 3) | 0549 | | | |
| 448 | SPIKE (SEQ ID | 0797- | LRMK-ava- | 0 | 0 |
| | NO: 3) | 0816 | FGGFNFSQILPDPSKPSKRS | | |
| 449 | SPIKE (SEQ ID | 0866- | LRMK-ava-TDEMIAQYTSALLA | 0 | 0 |
| | NO: 3) | 0879 | | | |
| 450 | SPIKE (SEQ ID | 0895- | LRMK-ava-LQIPFAMQMAYRFN | 2 | 0 |
| | NO: 3) | 0911 | GIGV | | |
| 451 | SPIKE (SEQ ID | 1152- | LRMK-ava-LDKYFKNHTSPDVDL | 0 | 0 |
| | NO: 3) | 1166 | | | |
| 452 | SPIKE (SEQ ID | 1169- | LRMK-ava-ISGINASWNIQKEI | 2 | 1 |
| | NO: 3) | 1183 | | | |
| | NO: 3) | 0102 | | | |
| 457 | SPIKE (SEQ ID | 0087- | LRMK-ava-NDGVYFASTEKSNIIR | | |
| | NO: 3) | 0102 | | | |
| 458 | SPIKE (SEQ ID | 0347- | LRMK-ava-FASVYAWNRKRISNCVAD | | |
| | NO: 3) | 0364 | | | |
| 459 | SPIKE (SEQ ID | 0536- | LRMK-ava-NKCVNFNFNGLTGT | | |
| | NO: 3) | 0549 | | | |

In aspects, of the instantly-disclosed Ii-key hybrid constructs, epitopes spanning the junctions between the various elements are encompassed within the scope of the present disclosure. Thus, for example, where a claim specifies that a portion of an epitope is contained within one of the instantly-disclosed Ii-key hybrid construct peptide elements or domains (e.g., the linker region), this necessarily implies that the remaining portion is found in a contiguous portion of a flanking portion or domain.

Previous work with Ii-key demonstrated that the mammalian Ii-key peptide LRMKLPKPPKPVSKMR (SEQ ID NO: 225), and a modified mammalian Ii-key peptide, YRMKLPKPPKPVSKMR (SEQ ID NO: 227), have the ability to alter presentation of certain MHC Class II-restricted, antigenic peptides to T lymphocyte-hybridomas which recognize those respective antigenic peptides (U.S. Pat. Nos. 5,559,028; 5,919,639, the disclosures of which are incorporated herein by reference). As noted in the literature, previous experimentation with modified versions of the Ii-key peptide have indicated that a wide variety of modifications can be made to this polypeptide without detriment to activity. Indeed, modifications often enhanced antigen presentation activity of the polypeptide.

For example, U.S. Pat. No. 6,432,409 (herein incorporated by reference in its entirety), indicate that all modified Ii-key peptides which retain antigen presentation enhancing activity will function in the enhancing hybrid of the present invention when appropriately incorporated. Modifications of the Ii-key peptide include deletion of one or more amino acids from the C-terminus, protection of the N-terminus, amino acid substitutions, and introduction of cyclical peptides. Deletions of the Ii-key peptide which retain at least 4 contiguous amino acids of the original sequence, or a substituted version thereof, exhibit functional activity. Various natural or non-natural amino acids may be substituted at respective residue positions. Some examples of molecules which may be substituted are peptidomimetic structures, D-isomer amino acids, N-methyl amino acids, L-isomer amino acids, modified L-isomer amino acids, and cyclized derivatives. In addition, procedures of medicinal chemistry may be applied by one skilled in the art using routine experimental methods to obtain additional modifications of the N-terminal segment of hybrids. Examples of such procedures are methods of rational drug design, molecular modeling based on structural information from X-ray diffraction data, nuclear magnetic resonance data, and other computational methods, and screening of products of combinatorial chemical syntheses, and isolations of natural products. Examples of modified versions of Ii-key peptides which are known to retain high activity, and which may be included in the instantly-disclosed Ii-key hybrid constructs, are LRMK (SEQ ID NO: 226), LRMKLPK (SEQ ID NO: 228), LRMKLPKS (SEQ ID NO: 229), LRMKLPKSAKP (SEQ ID NO: 230), and LRMKLPKSAKPVSK (SEQ ID NO: 231). Other modifications and modified versions of the Ii-key peptide are described in U.S. Pat. Nos. 5,919,639, and 5,559,028, both of which are incorporated by reference in their entireties. A modified version of the Ii-key peptide (YRMKLPKPPKPVSKMR, SEQ ID NO: 227) which is known to retain activity is referred to herein as an ‘Ii-key homolog’. Thus, an N-terminal element of the instantly-disclosed Ii-key hybrid constructs may comprise, consist of, or consist essentially of 4-16 residues of the mammalian Ii-Key peptide LRMKLPKPPKPVSKMR (SEQ ID NO: 225) and modifications of SEQ ID No: 210 that retain antigen presentation enhancing activity (which in aspects may comprise, consist of, or consist essentially of the residues LRMK (SEQ ID NO: 226) (amino acids 1-4) of SEQ ID NO.: 225 and 0-12 additional sequential residues of SEQ ID NO.: 225, and modifications of SEQ ID No: 225 that retain antigen presentation enhancing activity; or in other aspects may comprise, consist or, or consist essentially of LRMK (SEQ ID NO: 226)

In aspects of the instantly-disclosed Ii-key hybrid constructs, the C-terminal element comprising an MHC Class II and/or Class I-presented epitope in the form of a polypeptide or peptidomimetic structure which binds to the antigenic peptide binding site of an MHC Class II and/or Class I molecule(s) comprises one or more of the instantly disclosed peptides or polypeptides. As such, in aspects, the C-terminal element comprises, consists of, or consists essentially of an amino acid sequence of SEQ ID NOS. 4-224 and 454-456 (and/or fragments or variants thereof). In further aspects, the C-terminal element comprises, consists of, or consists essentially of a peptide or polypeptide comprising, consisting, or consisting essentially of an amino acid sequence of SEQ ID NOS. 4-224 and 454-456 (and/or fragments or variants thereof), and optionally 1 to 12 additional amino acids distributed in any ratio on the N terminus and/or C-terminus of the polypeptide of SEQ ID NOS. 4-224 and 454-456)). In aspects, the C-terminal element comprises, consists of, or consists essentially of a peptide or polypeptide comprising an amino acid sequence having at least 75%, 80%, 85%, 90%, or 95% homology to any one of SEQ ID NOS: 4-224 and 454-456 (and/or fragments thereof), wherein said polypeptide is still able to bind to a same HLA molecule (i.e., retain MEW binding propensity) and/or retain the same TCR specificity, and/or retain anti-SARS-CoV-2 activity. In aspects, a poly GS linker may be include on the N terminus of the C-terminal element. As previously described, an epitope may overlap the C-terminal element and the linker element. In some circumstances it may be possible for an additional epitope or determinant to overlap between the linker element and the N-terminal Ii-key moiety. In certain aspects, the C-terminal element comprising an MEW Class II and/or MHC Class I-presented epitope in the form of a polypeptide or peptidomimetic structure which binds to the antigenic peptide binding site of an MHC Class II and/or Class I molecule(s) (which in aspects comprises one or more of the instantly disclosed peptides or polypeptides (e.g., but not limited to, a peptide or polypeptide comprising, consisting, or consisting essentially of an amino acid sequence of SEQ ID NOS. 4-224 and 454-456 (and/or fragments or variants thereof), and optionally 1 to 12 additional amino acids distributed in any ratio on the N terminus and/or C-terminus of the polypeptide of SEQ ID NOS. 4-224 and 454-456)) of the hybrid Ii-key constructs of instant disclosure can be capped with an n-terminal acetyl and/or c-terminal amino group.

In aspects, the intervening chemical structure, or spacer, may comprises one or more epitopes/determinants, the overall length within defined limits is dictated to a large extent by the identity and of the epitope/determinant. In aspects, the intervening chemical structure is antigenically neutral, and the spacer is preferably less than the length of a peptidyl backbone of 9 amino acids linearly arranged. In aspects, spacer length is the length of a peptidyl backbone of between 4 and 6 amino acids, linearly arranged. In aspects, the spacer is unable to hydrogen bond in any spatially distinct manner to other distinct elements of the enhancing hybrid peptide. In aspects, the intervening chemical structure comprises, consists of, or consists essentially of a delta-aminovaleric acid, which is 5-aminopentanoic acid (and may be referred to as “ava”). In aspects, the intervening chemical structure may comprise, consist of, or consist essentially of ava, ala-ala-ala, gly-gly, or other biologically accepted functional equivalent of ava, ala-ala-ala, and gly-gly.

In aspects, and with respect to antigenically neutral spacer elements, various chemical groups may be incorporated in the spacer segment instead of amino acids. Examples are described in U.S. Pat. No. 5,910,300, the contents of which are incorporated herein by reference. In a preferred embodiment the spacer is comprised of an aliphatic chain optimally interrupted by heteroatoms, for example a C₂-C₆ alkylene, or ═N—(CH₂)₂₋₆—N═. Alternatively, a spacer may be composed of alternating units, for example of hydrophobic, lipophilic, aliphatic and aryl-aliphatic sequences, optionally interrupted by heteroatoms such as O, N, or S. Such components of a spacer are preferably chosen from the following classes of compounds: sterols, alkyl alcohols, polyglycerides with varying alkyl functions, alkyl-phenols, alkyl-amines, amides, hydroxyphobic polyoxyalkylenes, and the like. Other examples are hydrophobic polyanhydrides, polyorthoesters, polyphosphazenes, polyhydroxy acids, polycaprolactones, polylactic, polyglycolic polyhydroxy-butyric acids. A spacer may also contain repeating short aliphatic chains, such as polypropylene, isopropylene, butylene, isobutylene, pentamethlyene, and the like, separated by oxygen atoms.

Additional peptidyl sequences which can be used in a spacer are described in U.S. Pat. No. 5,856,456, the contents of which are incorporated herein by reference. In one embodiment, the spacer has a chemical group incorporated within which is subject to cleavage. Without limitation, such a chemical group may be designed for cleavage catalyzed by a protease, by a chemical group, or by a catalytic monoclonal antibody. In the case of a protease-sensitive chemical group, tryptic targets (two amino acids with cationic side chains), chymotryptic targets (with a hydrophobic side chain), and cathepsin sensitivity (B, D or S) are favored. The term ‘tryptic target’ is used herein to describe sequences of amino acids which are recognized by trypsin and trypsin-like enzymes. The term ‘chymotryptic target’ is used herein to describe sequences of amino acids which are recognized by chymotrypsin and chymotrypsin-like enzymes. In addition, chemical targets of catalytic monoclonal antibodies, and other chemically cleaved groups are well known to persons skilled in the art of peptide synthesis, enzymatic catalysis, and organic chemistry in general, and can be designed into the hybrid structure and synthesized, using routine experimental methods.

In aspects, the intervening chemical structure, or spacer, is selected from the group consisting of: 1) an WIC Class I epitope, or a portion thereof; and 2) an antibody-recognized determinant, or a portion thereof.

In aspects, the Ii-key hybrid constructs of the present disclosure vary from totally peptide in character to substantially non-peptide in character. In view of the fact that some homologs are substantially reduced or non-peptide in character, they will be more likely to have favorable properties, for example, penetration through cellular membranes, solubility, resistance to proteolysis, resistance to inactivation by conjugation, oral bioavailability, and longer half-life in vivo, as is known in the art.

In aspects, the instant disclosure is directed to one or more hybrid Ii-key constructs of Table 2, (and/or fragments or variants thereof). In aspects, the present disclosure provides a hybrid Ii-key construct with at least 60%, 70%, 80%, 90%, or 95% homology to a Ii-key construct of Table 2 (including SEQ ID NOS: 232-452 and 457-459). In aspects, the present disclosure provides a hybrid Ii-key construct having anti-SARS-CoV-2 activity, said hybrid Ii-key construct with at least 60%, 70%, 80%, 90%, or 95% homology to a Ii-key construct of Table 2 (including SEQ ID NOS: 232-452 and 457-459.

In aspects, the instant disclosure is directed to one or more hybrid Ii-key constructs comprising, consisting, or consisting essentially of an amino acid sequence of SEQ ID NOS: 438, 237, 439, 262, 440, 441, 277, 282, 287, 442, 443, 317, 322, 327, 444, 332, 337, 342, 445, 446, 370, 375, 447, 448, 449, 450, 409, 414, 419, 424, 451, and 452 (and/or fragments or variants thereof), and optionally 1 to 12 additional amino acids distributed in any ratio on the N-terminus and/or C-terminus of the polypeptide of SEQ ID NOS: 438, 237, 439, 262, 440, 441, 277, 282, 287, 442, 443, 317, 322, 327, 444, 332, 337, 342, 445, 446, 370, 375, 447, 448, 449, 450, 409, 414, 419, 424, 451, and 452 (and in aspects, on the N-terminus and/or C-terminus of the C-terminal element of such Ii-key constructs).

In aspects, the instant disclosure is directed to one or more hybrid Ii-key constructs comprising, consisting, or consisting essentially of an amino acid sequence of SEQ ID NOS: 446, 246, 444, 440, 317, 332, 262, 370, 449, 282, 414, 287, 442, 451, 447, 419, 452, 450, 438, 277, 332, 445, 441, 443, 337, 448, 375, 409, 424, 237, 342, and 327 (and/or fragments or variants thereof), and optionally 1 to 12 additional amino acids distributed in any ratio on the N-terminus and/or C-terminus of the polypeptide of SEQ ID NOS: 446, 246, 444, 440, 317, 332, 262, 370, 449, 282, 414, 287, 442, 451, 447, 419, 452, 450, 438, 277, 332, 445, 441, 443, 337, 448, 375, 409, 424, 237, 342, and 327 (and in aspects, on the N-terminus and/or C-terminus of the C-terminal element of such Ii-key constructs).

In aspects, the instant disclosure is directed to one or more hybrid Ii-key constructs comprising, consisting, or consisting essentially of an amino acid sequence of SEQ ID NOS: 438, 237, 246, 262, 440, 441, 272, 277, 282, 287, 442, 317, 322, 327, 444, 332, 337, 338, 342, 446, 370, 375, 448, 449, 404, 409, 414, 419, 424, 451, 452, 457, 458, and 459 (and/or fragments or variants thereof), and optionally 1 to 12 additional amino acids distributed in any ratio on the N-terminus and/or C-terminus of the polypeptide of SEQ ID NOS: 438, 237, 246, 262, 440, 441, 272, 277, 282, 287, 442, 317, 322, 327, 444, 332, 337, 338, 342, 446, 370, 375, 448, 449, 404, 409, 414, 419, 424, 451, 452, 457, 458, and 459 (and in aspects, on the N-terminus and/or C-terminus of the C-terminal element of such Ii-key constructs).

As used herein, two polypeptides (or a region of the polypeptides) are substantially homologous or identical when the amino acid sequences are at least about 45-55%, typically at least about 70-75%, more typically at least about 80-85%, more typically greater than about 90%, and more typically greater than 95% or more homologous or identical. To determine the percent homology or identity of two amino acid sequences, or of two nucleic acid sequences, the sequences are aligned for optimal comparison purposes (e.g., gaps can be introduced in the sequence of one polypeptide or nucleic acid molecule for optimal alignment with the other polypeptide or nucleic acid molecule). The amino acid residues or nucleotides at corresponding amino acid positions or nucleotide positions are then compared. When a position in one sequence is occupied by the same amino acid residue or nucleotide as the corresponding position in the other sequence, then the molecules are homologous at that position. As is known in the art, the percent identity between the two sequences is a function of the number of identical positions shared by the sequences, taking into account the number of gaps, and the length of each gap, which need to be introduced for optimal alignment of the two sequences. Sequence homology for polypeptides is typically measured using sequence analysis software. As used herein, amino acid or nucleic acid “homology” is equivalent to amino acid or nucleic acid “identity”. In aspects, the percent homology between the two sequences is a function of the number of identical positions shared by the sequences (e.g., percent homology equals the number of identical positions/total number of positions×100).

In aspects, the present disclosure also encompasses polypeptides having a lower degree of identity but having sufficient similarity so as to perform one or more of the same functions performed by a polypeptide of the instant disclosure (e.g., a polypeptide having a sequence comprising, consisting of, or consisting essentially of one or more of SEQ ID NOS: 4-224 and 454-456 and/or fragments and variants thereof, and optionally 1 to 12 additional amino acids distributed in any ratio on the N-terminus and/or C-terminus of the polypeptide of SEQ ID NOS: 4-224 and 454-456; concatemeric peptides as disclosed herein; hybrid Ii-key constructs as disclosed herein, including the hybrid Ii-key constructs of SEQ ID NOS: 232-452 and 457-459, and fragments and variants thereof). Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu, Met, and Ile; interchange of the hydroxyl residues Ser and Thr, exchange of the acidic residues Asp and Glu, substitution between the amide residues Asn and Gln, exchange of the basic residues His, Lys and Arg and replacements among the aromatic residues Trp, Phe and Tyr. Guidance concerning which amino acid changes are likely to be phenotypically silent are found (Bowie J U et al., (1990), Science, 247(4948):130610, which is herein incorporated by reference in its entirety).

In aspects, a variant polypeptide can differ in amino acid sequence by one or more substitutions, deletions, insertions, inversions, fusions, and truncations or a combination of any of these. Variant polypeptides can be fully functional (e.g., retain MHC binding propensity and/or TCR specificity, and/or retain anti-SARS-CoV-2 activity) or can lack function in one or more activities. Fully functional variants typically contain only conservative variation or variation in non-critical residues or in non-critical regions; in this case, typically MHC contact residues provided MHC binding is preserved. Functional variants can also contain substitution of similar amino acids that result in no change or an insignificant change in function (e.g., retain MHC binding propensity and/or TCR specificity, and/or retain anti-SARS-CoV-2 activity). Alternatively, such substitutions can positively or negatively affect function to some degree. Non-functional variants typically contain one or more non-conservative amino acid substitutions, deletions, insertions, inversions, or truncation or a substitution, insertion, inversion, or deletion in a critical residue or critical region; in this case, typically TCR contact residues. In aspects, a variant and/or a homologous polypeptide retains the desired anti-SARS-CoV-2 activity of the instant disclosure (e.g.: capable of stimulating, inducing, and/or expanding an immune response to SARS-CoV-2 (e.g., a cellular (CD4+ and/or CD8+ T-cell response) or humoral immune response to SARS-CoV-2) and/or associated diseases in a subject; capable of stimulating, inducing, and/or expanding a SARS-CoV-2-specific IFNγ response (e.g., by lymphocytes such as PMBC, or effector CD4+ and/or CD8+ T-cells); and/or capable of inhibiting SARS-CoV-2 viral replication or infectivity, and/or capable of inducing immunity against SARS-CoV-2). Alternatively, such substitutions can positively or negatively affect function to some degree. Non-functional variants typically contain one or more non-conservative amino acid substitutions, deletions, insertions, inversions, or truncation or a substitution, insertion, inversion, or deletion in a critical residue or critical region; in this case, typically TCR contact residues.

In aspects, functional variants of a polypeptide having a sequence (or a core sequence) comprising, consisting of, or consisting essentially of one or more of SEQ ID NOS: 4-224 and 454-456 as disclosed herein may contain one or more conservative substitutions, and in aspects one or more non-conservative substitutions, at amino acid residues which are not believed to be essential for functioning (with amino acid residues considered being essential for functioning, including, e.g., e.g., retain WIC binding propensity and/or TCR specificity, and/or retain anti-SARS-CoV-2 activity) of the instantly-disclosed polypeptides. For example, in aspects, a variant polypeptide having a sequence (or a core sequence) comprising, consisting of, or consisting essentially of one or more of SEQ ID NOS: 4-224 and 454-456, or fragments thereof as disclosed herein, or a concatemeric peptide or a fragment thereof as disclosed herein, or a hybrid Ii-key construct comprising, consisting of, or consisting essentially of one or more of SEQ ID NOS: 232-452 and 457-459 as disclosed herein, may contain one or more conservative substitutions (and in aspects, a neoconservative substitution) in one or more HLA contact residues, provided HLA binding is preserved. In should be understood, that in aspects, the C-terminal element of the Ii-key constructs may contain such one or more conservative substitutions (and in aspects, a neoconservative substitution) in one or more HLA contact residues, provided HLA binding is preserved. WIC binding assays are well known in the art. In aspects, such assays may include the testing of binding affinity with respect to WIC class I and class II alleles in in vitro binding assays, with such binding assays as are known in the art. Examples include, e.g., the soluble binding assays as disclosed in U.S. Pat. No. 7,884,184 or PCT/US2020/020089, both of which are herein incorporated by reference in their entireties. Additionally, in aspects, a fully functional variant polypeptide having a sequence (or a core sequence) comprising, consisting of, or consisting essentially of one or more of SEQ ID NOS: 4-224 and 454-456 as disclosed herein do not contain mutations at one or more critical residues or regions, such as TCR contact residues.

In aspects, the TCR-binding epitope (which can be referred to as TCR binding residues, TCR facing epitope, TCR facing residues, or TCR contacts) for a 9-mer identified epitope (which may be a 9-mer fragment of one or more of SEQ ID NOS: 4-224 and 454-456 as disclosed herein or a 9-mer fragment of a concatemeric peptide, or a 9-mer fragment of a hybrid Ii-key construct of one or more of SEQ ID NOS: 232-452 and 457-459, which in aspects may be the C-terminal element of the Ii-key construct) that bind to a MHC class II molecule are at position 2, 3, 5, 7, and 8 of the identified epitope, while the MHC-binding agretope (which can be referred to as MHC contacts, MHC facing residues, MHC-binding residues, or MHC-binding face) for a 9-mer identified epitope (which may be a 9-mer fragment of one or more of SEQ ID NOS: 4-224 and 454-456 as disclosed herein, or a 9-mer fragment of a hybrid Ii-key construct of one or more of SEQ ID NOS: 232-452 and 457-459, which in aspects may be the C-terminal element of the Ii-key construct) that bind to a MHC class II molecule are at position 1, 4, 6, and 9, both as counted from the amino terminal.

In aspects, the TCR binding epitope for a 9-mer identified epitope (which may be a 9-mer fragment of one or more of SEQ ID NOS: 4-224 and 454-456 or as disclosed herein, or a 9-mer fragment of a hybrid Ii-key construct of one or more of SEQ ID NOS: 232-452 and 457-459, which in aspects may be the C-terminal element of the Ii-key construct) that binds to a MHC class I molecule are at position 4, 5, 6, 7, and 8 of the identified epitope, while the MHC binding agretope for a 9-mer identified epitope (which may be a 9-mer fragment of one or more of SEQ ID NOS: 4-224 and 454-456 as disclosed herein, or a 9-mer fragment of a hybrid Ii-key construct of one or more of SEQ ID NOS: 232-452 and 457-459, which in aspects may be the C-terminal element of the Ii-key construct) that bind to a MHC class I molecule are at position 1, 2, 3, and 9, both as counted from the amino terminal.

In aspects, the TCR binding epitope for a 10-mer identified epitope that bind to a MHC class I molecule are at position 4, 5, 6, 7, 8, and 9 of the identified epitope (which may be a 10-mer fragment of one or more of SEQ ID NOS: 4-224 and 454-456 as disclosed herein, or a 10-mer fragment of a hybrid Ii-key construct of one or more of SEQ ID NOS: 232-452 and 457-459 as disclosed herein, which in aspects may be the C-terminal element of the Ii-key construct), while the MHC binding agretope for a 10-mer identified epitope (which may be a 10-mer fragment of one or more of SEQ ID NOS: 4-224 and 454-456 as disclosed herein, or a 10-mer fragment of a hybrid Ii-key construct of one or more of SEQ ID NOS: 232-452 and 457-459 as disclosed herein, which in aspects may be the C-terminal element of the Ii-key construct) that bind to a MHC class I molecule are at position 1, 2, 3, 9, and 10, both as counted from the amino terminal.

In aspects, the TCR-binding epitope for a 9-mer identified epitope (which may be a 9-mer fragment of one or more of SEQ ID NOS: 4-224 and 454-456 as disclosed herein, or a 9-mer fragment of a hybrid Ii-key construct of one or more of SEQ ID NOS: 232-452 and 457-459 as disclosed herein, which in aspects may be the C-terminal element of the Ii-key construct) that bind to a MHC class II molecule are at any combination of residues at positions 2, 3, 5, 7, and 8 (e.g., but not limited to, positions 3, 5, 7 and 8; positions 2, 5, 7, and 8; positions 2, 3, 5, and 7, etc.) of the identified epitope, while the MHC binding agretope for a 9-mer identified epitope (which may be a 9-mer fragment of one or more of SEQ ID NOS: 4-224 and 454-456 as disclosed herein, or a 9-mer fragment of a hybrid Ii-key construct of one or more of SEQ ID NOS: 232-452 and 457-459 as disclosed herein, which in aspects may be the C-terminal element of the Ii-key construct) is the complementary face to the TCR facing residues, both as counted from the amino terminal.

In aspects, the TCR binding epitope for 9-mer identified epitope (which may be a 9-mer fragment of one or more of SEQ ID NOS: 4-224 and 454-456 as disclosed herein, or a 9-mer fragment of a hybrid Ii-key construct of one or more of SEQ ID NOS: 232-452 and 457-459 as disclosed herein, which in aspects may be the C-terminal element of the Ii-key construct) that bind to a MHC class I molecule are at positions 4, 5, 6, 7, and 8; 1, 4, 5, 6, 7 and 8; or 1, 3, 4, 5, 6, 7, and 8 of the identified epitope, while the MHC binding agretope for a 9-mer identified epitope (which may be a 9-mer fragment of one or more of SEQ ID NOS: 4-224 and 454-456 as disclosed herein, or a 9-mer fragment of a hybrid Ii-key construct of one or more of SEQ ID NOS: 232-452 and 457-459 as disclosed herein, which in aspects may be the C-terminal element of the Ii-key construct) is the complementary face to the TCR facing residues, both as counted from the amino terminal.

In aspects, the TCR-binding epitope for a 10-mer identified epitope (which may be a 10-mer fragment of one or more of SEQ ID NOS: 4-224 and 454-456 as disclosed herein, or a 10-mer fragment of a hybrid Ii-key construct of one or more of SEQ ID NOS: 232-452 and 457-459 as disclosed herein, which in aspects may be the C-terminal element of the Ii-key construct) that bind to a MHC class I molecule are at any combination of residues at positions 1, 3, 4, 5, 6, 7, 8, and 9 of the identified epitope, while the MHC binding agretope for a 10-mer identified epitope (which may be a 10-mer fragment of one or more of SEQ ID NOS: 4-224 and 454-456 as disclosed herein, or a 10-mer fragment of a hybrid Ii-key construct of one or more of SEQ ID NOS: 232-452 and 457-459 as disclosed herein, which in aspects may be the C-terminal element of the Ii-key construct) is the complementary face to the TCR facing residues, both as counted from the amino terminal.

Based on the above, it should be understood that in aspects in which one or more 9-mers and/or 10-mer epitopes are contained within a longer polypeptide and are predicted to bind one or more Class I or Class II MHC molecules and are occurring in close proximity to each other in a naturally occurring sequence (e.g., wherein position 1 of each pair of binding 9-mers and/or 10-mers fall within, e.g., 3 amino acids of each other), such epitopes may be combined to form an epitope cluster. In a given cluster, any given amino acid may be, with respect to a given 9-mer epitope or 10-mer epitope, MHC facing and, with respect to another 9-mer epitope, TCR facing.

In aspects, the present disclosure also includes fragments of the instantly-disclosed polypeptides, concatemeric polypeptides, and Ii-key constructs. In aspects, the present disclosure also encompasses fragments of the variants of the instantly-disclosed polypeptides, concatemeric polypeptides, and Ii-key constructs as described herein. In aspects, as used herein, a fragment comprises at least about nine contiguous amino acids. In aspects, the present disclosure also encompasses fragments of the variants of the T-cell epitopes described herein. Useful fragments (and fragments of the variants of the polypeptides and concatemeric polypeptides described herein) include those that retain one or more of the biological activities, particularly: MHC binding propensity and/or TCR specificity, and/or anti-SARS-CoV-2 activity. Biologically active fragments are, for example, about 9, 10, 11, 12, 1, 14, 15, 16, 20, 25, 30, 40, 50, 60, 70, 80, 90, 100 or more amino acids in length, including any value or range therebetween. Fragments can be discrete (not fused to other amino acids or polypeptides) or can be within a larger polypeptide. Several fragments can be comprised within a single larger polypeptide. In aspects, a fragment designed for expression in a host can have heterologous pre- and pro-polypeptide regions fused to the amino terminus of the polypeptide fragment and an additional region fused to the carboxyl terminus of the fragment.

In aspects, the instantly disclosed polypeptides concatemeric polypeptides, and Ii-key constructs of the present disclosure can include allelic or sequence variants (“mutants”) or analogs thereof, or can include chemical modifications (e.g., pegylation, glycosylation). In aspects, a mutant retains the same function, particularly MHC binding propensity and/or TCR specificity, and/or anti-SARS-CoV-2 activity. In aspects, a mutant can provide for enhanced binding to MHC molecules. In aspects, a mutant can lead to enhanced binding to TCRs. In another instance, a mutant can lead to a decrease in binding to MHC molecules and/or TCRs. Also contemplated is a mutant that binds, but does not allow signaling via the TCR.

The manner of producing the polypeptides of the present disclosure will vary widely, depending upon the nature of the various elements comprising the molecule. For example, an isolated polypeptide can be purified from cells that naturally express it, purified from cells that have been altered to express it (recombinant), or synthesized using known protein synthesis methods. The synthetic procedures may be selected so as to be simple, provide for high yields, and allow for a highly purified stable product. For example, polypeptides of the instant disclosure can be produced either from a nucleic acid disclosed herein, or by the use of standard molecular biology techniques, such as recombinant techniques, mutagenesis, or other known means in the art. An isolated polypeptide can be purified from cells that naturally express it, purified from cells that have been altered to express it (recombinant), or synthesized using known protein synthesis techniques. In aspects, a polypeptide of the instant disclosure is produced by recombinant DNA or RNA techniques. In aspects, a polypeptide of the instant disclosure can be produced by expression of a recombinant nucleic acid of the instant disclosure in an appropriate host cell. For example, a nucleic acid molecule encoding the polypeptide is cloned into an expression cassette or expression vector, the expression cassette or expression vector introduced into a host cell and the polypeptide expressed in the host cell. The polypeptide can then be isolated from the cells by an appropriate purification scheme using standard protein purification techniques. Alternatively a polypeptide can be produced by a combination of ex vivo procedures, such as protease digestion and purification. Further, polypeptides of the instant disclosure can be produced using site-directed mutagenesis techniques, or other mutagenesis techniques known in the art (see e.g., James A. Brannigan and Anthony J. Wilkinson, 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety).

In aspects, the present disclosure also provides chimeric or fusion polypeptide compositions. In aspects, the present disclosure is directed to a chimeric or fusion polypeptide composition (which in aspects may be isolated, synthetic, or recombinant) comprising one or more peptides, polypeptides, concatemeric polypeptides, or Ii-key constructs of the present disclosure (e.g., one or more peptides or polypeptides of the present disclosure have a sequence, e.g. but not limited to, comprising, consisting, or consisting essentially of an amino acid sequence of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 224, and similarly may have a sequence, e.g., of the concatemeric peptides or polypeptides as disclosed herein, and the Ii-key constructs as described herein, including those of Table 1 (SEQ ID NOS: 232-452 and 457-459), and variants and fragments thereof). In aspects, a chimeric or fusion polypeptide composition of the present disclosure comprises one or more peptides, polypeptides, concatemeric polypeptides, and/or Ii-key constructs of the present disclosure joined to, linked to (e.g., fused in-frame, chemically-linked, or otherwise bound), and/or inserted into a heterologous polypeptide, such as an unrelated protein. As previously described, with respect to the one or more T-cell epitopes (e.g., peptide, polypeptides, concatemeric polypeptides, or Ii-key constructs of the instant disclosure), the term “heterologous polypeptide” is intended to mean that the one or more T-cell epitopes of the instant disclosure are heterologous to, or not included naturally, in the heterologous polypeptide. In aspects, a heterologous polypeptide may include, but are not limited to, e.g. monoclonal antibody, polyclonal antibody, mouse antibody, human antibody, humanized antibody, mono specific antibody, bispecific antibody, glycosylated antibody, Fc-modified antibody, or antibody-drug conjugates; an antibody of different class or subclass (e.g., IgG (e.g., IgG1, IgG2, IgG3, IgG4), IgM, IgA, IgD or IgE molecules) or antigen-specific antibody fragments thereof (including, but not limited to, a Fab, F(ab′)2, Fv, disulfide linked Fv, scFv, single domain antibody, closed conformation multispecific antibody, disulfide-linked scFv, diabody)). In aspects, one or more of the instantly-disclosed peptides, polypeptides, concatemeric polypeptides, or Ii-key constructs may be inserted into the heterologous polypeptide (e.g., through recombinant techniques, mutagenesis, or other known means in the art), may be added to the C-terminus (with or without the use of linkers, as is known in the art), and/or added to the N-terminus (with or without the use of linkers, as is known in the art) of the heterologous polypeptide. In aspects, one or more of the instantly-disclosed polypeptides may be inserted into or replace amino acids in a Fc domain as disclosed in U.S. Pat. Nos. 7,442,778, 7,645,861, 7,655,764, 7,655,765, and/or 7,750,128 (each of which are herein incorporated by reference in their entirety). For example, protein engineering by mutagenesis can be performed using site-directed mutagenesis techniques, or other mutagenesis techniques known in the art (see e.g., James A. Brannigan and Anthony J. Wilkinson, 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety). In aspects, chimeric or fusion polypeptides comprise one or more of the instantly-disclosed peptides, polypeptides, concatemeric polypeptides, or Ii-key constructs operatively linked to a heterologous polypeptide. “Operatively linked” indicates that the one or more of the instantly-disclosed peptides, polypeptides, or concatemeric peptides and the heterologous polypeptide are fused in-frame or chemically-linked or otherwise bound. For example, in aspects, the one or more of the instantly-disclosed polypeptides may be covalently bound to one or more internal conjugation site(s) in an Fc domain as disclosed in U.S. Pat. Nos. 8,008,453, 9,114,175, and/or 10,188,740 (each of which are herein incorporated by reference in their entirety). In aspects, the one or more peptides, polypeptides, concatemeric polypeptides, or Ii-key constructs of the instant disclosure may be joined to, linked to (e.g., fused in-frame, chemically-linked, or otherwise bound), and/or inserted into a heterologous polypeptide as a whole, although it may be made up from a joined to, linked to (e.g., fused in-frame, chemically-linked, or otherwise bound), and/or inserted amino acid sequence, together with flanking amino acids of the heterologous polypeptide. In aspects, a chimeric or fusion polypeptide composition comprises a peptide, polypeptide, concatemeric peptide, or hybrid Ii-key construct of the instant disclosure wherein said one or more of peptides, polypeptides, concatemeric peptides, or hybrid Ii-key constructs is not naturally included in the heterologous polypeptide and/or said one or more of peptides, polypeptides, concatemeric peptides, or hybrid Ii-key constructs is not located at its natural position in the heterologous polypeptide. For example, in aspects, the one or more peptides, polypeptides, concatemeric peptides, and/or Ii-key constructs of the present disclosure may be inserted into a SARS-CoV-2 sequence in which the SARS-CoV-2 sequence does not include the one or more peptides, polypeptides, concatemeric peptides, and/or Ii-key constructs of the present disclosure (e.g., the SARS-CoV-2 sequence does not include, or is mutated to not include, the one or more peptides, polypeptides, concatemeric peptides, and/or Ii-key constructs of the present disclosure) or the one or more peptides, polypeptides, and/or concatemeric peptides of the present disclosure is inserted into a SARS-CoV-2 sequence but not at its natural position. In aspects, the one or more of peptide or polypeptides of the present disclosure can be joined, linked to (e.g., fused in-frame, chemically-linked, or otherwise bound), and/or inserted into the heterologous polypeptide. In aspects, chimeric or fusion polypeptide compositions comprise one or more of the instantly-disclosed T-cell epitopes (e.g., peptides, polypeptides, concatemeric polypeptides, or Ii-key constructs of the instant disclosure) operatively linked to a heterologous polypeptide having an amino acid sequence not substantially homologous to the T-cell epitope. In aspects, the chimeric or fusion polypeptide does not affect function of the T-cell epitope per se. For example, the fusion polypeptide can be a GST-fusion polypeptide in which the T-cell epitope sequences are fused to the C-terminus of the GST sequences. Other types of fusion polypeptides include, but are not limited to, enzymatic fusion polypeptides, for example beta-galactosidase fusions, yeast two-hybrid GAL fusions, poly-His fusions and Ig fusions. Such fusion polypeptides, particularly poly-His fusions or affinity tag fusions, can facilitate the purification of recombinant polypeptide. In certain host cells (e.g., mammalian host cells), expression and/or secretion of a polypeptide can be increased by using a heterologous signal sequence. Therefore, in aspects, the chimeric or fusion polypeptide contains a heterologous signal sequence at its N-terminus. In aspects of the above chimeric or fusion polypeptide compositions, the heterologous polypeptide or polypeptide comprises a biologically active molecule. In aspects, the biologically active molecule is selected from the group consisting of an immunogenic molecule, a T cell epitope, a viral protein, and a bacterial protein. In aspects, the one or more peptides, polypeptides, concatemeric peptides, or hybrid Ii-key constructs of the instant disclosure can be joined or linked to (e.g., fused in-frame, chemically-linked, or otherwise bound) to a small molecule, drug, or drug fragment. For example, the one or more peptides, polypeptides, concatemeric peptides, or hybrid Ii-key constructs of the instant disclosure can be joined or linked to (e.g., fused in-frame, chemically-linked, or otherwise bound) to an unrelated peptide or protein, a small molecule (e.g., albumin or other known carriers and proteins), drug, or drag fragment, for example but not limited to, a drug or drug fragment that is binds with high affinity to defined HLAs. In aspects of the above-described chimeric or fusion polypeptide compositions, the chimeric or fusion polypeptide compositions can be recombinant, isolated, and/or synthetic.

A chimeric or fusion polypeptide composition can be produced by standard recombinant DNA or RNA techniques as are known in the art. For example, DNA or RNA fragments coding for the different polypeptide sequences may be ligated together in-frame in accordance with conventional techniques. In another embodiment, the fusion gene can be synthesized by conventional techniques including automated DNA synthesizers. Alternatively, polymerase chain reaction (PCR) amplification of nucleic acid fragments can be carried out using anchor primers which give rise to complementary overhangs between two consecutive nucleic acid fragments which can subsequently be annealed and re-amplified to generate a chimeric nucleic acid sequence (Short Protocols in Molecular Biology: A Compendium of Methods from Current Protocols in Molecular Biology, (2ND, 1992), F M Asubel et al. (eds), Green Publication Associates, New York, N.Y. (Publ), ISBN: 9780471566355, which is herein incorporated by reference in its entirety). Further, one or more peptides, polypeptides, concatemeric polypeptides, or Ii-key constructs of the instant disclosure (e.g., but not limited to, a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 4-224 and 454-456) can be inserted into a heterologous polypeptide or inserted into a non-naturally occurring position of a polypeptide through recombinant techniques, synthetic polymerization techniques, mutagenesis, or other standard techniques known in the art. For example, protein engineering by mutagenesis can be performed using site-directed mutagenesis techniques, or other mutagenesis techniques known in the art (see e.g., James A. Brannigan and Anthony J. Wilkinson, 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety).

In aspects, the polypeptides, concatemeric polypeptides, Ii-key constructs and chimeric or fusion polypeptides can be purified to homogeneity or partially purified. It is understood, however, that preparations in which the T-cell epitope compounds and compositions are not purified to homogeneity are useful. The critical feature is that the preparation allows for the desired function of the composition, even in the presence of considerable amounts of other components. Thus, the present disclosure encompasses various degrees of purity. In one embodiment, the language “substantially free of cellular material” includes preparations of the polypeptides, concatemeric polypeptides, Ii-key constructs and chimeric or fusion polypeptides having less than about 30% (by dry weight) other proteins (e.g., contaminating protein), less than about 20% other proteins, less than about 10% other proteins, less than about 5% other proteins, less than about 4% other proteins, less than about 3% other proteins, less than about 2% other proteins, less than about 1% other proteins, or any value or range therebetween.

In aspects, when a polypeptide, concatemeric polypeptide, Ii-key construct, and chimeric or fusion polypeptide of the present disclosure is recombinantly produced, the composition can also be substantially free of culture medium, for example, culture medium represents less than about 20%, less than about 10%, or less than about 5% of the volume of the polypeptides, concatemeric polypeptides, hybrid Ii-key constructs, and chimeric or fusion polypeptides preparation. The language “substantially free of chemical precursors or other chemicals” includes preparations of the polypeptides, concatemeric polypeptides, hybrid Ii-key constructs, and chimeric or fusion polypeptides in which it is separated from chemical precursors or other chemicals that are involved in the T-cell epitope's synthesis. The language “substantially free of chemical precursors or other chemicals” can include, for example, preparations of the polypeptides, concatemeric polypeptides, hybrid Ii-key constructs, and chimeric or fusion polypeptides having less than about 30% (by dry weight) chemical precursors or other chemicals, less than about 20% chemical precursors or other chemicals, less than about 10% chemical precursors or other chemicals, less than about 5% chemical precursors or other chemicals, less than about 4% chemical precursors or other chemicals, less than about 3% chemical precursors or other chemicals, less than about 2% chemical precursors or other chemicals, or less than about 1% chemical precursors or other chemicals.

In aspects, the present disclosure also includes pharmaceutically acceptable salts of the T-cell epitope compounds and compositions (including one or more of e.g., peptides or polypeptides as disclosed herein; concatemeric peptides as disclosed herein; hybrid Ii-key constructs as described herein; chimeric or fusion polypeptide compositions as disclosed herein (which in aspects may be isolated, synthetic, and/or recombinant). “Pharmaceutically acceptable salt” means a salt that is pharmaceutically acceptable and that possesses the desired pharmacological activity of the parent peptide or polypeptide (e.g., peptides, polypeptides, concatemeric peptides, hybrid Ii-key constructs, and/or chimeric or fusion polypeptides as disclosed herein). As used herein, “pharmaceutically acceptable salt” refers to derivative of the instantly-disclosed polypeptides, concatemeric polypeptides, and/or chimeric or fusion polypeptides, wherein such compounds are modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

### Nucleic Acids

In aspects, the present disclosure also provides for nucleic acids (e.g., DNAs (including cDNA, RNAs (such as, but limited to mRNA), vectors, viruses, or hybrids thereof, all of which may be isolated, synthetic, or recombinant) that encode in whole or in part one or more one or more peptides, polypeptides, concatemeric peptides, hybrid Ii-key constructs, and/or chimeric or fusion polypeptides of the present disclosure as described herein. In aspects, the nucleic acid further comprises, or is contained within, an expression cassette, a plasmid, and expression vector, or recombinant virus, wherein optionally the nucleic acid, or the expression cassette, plasmid, expression vector, or recombinant virus is contained within a cell, optionally a human cell or a non-human cell, and optionally the cell is transformed with the nucleic acid, or the expression cassette, plasmid, expression vector, or recombinant virus. In aspects, cells are transduced, transfected, or otherwise engineered to contain within one or more of e.g., polypeptides of the present disclosure (including concatemeric polypeptides and hybrid Ii-key constructs of the present disclosure); isolated, synthetic, or recombinant nucleic acids, expression cassettes, plasmids, expression vectors, or recombinant viruses as disclosed herein; and/or isolated, synthetic, or recombinant chimeric or fusion polypeptide compositions as disclosed herein. In aspects, the cell can be a mammalian cell, bacterial cell, insect cell, or yeast cell. In aspects, the nucleic acid molecules of the present disclosure can be inserted into vectors and used, for example, as expression vectors or gene therapy vectors. Gene therapy vectors can be delivered to a subject by, e.g., intravenous injection, local administration (U.S. Pat. No. 5,328,470) or by stereotactic injection (Chen S H et al., (1994), Proc Natl Acad Sci USA, 91(8):3054-7, which are herein incorporated by reference in their entirety). Similarly, the nucleic acid molecules of the present disclosure can be inserted into plasmids. The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. Such pharmaceutical compositions can be included in a container, pack, or dispenser together with instructions for administration. In aspects of the above nucleic acids (e.g., DNAs, RNAs. vectors, viruses, or hybrids thereof) that encode in whole or in part at least one or more peptides, polypeptides, concatemeric peptides, hybrid Ii-key constructs, and/or chimeric or fusion polypeptides as described herein, the nucleic acids encode one or more peptides or polypeptides of the instant disclosure as described above (e.g., but not limited to, a peptide or polypeptide comprising, consisting, or consisting essentially of an amino acid sequence of SEQ ID NOS. 4-224 and 454-456 (and/or fragments or variants thereof), and optionally 1 to 12 additional amino acids distributed in any ratio on the N terminus and/or C-terminus of the polypeptide of SEQ ID NOS. 4-224 and 454-456; as well as Ii-key constructs as disclosed in Table 2, and include SEQ ID NOS: 232-452 and 457-459). In aspects, the present disclosure is directed to a vector comprising a nucleic acid of the present disclosure encoding one or more polypeptides of the present disclosure (including concatemeric polypeptides and hybrid Ii-key constructs of the present disclosure) or chimeric or fusion polypeptide composition of the present disclosure. In aspects, the present disclosure is directed to a cell comprising a vector of the present disclosure. In aspects, the cell can be a mammalian cell, bacterial cell, insect cell, or yeast cell.

The nucleic acid of the instant disclosure may be DNAs (including, but not limited to, cDNA) or RNAs (including, but not limited to, mRNA), single- or double-stranded. The nucleic acid is typically DNA or RNA (including mRNA). The nucleic acid may be produced by techniques well known in the art, such as synthesis, or cloning, or amplification of the sequence encoding the immunogenic polypeptide; synthesis, or cloning, or amplification of the sequence encoding the cell membrane addressing sequence; ligation of the sequences and their cloning/amplification in appropriate vectors and cells. The nucleic acids provided herein (whether RNAs, DNAs, vectors, viruses or hybrids thereof) that encode in whole or in part one or more peptides, polypeptides, concatemeric peptides, and/or chimeric or fusion polypeptides as described herein can be isolated from a variety of sources, genetically engineered, amplified, synthetically produced, and/or expressed/generated recombinantly. Recombinant polypeptides generated from these nucleic acids can be individually isolated or cloned and tested for a desired activity. Any recombinant expression system can be used, including e.g. in vitro, bacterial, fungal, mammalian, yeast, insect or plant cell expression systems. In aspects nucleic acids provided herein are synthesized in vitro by well-known chemical synthesis techniques (as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066, all of which are herein incorporated by reference in their entirety). Further, techniques for the manipulation of nucleic acids provided herein, such as, e.g., subcloning, labeling probes (e.g., random-primer labeling using Klenow polymerase, nick translation, amplification), sequencing, hybridization and the like are well described in the scientific and patent literature (see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993), all of which are herein incorporated by reference in their entirety).

A further object of the invention relates to a nucleic acid molecule encoding one or more peptides, polypeptides, concatemeric peptides, hybrid Ii-key constructs, and/or chimeric or fusion polypeptides as described herein. The nucleic acid may be used to produce the one or more peptides, polypeptides, concatemeric peptides, hybrid Ii-key constructs, and/or chimeric or fusion polypeptides as described herein in vitro or in vivo, or to produce cells expressing the polypeptide on their surface, or to produce vaccines wherein the active agent is the nucleic acid or a vector containing the nucleic acid. The nucleic acid may be, e.g., DNA, cDNA, PNA, CNA, RNA, either single- and/or double-stranded, or native or stabilized forms of polynucleotides as are known in the art.

As previously mentioned, the nucleic acid molecules according to the present disclosure may be provided in the form of a nucleic acid molecule per se such as naked nucleic acid molecules; a plasmid, a vector; virus or host cell, etc., either from prokaryotic or eukaryotic origin. Vectors include expression vectors that contain a nucleic acid molecule of the invention. An expression vector capable of expressing a polypeptide can be prepared. Expression vectors for different cell types are well known in the art and can be selected without undue experimentation. Generally, the (e.g., cDNA, or RNA, including mRNA) is inserted into an expression vector, such as a plasmid, in proper orientation and correct reading frame for expression. If necessary, the DNA (e.g., cDNA, or RNA, including mRNA) may be linked to the appropriate transcriptional and translational regulatory control nucleotide sequences recognized by the desired host (e.g., bacteria), although such controls are generally available in the expression vector. The vector is then introduced into the host bacteria for cloning using standard techniques. The vectors of the present invention may, for example, comprise a transcriptional promoter, and/or a transcriptional terminator, wherein the promoter is operably linked with the nucleic acid molecule, and wherein the nucleic acid molecule is operably linked with the transcription terminator. One or more peptides or polypeptides of the present disclosure may be encoded by a single expression vector. Such nucleic acid molecules may act as vehicles for delivering peptides/polypeptides to the subject in need thereof, in vivo, in the form of, e.g., DNA/RNA vaccines.

In aspects, the vector may be a viral vector comprising a nucleic acid as defined above. The viral vector may be derived from different types of viruses, such as, Swinepox, Fowlpox, Pseudorabies, Aujezky's virus, Salmonella, vaccinia virus, BHV (Bovine Herpes Virus), HVT (Herpes Virus of Turkey), adenovirus, TGEV (Transmissible Gastroenteritidis Coronavirus), Erythrovirus, and SIV (Simian Immunodeficiency Virus). Other expression systems and vectors may be used as well, such as plasmids that replicate and/or integrate in yeast cells.

The instant disclosure also relates to a method for preparing a peptide, polypeptide, concatemeric peptide, hybrid Ii-key constructs, and/or chimeric or fusion polypeptide of the instant disclosure, the method comprising culturing a host cell containing a nucleic acid or vector as defined above under conditions suitable for expression of the nucleic acid and recovering the peptide, polypeptide, concatemeric peptide, hybrid Ii-key constructs, and/or chimeric or fusion polypeptide. As indicated above, the proteins and peptides may be purified according to techniques known per se in the art.

### Pharmaceutical Compositions and Formulations

In aspects, the T-cell epitope compositions of the present disclosure (including one or more of e.g., polypeptides as disclosed herein; concatemeric peptides as disclosed herein; hybrid Ii-key constructs as disclosed herein; chimeric or fusion polypeptide compositions as disclosed herein; nucleic acids as disclosed herein, including nucleic acids encoding such peptides, polypeptides, concatemeric peptides, hybrid Ii-key constructs, or chimeric or fusion polypeptide compositions as disclosed herein; expression cassettes, plasmids, expression vectors, recombinant viruses, or cells as disclosed herein, and vaccines as disclosed herein; hereafter referred to as “T-cell epitope compounds and compositions of the present disclosure”) may be comprised in a pharmaceutical composition or formulation. In aspects, the instantly-disclosed pharmaceutical compositions or formulations generally comprise a T-cell epitope composition of the present disclosure and a pharmaceutically-acceptable carrier and/or excipient. In aspects, a pharmaceutical composition or formulation includes an adjuvant. In aspects, said pharmaceutical compositions are suitable for administration. Pharmaceutically-acceptable carriers and/or excipients are determined in part by the particular composition being administered, as well as by the particular method used to administer the composition. Accordingly, there is a wide variety of suitable formulations of pharmaceutical compositions for administering the instantly-disclosed T-cell epitope compositions (see, e.g., Remington's Pharmaceutical Sciences, (18′ Ed, 1990), Mack Publishing Co., Easton, Pa. Publ)). In aspects, the pharmaceutical compositions are generally formulated as sterile, substantially isotonic, and in full compliance with all Good Manufacturing Practice (GMP) regulations of the U.S. Food and Drug Administration. Pharmaceutical compositions as disclosed herein are suitable for use in stimulating, inducing, and/or expanding an immune response to a SARS-CoV-2 infection and/or related diseases caused by SARS-CoV-2, including COVID-19, in a subject, and can be used in methods of treating and/or preventing SARS-CoV-2 infection and/or related diseases caused by SARS-CoV-2, including COVID-19, in a subject, such as a human.

The terms “pharmaceutically-acceptable,” “physiologically-tolerable,” and grammatical variations thereof, as they refer to compositions, carriers, excipients, and reagents, are used interchangeably and represent that the materials are capable of administration to or upon a subject without the production of undesirable physiological effects to a degree that would prohibit administration of the composition. For example, “pharmaceutically-acceptable excipient” means, for example, an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. A person of ordinary skill in the art would be able to determine the appropriate timing, sequence and dosages of administration for particular T-cell epitope compositions of the present disclosure.

In aspects, preferred examples of such carriers or diluents include, but are not limited to, water, saline, Ringer's solutions, dextrose solution, and 5% human serum albumin. Liposomes and non-aqueous vehicles such as fixed oils can also be used. The use of such media and compounds for pharmaceutically active substances is well known in the art. Except insofar as any conventional media or compound is incompatible with the T-cell epitope compounds and compositions of the present disclosure and as previously described above, use thereof in the compositions is contemplated. Supplementary active compounds can also be incorporated into the compositions.

In aspects, T-cell epitope compounds and compositions of the present disclosure are formulated to be compatible with its intended route of administration. The T-cell epitope compounds and compositions of the present disclosure can be administered by parenteral, topical, intravenous, oral, subcutaneous, intraarterial, intradermal, transdermal, rectal, intracranial, intrathecal, intraperitoneal, intranasal; vaginally; intramuscular route or as inhalants. In aspects, T-cell epitope compounds and compositions of the present disclosure can be injected directly into a particular tissue where deposits have accumulated, e.g., intracranial injection. In other aspects, intramuscular injection or intravenous infusion may be used for administration of T-cell epitope compounds and compositions of the present disclosure. In some methods, T-cell epitope compounds and compositions of the present disclosure are administered as a sustained release composition or device, such as but not limited to a Medipad™ device. In aspects, T-cell epitope compounds and compositions of the present disclosure are administered intradermally, e.g., by using a commercial needle-free high-pressure device such as Pulse NeedleFree technology (Pulse 50™ Micro Dose Injection System, Pulse NeedleFree Systems; Lenexa, Kans., USA). In aspects, said commercial needle-free high-pressure device (e.g., Pulse NeedleFree technology) confers one or more of the following benefits: non-invasive, reduces tissue trauma, reduces pain, requires a smaller opening in the dermal layer to deposit the composition in the subject (e.g., only requires a micro skin opening), instant dispersion of the composition, better absorption of the composition, greater dermal exposure to the composition, and/or reduced risk of sharps injury.

In aspects, T-cell epitope compounds and compositions of the present disclosure can optionally be administered in combination with other agents that are at least partly effective in treating various medical conditions as described herein.

In aspects, solutions or suspensions used for parenteral, intradermal, or subcutaneous application can include, but are not limited to, the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. Examples of excipients can include starch, glucose, lactose, sucrose, gelatin, malt, rice, flour, chalk, silica gel, water, ethanol, DMSO, glycol, propylene, dried skim milk, and the like. The composition can also contain pH buffering reagents, and wetting or emulsifying agents.

In aspects, pharmaceutical compositions or formulations suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition is sterile and should be fluid to the extent that easy syringeability exists. It is stable under the conditions of manufacture and storage and is preserved against the contaminating action of microorganisms such as bacteria and fungi. In aspects, formulations including a T-cell epitope compound or composition of the present disclosure may include aggregates, fragments, breakdown products and post-translational modifications, to the extent these impurities bind HLA and present the same TCR face to cognate T cells they are expected to function in a similar fashion to pure T-cell epitopes. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be preferable to include isotonic compounds, e.g., sugars, polyalcohols such as manitol, sorbitol, sodium chloride in the composition. Prolonged absorption of the injectable compositions can be brought about by including in the composition a compound that delays absorption, e.g., aluminum monostearate and gelatin.

In aspects, sterile injectable solutions (e.g., sterile solutions suitable for injectable and/or intradermal needle-free high-pressure device) can be prepared by incorporating the T-cell epitope compounds and compositions of the present disclosure in the required amount in an appropriate solvent with one or a combination of ingredients enumerated above, as required, followed by filtered sterilization. Generally, dispersions are prepared by incorporating the binding agent into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. Further, T-cell epitope compounds and compositions of the present disclosure can be administered in the form of a depot injection or implant preparation that can be formulated in such a manner as to permit a sustained or pulsatile release of the active ingredient.

In aspects, oral compositions generally include an inert diluent or an edible carrier and can be enclosed in gelatin capsules or compressed into tablets. In aspects, for the purpose of oral therapeutic administration, the binding agent can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. In aspects, the tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel or corn starch; a lubricant such as magnesium stearate or Sterotes; a glidant such as colloidal silicon dioxide; a sweetening compound such as sucrose or saccharin; or a flavoring compound such as peppermint, methyl salicylate or orange flavoring.

For administration by inhalation, T-cell epitope compounds and compositions of the present disclosure can be delivered in the form of an aerosol spray from pressured container or dispenser that contains a suitable propellant, e.g., a gas such as carbon dioxide, or a nebulizer.

In aspects, systemic administration of the T-cell epitope compounds and compositions of the present disclosure can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the T-cell epitope compounds and compositions may be formulated into ointments, salves, gels, or creams and applied either topically or through transdermal patch technology as generally known in the art.

In aspects, the T-cell epitope compounds and compositions of the present disclosure can also be prepared in the form of suppositories (e.g., with conventional suppository bases such as cocoa butter and other glycerides) or retention enemas for rectal delivery.

In aspects, the T-cell epitope compounds and compositions of the present are prepared with carriers that protect the T-cell epitope compounds and compositions against rapid elimination from the body, such as a controlled-release formulation, including implants and microencapsulated delivery systems. Biodegradable, biocompatible polymers can be used, such as, for example, ethylene vinyl acetate, polyanhydrides, polyglycolic acid, collagen, polyorthoesters, and polylactic acid. Methods for preparation of such formulations will be apparent to those skilled in the art. The materials can also be obtained commercially, e.g., from Alza Corporation and Nova Pharmaceuticals, Inc. Liposomal suspensions (including liposomes targeted to infected cells with monoclonal antibodies to viral antigens) can also be used as pharmaceutically-acceptable carriers. These can be prepared according to methods known to those skilled in the art (U.S. Pat. No. 4,522,811, which is herein incorporated by reference in its entirety). In aspects, the T-cell epitope compounds and compositions of the present disclosure can be implanted within or linked to a biopolymer solid support that allows for the slow release of the T-cell epitope compounds and compositions to the desired site.

In aspects, it is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of binding agent calculated to produce the desired therapeutic effect in association with the required pharmaceutical carrier. The specification for the dosage unit forms of the instant disclosure are dictated by and directly dependent on the unique characteristics of the binding agent and the particular therapeutic effect to be achieved, and the limitations inherent in the art of compounding such T-cell epitope compounds and compositions for the treatment of a subject.

In aspects of a pharmaceutical composition as described herein, the composition may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the instantly-disclosed peptides or polypeptides (including concatemeric polypeptides and hybrid Ii-key constructs) or nucleic acids encoding such peptides or polypeptides (including concatemeric polypeptides and hybrid Ii-key constructs). For example, in aspects, a pharmaceutical composition can comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 peptides or polypeptides (including up to 40 peptides or polypeptides), including any value or range therebetween, comprising, consisting of, or consisting essentially of one or more peptides or polypeptides having an amino acid sequence comprising, consisting of, or consisting essentially of one or more of SEQ ID NOS: 4-224 and 454-456 and/or fragments and variants thereof, and optionally 1 to 12 additional amino acids distributed in any ratio on the N terminus and/or C-terminus of the polypeptide of SEQ ID NOS: 4-224 and 454-456; concatemeric peptides as disclosed herein; hybrid Ii-key constructs as disclosed herein, including the hybrid Ii-key constructs of SEQ ID NOS: 232-452 and 457-459 and nucleic acids (e.g., RNA mRNA, DNA, cDNA) encoding such peptides, polypeptides, or concatemeric peptides, and/or fragments and variants thereof, as described herein.

### Vaccine Compositions

The term “vaccine” as used herein includes an agent which may be used to cause, stimulate or amplify the immune system of animals (e.g., humans) against a pathogen. Vaccines of the invention are able to cause or stimulate or amplify immunity against a SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19.

The term “immunization” includes the process of delivering an immunogen to a subject. Immunization may, for example, enable a continuing high level of antibody and/or cellular response in which T-lymphocytes can kill or suppress the pathogen in the immunized animal, such as a human, which is directed against a pathogen or antigen to which the animal has been previously exposed.

Vaccines of the invention comprise an immunologically effective amount of a T-cell epitope compound or composition of the instant disclosure as described above, and in aspects in a pharmaceutically acceptable vehicle and optionally with additional excipients and/or an adjuvant. As a result of the vaccination with a composition of the present disclosure, animals, and in aspects humans, become at least partially or completely immune to SARS-CoV-2 infection and/or related diseases caused by SARS-CoV-2, including COVID-19, or resistant to developing moderate or severe SARS-CoV-2 infection and/or related diseases caused by SARS-CoV-2, including COVID-19. The instantly disclosed vaccines may be used to elicit a humoral and/or a cellular response, including CD4+ and CD8+ T effector cell responses. In aspects, an animal subject, such as a human, is protected to an extent to which one to all of the adverse physiological symptoms or effects of SARS-CoV-2 infection and/or related diseases caused by SARS-CoV-2, including COVID-19, are significantly reduced, ameliorated or totally prevented.

In practice, the exact amount required for an immunologically effective dose may vary from subject to subject depending on factors such as the age and general condition of the subject, the nature of the formulation and the mode of administration. An appropriate “effective amount” may be determined by one of ordinary skill in the art using only routine experimentation. For instance, methods are known in the art for determining or titrating suitable dosages of a vaccine to find minimal effective dosages based on the weight of the animal subject, including human subject, concentration of the vaccine and other typical factors. The dosage of the vaccines of the present disclosure will depend on the species, breed, age, size, vaccination history, and health status of the animal (e.g., swine/pig) to be vaccinated, as well as the route of administration, e.g., subcutaneous, intradermal, oral intramuscular or intravenous administration. The vaccines of the instant disclosure can be administered as single doses or in repeated doses. The vaccines of the instant disclosure can be administered alone, or can be administered simultaneously or sequentially administered with one or more further compositions, such as other porcine immunogenic or vaccine compositions. Where the compositions are administered at different times, the administrations may be separate from one another or overlapping in time. In aspects, the vaccine comprises a unitary dose of between 0.1-3000 including any value or range therebetween of polypeptide and/or nucleic acid of the instant disclosure. The dosage of the vaccine, concentration of components therein and timing of administering the vaccine, which elicit a suitable immune response, can be determined by methods such as by antibody titrations of sera, e.g., by ELISA and/or seroneutralization assay analysis and/or by vaccination challenge evaluation.

In aspects, the vaccine comprises a novel, therapeutic T cell epitope compounds or compositions as disclosed herein; and/or pharmaceutical compositions or formulations as disclosed herein) in purified form, optionally in combination with any suitable excipient, carrier, adjuvant, and/or additional protein antigen.

In another aspect, the vaccine comprises a nucleic acid as defined above, optionally in combination with any suitable excipient, carrier, adjuvant, and/or additional protein antigen. In aspects, the vaccine comprises a viral vector containing a nucleic acid as defined above. In aspects, the vaccine comprises one or more plasmid vectors.

Vaccine constructs including a T-cell epitope compound or composition of the present disclosure upon administration to a subject may initiate a strong T-cell mediated immune response but may not induce a humoral immune response. Therefore, aspects of a vaccine against SARS-CoV-2 infection and/or related diseases caused by SARS-CoV-2, including COVID-19, contains a combination of the putative T-cell epitopes together with either live attenuated virus (LAV, for example live attenuated SARS-CoV-2) or inactivated virus (for example inactivated SARS-CoV-2). This vaccine composition (including both the putative T-cell epitopes and an LAV or inactivated virus) upon administration to a subject may induce both cellular and humoral immune responses, thereby conferring comprehensive immunity against SARS-CoV-2 infection and/or related diseases caused by SARS-CoV-2, including COVID-19, in the animals, including humans.

Vaccines may comprise other ingredients, known per se by one of ordinary skill in the art, such as pharmaceutically acceptable carriers, excipients, diluents, adjuvants, freeze drying stabilizers, wetting or emulsifying agents, pH buffering agents, gelling or viscosity enhancing additives, and preservatives, depending on the route of administration.

Examples of pharmaceutically acceptable carriers, excipients or diluents include, but are not limited to demineralised or distilled water; saline solution; vegetable based oils such as peanut oil, Arachis oil, safflower oil, olive oil, cottonseed oil, maize oil, sesame oil, or coconut oil; silicone oils, including polysiloxanes, such as methyl polysiloxane, phenyl polysiloxane and methylphenyl polysolpoxane; volatile silicones; mineral oils such as light liquid paraffin oil, or heavy liquid paraffin oil; squalene; cellulose derivatives such as methylcellulose, ethylcellulose, carboxymethylcellulose, carboxymethylcellulose sodium salt, or hydroxypropyl methylcellulose; lower alkanols, for example ethanol or isopropanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrrolidone; agar; carrageenan; gum tragacanth or gum acacia; and petroleum jelly. Typically, the carrier or carriers will form from 10% to 99.9% by weight of the vaccine composition and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

Examples of adjuvants include, but are not limited to, oil in water emulsions, aluminum hydroxide (alum), immunostimulating complexes, non-ionic block polymers or copolymers, cytokines (like IL-1, IL-2, IL-7, IFN-α, IFN-β, IFN-γ, etc.), saponins, monophosphoryl lipid A (MLA), muramyl dipeptides (MDP), MCA, and the like. Other suitable adjuvants include, for example, aluminum potassium sulfate, heat-labile or heat-stable enterotoxin(s) isolated from Escherichia coli, cholera toxin or the B subunit thereof, diphtheria toxin, tetanus toxin, pertussis toxin, Freund's incomplete or complete adjuvant, etc. Toxin-based adjuvants, such as diphtheria toxin, tetanus toxin and pertussis toxin may be inactivated prior to use, for example, by treatment with formaldehyde.

Examples of freeze-drying stabilizer may be for example carbohydrates such as sorbitol, mannitol, starch, sucrose, dextran or glucose, proteins such as albumin or casein, and derivatives thereof.

Vaccines may additionally comprise at least one immunogen from at least one additional pathogen, e.g., a pig pathogen such as Actinobacillus pleuropneunomia; Adenovirus; Alphavirus such as Eastern equine encephalomyelitis viruses; Balantidium coli; Bordetella bronchiseptica; Brachyspira spp., preferably B. hyodyentheriae, B. pilosicoli, B. innocens, Brucella suis, preferably biovars 1, 2 and 3; Classical swine fever virus, Chlamydia and Chlamydophila spp., preferably C. pecorum and C. abortus; Clostridium spp., preferably Cl. difficile, Cl. perfringens types A, B and C, Cl. novyi, Cl. septicum, Cl. tetani; Digestive and respiratory Coronavirus; Cryptosporidium parvum; Eimeria spp.; Eperythrozoonis suis currently named Mycoplasma haemosuis; Erysipelothrix rhusiopathiae; Escherichia coli; Haemophilus parasuis, preferably subtypes 1, 7 and 14; Hemagglutinating encephalomyelitis virus; Isospora suis; Japanese Encephalitis virus; Lawsonia intracellulars; Leptospira spp., preferably Leptospira australis, Leptospira canicola, Leptospira grippotyphosa, Leptospira icterohaemorrhagicae, Leptospira interrogans, Leptospira pomona and Leptospira tarassovi; Mannheimia haemolytica; Mycobacterium spp., preferably M. avium, M. intracellular and M. bovis: Mycoplasma hyponeumoniae; Parvovirus; Pasteurella multocida; Porcine circovirus; Porcine cytomegolovirus; Porcine parovirus, Porcine reproductive and respiratory syndrome virus: Pseudorabies virus; Rotavirus; Sagiyama virus; Salmonella spp., preferably S. thyhimurium and S. choleraesuis; Staphylococcus spp., preferably S. hyicus; Streptococcus spp., preferably Strep suis; Swine cytomegalovirus; Swine herpes virus; Swine influenza virus; Swinepox virus; Toxoplasma gondii; Vesicular stomatitis virus and virus of exanthema of swine; or other isolates and subtypes of porcine circovirus.

The vaccine compositions of the invention may be liquid formulations such as an aqueous solution, water-in-oil or oil-in-water emulsion, syrup, an elixir, a tincture, or a preparation for parenteral, subcutaneous, intradermal, intramuscular or intravenous administration (e.g., injectable administration), such as sterile suspensions or emulsions. Such formulations are known in the art and are typically prepared by dissolution of the antigen and other typical additives in the appropriate carrier or solvent systems. Liquid formulations also may include suspensions and emulsions that contain suspending or emulsifying agents.

The route of administration can be percutaneous, via mucosal administration, or via a parenteral route (intradermal, intramuscular, subcutaneous, intravenous, or intraperitoneal). Vaccine compositions according to the present disclosure may be administered alone, or can be co-administered or sequentially administered with other treatments or therapies. A vaccine of the present disclosure can conveniently be administered intranasally, transdermally (i.e., applied on or at the skin surface for systemic absorption), parenterally, ocularly, etc. The parenteral route of administration includes, but is not limited to, intramuscular, intravenous, intradermal, and intraperitoneal routes and the like. In aspects, vaccines of the present disclosure are administered intradermally, e.g., by using a micro needle patch as is known in the art or by using a commercial needle-free high-pressure device such as Pulse NeedleFree technology (Pulse 50™ Micro Dose Injection System, Pulse NeedleFree Systems; Lenexa, Kans., USA. The present disclosure also relates to methods of immunizing or inducing an immune response in animals (e.g., humans) comprising administering to said animal a peptide, polypeptide, concatemeric peptide, hybrid-lil key constructs, chimeric or fusion polypeptide, nucleic acid, cell, vector, pharmaceutical, or vaccine as described above.

The present disclosure also relates to methods of treating and/or preventing SARS-CoV-2 infection and/or related diseases caused by SARS-CoV-2, including COVID-19, diseases in animals (e.g., humans) comprising administering to said animal a peptide, polypeptide, concatemeric peptide, hybrid Ii-key construct, chimeric or fusion polypeptide, nucleic acid, cell, vector, pharmaceutical, or vaccine as described above.

A vaccine of the present disclosure can conveniently be administered intranasally, transdermally (i.e., applied on or at the skin surface for systemic absorption), parenterally, ocularly, etc. The parenteral route of administration includes, but is not limited to, intramuscular, intravenous, and intraperitoneal routes and the like.

The dosage of the vaccines of the present disclosure will depend on the species, breed, age, size, vaccination history, and health status of the animal (e.g., human) to be vaccinated, as well as the route of administration, e.g., subcutaneous, intradermal, oral intramuscular or intravenous administration. The vaccines of the present disclosure can be administered as single doses or in repeated doses. The vaccines of the present disclosure can be administered alone, or can be administered simultaneously or sequentially administered with one or more further compositions, such as other porcine immunogenic or vaccine compositions. Where the compositions are administered at different times, the administrations may be separate from one another or overlapping in time.

In aspects, the present disclosure includes multiple rounds of administration of the instantly-disclosed vaccine compositions. For example, the vaccine can be boosted at one, two, three, and/or four week intervals. Such are known in the art to improve or boost the immune system to improve protection against the pathogen. Additionally, the present disclosure may also include assessing a subject's immune system to determine if further administrations of the instantly-disclosed vaccine compositions is warranted. In some aspects, multiple administrations may include the development of a prime boosting strategy of vaccination using the instantly-disclosed vaccines (e.g., polypeptide based or nucleic acid based as disclosed herein). Such may provide an opportunity to produce sequential immunogenic responses against SARS-CoV-2 infection and/or related diseases caused by SARS-CoV-2, including COVID-19. In some aspects, the vaccine can be boosted at 1, 2, 3, 4, 5, or 6 week intervals. In some aspects, the vaccine is boosted at 2 week intervals. In some aspects, the vaccine is boosted at 3 week intervals. In some aspects, peptide based vaccine and nucleic acid (e.g., RNA or DNA) vaccinations can be achieved in an alternative manner to provide a regimen of immunization with the same immunogen presented in different fashions to the subject's immune system.

In one aspect, the vaccine compositions of the present disclosure are administered to a subject susceptible to or otherwise at risk for 2019-nCoV infection to enhance the subject own immune response capabilities. The subject to which the vaccine is administered is, in one aspect, a human. The animal may be susceptible to infection by 2019-nCoV or a closely related virus.

In one aspect, the vaccine compositions of the present disclosure are administered to a subject susceptible to or otherwise at risk for SARS-CoV-2 infection and/or related diseases caused by SARS-CoV-2, including COVID-19, to enhance the subject own immune response capabilities. The subject to which the vaccine is administered is, in one aspect, a human. The animal may be susceptible to infection by SARS-CoV-2 infection (or a closely related virus) and/or related diseases caused by SARS-CoV-2, including COVID-19.

In aspects of a vaccine as described herein, the vaccine may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the instantly-disclosed peptides or polypeptides (including concatemeric polypeptides and/or hybrid Ii-key constructs) or nucleic acids encoding such peptides or polypeptides (including concatemeric polypeptides and/or hybrid Ii-key constructs). For example, in aspects, a vaccine can comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 peptides or polypeptides (including up to 40 peptides or polypeptides), including any value or range therebetween, comprising, consisting of, or consisting essentially of one or more peptides or polypeptides having an amino acid sequence comprising, consisting of, or consisting essentially of one or more of SEQ ID NOS: 4-224 and 454-456 and/or fragments and variants thereof, and optionally 1 to 12 additional amino acids distributed in any ratio on the N terminus and/or C-terminus of the polypeptide of SEQ ID NOS: 4-224 and 454-456; concatemeric peptides as disclosed herein; hybrid Ii-key constructs, including the hybrid Ii-key constructs of SEQ ID NOS: 232-452 and 457-459 and nucleic acids (e.g., RNA mRNA, DNA, cDNA) encoding such peptides, polypeptides, or concatemeric peptides, hybrid Ii-key constructs, and/or fragments and variants thereof, as described herein.

The present disclosure also provides a container comprising an immunologically effective amount of a polypeptide (including concatemeric polypeptides, hybrid Ii-key constructs, and chimeric or fusion polypeptides), nucleic acid or vaccine as described above. The present disclosure also provides vaccination kits comprising an optionally sterile container comprising an immunologically effective amount of the vaccine, means for administering the vaccine to animals, and optionally an instruction manual including information for the administration of the immunologically effective amount of the composition for treating and/or preventing SARS-CoV-2 infection (or a closely related virus) and/or related diseases caused by SARS-CoV-2, including COVID-19.

### Methods of Treatment

Stimulating T-cells with T-cell epitope compounds and compositions of the present disclosure can stimulate, induce, and/or expand a corresponding naturally occurring immune response, e.g., stimulating, inducing, and/or expanding corresponding naturally occurring immune response to a SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19, including COVID-19, including CD4+ and/or CD8+ T cell responses, and in aspects results in increased secretion of one or more cytokines and chemokines. In aspects, T-cells activated by the T-cell epitope compounds and compositions of the present disclosure stimulate cell-mediated immunity against SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19, in a subject.

In aspects, T cells activated by the T-cell epitope compounds and compositions of the present disclosure stimulate cell-mediated immunity against SARS-CoV-2 infection (or a closely related virus) and/or related diseases caused by SARS-CoV-2, including COVID-19 in a subject.

In aspects, the present disclosure is directed to a method of stimulating, inducing, and/or expanding an immune response, e.g., against SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19, in a subject in need thereof by administering to the subject a therapeutically effect amount of a T-cell epitope compound or composition of the present disclosure.

In aspects, the present disclosure is directed to a method of preventing, treating, or ameliorating a disease by SARS-CoV-2 infection (or a closely related virus such as Severe Acute Respiratory Syndrome (SARS) or Middle East respiratory syndrome coronavirus (MERS-CoV))) such as COVID-19, in a subject in need thereof by administering to the subject a therapeutically effect amount of a T-cell epitope compound or composition of the present disclosure.

### Assays, Methods, and Kits of the Instant Disclosure

In aspects, the present disclosure is directed to methods of measuring an immune response, including a CMI response, in a subject by incubating a sample from the subject which comprises T-cells or other cells of the immune system with one or more peptides or polypeptides (e.g., including one or more of the Ii-key constructs) of the instant disclosure. In aspects, production of IFN-γ or other cytokine or immune effector molecule(s) is then detected. The presence or level of immune effector is then indicative of the level of cell mediated responsiveness of the subject. In aspects, preferably, the sample is whole blood which is collected in a suitable container comprising the antigen. Optionally, a simple sugar such as dextrose is added to the incubation mixture. Accordingly, one aspect of the present disclosure relates to a method for measuring a CMI response in a subject, preferably a human subject and more preferably a human subject potentially infected with SARS-CoV-2 or a related coronavirus (such as Severe Acute Respiratory Syndrome (SARS) or Middle East respiratory syndrome coronavirus (MERS-CoV)), said method comprising collecting a sample from said subject wherein said sample comprises cells of the immune system which are capable of producing immune effector molecules following stimulation by an antigen, incubating said sample with one or more peptides of the instant disclosure and then measuring the presence of or elevation in the level of an immune effector molecule wherein the presence or level of said immune effector molecule is indicative of the capacity of said subject to mount a cell-mediated immune response. In aspects, the presence of or elevation in the level of an immune effector molecule wherein the presence or level of said immune effector molecule is indicative of the capacity of said subject to mount a cell-mediated immune response against SARS-CoV-2 or a related coronavirus infection.

Reference to a “subject” includes a human or non-human species including primates, livestock animals (e.g. sheep, cows, pigs, horses, donkey, goats), laboratory test animals (e.g. mice, rats, rabbits, guinea pigs, hamsters), companion animals (e.g. dogs, cats), avian species (e.g. poultry birds, aviary birds), reptiles and amphibians. The present disclosure has applicability, therefore, in human medicine as well as having livestock and veterinary and wild life applications. Most preferably, however, the subject is a human and the CMI response assay has applications in screening for responsiveness to COVID-19 or related coronavirus infections.

Reference to “immune cells” includes cells such as lymphocytes including natural killer (NK) cells, T-cells, (CD4+ and/or CD8+ cells), B-cells, macrophages and monocytes, dendritic cells or any other cell which is capable of producing an effector molecule in response to direct or indirect antigen stimulation. Conveniently, the immune cells are lymphocytes and more particularly T-lymphocytes.

The immune effector molecules may be any of a range of molecules which are produced in response to cell activation or stimulation by an antigen. Although an interferon (IFN) such as IFN-γ is a particularly useful immune effector molecule, others include a range of cytokines such as interleukins (IL), e.g. IL-2, IL-4, IL-10 or IL-12, tumor necrosis factor alpha (TNF-α), a colony stimulating factor (CSF) such as granulocyte (G)-CSF or granulocyte macrophage (GM)-CSF amongst many others such as complement or components in the complement pathway.

Accordingly, in aspects, the present disclosure provides a method for measuring a CMI response in a subject, said method comprising collecting a sample from said subject wherein said sample comprises cells of the immune system which are capable of producing IFN-γ molecules following stimulation by one or more peptides (including Ii-key constructs) of the instant disclosure, incubating said sample with one or more peptides of the instant disclosure and then measuring the presence of or elevation in the level of an IFN-γ molecule wherein the presence or level of said IFN-γ molecule is indicative of the capacity of said subject to mount a cell-mediated immune response.

The sample collected from the subject is generally deposited into a blood collection tube. A blood collection tube includes a blood draw tube or other similar vessel. Conveniently, when the sample is whole blood, the blood collection tube is heparinized. Alternatively, heparin is added to the tube after the blood is collected. Notwithstanding that whole blood is the preferred and most convenient sample, the present invention extends to other samples containing immune cells such as lymph fluid, cerebral fluid, tissue fluid and respiratory fluid including nasal and pulmonary fluid. The use of blood collection tubes is compatible with standard automated laboratory systems and these are amenable to analysis in large-scale and random access sampling. Blood collection tubes also minimize handling costs and reduce laboratory exposure to whole blood and plasma and, hence, reduce the risk of laboratory personnel from contracting a pathogenic agent. Combining the incubation step with the collection tube is particularly efficacious and enhances the sensitivity of the assay as does the optional feature of incubating the cells in the presence of a sample sugar such as dextrose.

The incubation step may be from 5 to 72 hours, more preferably 5 to 40 hours and even more preferably 8 to 24 hours or any value or range therebetween. In aspects, the incubation step is conducted in the presence of a simple sugar such as dextrose.

Detection of the immune effector molecules may be made at the protein or nucleic acid levels. Consequently, reference to “presence or level of said immune effector molecule” includes direct and indirect data. For example, high levels of IFN-γ mRNA is indirect data showing increased levels of IFN-γ.

Ligands to the immune effectors are particularly useful in detecting and/or quantitating these molecules. Antibodies to the immune effectors are particularly useful. Techniques for the assays contemplated herein are known in the art and include, for example, sandwich assays, ELISA and ELISpot. Reference to “antibodies” includes parts of antibodies, mammalianized (e.g. humanized) antibodies, recombinant or synthetic antibodies and hybrid and single chain antibodies. It should be understood that a wide range of immunoassay techniques as are known in the art are compatible with the instant disclosure, such as those disclosed in U.S. Pat. No. 7,608,392 (herein incorporated by reference in its entirety).

In aspects, the present disclosure is directed to methods of assaying for SARS-CoV-2 or a related coronavirus (such as Severe Acute Respiratory Syndrome (SARS) or Middle East respiratory syndrome coronavirus (MERS-CoV)) peptide-specific T-cells, the method comprising providing a fluid containing T-cells, adding one or more peptides or polypeptides of the instant disclosure (e.g., including one or more of the Ii-key constructs) to the fluid, incubating the fluid to cause cytokine release, and detecting the released cytokine. Preferably the method comprises providing the fluid containing T-cells in contact with a surface carrying an immobilized first antibody to the cytokine, adding the peptide to the fluid, incubating the resulting fluid mixture under conditions to cause any peptide-specific T-cells that have been pre-sensitized in vivo to the peptide to secrete the cytokine, and detecting any secreted cytokine bound to the immobilized first antibody.

In aspects, the cells are preferably peripheral blood mononuclear cells (PMBC). They may suitably be taken from a patient known to be suffering, or to have suffered, from COVID-19 infection or a related coronavirus infection. In aspects, the cells used are fresh. In aspects, the assay is used to identify or quantitate peptide-specific T-cells e.g. CD8+ or CD4+ cells that have been activated or pre-sensitized in vivo to a particular peptide. In aspects, these are unrestimulated T-cells, i.e. cells capable of immediate effector function without the need to effect division/differentiation by in vitro culture. When a peptide in question (e.g., including one or more of the Ii-key constructs) is presented to such cells, the cells secrete various cytokines, of which any one may be selected for the purposes of this assay. In aspects, the cytokine selected is interferon-γ (IFN γ). However, although an interferon (IFN) such as IFN-γ is a particularly useful immune effector molecule, others include a range of cytokines such as interleukins (IL), e.g. IL-2, IL-4, IL-10 or IL-12, tumor necrosis factor alpha (TNF-α), a colony stimulating factor (CSF) such as granulocyte (G)-CSF or granulocyte macrophage (GM)-CSF amongst many others.

The secreted cytokine can be detected by any of a variety of methods known in the literature. Preferably the assay method involves providing a surface carrying an immobilized first antibody to the IFN-γ or other cytokine. A fluid containing the PBMC or other fresh cells is placed in contact with that immobilized antibody. About 30% of the PBMC are CD8+ cells.

In aspects, the method comprises adding a peptide or polypeptide of the instant disclosure (e.g., including one or more of the Ii-key constructs) to the fluid. If activated or pre-sensitized peptide-specific T-cells (CD4+ and/or CD8+ T cells) are present in the test fluid, they respond by secreting appropriate effector cytokines, such as IFN-γ or other cytokine, which then becomes bound to the immobilized antibody. In aspects, the one or more peptides or polypeptides of the instant disclosure may be added in uncombined form to the fresh cells. While it is possible to add cultured cells that have been pulsed with such peptides or polypeptides, this is not necessary when using defined peptide/polypeptide epitopes. The peptides/polypeptides should be added in an amount sufficient to generate an observable signal; in aspects a preferred concentration range in the fluid is 0.01 up to 100 μM particularly 0.5-5.0 μM.

Incubation should be continued for a time sufficient to permit CD8+ and/or CD4+ cells that have been pre-sensitized in vivo to the particular peptide/polypeptide (e.g., including one or more of the Ii-key constructs) chosen to secrete the IFN-γ or other cytokine. In aspects, the incubation should not continue for so long that quiescent CD8+ and/or CD4+ cells have time to differentiate and become activated by the peptide and start to secrete cytokines. This suggests an incubation time of 4-24 hours, more particularly 6-16 hours. It is an advantage of the invention that the incubation part of the test can be performed in a single working day or overnight, and without the use of sterile conditions required for cell culture in vitro.

In aspects, during the incubation, any IFN-γ or other cytokine secreted by CD8+ and/or CD4+ cells becomes bound to the first antibody immobilized on the surface. After incubation, the surface may be washed to remove unbound material. For detection, in aspects a labelled second antibody to the cytokine is used. When this is applied to the surface it becomes bound to any cytokine present. In aspects, the second antibody should recognize a different epitope from the first antibody. In aspects, one or both of the first and second antibodies may be monoclonal. The label may be any that is conventionally used in the field, including radioisotopes, enzymes to generate color or chemiluminescence, fluorescent groups or groups for detection by mass spectrometry or refractive index (e.g. by surface plasmon resonance). It is convenient but not necessary to use a labelled antibody, any reagent that binds specifically to the cytokine could be labelled and used. Detection and perhaps quantitation of the label is effected by means well known in the field and appropriate to the nature of the label used, and may be those as disclosed in U.S. Pat. No. 7,608,392 (herein incorporated by reference in its entirety).

In aspects, the assay may conveniently be carried out in a multi-well plate. Each well of the plate has a surface carrying a bound first antibody. To each well is added a fluid containing an appropriate number, e.g. 103-106 of cells. Different peptides and/or controls are added to individual wells of the plate. Cells that secrete a cytokine during incubation show up as spots (spot forming cells or SFCs) and the number or density of these in each well can readily be determined.

In aspects, the present disclosure provides a method of detecting an anti-SARS-CoV-2 (or related coronavirus, such as Severe Acute Respiratory Syndrome (SARS) or Middle East respiratory syndrome coronavirus (MERS-CoV)) T cell response (which in aspects can included CD4+ and/or CD8+ T cell response) comprising contacting a population of T cells of an individual with a peptide (e.g., including one or more of the Ii-key constructs) of the instant disclosure, wherein one or more of said peptides may be substituted by an analogue which binds a T cell receptor that recognizes the peptide, and determining whether T cells of the T cell population recognize the peptide(s). Further, in aspects, the present disclosure provides a method of diagnosing a SARS-CoV-2 or related coronavirus infection (such as Severe Acute Respiratory Syndrome (SARS) or Middle East respiratory syndrome coronavirus (MERS-CoV)) and/or related diseases caused by SARS-CoV-2, including COVID-19, in a host, or exposure of a host, to SARS-CoV-2 or related coronavirus, the method comprising (i) contacting a population of T cells from the host with one or more peptides or analogues as disclosed here, and analogues thereof which can bind a T cell receptor which recognizes any of the said peptides; and (ii) determining whether the T cells of said T cell population recognize the peptide(s) and/or analogue(s).

In aspects, the host is generally a human but may be an animal, typically one which can be naturally or artificially infected by a Mycobacterium. The host may be a mammal, such as a primate, cow, sheep, pig, badger or rodent, e.g. a mouse or rat. The host typically has an active or latent SARS-CoV-2 infection or related coronavirus infection, or has had such an infection recently. The host may be a healthy contact who has been exposed to SARS-CoV-2 infection or related coronavirus infection. Thus the method may be used to trace the healthy contacts of individuals with such SARS-CoV-2 infection or related coronavirus infection. The method may also be used to carry out population surveys to measure the number of individuals in a population who have a SARS-CoV-2 infection or related coronavirus infection or are healthy contacts.

In aspects, the T cells which recognize the peptide (e.g., including one or more of the Ii-key constructs) in the method are generally T cells which have been pre-sensitized in vivo to antigen from SARS-CoV-2 or related coronavirus. These antigen-experienced T cells are generally present in the peripheral blood of a host which has been exposed to the SARS-CoV-2 infection or related coronavirus infection. The T cells may be CD4+ and/or CD8+ T cells.

It is understood that the term ‘peptide’ or ‘polypeptide’ also includes the analogue of that peptide (which may not be a peptide as defined by the ordinary use of the term) unless the context requires otherwise.

In aspects, the T cells can be contacted with the peptides (e.g., including one or more of the Ii-key constructs) in vitro or in vivo, and determining whether the T cells recognize the peptide can be done in vitro or in vivo. In aspects, determination of whether the T cells recognize the peptide or polypeptide of the instant disclosure is generally done by detecting a change in the state of the T cells in the presence of the peptide or polypeptide or determining whether the T cells bind the peptide or polypeptide. The change in state is generally caused by antigen specific functional activity of the T cell after the T cell receptor binds the peptide or polypeptide. Generally when binding the T cell receptor the peptide is bound to an MHC class II or MHC class I molecule, which is typically present on the surface of an antigen presenting cell (APC).

In aspects, the change in state of the T cell may be the start of or increase in secretion of a substance from the T cell, such as a cytokine, especially IFN-γ, IL-2 or TNF-α. In aspects, the cytokine selected is interferon-γ (IFN γ). However, although an interferon (IFN) such as IFN-γ is a particularly useful immune effector molecule, others include a range of cytokines such as interleukins (IL), e.g. IL-2, IL-4, IL-10 or IL-12, tumor necrosis factor alpha (TNF-α), a colony stimulating factor (CSF) such as granulocyte (G)-CSF or granulocyte macrophage (GM)-CSF amongst many others. The substance can typically be detected by allowing it to bind to a specific binding agent and then measuring the presence of the specific binding agent/substance complex. The specific binding agent is typically an antibody, such as polyclonal or monoclonal antibodies. Antibodies to cytokines are commercially available, or can be made using standard techniques.

In aspects, the specific binding agent is immobilized on a solid support. After the substance is allowed to bind the solid support can optionally be washed to remove material which is not specifically bound to the agent. In aspects, the agent/substance complex may be detected by using a second binding agent which will bind the complex. Typically the second agent binds the substance at a site which is different from the site which binds the first agent. In aspects, the second agent is preferably an antibody and is labelled directly or indirectly by a detectable label.

Thus the second agent may be detected by a third agent which is typically labelled directly or indirectly by a detectable label. For example the second agent may comprise a biotin moiety, allowing detection by a third agent which comprises a streptavidin moiety and typically alkaline phosphatase as a detectable label.

In aspects the detection system which is used is the ex-vivo ELISPOT assay as is known in the art. In such an assay, IFN-γ secreted from the T cell is bound by a first IFN-γ specific antibody which is immobilized on a solid support. The bound IFN-γ is then detected using a second IFN-γ specific antibody which is labelled with a detectable label.

In aspects, the change in state of the T cell which can be measured may be the increase in the uptake of substances by the T cell, such as the uptake of thymidine. The change in state may be an increase in the size of the T cells, or proliferation of the T cells, or a change in cell surface markers on the T cell.

In aspects, the T cells which are contacted in the assay/methods are taken from the host in a blood sample, although other types of samples which contain T cells can be used. The sample may be added directly to the assay or may be processed first. Typically the processing may comprise diluting of the sample, for example with water or buffer. Typically the sample is diluted from 1.5 to 100 fold, for example 2 to 50 or 5 to 10 fold. In aspects, the processing may comprise separation of components of the sample. Typically mononuclear cells (MCs) are separated from the samples. The MCs will comprise the T cells and APCs. Thus in the method the APCs present in the separated MCs can present the peptide to the T cells. In aspects, only T cells, such as only CD4+ and/or only CD8+ T cells, can be purified from the sample. PBMCs, MCs and T cells can be separated from the sample using techniques known in the art.

In aspects, the T cells used in the assay/methods are in the form of unprocessed or diluted samples, or are freshly isolated T cells (such as in the form of freshly isolated MCs or PBMCs) which are used directly ex vivo, i.e. they are not cultured before being used in the method. However the T cells can be cultured before use, for example in the presence of one or more of the peptides (e.g., including one or more of the Ii-key constructs), and generally also exogenous growth promoting cytokines. During culturing the peptides are typically present on the surface of APCs, such as the APC used in the method. Pre-culturing of the T cells may lead to an increase in the sensitivity of the method. Thus the T cells can be converted into cell lines, such as short term cell lines.

In aspects, the APC which is typically present in the assays/methods may from the same host as the T cell or from a different host. In aspects, the APC may be a naturally occurring APC or an artificial APC. In aspects, the APC is a cell which is capable of presenting the peptide to a T cell. It is typically a B cell, dendritic cell or macrophage. It is typically separated from the same sample as the T cell and is typically co-purified with the T cell. Thus the APC may be present in MCs or PBMCs. In aspects, the APC is typically a freshly isolated ex vivo cell or a cultured cell. It may be in the form of a cell line, such as a short term or immortalized cell line. The APC may express empty MHC class II molecules or MHC class I molecules on its surface.

In aspects of the assays/methods, the T cells derived from the sample can be placed into an assay with all the peptides (i.e. a pool of the peptides, which may include, e.g., one or more of the Ii-key constructs)) which it is intended to test (the relevant panel) or the T cells can be divided and placed into separate assays each of which contain one or more of the peptides. In aspects, in the in vitro or in vivo forms of the methods/assays at least one or more of the instantly disclosed peptides/polypeptides as described herein or analogues thereof are used.

In aspects, one or more peptides or polypeptides (e.g., including one or more of the Ii-key constructs) as disclosed herein is added directly to an assay comprising T cells and APCs. As discussed above the T cells and APCs in such an assay could be in the form of MCs. When peptides which can be recognized by the T cell without the need for presentation by APCs are used then APCs are not required. Analogues which mimic the original peptide bound to a MHC molecule are an example of such a peptide. In aspects, the peptide or polypeptide is provided to the APC in the absence of the T cell. The APC is then provided to the T cell, typically after being allowed to present the peptide on its surface. The peptide may have been taken up inside the APC and presented, or simply be taken up onto the surface without entering inside the APC.

In aspects, the duration for which the peptide or polypeptide (e.g., including one or more of the Ii-key constructs) is contacted with the T cells will vary depending on the method used for determining recognition of the peptide. Typically 10⁵ to 10⁷, preferably 5×10⁵ to 10⁶ PBMCs are added to each assay. In the case where a peptide is added directly to the assay its concentration is from 10⁻¹ to 10³ μg/ml, preferably 0.5 to 50 g/ml or 1 to 10 μg/ml. In aspects, the length of time for which the T cells are incubated with a peptide or polypeptide is from 4 to 24 hours, preferably 6 to 16 hours.

In aspects, the determination of the recognition of a peptide or polypeptide (e.g., including one or more of the Ii-key constructs) of the instant disclosure by the T cells may be done by measuring the binding of the peptide to the T cells. Typically T cells which bind the peptide can be sorted based on this binding, for example using a FACS machine. The presence of T cells which recognize the peptide will be deemed to occur if the frequency of cells sorted using the peptide is above a ‘control’ value. The frequency of antigen-experienced T cells is generally 1 in 10⁶ to 1 in 10³, and therefore whether or not the sorted cells are antigen-experienced T cells can be determined.

In aspects, the determination of the recognition of the peptide (e.g., including one or more of the Ii-key constructs) by the T cells may be measured in vivo. In aspects, a peptide or polypeptide of the instant disclosure is administered to the host and then a response which indicates recognition of the peptide or polypeptide may be measured. In aspects, the peptide is administered intradermally, typically in a similar manner to the Mantoux test. In aspects, the peptide may be administered epidermally. In aspects, peptide is administered by needle, such as by injection, but can be administered by other methods such as ballistics, for example the ballistics techniques which have been used to deliver nucleic acids. EP-A-0693119 describes techniques which can typically be used to administer the peptide. In aspects, from 0.001 to 1000 for example from 0.01 to 100 μg or 0.1 to 10 μg of peptide is administered.

Alternatively an agent can be administered which is capable of providing the peptides (e.g., including one or more of the Ii-key constructs) in vivo. Thus a polynucleotide capable of expressing the peptide can be administered, typically in any of the ways described above for the administration of the peptide. In aspects, the polynucleotide has any of the characteristics of the polynucleotide provided by the invention which is discussed below. In aspects, the peptide is expressed from the polynucleotide in vivo and recognition of the peptide in vivo is measured. In aspects, from 0.001 to 1000 for example from 0.01 to 100 m or 0.1 to 10 μg of polynucleotide is administered. Recognition of the peptide in vivo is typically indicated by the occurrence of a response.

The analogue which can be used in the assays/methods can bind to a T cell receptor which recognizes the equivalent peptide or polypeptide (e.g., including one or more of the Ii-key constructs) of the instant disclosure. Therefore generally when the analogue is added to T cells in the presence of the equivalent said peptide or polypeptide, typically also in the presence of an APC, the analogue inhibits the recognition of the equivalent peptide or polypeptide. In aspects, the binding of the analogue to the said T cell receptors can be tested by standard techniques. For example T cell receptors can be isolated from T cells which have been shown to recognize the peptide or polypeptide (e.g. using the assays/methods of the instant disclosure). In aspects, demonstration of the binding of the analogue to the T cell receptors can then shown by determining whether the T cell receptors inhibit the binding of the analogue to a substance that binds the analogue, e.g. an antibody to the analogue. In aspects, the analogue is bound in an MHC molecule in such an inhibition of binding assay.

In aspects, the analogue inhibits the binding of the peptide (e.g., including one or more of the Ii-key constructs) to a T cell receptor. In this case the amount of peptide which can bind the T cell receptor in the presence of the analogue is decreased. This is because the analogue is able to bind the T cell receptor and therefore competes with the peptide for binding to the T cell receptor.

T cells for use in the above binding experiments can be isolated from patients with COVID-19 or related coronavirus infection, for example with the aid of the method of the instant disclosure.

Other binding characteristics of the analogue are also the same as the corresponding peptide or polypeptide (e.g., including one or more of the Ii-key constructs) of the instant disclosure, and thus typically the analogue binds to the same MHC class II molecule or MHC class I molecule which the peptide or polypeptide of the instant disclosure binds.

The analogue is typically a peptide or polypeptide. It may have homology with the equivalent original peptide or polypeptide (e.g., including one or more of the Ii-key constructs) of the instant disclosure. A peptide or polypeptide which is homologous to another peptide or polypeptide is typically at least 70% homologous to the peptide, preferably at least 80 or 90% and more preferably at least 95%, 97% or 99% homologous thereto, for example over a region of at least 15, preferably at least 30, for instance at least 40, 60 or 100 or more contiguous amino acids. Methods of measuring protein homology are well known in the art and it will be understood by those of skill in the art that in the present context, homology is calculated on the basis of amino acid identity (sometimes referred to as “hard homology”). For example the UWGCG Package provides the BESTFIT program which can be used to calculate homology (for example used on its default settings) (Devereux et al (1984) Nucleic Acids Research 12, p 387-395).

The homologous peptides or polypeptides may differ by substitution, insertion or deletion, for example from 1, 2, 3, 4, 5, 6, 7, 8 or more substitutions, deletions or insertions, which can be at the N or C terminal or at any other position in the sequence. The substitutions are preferably conservative. Typically seen as conservative substitutions are the replacements, one for another, among the aliphatic amino acids Ala, Val, Leu, Met, and Ile; interchange of the hydroxyl residues Ser and Thr, exchange of the acidic residues Asp and Glu, substitution between the amide residues Asn and Gln, exchange of the basic residues His, Lys and Arg and replacements among the aromatic residues Trp, Phe and Tyr.

The analogue is typically from 8 to 80 amino acids in length, such as 10 to 60 or 12 to 50, preferably 15 to 30 or 20 to 25. In aspects, the amino acids in the analogue at the equivalent positions to amino acids in the original peptide or polypeptide which contribute to binding the MHC molecule or are responsible for the recognition by the T cell receptor, are the same or are conserved.

In aspects, the analogue peptide comprises one or more modifications, which may be natural post-translation modifications or artificial modifications. The modification may provide a chemical moiety (typically by substitution of a hydrogen, e.g. of a C—H bond), such as an amino, acetyl, hydroxy or halogen (e.g. fluorine) group or carbohydrate group. In aspects, the modification is present on the N or C terminus. In aspects, the analogue may comprise one or more non-natural amino acids, for example amino acids with a side chain different from natural amino acids. Generally, the non-natural amino acid will have an N terminus and/or a C terminus. The non-natural amino acid may be an L-amino acid. In aspects, the analogue has a shape, size, flexibility or electronic configuration which is substantially similar to the original peptide or polypeptide. It is typically a derivative of the original peptide or polypeptide.

In aspects, the analogue is or mimics the original peptide bound to a MHC class II molecule or a MHC class I molecule. In aspects, the analogue may be or may mimic the original peptide bound to 2, 3, 4 or more MHC class II molecules or MHC class I molecules associated or bound to each other. In aspects, these MHC molecules may be bound together using a biotin/streptavidin based system, in which typically 2, 3 or 4 biotin labelled MHC molecules bind to a streptavidin moiety. This analogue typically inhibits the binding of the peptides or polypeptides (e.g., including one or more of the Ii-key constructs). In aspects, class II or class I complex to a T cell receptor or antibody which is specific for the complex. In aspects, the analogue is an antibody or a fragment of an antibody, such as a Fab or (Fab)2 fragment.

In aspects, the analogue may be immobilized on a solid support, particularly an analogue which mimics peptide bound to a MHC molecule.

In aspects, the analogue is designed by computational means and then synthesized using methods known in the art. Alternatively the analogue can be selected from a library of compound. The library may be a combinatorial library or a display library, such as a phage display library. The library of compounds may be expressed in the display library in the form of being bound to a MHC class II molecule or MHC class I molecule, such as the MHC molecule which the original peptide binds. Analogues are generally selected from the library based on their ability to mimic the binding characteristics of the original peptides. Thus they may be selected based on ability to bind a T cell receptor or antibody which recognizes the original peptide.

The present disclosure also provides a kit for carrying out the above-methods and assays comprising one or more of the peptides (e.g., including one or more of the Ii-key constructs) or analogues as disclosed herein, and optionally a means to detect the recognition of the peptide by the cells of the immune system, such as T cells. In aspects, the means to detect recognition allows or aids detection based on the techniques discussed above, however other detection means in the art may used, such as those disclosed in U.S. Pat. No. 7,608,392 (herein incorporated by reference in its entirety). Thus the means may allow detection of a substance secreted by the T cells after recognition. The kit may thus additionally include a specific binding agent for the substance, such as an antibody. In aspects, the agent is specific for IFN-γ, however agents listed for other cytokines as described above may be used. In aspects, the agent is immobilized on a solid support, which means that after binding the agent the substance will remain in the vicinity of the T cell which secreted it. Thus ‘spots’ of substance/agent complex are formed on the support, each spot representing a T cell which is secreting the substance. Quantifying the spots, and typically comparing against a control, allows determination of recognition of the peptide.

In aspects, the kit may also comprise a means to detect the substance/agent complex. A detectable change may occur in the agent itself after binding the substance, such as a color change. Alternatively a second agent directly or indirectly labelled for detection may be allowed to bind the substance/agent complex to allow the determination of the spots. As discussed above the second agent may be specific for the substance, but binds a different site on the substance than the first agent. In aspects, the means to detect recognition allows or aids detection based on the techniques discussed above, however other detection means in the art may be used, such as those disclosed in U.S. Pat. No. 7,608,392 (herein incorporated by reference in its entirety). In aspects, the immobilized support may be a plate with wells, such as a microtitre plate. Each assay can therefore be carried out in a separate well in the plate.

In aspects, the kit may additionally comprise medium for the cells of the immune system, such as T cells, detection agents, and/or washing buffers to be used in the detection steps. In aspects, the kit may additionally comprise reagents suitable for the separation from the sample, such as the separation of PBMCs or T cells from the sample. In aspects, the kit may be designed to allow detection of the T cells directly in the sample without requiring any separation of the components of the sample.

In aspects, the kit may comprise an instrument which allows administration of the peptide (e.g., including one or more of the Ii-key constructs), such as intradermal or epidermal administration. Typically such an instrument comprises one or more needles. The instrument may allow ballistic delivery of the peptide. The peptides or polypeptides in the kit may be in the form of a pharmaceutical composition.

In aspects, the kit may also comprise controls, such as positive or negative controls. In aspects, the positive control may allow the detection system to be tested. Thus the positive control typically mimics recognition of the peptides or polypeptides (e.g., including one or more of the Ii-key constructs) in any of the above assays or methods. In aspects of the kit designed to determine recognition in vitro the positive control is a cytokine. In aspects of the kit designed to detect in vivo recognition of the peptide the positive control may be antigen to which most individuals should response.

In aspects, the kit may also comprise a means to take a sample containing immune cells, such as T cells, from the host/subject, such as a blood sample. In aspects, the kit may comprise a means to separate mononuclear cells or T cells from a sample from the host. In aspect, the kit is conveniently in compartmental form with one or more compartments adapted to receive a sample from a subject such as whole blood. That compartment or another compartment may also be adapted to contain heparin where the sample is whole blood with or without a simple sugar such as dextrose. The simple sugar may also be maintained in a separate container.

In aspects, the kit is in a form which is packaged for sale with a set of instructions. The instructions would generally be in the form to conduct the assays and/or methods as disclosed herein.

Although any assay, methods, and materials similar or equivalent to those described herein can be used in the practice or testing of the present disclosure, the preferred methods and materials are described. Other features, objects, and advantages of the present disclosure will be apparent from the description and the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All references cited herein are incorporated herein by reference in their entirety and for all purposes to the same extent as if each individual publication, patent, or patent application was specifically and individually indicated to be incorporated by reference in its entirety for all purposes.

### Aspects

A 1st aspect is directed to a polypeptide consisting of an amino acid sequence selected from the group consisting of SEQ ID NOS: 4-224 and 454-456, and/or fragments and variants thereof, and optionally 1 to 12 additional amino acids distributed in any ratio on the N terminus and/or C-terminus of the polypeptide of SEQ ID NOS: 4-224 and 454-456.

A 2nd aspect is directed to a polypeptide consisting essentially of an amino acid sequence selected from the group consisting of SEQ ID NOS: 4-224 and 454-456, and/or fragments and variants thereof, and optionally 1 to 12 additional amino acids distributed in any ratio on the N terminus and/or C-terminus of the polypeptide of SEQ ID NOS: 4-224 and 454-456.

A 3rd aspect is directed to a polypeptide comprising an amino acid sequence selected from the group consisting of SEQ ID NOS: 4-224 and 454-456, and/or fragments and variants thereof, and optionally 1 to 12 additional amino acids distributed in any ratio on the N terminus and/or C-terminus of the polypeptide of SEQ ID NOS: 4-224 and 454-456.

A 4th aspect is directed to a polypeptide according to any one of aspects 1-3, wherein said variant or fragment of an amino acid sequence selected from the group consisting of SEQ ID NOS: 4-224 and 454-456 retains MHC binding propensity and TCR specificity, and/or retains anti-SARS-CoV-2 activity.

A 5^{th} aspect is directed to a polypeptide consisting of an amino acid sequence having at least 75%, 80%, 85%, 90%, or 95% homology to any one of SEQ ID NOS: 4-224 and 454-456, and fragments thereof, wherein said polypeptide retains MHC binding propensity and the same TCR specificity, and/or retains anti-SARS-CoV-2 activity.

A 6^{th} aspect is directed to a polypeptide consisting essentially of an amino acid sequence having at least 75%, 80%, 85%, 90%, or 95% homology to any one of SEQ ID NOS: 4-224 and 454-456, and fragments thereof, wherein said polypeptide retains MHC binding propensity and the same TCR specificity, and/or retains anti-SARS-CoV-2 activity.

A 7^{th} aspect is directed to a polypeptide comprising an amino acid sequence having at least 75%, 80%, 85%, 90%, or 95% homology to any one of SEQ ID NOS: 4-224 and 454-456, and fragments thereof, wherein said polypeptide retains MHC binding propensity and the same TCR specificity, and/or retains anti-SARS-CoV-2 activity.

An 8^{th} aspect is directed to a polypeptide comprising an amino acid sequence selected from the group consisting of SEQ ID NOS: 232-452 and 457-459, and fragments or variants thereof.

A 9^{th} aspect is directed to a polypeptide according to aspect 8, wherein said fragment or variant of a polypeptide comprising an amino acid sequence selected from the group consisting of SEQ ID NOS: 232-452 and 457-459 retains anti-SARS-CoV-2 activity.

A 10^{th} aspect is directed to a nucleic acid encoding a polypeptide consisting of an amino acid sequence selected from the group consisting of SEQ ID NOS: 4-224 and 454-456, and/or fragments and variants thereof, and optionally 1 to 12 additional amino acids distributed in any ratio on the N terminus and/or C-terminus of the polypeptide of SEQ ID NOS: 4-224 and 454-456.

A 11^{th} aspect is directed to a nucleic acid encoding a polypeptide consisting essentially of an amino acid sequence selected from the group consisting of SEQ ID NOS: 4-224 and 454-456, and/or fragments and variants thereof, and optionally 1 to 12 additional amino acids distributed in any ratio on the N terminus and/or C-terminus of the polypeptide of SEQ ID NOS: 4-224 and 454-456.

A 12^{th} aspect is directed to a nucleic acid encoding a polypeptide comprising an amino acid sequence selected from the group consisting of SEQ ID NOS: 4-224 and 454-456, and/or fragments and variants thereof, and optionally 1 to 12 additional amino acids distributed in any ratio on the N terminus and/or C-terminus of the polypeptide of SEQ ID NOS: 4-224 and 454-456.

A 13^{th} aspect is directed to a nucleic acid encoding a polypeptide comprising an amino acid selected from the group consisting of SEQ ID NOS: 232-452 and 457-459 and/or fragments and variants thereof.

A 14^{th} aspect is directed to a nucleic acid consisting of a sequence selected from the group consisting of SEQ ID NOS: 232-452 and 457-459 and fragments or variants thereof.

A 15^{th} aspect is directed to a nucleic acid consisting essentially of a sequence selected from the group consisting of SEQ ID NOS: 232-452 and 457-459, and fragments or variants thereof.

A 16^{th} aspect is directed to a nucleic acid comprising a sequence selected from the group consisting of SEQ ID NOS: 232-452 and 457-459, and fragments or variants thereof.

A 17^{th} aspect is directed to a nucleic acid of any one of aspects 10-12, wherein said fragment or variant of the nucleic acid encoding a polypeptide comprising an amino acid sequence selected from the group consisting of SEQ ID NOS: 4-224 and 454-456 encodes a polypeptide that retains anti-SARS-CoV-2 activity.

A 18^{th} aspect is directed to a nucleic acid of aspect 13, wherein said fragment or variant of the nucleic acid encoding a polypeptide comprising an amino acid selected from the group consisting of SEQ ID NOS: 232-452 and 457-459 encodes a polypeptide that retains anti-SARS-CoV-2 activity.

A 19^{th} aspect is directed to a polypeptide or nucleic acid of any one of aspects 1-7, 10-12, or 17, wherein said polypeptide is one or more Class II polypeptides (“clusters”) of Table 1, (and/or fragments or variants thereof), and optionally 1 to 12 additional amino acids distributed in any ratio on the N terminus and/or C-terminus of a polypeptide of Table 1.

A 20^{th} aspect is directed to a polypeptide or nucleic acid of any one of aspects 1-7, 10-12, or 17, wherein said polypeptide is one or more Class II polypeptides (“clusters”) comprising, consisting, or consisting essentially of an amino acid sequence of SEQ ID NOS: 210, 9, 211, 34, 212, 213, 49, 54, 59, 214, 215, 89, 94, 99, 216, 104, 109, 114, 217, 218, 142, 147, 219, 220, 221, 222, 181, 186, 191, 196, 223, and 224 (and/or fragments or variants thereof), and optionally 1 to 12 additional amino acids distributed in any ratio on the N terminus and/or C-terminus of the polypeptide of SEQ ID NOS: 210, 9, 211, 34, 212, 213, 49, 54, 59, 214, 215, 89, 94, 99, 216, 104, 109, 114, 217, 218, 142, 147, 219, 220, 221, 222, 181, 186, 191, 196, 223, and 224.

A 21^{st} aspect is directed to a polypeptide or nucleic acid of any one of aspects 1-7, 10-12, or 17, wherein said polypeptide is one or more Class II polypeptides (“clusters”) comprising, consisting, or consisting essentially of an amino acid sequence of SEQ ID NOS: 218, 19, 216, 212, 89, 94, 34, 142, 221, 54, 186, 59, 214, 223, 219, 191, 224, 222, 210, 49, 104, 217, 213, 215, 109, 220, 147, 181, 196, 9, 114, and 99 (and/or fragments or variants thereof), and optionally 1 to 12 additional amino acids distributed in any ratio on the N terminus and/or C-terminus of the polypeptide of SEQ ID NOS: 218, 19, 216, 212, 89, 94, 34, 142, 221, 54, 186, 59, 214, 223, 219, 191, 224, 222, 210, 49, 104, 217, 213, 215, 109, 220, 147, 181, 196, 9, 114, and 99.

A 22^{nd} aspect is directed to a polypeptide or nucleic acid of any one of aspects 1-7, 10-12, or 17, wherein said polypeptide is one or more Class II polypeptides (“clusters”) comprising, consisting, or consisting essentially of an amino acid sequence of SEQ ID NOS: 210, 9, 19, 34, 212, 44, 49, 54, 59, 214, 89, 94, 99, 216, 104, 109, 110, 114, 218, 142, 147, 220, 221, 176, 181, 186, 191, 196, 223, 224, 454, 455, and 456 (and/or fragments or variants thereof), and optionally 1 to 12 additional amino acids distributed in any ratio on the N terminus and/or C-terminus of the polypeptide of SEQ ID NOS: 210, 9, 19, 34, 212, 44, 49, 54, 59, 214, 89, 94, 99, 216, 104, 109, 110, 114, 218, 142, 147, 220, 221, 176, 181, 186, 191, 196, 223, 224, 454, 455, and 456.

A 23^{rd} aspect is directed to a polypeptide or nucleic acid of any one of aspects 8-9, or 13-16, or 18, wherein said polypeptide is one or more polypeptides comprising, consisting, or consisting essentially of an amino acid sequence of one or more of SEQ ID NOS: 438, 237, 246, 262, 440, 441, 272, 277, 282, 287, 442, 317, 322, 327, 444, 332, 337, 338, 342, 446, 370, 375, 448, 449, 404, 409, 414, 419, 424, 451, 452, 457, 458, and 459 (and fragments and variants thereof).

A 24^{th} aspect is directed to a polypeptide or nucleic acid of any one of aspects 8-9, or 13-16, or 18, wherein said polypeptide is one or more polypeptides comprising, consisting, or consisting essentially of an amino acid sequence of one or more of SEQ ID NOS: 438, 237, 246, 262, 440, 441, 272, 277, 282, 287, 442, 317, 322, 327, 444, 332, 337, 338, 342, 446, 370, 375, 448, 449, 404, 409, 414, 419, 424, 451, and 452 (and/or fragments or variants thereof), or wherein said polypeptide is one or more polypeptides comprising, consisting, or consisting essentially of an amino acid sequence of SEQ ID NOS: 446, 246, 444, 440, 317, 332, 262, 370, 449, 282, 414, 287, 442, 451, 447, 419, 452, 450, 438, 277, 332, 445, 441, 443, 337, 448, 375, 409, 424, 237, 342, and 327 (and/or fragments or variants thereof).

A 25^{th} aspect is directed to a plasmid comprising a nucleic acid of any one of aspects 10-24.

A 26^{th} aspect is directed to a vector comprising a nucleic acid according to any one of aspects 10-24.

A 27^{th} aspect is directed to a pharmaceutical composition comprising a polypeptide according to any one of aspects 1-9 or 19-24 and a pharmaceutically-acceptable carrier and/or excipient.

A 28^{th} aspect is directed to a pharmaceutical composition comprising a nucleic acid according to any one of aspects 10-24 and a pharmaceutically-acceptable carrier and/or excipient.

A 29^{th} aspect is directed to a pharmaceutical composition comprising a plasmid according to aspect 25 and a pharmaceutically-acceptable carrier and/or excipient.

A 30^{th} aspect is directed to a pharmaceutical composition comprising a vector according to aspect 26 and a pharmaceutically-acceptable carrier and/or excipient.

A 31^{st} aspect is directed to a vaccine comprising a polypeptide according to any one of aspects 1-9 or 19-24 and a pharmaceutically-acceptable excipient, carrier, and/or adjuvant.

A 32^{nd} aspect is directed to a vaccine comprising a nucleic acid according to any one of aspects 10-24 and a pharmaceutically-acceptable excipient, carrier, and/or adjuvant.

A 33^{rd} aspect is directed to a vaccine comprising a plasmid according to aspect 25 and a pharmaceutically-acceptable excipient, carrier, and/or adjuvant.

A 34^{th} aspect is directed to a vaccine comprising a vector according to aspect 26 and a pharmaceutically-acceptable excipient, carrier, and/or adjuvant.

A 35^{th} aspect is directed to a method for inducing immunity against SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19 in a subject in need thereof, the method comprising administering to the subject a therapeutically effective amount of a polypeptide according to any one of aspects 1-9 or 19-24.

A 36^{th} aspect is directed to a method for inducing immunity against a SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19 in a subject in need thereof, the method comprising administering to the subject a therapeutically effective amount of a nucleic acid according to any one of aspects 10-24.

A 37^{th} aspect is directed to a method for inducing immunity against a SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19 in a subject in need thereof, the method comprising administering to the subject a therapeutically effective amount of a plasmid according to aspect 25.

A 38^{th} aspect is directed to a method for inducing immunity SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19 in a subject in need thereof, the method comprising administering to the subject a therapeutically effective amount of a vector according to aspect 26.

A 39^{th} aspect is directed to a method for inducing immunity against SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19 in a subject in need thereof, the method comprising administering to the subject a therapeutically effective amount of a pharmaceutical composition according to any one of aspects 27-30.

A 40^{th} aspect is directed to a method for inducing immunity against SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19 in a subject in need thereof, the method comprising administering to the subject a therapeutically effective amount of a vaccine composition according to any one of aspects 31-34.

A 41^{st} aspect is directed to a method according to any one of aspects 35-40, wherein the step of administration additionally includes administration of an SARS-CoV-2 virus, wherein the virus is a live attenuated virus or inactivated virus.

A 42^{nd} aspect is directed to a method for inducing an immune response against SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19 in a subject in need thereof, the method comprising administering to the subject a therapeutically effective amount of one or more of a polypeptide according to any one of aspects 1-9 or 10-24.

A 43^{rd} aspect is directed to a method for inducing an immune response against against SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19 in a subject in need thereof, the method comprising administering to the subject a therapeutically effective amount of a nucleic acid according to any one of aspects 10-24.

A 44^{th} aspect is directed to a method for inducing an immune response against against SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19 in a subject in need thereof, the method comprising administering to the subject a therapeutically effective amount of a plasmid according to aspect 25.

A 45^{th} aspect is directed to a method for inducing an immune response against against SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19 in a subject in need thereof, the method comprising administering to the subject a therapeutically effective amount of a vector according to aspect 26.

A 46^{th} aspect is directed to a method for inducing an immune response against SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19 in a subject in need thereof, the method comprising administering to the subject a therapeutically effective amount of a pharmaceutical composition according to any one of aspects 27-30.

A 47^{th} aspect is directed to a method for inducing an immune response against against SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19, in a subject in need thereof, the method comprising administering to the subject a therapeutically effective amount of a vaccine composition according to any one of aspects 31-34.

A 48^{th} aspect is directed to a method according to any one of aspects 42-47, wherein the step of administration additionally includes administration of a SARS-CoV-2 virus, wherein the virus is a live attenuated virus or inactivated virus.

A 49^{th} aspect is directed to a chimeric or fusion polypeptide comprising a polypeptide of any one of aspects 1-9 or 10-24, wherein said polypeptide is joined, linked, or inserted into a heterologous polypeptide.

A 50^{th} aspect is directed to a method for measuring a CMI response against SARS-CoV-2 or a related coronavirus in a subject, said method comprising; collecting a whole blood sample from said subject wherein said whole blood sample comprises cells of the immune system which are capable of producing immune effector molecules following stimulation by an antigen, incubating a mixture comprising the whole blood sample, at least one polypeptide according to any one of aspects 1-9 or 10-24, optionally an amount of an isolated simple sugar effective to enhance the stimulation by the antigen, and optionally heparin, and measuring the presence of, or elevation in, the level of an immune effector molecule wherein the presence or level of said immune effector molecule is indicative of the capacity of said subject to mount a cell-mediated immune response.

A 51^{st} aspect is directed to a method of aspect 50 wherein the subject is a human.

A 52^{nd} aspect is directed to a method of aspect 50 wherein the whole blood is collected in a tube comprising the at least one polypeptide.

A 53^{rd} aspect is directed to a method of aspect 50 wherein the whole blood is collected in a tube comprising heparin.

A 54^{th} aspect is directed to a method of aspect 52 wherein the tube comprises heparin.

A 55^{th} aspect is directed to a method of aspect 50 wherein the whole blood sample is incubated with the at least one polypeptide for from about 5 to about 50 hours.

A 56^{th} aspect is directed to a method of aspect 50 wherein the immune effector molecule is a cytokine.

A 57^{th} aspect is directed to a method of aspect 56 wherein the cytokine is IFN-γ.

A 58^{th} aspect is directed to a method of aspect 56 wherein the cytokine is GM-CSF.

A 59^{th} aspect is directed to a method of aspect 56 wherein the cytokine is an interleukin.

A 60^{th} aspect is directed to a method of aspect 56 wherein the cytokine is a TNF-α.

A 61^{st} aspect is directed to a method of either aspect 50 or 51 wherein the subject is infected by SARS-CoV-2 or a related coronavirus.

A 62^{nd} aspect is directed to a method of aspect 50 wherein the immune cells are selected from NK cells, T-cells, B-cells, dendritic cells, macrophages or monocytes.

A 63^{rd} aspect is directed to a method of aspect 52 wherein the immune cells are T-cells.

A 64^{th} aspect is directed to a method of aspect 50 wherein the simple sugar is dextrose.

A 65^{th} aspect is directed to a method of aspect 50 wherein the immune effectors are detected with antibodies specific for same.

A 66^{th} aspect is directed to a method of aspect 65 wherein the immune effectors are detected using ELISA.

A 67^{th} aspect is directed to a method of aspect 65 wherein the immune effectors are detected using ELISpot.

A 68^{th} aspect is directed to an assay for identifying SARS-CoV-2 or a related coronavirus-specific immediate effector T cells in a subject, comprising: (a) providing a sample from said subject containing T cells; (b) exposing said T cells to an immunogenic amount of at least one polypeptide according to any one of aspects 1-9 or 10-24; and (c) prior to the generation of new immediate effector T cells in the sample, determining whether said T cells are activated by said polypeptide by measuring secretion of a cytokine from said T cells; wherein activation of said T cells identifies the presence of SARS-CoV-2 or a related coronavirus-specific immediate effector T cells that were present in the original sample, in said subject.

A 69^{th} aspect is directed to a method of aspect 68, wherein said T cells are peripheral blood mononuclear cells.

A 70^{th} aspect is directed to a method of aspect 68, wherein the activation of said T cells is determined by measuring secretion of interferon-γ from said T cells.

A 71^{st} aspect is directed to a method of aspect 66, wherein said subject is known to be suffering, or to have suffered from, infection with SARS-CoV-2 or a related coronavirus.

A 72^{nd} aspect is directed to a method of aspect 71, wherein said infection is monitored.

A 73^{rd} aspect is directed to an assay for identifying SARS-CoV-2 or a related coronavirus-specific immediate effector T cells in a subject, comprising: (a) providing a sample from said subject containing T cells; (b) exposing said T cells to an immunogenic amount of at least one polypeptide according to any one of aspects 1-9 or 10-24; (c) incubating said T cells for a period of time which is not sufficient to effect differentiation of quiescent T cells to immediate effector T cells; and (d) determining whether said T cells are activated by said polypeptide by measuring secretion of a cytokine from said T cells, wherein activation of said T cells identifies the presence of SARS-CoV-2 or a related coronavirus-specific immediate effector T cells in said subject.

A 74^{th} aspect is directed to a method of either aspect 72 or 73, wherein said T cells are exposed to said polypeptide at around 37° C.

A 75^{th} aspect is directed to a method of aspect 73, wherein said incubation time is from 4 hours to 24 hours.

A 76^{th} aspect is directed to a method of aspect 73, wherein said incubation time is from 6 hours to 16 hours.

A 77^{th} aspect is directed to a method of detecting an anti-SARS-CoV-2 or a related coronavirus CD8+ and/or CD4+ T cell response comprising contacting a population of CD8+ and/or CD4+ T cells of a human individual with one or more polypeptides according to any one of aspects 1-9 or 10-24, wherein one or more polypeptides may be substituted by an analogue which binds a T cell receptor that recognizes the peptide, and determining whether CD8+ and/or CD4+ T cells of the CD8+ and/or CD4+ T cell population recognize the peptide(s).

A 78^{th} aspect is directed to a method according to aspect 77 wherein a peptide panel is employed, wherein said panel includes said one or more polypeptide or said analogues thereof.

A 79^{th} aspect is directed to a method according to aspect 77 wherein any analogue which is used is (i) at least 70% homologous, preferably at least 80% homologous, more preferably at least 90% homologous, to the entire polypeptide, and/or (ii) has one or more deletions at the N-terminus and/or C-terminus in comparison to the polypeptide, and/or (iii) has one or more conservative substitutions compared to the polypeptide.

A 80^{th} aspect is directed to a method according to aspect 77 in which the recognition of the polypeptide(s) by the CD8+ and/or CD4+ T cells is determined by measuring secretion of a cytokine from the CD8+ and/or CD4+ T cells.

A 81^{st} aspect is directed to a method according to aspect 80 in which IFN-γ secretion from the T cells is measured.

A 82^{nd} aspect is directed to a method according to aspect 81 in which IFN-γ secretion from the CD8+ and/or CD4+ T cells is determined by allowing secreted IFN-γ to bind an immobilized antibody specific to the cytokine and then determining the presence of antibody/cytokine complex.

A 83^{th} aspect is directed to a method according to aspect 82 in which the CD8+ and/or CD4+ T cells are freshly isolated ex vivo cells from peripheral blood.

A 84^{th} aspect is directed a method according to aspect 77 in which CD8+ and/or CD4+ T cells are pre-cultured in vitro with the peptide(s).

A 85^{th} aspect is directed to a method according to aspect 77 wherein the population of CD8+ and/or CD4+ T cells is from an individual to whom an anti-SARS-CoV-2 or related coronavirus vaccine has been administered.

A 86^{th} aspect is directed to a method according to aspect 77 which is carried out in vitro.

A 87th aspect is directed to a method of diagnosing infection in a human host by, or exposure of a human host to, a SARS-CoV-2 or a related coronavirus, which method comprises the steps of: (i) contacting a population of T cells from the host with one or more polypeptides according to any one of aspects 1-9 or 10-24; and (ii) determining in vitro whether the T cells of said T cell population show a recognition response to said polypeptide.

A 88^{th} aspect is directed to a method of aspect 87, wherein the T cells are freshly isolated.

A 89^{th} aspect is directed to a method of aspect 87, wherein the T cells are isolated from blood.

A 90^{th} aspect is directed to a method of aspect 87, wherein the T cell population comprises CD4+ and/or CD8+ T cells.

A 91^{st} aspect is directed to a method of aspect 87, wherein the host is a healthy human host who has been exposed to SARS-CoV-2 or a related coronavirus.

A 92^{nd} aspect is directed to a kit comprising one or more polypeptides according to any one of aspects 1-9 or 10-24, wherein said one or more polypeptides may be substituted by an analogue which binds a T cell receptor which recognizes the polypeptide, and optionally a means to detect recognition of the polypeptide(s) by CD8+ and/or CD4+ T cells.

A 93^{rd} aspect is directed to a kit according to aspect 92 which includes an antibody to IFN-γ.

A 94^{th} aspect is directed to a kit according to aspect 92 wherein said antibody is immobilized on a solid support and which optionally also includes a means to detect any antibody/IFN-γ complex.

A 95^{th} aspect is directed to a kit according to aspect 92 which includes the means to detect recognition of the peptide(s) by CD8+ and/or CD4+ T cells.

A 96^{th} aspect is directed to a kit according to aspect 94 which includes the means to detect any antibody/IFN-γ complex.

A 97^{th} aspect is directed to a method of preventing, treating, or ameliorating a disease by SARS-CoV-2 infection, such as COVID-19 in a subject in need thereof, the method comprising administering to the subject a therapeutically effective amount of one or more of a polypeptide according to any one of aspects 1-9 or 10-24.

A 98^{th} aspect is directed to a method of preventing, treating, or ameliorating a disease by SARS-CoV-2 infection, such as COVID-19 in a subject in need thereof, the method comprising administering to the subject a therapeutically effective amount of a nucleic acid according to any one of aspects 10-24.

A 99^{th} aspect is directed to a method of preventing, treating, or ameliorating a disease by SARS-CoV-2 infection, such as COVID-19 in a subject in need thereof, the method comprising administering to the subject a therapeutically effective amount of a plasmid according to aspect 25.

A 100^{th} aspect is directed to a method of preventing, treating, or ameliorating a disease by SARS-CoV-2 infection, such as COVID-19 in a subject in need thereof, the method comprising administering to the subject a therapeutically effective amount of a vector according to aspect 26.

A 101^{st} aspect is directed to a method of preventing, treating, or ameliorating a disease by SARS-CoV-2 infection, such as COVID-19 in a subject in need thereof, the method comprising administering to the subject a therapeutically effective amount of a pharmaceutical composition according to any one of aspects 27-30.

A 102^{nd} aspect is directed to a method of preventing, treating, or ameliorating a disease by SARS-CoV-2 infection, such as COVID-19 in a subject in need thereof, the method comprising administering to the subject a therapeutically effective amount of a vaccine composition according to any one of aspects 31-34.

A 103^{rd} aspect is directed to a method according to any one of aspects 42-47, wherein the step of administration additionally includes administration of a SARS-CoV-2 virus, wherein the virus is a live attenuated virus or inactivated virus.

A 104^{th} aspect is directed to a polypeptide according to any one of aspects 1-9 or 10-24, wherein said polypeptide has one or more conservative substitutions compared to the polypeptide.

A 105^{th} aspect is directed to a polypeptide according to aspect 104, wherein said polypeptide retains MHC binding propensity and TCR specificity, and/or retains anti-SARS-CoV-2 activity. Further aspects and advantages of the instant disclosure are provided in the following section, which should be considered as illustrative only.

### EXAMPLES

The examples that follow are not to be construed as limiting the scope of the invention in any manner. In light of the present disclosure, numerous embodiments within the scope of the claims will be apparent to those of ordinary skill in the art.

### Example 1: In-Silico Identification of Potential Epitopes for HLA

T-cells specifically recognize epitopes presented by cells in the context of MHC (Major Histocompatibility Complex) Class I and II molecules. These T-cell epitopes can be represented as linear sequences comprising 7 to 30 contiguous amino acids that fit into the MHC Class I or II binding groove. A number of computer algorithms have been developed and used for detecting Class I and II epitopes within protein molecules of various origins (De Groot A S et al., (1997), AIDS Res Hum Retroviruses, 13(7):539-41; Schafer J R et al., (1998), Vaccine, 16(19):1880-4; De Groot A S et al., (2001), Vaccine, 19(31):4385-95; De Groot A S et al., (2003), Vaccine, 21(27-30):4486-504). These “in silico” predictions of T-cell epitopes have been successfully applied to the design of vaccines and the de-immunization of therapeutic proteins, i.e. antibody-based drugs, Fc fusion proteins, anticoagulants, blood factors, bone morphogenetic proteins, engineered protein scaffolds, enzymes, growth factors, hormones, interferons, interleukins, and thrombolytics (Dimitrov D S, (2012), Methods Mol Biol, 899:1-26).

The Conservatrix system (EpiVax, Providence, R.I.) is an algorithm useful for identifying 9-mer polypeptide sequences from a larger set of data. The Conservatrix system parses input sequences into 9-mer sequences that are conserved amongst multiple inputted whole sequences, such as multiple strains of the same pathogen, for even the most mutable of potential vaccine targets. These 9-mer sequences may be searched for identically matched 9-mer sequences across data sets.

The EpiMatrix™ system (EpiVax, Providence, R.I.) is a set of predictive algorithms encoded into computer programs useful for predicting class I and class II HLA ligands and T cell epitopes. The EpiMatrix™ system uses matrices in order to model the interaction between specific amino acids and binding positions within the HLA molecule. In order to identify putative epitopes resident within any given input protein, the EpiMatrix™ System first parses the input protein into a set of overlapping n-mer frames (n=length of amino acids of epitope peptide being screen; e.g., n=9 or n=10) where each frame overlaps the last by n−1 amino acids. Each frame is then scored for predicted affinity to one or more common alleles of the HLA molecules. Briefly, for any given n-mer peptide specific amino acid codes (one for each of 20 naturally occurring amino acids) and relative binding positions (1 to n) are used to select coefficients from the predictive matrix. Individual coefficients are derived using a proprietary method similar to, but not identical to, the pocket profile method first developed by Sturniolo (Sturniolo T et al., 1999, Nat Biotechnol, 17(6):555-61). Individual coefficients are then summed to produce a raw score. EpiMatrix™ raw scores are then normalized with respect to a score distribution derived from a very large set of randomly generated peptide sequences. The resulting “Z” scores are normally distributed and directly comparable across alleles. It was determined that any peptide scoring above 1.64 on the EpiMatrix™ “Z” scale (approximately the top 5% of any given peptide set) has a significant chance of binding to the MHC molecule for which it was predicted. Peptides scoring above 2.32 on the scale (the top 1%) are extremely likely to bind.

Peptides containing clusters of putative T cell epitopes are more likely to test positive in validating in vitro and in vivo assays. In aspects, the results of the initial EpiMatrix™ analysis is further screened for the presence of putative T cell epitope “clusters” using a second proprietary algorithm known as Clustimer™ algorithm. The Clustimer™ algorithm identifies sub-regions contained within any given amino acid sequence that contains a statistically unusually high number of putative T cell epitopes. Typical T-cell epitope “clusters” range from about 9 to roughly 30 amino acids in length and, considering their affinity to multiple alleles and across multiple 9-mer frames, can contain anywhere from about 4 to about 40 putative T cell epitopes. Table I discloses: an overview of MHC class II cluster selection from the envelope (SEQ ID NO: 1) of 2019-nCoV, including the instantly-disclosed T cell epitopes of SEQ ID NOS: 4-8; an overview of MHC class II cluster selection from the membrane (SEQ ID NO: 2) of 2019-nCoV, including the instantly-disclosed T cell epitopes of SEQ ID NOS: 9-63; and an overview of MHC class II cluster selection from the spike (SEQ ID NO: 3) of 2019-nCoV, including the instantly-disclosed T cell epitopes of SEQ ID NOS: 64-224 and 454-456. Table 1 discloses the cluster sequence (bolded) of each the instantly-disclosed T cell epitopes of SEQ ID NOS: 4-224 and 454-456, the cluster score (without flanks), the number of EpiMatrix hits for class I MHC (which is the number of hits is the number of EpiMatrix Z-scores above 1.64 or top 5% found within the sequence), and the number of EpiMatrix hits for class II MHC (which is the number of hits is the number of EpiMatrix Z-scores above 1.64 or top 5% found within the sequence). Each epitope cluster identified an aggregate EpiMatrix™ score is calculated by summing the scores of the putative T cell epitopes and subtracting a correcting factor based on the length of the candidate epitope cluster and the expected score of a randomly generated cluster of the same length. EpiMatrix™ cluster scores in excess of +10 are considered significant. In aspects, the T-cell epitopes of the instant disclosure contain several putative T-cell epitopes forming a pattern known as a T-cell epitope cluster.

The JanusMatrix system (EpiVax, Providence, R.I.) useful for screening peptide sequences for cross-conservation with a host proteome. JanusMatrix is an algorithm that predicts the potential for cross-reactivity between peptide clusters and the host genome or proteome, based on conservation of TCR-facing residues in their putative MHC ligands. The JanusMatrix algorithm first considers all the predicted epitopes contained within a given protein sequence and divides each predicted epitope into its constituent agretope and epitope. Each sequence is then screened against a database of host proteins. Peptides with a compatible MHC-facing agretope (i.e., the agretopes of both the input peptide and its host counterparty are predicted to bind the same MHC allele) and exactly the same TCR-facing epitope are returned. The JanusMatrix Homology Score suggests a bias towards immune tolerance. In the case of a therapeutic protein, cross-conservation between autologous human epitopes and epitopes in the therapeutic may increase the likelihood that such a candidate will be tolerated by the human immune system. In the case of a vaccine, cross-conservation between human epitopes and the antigenic epitopes may indicate that such a candidate utilizes immune camouflage, thereby evading the immune response and making for an ineffective vaccine. When the host is, for example, a human, the peptide clusters are screened against human genomes and proteomes, based on conservation of TCR-facing residues in their putative HLA ligands. The peptides are then scored using the JanusMatrix Homology Score. In aspects, peptides with a JanusMatrix Homology Score below 2.5 or below 3.0 indicate low tolerogenicity potential and may be useful for vaccines. In aspects, peptides with a JanusMatrix Homology Score above 3.0 indicate high tolerogenicity potential and may not be useful for vaccines, and in aspects may be excluded from the T cell epitope compositions of the present disclosure. Table 1 discloses the JanusMatrix Homology score of the instantly-disclosed T cell epitopes of SEQ ID NOS: 4-224 and 454-456, all of which are well below 2.5.

In aspects, the VaccineCAD system is useful for arranging potential epitopic vaccine candidates into a string to avoid creation of novel epitopes upon joining of the vaccine candidate sequences. Specifically, VaccineCAD designs potential vaccine candidates into a string-of-beads vaccine while minimizing any deleterious junctional epitopes that may appear in the joining process. VaccineCAD may use EpiMatrix to predict junctional epitopes.

### Example 2: Administration of SARS-CoV-2 Vaccine

Vaccine construct designs are developed, such as is demonstrated in the specification. As described throughout, such vaccines may be used for stimulating, inducing, and/or expanding an immune response against SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19. In aspects, such vaccines initiate a strong T-cell mediated immune response, and has potential of inducing a humoral immune response.

Therefore, a vaccine as disclosed herein, optionally together with either live attenuated virus (LAV) or inactivated virus is administered in an immunization trial in an appropriate animal model, e.g., mice, rats, rabbits, hamsters, etc., or even humans, as are known in the art. Data from administration of this combination vaccine provides positive results on the safety and effectiveness of the vaccine. This vaccination approach is expected to induce both cellular and humoral immune responses, thereby stimulating, inducing, and/or expanding an immune response against SARS-CoV-2 infection (or a closely related virus such as Severe Acute Respiratory Syndrome (SARS) or Middle East respiratory syndrome coronavirus (MERS-CoV)) and/or related diseases caused by SARS-CoV-2, including COVID-19, in humans.

### Example 3: Polypeptide Binding to MHC

### Methods for the Assessment Polypeptides as Disclosed Herein Binding to Soluble MHC.

Synthesis of peptides. The polypeptides of the present disclosure (e.g., but not limited to, a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 4-224 and 454-456), as well as the Ii-Key constructs disclosed herein, such as those of Table 2) can be produced by direct chemical synthesis or by recombinant methods (J Sambrook et al., Molecular Cloning: A Laboratory Manual, (2ED, 1989), Cold Spring Harbor Laboratory Press, Cold Springs Harbor, N.Y. (Publ)). Every peptide undergoes rigorous quality control characterization before release to determine purity, mass, and correct sequence. Peptides are assessed for purity by reversed phase high-pressure liquid chromatography (RP-HPLC). Peptides are >90% pure, and each preparation will undergo Amino Acid Analysis to ensure that the equivalent molar amounts are used in assays for consistency and reproducibility between different lots of peptides, and will also allow for reliable comparison studies between peptide efficacy. Peptides are assessed for mass and correct sequence using tandem mass spectrometry and MS CheckT analysis. In certain aspects, the polypeptides as disclosed herein are capped with an n-terminal acetyl and/or c-terminal amino group. HPLC, mass spectrometry and UV scan (ensuring purity, mass and spectrum, respectively) analysis of the selected polypeptides will indicate >80% purity.

HLA Binding Assay. Binding activity is analyzed at EpiVax (Providence, R.I.) and is conducted for any polypeptides of the present disclosure (e.g., but not limited to, a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 4-224 and 454-456). The binding assay used (Steere A C et al., (2006), J Exp Med, 2003(4):961-71) yields an indirect measure of peptide-MHC affinity. Soluble HLA molecules are loaded onto a 96-well plate with the unlabeled experimental polypeptides and labeled control peptide. Once the binding mixture reaches steady equilibrium (at 24 hours), the HLA-polypeptide complexes are captured on an ELISA plate coated with anti-human DR antibody and detected with a Europium-linked probe for the label (PerkinElmer, Waltham, Mass.). Time-resolved fluorescence measuring bound labeled control peptide is assessed by a SpectraMax® M5 unit (Spectramax, Radnor, Pa.). Binding of experimental polypeptides is expressed as the percent inhibition of the labeled control peptide (experimental fluorescence/control fluorescence multiplied by 100). The percent inhibition values for each experimental polypeptide (across a range of molar concentrations) is used to calculate the concentration at which it inhibits 50% of the labeled control polypeptide's specific binding, i.e., the polypeptides's IC50.

Select experimental polypeptides are solvated in DMSO. The diluted polypeptide is then mixed with binding reagents in aqueous buffering solution, yielding a range of final concentrations from 100,000 nM down to 100 nM. The select polypeptides are then assayed against a panel of eight common Class II HLA alleles: DRB1*0101, DRB1*0301, DRB1*0401, DRB1*0701, DRB1*0801, DRB1*1101, DRB1*1301, and DRB1*1501. From the percent inhibition of labeled control peptide at each concentration, IC50 values are derived for each polypeptide/allele combination using linear regression analysis.

In this assay, the experimental polypeptides are considered to bind with very high affinity if they inhibit 50% of control peptide binding at a concentration of 100 nM or less, high affinity if they inhibit 50% of control peptide binding at a concentration between 100 nM and 1,000 nM, and moderate affinity if they inhibit 50% of control peptide binding at a concentration between 1,000 nM and 10,000 nM. Low affinity peptides inhibit 50% of control peptide binding at concentrations between 10,000 nM and 100,000 nM. Peptides that fail to inhibit at least 50% of control peptide binding at any concentration below 100,000 nM and do not show a dose response are considered non-binders (NB).

### Peptide Characterization by Binding to HLA Class II Molecules

Soluble MHC binding assays are performed on any of the instantly disclosed polypeptides (e.g., but not limited to, a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 4-224 and 454-456, as well as the Ii-key constructs as disclosed herein, including those of Table 2). Soluble MHC binding assays are performed on selected polypeptides as disclosed herein according to the methods described previously. IC50 values (nM) are derived from a six-point inhibition curve. EpiMatrix™ Predictions, calculated IC50 values, and results classifications are reported for each polypeptide and HLA allele. Binding curves are generated for certain polypeptides against the selected Class II HLA alleles, such as for the HLA DRB1*0801 assay and the HLA DRB1*1501 assay.

### Example 4: Peptide Exposed APCs

### Methods for Assessing the Phenotype of Peptide-Exposed APC

Surface expression of Class II HLA (HLA-DR) and CD86 by professional antigen presenting cells (APCs) is one way APCs modulate T cell response. In this assay, candidate polypeptides are tested for their ability to effect (e.g., upregulate) the expression of Class II HLA and the co-stimulatory molecule CD86 on the surface of professional APCs, specifically dendritic cells. In aspects, it is expected that the polypeptides as disclosed herein with mutations to anchor residues will result in increased expression of Class II HLA surface marker and the co-stimulatory molecule CD86.

Polypeptides of the present disclosure (e.g., but not limited to, a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 4-224 and 454-456, as well as Ii-key constructs of the present disclosure, including those of Table 2) are individually tested for effector potential using a proprietary APC phenotyping assay previously developed at EpiVax (EpiVax, Providence, R.I.). Previously harvested and frozen PBMC are thawed and suspended in chRPMI by conventional means. HLA typing is conducted on small, extracted samples of cellular material, provided by EpiVax, by Hartford Hospital (Hartford, Conn.). On assay day 0, 0.5×10⁶ cells are extracted, screened for the presence of surface marker CD11c (a marker specific to dendritic cells) and are analyzed for the presence of surface markers HLA-DR and CD86 by flow cytometry. The remaining cells are plated (4.0×10⁶ cell per ml in chRPMI plus 800 μl media) and are stimulated (50 μg/mL) with one of the selected peptides or positive and negative controls including buffer only (negative control), Tregitope 167 (negative control for effector activity) (21st Century Biochemicals, Marlboro, Mass.), Flu-HA 306-318 (positive control) (21^{ST} Century Biochemicals, Marlboro, Mass.) and Ova 323-339 (negative control) (21st Century Biochemicals, Marlboro, Mass.). Plated cells will be incubated for seven days at 37° C. On assay day 7, incubated cells are screened by flow cytometry for the presence of surface marker CD11c. CD11c positive cells are then analyzed for the presence of surface markers HLA-DR and CD86. The experimental peptides are tested in samples drawn from five different human donors.

Leukocyte Reduction Filters are obtained from the Rhode Island Blood Center (Providence, R.I.) to filter white blood cells from whole blood obtained from healthy donors. After the whole blood is run through the filters, the filters are flushed in the opposite direction to push collected white blood cells out of the filter. The white blood cells are then isolated using a conventional Ficoll™ separation gradient (GE Healthcare). The collected white blood cells are thereafter frozen for future use. When needed for use in an assay, the frozen white blood cells are thawed using conventional methods. For the GvHD studies discussed below, PBMCs are obtained (e.g., from HemaCare, Van Nuys, Calif.).

Exposure to the instantly disclosed polypeptides as disclosed herein on the phenotypes of dendritic cells is measured by multiple means. First, for each experimental condition, dot-plots, contrasting surface expression of CD11c and HLA-DR, are produced. Dot-plots of cells exposed to all control and experimental peptides are overlaid onto dot-plots produced from control cells exposed to only the culture media. The overlay provides an effective method to visually observe shifts in HLA-DR distribution between polypeptide stimulated, and unstimulated CD11c-high cells (data not shown). Observed shifts in the distribution of HLA-DR are reported as a qualitative measure. Next, the change in intensity of HLA-DR expression for the CD11c-high segment of each dot-plot is calculated. Percent change in intensity of HLA-DR expression equals Mean Florescence Index (MFI) of HLA-DR expression for peptide exposed cells minus MFI of HLA-DR expression for media exposed cells divided by MFI of HLA-DR expression for media exposed cells, times 100 (HLA-DRMFIpeptide−HLA-DRMFImedia/HLA-DRMFImedia*100). Next, the percent change in the percentage of HLA-DR-low cells present among the CD11c high population is calculated for each peptide relative to media control. Percent change in the percentage of HLA-DR-low cells is calculated, and equals the percent of HLA-DR-low for peptide exposed cells minus the percent of HLA-DR-low for media exposed cells divided by percent of HLA-DR-low for media exposed cells times 100 (HLA-DR-low % peptide−HLA-DR-low % media/HLA-DR-low % media*100). In this assay, a negative change in observed HLA-DR MFI and a positive change in percentage of HLA-DR-low cells present in the CD11c-high population indicates reduced expression of HLA and a shift to a regulatory APC phenotype. In this assay, a positive change in observed HLA-DR MFI and a negative change in percentage of HLA-DR-low cells present in the CD11c-high population indicates increased expression of HLA and a shift to an effector APC phenotype.

A similar process is used to assess the impact of the instantly-disclosed polypeptides exposure on surface expression of CD86, which is a costimulatory molecule known to promote T cell activation. First, for each experimental condition, dot plots contrasting surface expression of CD11c and CD86 are produced. Dot plots of cells exposed to all control and experimental Tregitopes are overlaid onto dots plots produced from control cells exposed to only the culture media. The overlay provides an effective method to visually observe shifts in CD86 distribution between polypeptide stimulated and un-stimulated CD11c-high cells. Observed shifts in the distribution of CD86 are reported as a qualitative measure. Next, the change in intensity of CD86-high expression for the CD11c-high segment of each dot plot is calculated. Percent change in intensity of CD86-high expression equals Mean Florescence Index (MFI) of CD86 expression for peptide exposed cells minus MFI of CD86-high expression for media exposed cells divided by MFI of CD86 expression for media exposed cells, times 100 (CD86-highMFIpeptide−CD86-highMFImedia/CD86-highMFImedia*100). Next, the percent change in the percentage of CD86-low cells present among the CD11c high population is calculated. Percent change in the percentage of CD86-high cells equals the percent of CD86-high for peptide exposed cells minus the percent of CD86-high for media exposed cells divided by percent of CD86-high for media exposed cells, times 100 (CD86-low % Ipeptide−CD86-low % media/CD86-low % media*100). In this assay, a negative change in observed CD86 MFI and a positive change in percentage of CD86-low cells present in the CD11c-high population indicates reduced expression of CD86 and a shift to a regulatory APC phenotype. In this assay, a positive change in observed CD86 MFI and a negative change in percentage of CD86-low cells present in the CD11c-high population indicates increased expression of CD86 and a shift to an effector APC phenotype.

### Characterization of Peptide Exposed APC

Dendritic cell phenotyping assays are performed on the polypeptides of the instant disclosure according to the methods described previously.

Dot plots representing the surface expression of CD11 vs HLA-DR are analyzed on assay day 7 across the five donors in the presence of various peptide stimulants. In aspects, it is expected that upward movement of the CD11c+/HLA-DR+ population will apparent in the samples treated with the polypeptides of the instant disclosure (e.g., but not limited to, a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 4-224 and 454-456, as well as Ii-key constructs of the present disclosure, including those of Table 2) as compared to media control indicating an acquired effector phenotype.

Dot plots representing the surface expression of CD11c vs CD86 are analyzed on assay day 7 across the five donors in the presence of various peptide stimulants. It is expected that an increase in CD86-hi cells present in the samples treated with polypeptides of the instant disclosure as compared to media control, which indicates a shift to the acquired effector phenotype. In aspects, it is expected that the polypeptides as disclosed herein (e.g., but not limited to, a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 4-224 and 454-456, as well as Ii-key constructs of the present disclosure, including those of Table 2) will increase expression of HLA-DR as compared to control media.

### Example 6: Memory T Cell Responses to SARS-CoV-2 in COVID-19 Convalescents

### Materials and Methods

Peptide synthesis. Synthetic peptides were manufactured using 9-fluoronylmethoxycarbonyl (Fmoc) chemistry by 21st Century Biochemicals (Marlboro, Mass.). Peptide purity was >90% as ascertained by analytical reversed phase HPLC. Peptide mass was confirmed by tandem mass spectrometry.

SARS-CoV-2 convalescent donors. Convalescent patients were recruited by Sanguine Biosciences, a clinical services group that identified, consented and enrolled participants. Inclusion criteria included subjects (i) willing and able to provide written informed consent and photo identification, (ii) aged 18-60, both male or female, (iii) confirmed COVID-19 diagnosis (recovered) with date of diagnosis a minimum of 30 days from blood collection, and (iv) positive COVID-19 PCR based-kit documented by time-stamped medical record and/or diagnostic test report and test kit used identified. Exclusion criteria included subjects who (i) are pregnant or nursing, (ii) have a known history of HIV, hepatitis or other infectious diseases, (iii) have autoimmune diseases, (iv) in vulnerable patient population (prisoners, mentally impaired), (v) have medical conditions impacting their ability to donate blood (i.e. anemia, acute illness) (vi) received immunosuppressive therapy or steroids within the last 6 months, (vii) received an investigational product in the last 30 days, (viii) experienced excess blood loss including blood donation defined as 250 mL in the last month or 500 mL in the last two months, or (ix) had a positive COVID-19 PCR test, but were asymptomatic. Samples were collected in accordance with NIH regulations and with IRB approval.

Healthy unexposed donors. Samples were obtained from leukocyte reduction filters from the Rhode Island Blood Center for unrelated studies prior to the SARS-CoV-2 outbreak in December 2019. Samples were collected in accordance with NIH regulations and with IRB approval.

PBMC culture. Thawed whole PBMCs (normal healthy donors) will be rested overnight and expanded by antigen stimulation (including the detolerized SARS-CoV-2 polypeptides of the instant disclosure) over nine days at 37° C. under a 5% CO2 atmosphere. In a 48-well plate, 5×10\hat{ }6 cells in 150 μl RPMI medium supplemented with human AB serum will be stimulated with pools of peptides at 10 μg/ml on Day 1. Three days later, IL-2 will be added to 10 ng/ml and the culture volume raised to 300 μl. On Day 7, cells will be supplemented with 10 ng/ml IL-2 by half media replacement. Two days later, PBMCs will be collected and washed in preparation to measure immune recall responses.

FluoroSpot Assay. Interferon-gamma (IFNg) Fluorospot assays will be performed ex vivo and following culture using kits purchased from Mabtech and performed according to the manufacturer's specifications. Peptides will be added individually at 10 μg/ml and pooled at 10 μg/ml (8 peptides, 1.25 μg/mL) to triplicate wells containing 250,000 PBMCs (ex vivo) or 100,000 PBMCs (cultured) in RPMI medium supplemented with 10% human AB serum. Triplicate wells will be plated with ConA (10 μg/ml) as a positive control, and six wells containing no antigen stimulus were used for background determination. Cells will incubated for 40-48 hours at 37° C. under a 5% CO2 atmosphere. Plates will be developed according to the manufacturer's directions using FITC-labeled anti-IFN-γ detection antibody.

Raw spot counts will be recorded by ZellNet Consulting, Inc. using a FluoroSpot reader system (iSpot Spectrum, AID, Strassberg, Germany) with software version 7.0, build 14790, where fluorescent spots will be counted utilizing separate filters for FITC, Cy3, and Cy5. Camera exposure and gain settings will be adapted for each filter to obtain high quality spot images preventing over- or underexposure. Fluorophore-specific spot parameters will be defined using spot size, spot intensity and spot gradient (fading of staining intensity from center to periphery of spot), and a spot separation algorithm will be applied for optimal spot detection.

Results will be calculated as the average number of spots in the peptide wells, adjusted to spots per one million cells. Responses meeting the following criteria are positive when the number of spots is (i) at least twice background, (ii) greater than 50 spot forming cells per well above background (1 response per 20,000 PBMCs), and (iii) statistically different (p<0.05) from the media-only control by the Student's t test.

It is expected that the polypeptides of the present disclosure (e.g., but not limited to, a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 4-224 and 454-456), as well as the instantly disclosed hybrid Ii-key constructs (including those of Table 2) will be recognized by T cells raised in natural infection, stimulate Th1 cytokine production, may stimulate pre-existing immunity to common cold coronaviruses, and may boost memory immunity in clinical trials.

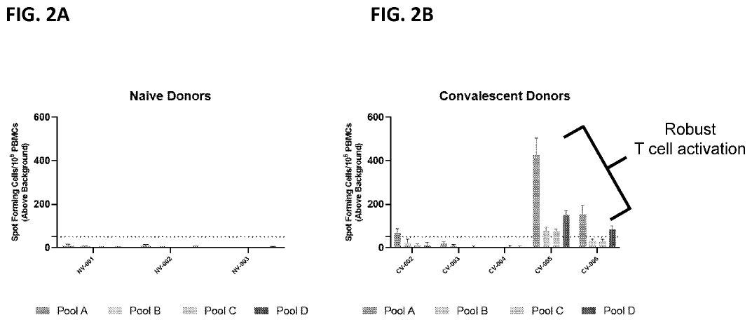

For FIG. 2A-FIG. 7, peptide 1 (or rank 1) is SEQ ID NO: 218; peptide 2 (or rank 2) is SEQ ID NO: 19; peptide 3 (or rank 3) is SEQ ID NO: 216; peptide 4 (or rank 4) is SEQ ID NO: 212; peptide 5 (or rank 5) is SEQ ID NO: 89; peptide 6 (or rank 6) is SEQ ID NO: 94; peptide 7 (or rank 7) is SEQ ID NO: 34; peptide 8 (or rank 8) is SEQ ID NO: 142; peptide 9 (or rank 9) is SEQ ID NO: 221; peptide 10 (or rank 10) is SEQ ID NO: 54; peptide 11 (or rank 11) is SEQ ID NO: 186; peptide 12 (or rank 12) is SEQ ID NO: 59; peptide 13 (or rank 13) is SEQ ID NO: 214; peptide 14 (or rank 14) is SEQ ID NO: 223; peptide 15 (or rank 15) is SEQ ID NO: 219; peptide 16 (or rank 16) is SEQ ID NO: 191; peptide 17 (or rank 17) is SEQ ID NO: 224; peptide 18 (or rank 18) is SEQ ID NO: 222; peptide 19 (or rank 19) is SEQ ID NO: 210; peptide 20 (or rank 20) is SEQ ID NO: 49; peptide 21 (or rank 21) is SEQ ID NO: 104; peptide 22 (or rank 22) is SEQ ID NO: 217; peptide 23 (or rank 23) is SEQ ID NO: 213; peptide 24 (or rank 24) is SEQ ID NO: 215; peptide 25 (or rank 25) is SEQ ID NO: 109; peptide 26 (or rank 26) is SEQ ID NO: 220; peptide 27 (or rank 27) is SEQ ID NO: 147; peptide 28 (or rank 28) is SEQ ID NO: 181; peptide 29 (or rank 29) is SEQ ID NO: 196; peptide 30 (or rank 30) is SEQ ID NO: 9; peptide 31 (or rank 31) is SEQ ID NO: 114; and peptide 32 (or rank 32) is SEQ ID NO: 99. Additionally, pool A includes the following: SEQ ID NO: 218; SEQ ID NO: 19; SEQ ID NO: 216; SEQ ID NO: 212; SEQ ID NO: 89; SEQ ID NO: 94; SEQ ID NO: 34; SEQ ID NO: 142. Pool B includes the following: SEQ ID NO: 221; SEQ ID NO: 54; SEQ ID NO: 186; SEQ ID NO: 59; SEQ ID NO: 214; SEQ ID NO: 223; SEQ ID NO: 219; SEQ ID NO: 220. Pool C includes the following: SEQ ID NO: 181; SEQ ID NO: 222; SEQ ID NO: 210; SEQ ID NO: 49; SEQ ID NO: 104; SEQ ID NO: 217; SEQ ID NO: 213; SEQ ID NO: 215. Pool D includes the following: SEQ ID NO: 109; SEQ ID NO: 220; SEQ ID NO: 147; SEQ ID NO: 181; SEQ ID NO: 196; SEQ ID NO: 9; SEQ ID NO: 114; and SEQ ID NO: 99.

As shown in FIG. 2A and FIG. 2B, ex vivo immune recall responses differentiate SARS-CoV-2 naïve and experienced individuals and exhibit different COVID-19 immunotypes. Robust and failed immune responses in convalescent donors may represent different immunotypes characterized in a deep immune profiling study of SARS-CoV-2 experienced humans (Giles et al. Deep immune profiling of COVID-19 patients reveals distinct immunotypes with therapeutic implications. Science. 2020 Jul. 15:eabc8511. doi: 10.1126/science.abc8511. PMID: 32669297, herein incorporated by reference in its entirety). In aspects, a polypeptide of the instant disclosure (e.g., a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N-terminus and/or C-terminus of the polypeptide of SEQ ID NOS: 4-224 and 454-456), including the hybrid Ii-key constructs as disclosed herein, such as those of Table 2 (SEQ ID NOS: 232-452 and 457-459), are expected to elicit a strong effector immune recall response (such as production of effector cytokines, e.g., IFN-γ).

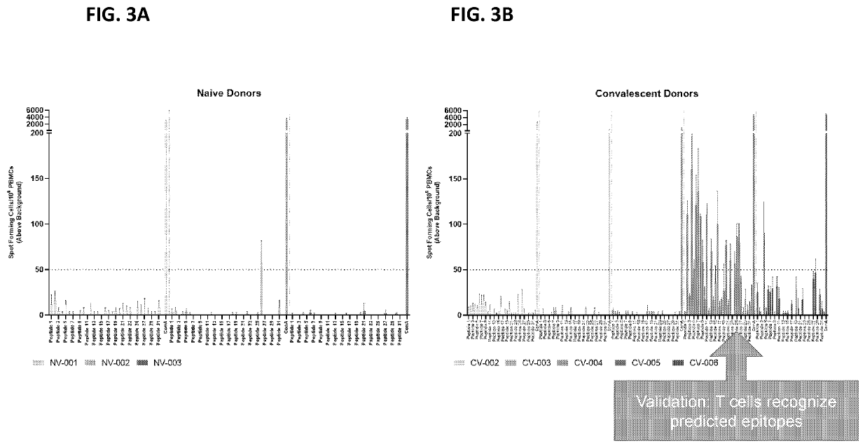

As shown in FIG. 3A and FIG. 3B, strong ex vivo immune recall responses are found in SARS-CoV-2 experienced individuals using polypeptides of the instant disclosure. In aspects, a polypeptide of the instant disclosure (e.g., a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 4-224 and 454-456), including the hybrid Ii-key constructs as disclosed herein, such as those of Table 2 (SEQ ID NOS: 232-452 and 457-459), are expected to elicit a strong ex vivo immune recall response.

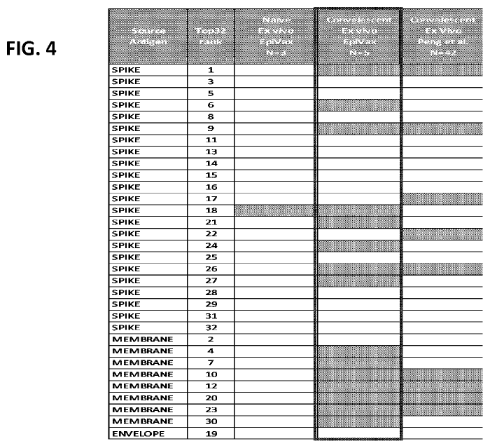

As shown in FIG. 4, polypeptides of the instant disclosure stimulate ex vivo immune recall response in natural SARS-CoV-2 infection. In this exemplary study, of 32 tested peptides, 15 testes peptides demonstrated positive responses, as shown in green, in at least one donor. External data is shown from the preprint of Peng et al. bioRxiv [Preprint]. 2020 Jun. 8 PMID: 32577665; PMCID: PMC7302222. In aspects, a polypeptide of the instant disclosure (e.g., a peptide or polypeptide comprising, consisting, or consisting essentially of an amino acid sequence of SEQ ID NOS: 2-224 (and/or fragments or variants thereof), and optionally 1 to 12 additional amino acids distributed in any ratio on the N terminus and/or C-terminus of the polypeptide of SEQ ID NOS: 2-224), the hybrid Ii-key constructs as disclosed herein, such as those of Table 2 (SEQ ID NOS: 232-452 and 457-459), are expected to elicit a strong effector immune recall response (such as production of effector cytokines, e.g., IFN-γ).

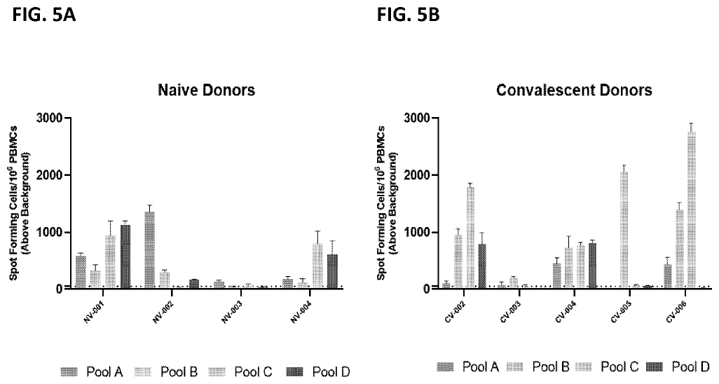

As shown in FIG. 5A and FIG. 5B, polypeptides of the instant disclosure stimulate higher IFN-γ responses in naïve and COVID-19 convalescent donors following expansion in culture. Response in naïve donors suggest such polypeptides of the instant disclosure expand low frequency cold coronavirus cross-reactive T cells. Further, differences between responses by pool in ex vivo and cultured assay may reflect variable phenotypes and/or proliferative capacities of epitope-specific T cells when they are put into culture. In aspects, a polypeptide of the instant disclosure (e.g., a peptide or polypeptide comprising, consisting, or consisting essentially of an amino acid sequence of SEQ ID NOS: 2-224 (and/or fragments or variants thereof), and optionally 1 to 12 additional amino acids distributed in any ratio on the N terminus and/or C-terminus of the polypeptide of SEQ ID NOS: 2-224), including the hybrid Ii-key constructs as disclosed herein, such as those of Table 2 (SEQ ID NOS: 232-452 and 457-459), are expected to elicit a strong effector immune recall response (such as production of effector cytokines, e.g., IFN-γ).

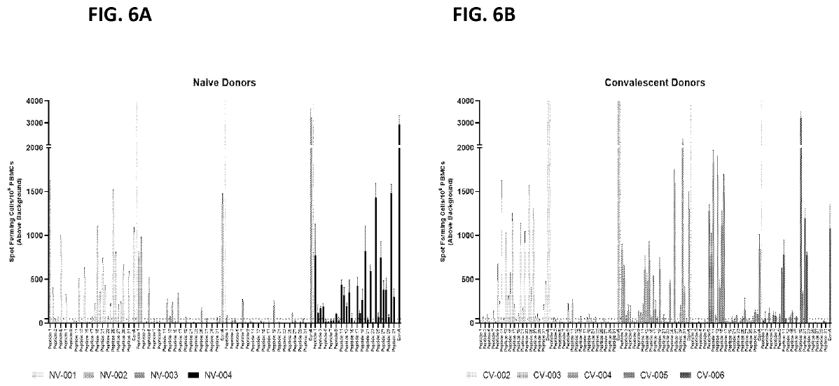

As shown in FIG. 6A and FIG. 6B, polypeptides of the instant disclosure stimulate low frequency epitope-specific T cells following expansion in culture in naïve and COVID-19 convalescent donors. Differences between responses to spike and membrane peptides in ex vivo and cultured assay may reflect variable phenotypes and/or proliferative capacities of epitope-specific T cells when they are put into culture. In aspects, a polypeptide of the instant disclosure (e.g., a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 4-224 and 454-456), including the hybrid Ii-key constructs as disclosed herein, such as those of Table 2 (SEQ ID NOS: 232-452 and 457-459), are expected to elicit a strong effector immune recall response (such as production of effector cytokines, e.g., IFN-γ).

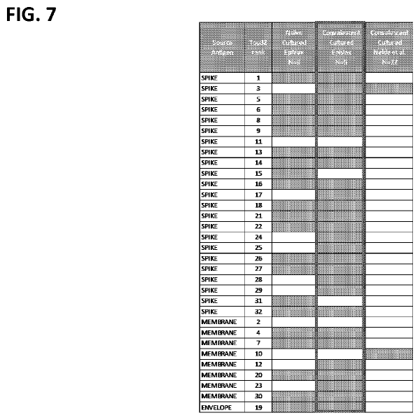

As shown in FIG. 7, polypeptides of the instant disclosure stimulate low frequency epitope-specific T cells following expansion in culture in naïve and COVID-19 convalescent donors. Of 32 tested peptides, 27 testes peptides demonstrated positive responses, as shown in green, in at least one donor. Further, predicted spike epitope cross-conservation with common cold coronaviruses were confirmed in naïve donors. External data is shown from the preprint of Nelde et al. Research Square [preprint]: 2020 Jun. 17 doi: 10.21203/rs.3.rs-35331/v1.

As such, the data in FIG. 2A-FIG. 7 demonstrates that polypeptides of the instant disclosure are recognized by T cells raised in natural infection, stimulate Th1 cytokine production, may stimulate pre-existing immunity to common cold coronaviruses, and memory may boost immunity in clinical trials. Again, in aspects, a polypeptide of the instant disclosure (e.g., a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 4-224 and 454-456), including the hybrid Ii-key constructs as disclosed herein, such as those of Table 2 (SEQ ID NOS: 232-452 and 457-459), are expected to elicit a strong effector immune recall response (such as production of effector cytokines, e.g., IFN-γ).

### Example 7—T Cell Epitope Mapping in Humans with and without SARS-CoV-2 Experience: Identification of Candidate Epitopes for Rational COVID-19 Vaccine Design

### Materials and Methods

SARS-CoV-2 sequences. SARS-CoV-2 (taxid: 2697049), SARS-CoV-1 (taxid: 694009), MERS-CoV (taxid: 1335626), and human CoV (taxids: 11137, 443239, 277944 and 31631) antigen sequences isolated from human hosts were obtained from GenBank at the National Center for Biotechnology Information. SARS-CoV-2 epitopes were compared across sequences obtained from isolates with fully sequenced genomes isolated from December 2019 to December 2020. SARS-CoV-2 Wuhan-Hu-1 (GenBank id: MN908947) was selected as the reference strain.

T cell epitope mapping. Each antigen sequence was parsed into all possible linear 9-mer sequences. Each 9-mer was scored for likelihood of binding to panels of class I and class II HLA alleles using EpiMatrix version 1.3, a matrix-based algorithm for mapping T cell epitopes. Class II epitopes were identified for nine supertype alleles: DRB1*0101, DRB1*0301, DRB1*0401, DRB1*0701, DRB1*0801, DRB1*0901, DRB1*1101, DRB1*1301 and DRB1*1501. Class I epitope 9-mers (and 10-mers) were identified for potential binding to six supertype alleles: A*0101, A*0201, A*0301, A*2402, B*0702, B*4401. Each allele set covers >95% of the human population. (Southwood S, Sidney J, Kondo A, et al. Several common HLA-DR types share largely overlapping peptide binding repertoires. J Immunol. 1998; 160(7):3363-337; and Sette A, Sidney J. Nine major HLA class I supertypes account for the vast preponderance of HLA-A and -B polymorphism. Immunogenetics. 1999; 50(3-4):201-212, both incorporated by reference in their entireties). Promiscuous class II HLA epitopes representing regions of a protein with a high density of T cell immunogenicity potential were identified using the ClustiMer algorithm. Clusters scoring an overall EpiMatrix score≥10 after subtracting the average expected sum of scores for a random sequence of equal length have significant immunogenicity potential (e.g., Moise et al. iVax: An integrated toolkit for the selection and optimization of antigens and the design of epitope-driven vaccines, Human Vaccines & Immunotherapeutics 11:9, 2312-2321 (2015), herein incorporated by reference in its entirety).

T cell epitope homology analysis. The JanusMatrix algorithm (Moise L, Gutierrez A H, Bailey-Kellogg C, et al. The two-faced T cell epitope: Examining the host-microbe interface with JanusMatrix. Hum Vaccines Immunother. 2013; 9(7): 1577-1586. doi:10.4161/hv.24615, herein incorporated by reference in its entirety) was used to identify SARS-CoV-2 epitopes that share T cell receptor (TCR)-face conservation (positions 2, 3, 5, 7, and 8) with epitopes restricted by the same alleles, but found in the human proteome, and other (alpha- and beta-) coronavirus epitopes. Epitopes with identical TCR-facing residues, which are also predicted to bind to the same MHC allele, are more likely to induce cross-reactive T cells. A JanusMatrix score threshold of two (cross-conserved HLA-allele-specific epitopes averaged over the length of the sequence) for cross-conservation with human (self) proteins was applied to identify epitopes with elevated potential to be tolerated or actively regulatory. To investigate the conservation of SARS-CoV-2 epitopes with related highly pathogenic coronaviruses (SARS-CoV and MERS-CoV) and low pathogenicity common cold coronaviruses (CCCs; 0C43, HKU1, NL62, and 229E), JanusMatrix was applied requiring identical TCR-facing residues at five positions and at positions 2 and 8 only. A separate homology analysis of iVAX-predicted SARS-CoV-2 T cell epitopes with published coronavirus epitopes deposited in the Immune Epitope Database was performed using BLAST and a cutoff of 80% similarity.

Peptide synthesis. Synthetic peptides were manufactured using 9-fluoronylmethoxycarbonyl (Fmoc) chemistry by 21^{st} Century Biochemicals (Marlboro, Mass.). Peptide purity was >90% as ascertained by analytical reversed phase HPLC. Peptide mass was confirmed by tandem mass spectrometry.

SARS-CoV-2 convalescent donors. Convalescent patients were recruited by Sanguine Biosciences, a clinical services group that identified, consented and enrolled participants. Inclusion criteria included subjects (i) willing and able to provide written informed consent, (ii) aged 18-80 years of age, both male or female, and (iii) PCR-confirmed COVID-19 diagnosis (recovered) with date of diagnosis a minimum of 30 days from blood collection. Exclusion criteria included subjects who (i) were pregnant or nursing, (ii) had a known history of HIV, hepatitis or other infectious diseases, (iii) had autoimmune diseases, (iv) were members of vulnerable patient population (prisoners, mentally impaired), (v) had medical conditions impacting their ability to donate blood (i.e. anemia, acute illness) (vi) had received immunosuppressive therapy or steroids within the last 6 months, (vii) had received an investigational product in the last 30 days, (viii) had experienced excess blood loss including blood donation defined as 250 mL in the last month or 500 mL in the last two months, or (ix) had a positive COVID-19 PCR test, but were asymptomatic. Samples were collected in accordance with NIH regulations and with the approval of an independent external institutional review board.

Healthy unexposed donors. Deidentified samples were obtained from leukocyte reduction filters from the Rhode Island Blood Center for unrelated studies prior to the SARS-CoV-2 outbreak in December 2019 (Date of samples: February 2016-November 2019). Samples were obtained in accordance with NIH regulations and with the approval of an independent external institutional review board (Ethical & Independent Review Services, Independence, Mo.).

PBMC culture. PBMCs from COVID-19 convalescents were put into culture directly following Ficoll separation. Cells from normal healthy donors were thawed and rested overnight before placing into culture. Samples were allocated for ex vivo assay and antigen-specific cell expansion followed by cultured assay. Antigen stimulated cells were cultured over eight days at 37° C. under a 5% CO₂ atmosphere. In a 48-well plate, 5×10⁶ cells in 150 μl RPMI medium supplemented with human AB serum were stimulated with pools of peptides at 10 μg/ml on Day 1. Three days later, IL-2 was added to 10 ng/ml and the culture volume raised to 300 μl. On Day 7, cells were supplemented with 10 ng/ml IL-2 by half media replacement. Two days later, PBMCs were collected and washed in preparation for measurement of immune recall responses.

Ex vivo and cultured human Fluorospot assay. Interferon-gamma (IFNγ) Fluorospot assays were performed using ex vivo and cultured PBMC using assay kits purchased from Mabtech and executed according to the manufacturer's specifications. For ex vivo assays, unless otherwise noted, peptides were added individually at 20 μg/ml or pooled at 10 μg/mL per peptide in triplicate wells containing 250,000 PBMCs in RPMI medium supplemented with 10% human AB serum. For cultured assays, peptides were added individually at 10 μg/ml or pooled at a total peptide concentration of 10 ug/mL (32 peptides, 0.313 μg/mL) and added to triplicate wells containing 100,000 PBMCs. Triplicate wells were plated with ConA (5 μg/ml) as a positive control, and six wells containing no antigen stimulus (0.2-0.4% DMSO) were used for background determination. Cells were incubated for 40-48 hours at 37° C. under a 5% CO2 atmosphere. Plates were developed according to the manufacturer's directions using FITC-labeled anti-IFN-γ detection antibody.

Raw spot counts were recorded by ZellNet Consulting, Inc. using a FluoroSpot reader system (iSpot Spectrum, AID, Strassberg, Germany) with software version 7.0, build 14790, where fluorescent spots were counted utilizing FITC and Cy3 filters. Camera exposure and gain settings were adapted for each filter to obtain high quality spot images preventing over- or underexposure. Fluorophore-specific spot parameters were defined using spot size, spot intensity and spot gradient (fading of staining intensity from center to periphery of spot), and a spot separation algorithm was applied for optimal spot detection.

Results were calculated as the average number of spots in the peptide wells, adjusted to spots per one million cells. Responses meeting the following criteria were considered to be positive when the number of spots was (i) at least five times background, (ii) greater than 25 spot forming cells per well above background (1 response per 40,000 PBMCs), and (iii) statistically different (p<0.05) from the media-only control by the Student's t test.

HLA typing. Donor HLA Class II types were determined using the One Lambda Micro SSP™ High Resolution HLA Class II kit at the Hartford Hospital Transplant Immunology Laboratory.

Mice. HLA-DR3 transgenic mice were obtained from Dr. Chella David (Mayo Clinic) under commercial license. The mice express the HLA-DRA and DRB1*0301 genes on a B.10-Ab⁰ mouse class II-negative background. Animal research protocols for mouse studies were reviewed and approved by the Absorption Systems Inc. Institutional Animal Care and Use Committee.

Peptide vaccine preparation. Per dose, a pool of 20 peptides at 1.25 or 5.0 μg/peptide was admixed with 50 μg poly-ICLC (Hiltonol™; Oncovir) in 50 μL.

Vaccinations. Vaccine- and sham-treated HLA-DR3 mice (N=5/group) were female and 6-8 weeks old at the start of immunizations. Mice were primed and boosted two weeks later by intradermal immunization with peptide/poly-ICLC vaccine. Control groups received sterile water (N=3) or poly-ICLC alone (N=5). Mice were sacrificed nine days after the boost immunization. Blood at baseline and termination and spleens were harvested for immune monitoring. One mouse in the group that received 5 μg/peptide was excluded following splenocyte isolation due to insufficient recovery and poor viability for reasons thought to be unrelated to vaccination.

Ex vivo FluoroSpot assay in mouse splenocytes. The frequency of vaccine-specific splenocytes was determined by dual cytokine IFNγ/IL-4 FluoroSpot assay using the Mabtech mouse IFNγ/IL-4 FluoroSpot Kit with pre-coated plates according to the manufacturer's protocol. Washed splenocytes in RPMI 1640 (Gibco) supplemented with 10% fetal calf serum (FCS, Atlanta Biologicals) were added at 250,000 cells per well. Antigen stimulations included pools of all 20 vaccine peptides, as well as spike-derived vaccine peptides, and membrane-derived vaccine peptides. Peptide pools were added at 0.5 μg/ml per peptide. Triplicate wells were stimulated with 2 μg/ml Concanavalin A (ConA; Sigma Aldrich) as a positive control, and six replicate wells with medium containing 0.2% DMSO were used for background determination. Raw spot counts were recorded by ZellNet Consulting, Inc. and results were calculated as described above for human FluoroSpot assays.

Flow cytometry in mouse splenocytes. Splenocytes were plated at 300,000 cells per well and stimulated in triplicate over six hours with a pool of all 20 vaccine peptides at 0.5 μg/mL per peptide and 4 μg/mL co-stimulatory anti-CD28 antibody. Triplicate wells were stimulated with PMA (50 ng/mL)+ionomycin (1 μg/mL) as a positive control. For background determinations, triplicate wells were treated with medium containing 0.2% DMSO only and medium containing 0.2% DMSO and 4 μg/mL co-stimulatory anti-CD28 antibody. Brefeldin A (5 ng/μL) and 2 μM monensin were added with stimulations to enable detection of intracellular cytokines. Following stimulation, cells were incubated with fixable viability stain 450 to discriminate dead from live cells, and then stained with the following surface marker antibody panel: CD3e-AF700 (clone 500A2), CD4−APC/Fire750 (clone GK1.5), CD8a-FITC (clone 53-6.7), CD62L-APC (clone MEL-14) (BioLegend), CD44-eFLuor506 (clone IM7) (Thermo). To detect intracellular cytokine expression, cells were fixed and permeabilized and immunostained using IFN□-BV605 (clone XMG1.2), IL-4-PerCP/Cy5.5 (clone 11B11) and IL-5-PE (clone TRFK5) (BioLegend) antibodies. Flow cytometry measurements were made on an Invitrogen Attune cytometer and collected data analyzed using FlowJo software (Version 10.6.2). Cells were gated on lymphocyte/singlet/live events. Recalled Th1 and Th2 cells were defined, respectively, as IFNγ-producing CD3⁺CD4⁺CD44⁺ T cells and IL-4- and/or IL-5-producing CD3⁺CD4⁺CD44⁺ T cells. Tc1 and Tc2 cells were defined, respectively, as IFNγ-producing CD3⁺CD8⁺CD44⁺ T cells and IL-4- and/or IL-5-producing CD3⁺CD8⁺CD44⁺ T cells.

### Results

### In Silico Prediction of SARS-CoV-2 T Cell Targets

To identify SARS-CoV-2 sequences recognized by T cells capable of inducing protective responses in natural infection, we analyzed the T cell immunogenicity potential of the SARS-CoV-2 surface antigens, spike, membrane and envelope, using immunoinformatic tools. As a group, these antigens are structural proteins, potential antibody targets, and estimated to be produced at higher abundance than other antigens in infected cells. Initially, it was predicted CD4⁺ T cell immunogenicity potential using the EpiMatrix T cell epitope mapping algorithm and the Wuhan-Hu-1 strain as a reference sequence. Predictions were made using nine HLA class II and six HLA class I supertype alleles representing >95% of the human population. For each antigen, regions of high class II HLA epitope density, called clusters, were identified across multiple supertype alleles. A total of 52 epitope clusters containing between 6 and 60 binding motifs each were identified (Table 57). These epitope clusters contain more than 100 total individual binding motifs for each of the nine supertype alleles, ranging from 109 for DRB1*0301 to 180 for DRB1*0901.

The CD8+ T cell immunogenicity potential of these clusters was evaluated and showed that multiple putative class I HLA epitopes overlap in regions of high class II HLA epitope density (data not shown). These epitope clusters are therefore expected to recall both CD4⁺ and CD8⁺ T cell responses in individuals with SARS-CoV-2 history and may stimulate both CD4⁺ and CD8⁺ T cell immunity in a T cell-directed vaccine. Also investigated was the conservation of these clusters in other SARS-CoV-2 isolates and it was determined that the clusters are identical to clusters found in >98.38% of strains isolated between January and September 2020 (data not shown). Highly conserved peptides such as these are useful for vaccines and as reagents for assays that interrogate T cell responses using samples from natural infection and immunization.

HLA class II ligands may stimulate effector or regulatory CD4⁺ T cells leading to divergent immunological outcomes. HLA binding predictions do not distinguish between these possibilities. They account for peptide interactions with the HLA binding groove and overlook potential interactions with the T cell receptor (TCR).

As the T cell repertoire is shaped by training on human T cell epitopes, we routinely assess potential for regulatory T cell induction by screening the TCR-face of epitopes for homology with self antigens using the JanusMatrix algorithm. For each cluster, the average depth of coverage in the human proteome was calculated holding all TCR-facing positions fixed and allowing HLA-facing positions to vary while requiring human sequences to bind to the same HLA alleles as the SARS-CoV-2 sequences. JanusMatrix analysis revealed that each SARS-COV-2 protein contains clusters with significant human homology scores (>2). This was also true for some of the 52 selected clusters. 17 (32.7%) were found to have elevated regulatory T cell induction potential based on high JanusMatrix homology with the human proteome, and in contrast, 35 (67.3%) were considered more likely to induce effector T cell responses. The results suggest that different CD4⁺ T cell subsets may be activated by these epitopes in the course of SARS-CoV-2 infection and measurement of recall responses in vitro.

32 peptides for synthesis were designed by manually editing clusters to center effector epitopes and remove epitopes with significant human homology. These 32 peptides were used to probe T-cell recognition of the putative effector T-cell epitopes. The peptides included one envelope sequence, eight sourced from membrane, and 23 from spike (Table 3A and Table 3B). Spike peptides comprise 14 in the 51 domain, including five RBD sequences, and nine in the S2 domain.

TABLE 3A
Coronavirus cross-conservation features of effector
T cell epitope peptides screened in immunoassays

| | | | Scaled Class II | | | | | | |
|---|---|---|---|---|---|---|---|---|---|
| | | | Janus CoV Homology Score^{a} | | | | | | |
| Input | SEQ ID | Cluster | SARS- | SARS- | MERS- | hCoV- | hCoV- | hCoV- | hCoV- |
| Sequence | NO: | Sequence | CoV-2 | CoV-1 | CoV | HKU1 | OC43 | NL63 | 229E |
| ENVELOPE | 210 | MYSFVSEETGTLIVN | 1.00 | 1.00 | 0.00 | 0.00 | 0.00 | 0.00 | 0.00 |
| MEMBRANE | 9 | LKKLLEQWNLVIGFL | 1.00 | 1.09 | 0.00 | 0.00 | 0.00 | 0.00 | 0.00 |
| MEMBRANE | 19 | ICLLQFAYANRNRFLYI | 1.00 | 0.89 | 0.00 | 0.00 | 0.00 | 0.00 | 0.00 |
| MEMBRANE | 34 | LSYFIASFRLFARTR | 1.00 | 0.22 | 0.00 | 0.00 | 0.00 | 0.00 | 0.00 |
| MEMBRANE | 212 | IASFRLFARTRSMWSFN | 1.00 | 1.02 | 0.00 | 0.00 | 0.00 | 0.00 | 0.00 |
| MEMBRANE | 44 | RGHLRIAGHHLGRCD | 1.00 | 0.09 | 0.00 | 0.00 | 0.00 | 0.00 | 0.00 |
| MEMBRANE | 49 | PKEITVATSRTLSYY | 1.00 | 0.73 | 0.00 | 0.00 | 0.00 | 0.00 | 0.00 |
| MEMBRANE | 54 | TLSYYKLGASQRVAGD | 1.00 | 0.92 | 0.00 | 0.00 | 0.00 | 0.00 | 0.00 |
| MEMBRANE | 59 | IGNYKLNTDHSSSSDNI | 1.00 | 0.20 | 0.00 | 0.00 | 0.00 | 0.00 | 0.00 |
| SPIKE | 214 | PPAYTNSFTRGVYY | 0.97 | 0.00 | 0.00 | 0.00 | 0.00 | 0.00 | 0.00 |
| SPIKE | 454 | NDGVYFASTEKSNIIR | 1.00 | 0.26 | 0.00 | 0.00 | 0.00 | 0.00 | 0.00 |
| SPIKE | 89 | FLGVYYHKNNKSWMESE | 1.00 | 0.00 | 0.00 | 0.00 | 0.00 | 0.00 | 0.00 |
| SPIKE | 94 | ESEFRVYSSANNCTFEYV | 1.00 | 0.00 | 0.00 | 0.00 | 0.00 | 0.00 | 0.00 |
| SPIKE | 99 | DGYFKIYSKHTPINL | 1.00 | 0.00 | 0.00 | 0.00 | 0.00 | 0.00 | 0.00 |
| SPIKE | 216 | QTLLALHRSYLTPGDSS | 0.98 | 0.00 | 0.00 | 0.00 | 0.00 | 0.00 | 0.00 |
| SPIKE | 104 | PRTFLLKYNENGTITDA | 1.00 | 0.14 | 0.00 | 0.00 | 0.00 | 0.00 | 0.00 |
| SPIKE | 109 | TSNFRVQPTESIVRF | 1.00 | 0.00 | 0.00 | 0.00 | 0.00 | 0.00 | 0.00 |
| SPIKE | 110 | FGEVFNATRFASVYA | 1.00 | 0.11 | 0.00 | 0.00 | 0.00 | 0.00 | 0.00 |
| SPIKE | 455 | FASVYAWNRKRISNCVAD | 1.00 | 0.11 | 0.00 | 0.00 | 0.00 | 0.00 | 0.00 |
| SPIKE | 114 | GGNYNYLYRLFRKSNLKP | 1.00 | 0.00 | 0.00 | 0.00 | 0.00 | 0.00 | 0.00 |
| | | FERDI | | | | | | | |
| SPIKE | 218 | VEGFNCYFPLQSYGFQPT | 1.00 | 0.00 | 0.00 | 0.00 | 0.00 | 0.00 | 0.00 |
| SPIKE | 142 | LQSYGFQPTNGVGYQPY | 1.00 | 0.00 | 0.00 | 0.00 | 0.00 | 0.00 | 0.00 |
| SPIKE | 456 | NKCVNFNFNGLTGT | 1.00 | 0.92 | 0.00 | 0.00 | 0.00 | 0.00 | 0.00 |
| SPIKE | 147 | FGGFNFSQILPDPSKPSKRS | 1.00 | 0.46 | 0.00 | 0.00 | 0.00 | 0.00 | 0.00 |
| SPIKE | 220 | TDEMIAQYTSALLA | 1.00 | 0.00 | 0.00 | 0.00 | 0.00 | 0.00 | 0.00 |
| SPIKE | 221 | QIPFAMQMAYRFNGIGV | 1.00 | 0.92 | 0.00 | 0.00 | 0.00 | 0.00 | 0.00 |
| SPIKE | 176 | QKLIANQFNSAIGKI | 1.00 | 0.00 | 0.00 | 0.00 | 0.00 | 0.00 | 0.00 |
| SPIKE | 181 | ANQFNSAIGKIQDSL | 1.00 | 0.00 | 0.00 | 0.00 | 0.00 | 0.00 | 0.00 |
| SPIKE | 191 | TGRLQSLQTYVTQQL | 1.00 | 0.92 | 0.00 | 0.00 | 0.00 | 0.00 | 0.00 |
| SPIKE | 196 | GKGYHLMSFPQSAPH | 1.00 | 0.46 | 0.00 | 0.00 | 0.00 | 0.00 | 0.00 |
| SPIKE | 223 | LDKYFKNHTSPDVDL | 1.00 | 0.92 | 0.00 | 0.00 | 0.00 | 0.00 | 0.00 |
| SPIKE | 224 | ISGINASVVNIQKEI | 1.00 | 0.92 | 0.00 | 0.00 | 0.00 | 0.00 | 0.00 |

TABLE 3B
Coronavirus cross-conservation features of effector T cell epitope peptides
screened in immunoassays

| | | | Scaled Class II | | | | | | |
|---|---|---|---|---|---|---|---|---|---|
| | | | Janus CoV TCRf 5, 8 Homology Score^{b} | | | | | | |
| Input | SEQ ID | Cluster | SARS- | SARS- | MERS- | hCoV- | hCoV- | hCoV- | hCoV- |
| Sequence | NO: | Sequence | CoV-2 | CoV-1 | CoV | HKU1 | OC43 | NL63 | 229E |
| ENVELOPE | 210 | MYSFVSEETGTLIVN | 1.00 | 1.00 | 0.00 | 0.00 | 0.00 | 0.00 | 0.00 |
| MEMBRANE | 9 | LKKLLEQWNLVIGFL | 1.00 | 1.09 | 0.00 | 0.00 | 0.00 | 0.20 | 0.80 |
| MEMBRANE | 19 | ICLLQFAYANRNRFLYI | 1.00 | 1.09 | 0.20 | 0.47 | 0.34 | 0.13 | 0.27 |
| MEMBRANE | 34 | LSYFIASFRLFARTR | 1.19 | 0.97 | 0.25 | 0.87 | 0.26 | 0.18 | 0.44 |
| MEMBRANE | 212 | IASFRLFARTRSMWSFN | 2.00 | 1.99 | 0.00 | 0.00 | 0.00 | 0.00 | 0.27 |
| MEMBRANE | 44 | RGHLRIAGHHLGRCD | 1.00 | 0.95 | 0.00 | 0.29 | 0.00 | 0.00 | 0.14 |
| MEMBRANE | 49 | PKEITVATSRTLSYY | 0.43 | 0.36 | 0.00 | 0.43 | 0.00 | 0.29 | 0.29 |
| MEMBRANE | 54 | TLSYYKLGASQRVAGD | 2.00 | 1.92 | 0.00 | 0.00 | 0.00 | 0.00 | 0.00 |
| MEMBRANE | 59 | IGNYKLNTDHSSSSDNI | 1.00 | 0.20 | 0.00 | 0.11 | 0.03 | 0.00 | 0.00 |
| SPIKE | 214 | PPAYTNSFTRGVYY | 0.97 | 0.00 | 0.17 | 0.83 | 0.67 | 0.00 | 0.00 |
| SPIKE | 454 | NDGVYFASTEKSNIIR | 1.14 | 0.39 | 0.00 | 1.12 | 0.71 | 1.14 | 0.57 |
| SPIKE | 89 | FLGVYYHKNNKSWMESE | 1.00 | 0.13 | 0.36 | 0.65 | 0.57 | 0.58 | 0.57 |
| SPIKE | 94 | ESEFRVYSSANNCTFEYV | 1.18 | 0.33 | 0.18 | 0.53 | 0.38 | 0.70 | 0.36 |
| SPIKE | 99 | DGYFKIYSKHTPINL | 1.08 | 0.08 | 0.25 | 0.02 | 0.00 | 0.08 | 0.08 |
| SPIKE | 216 | QTLLALHRSYLTPGDSS | 1.39 | 0.43 | 0.13 | 0.28 | 0.13 | 0.21 | 0.20 |
| SPIKE | 104 | PRTFLLKYNENGTITDA | 1.31 | 0.49 | 0.16 | 0.53 | 0.75 | 0.61 | 0.38 |
| SPIKE | 109 | TSNFRVQPTESIVRF | 1.50 | 0.15 | 1.16 | 0.19 | 0.00 | 0.00 | 0.07 |
| SPIKE | 110 | FGEVFNATRFASVYA | 1.00 | 0.46 | 0.62 | 0.81 | 0.49 | 0.25 | 0.13 |
| SPIKE | 455 | FASVYAWNRKRISNCVAD | 1.25 | 0.23 | 0.25 | 0.27 | 0.13 | 0.39 | 0.13 |
| SPIKE | 114 | GGNYNYLYRLFRKSNLKPF | 1.19 | 0.21 | 0.26 | 0.38 | 0.19 | 0.30 | 0.53 |
| | | ERDI | | | | | | | |
| SPIKE | 218 | VEGFNCYFPLQSYGFQPT | 1.00 | 0.08 | 0.36 | 0.10 | 0.18 | 0.26 | 0.00 |
| SPIKE | 142 | LQSYGFQPTNGVGYQPY | 1.14 | 0.13 | 0.44 | 0.12 | 0.13 | 0.43 | 0.29 |
| SPIKE | 456 | NKCVNFNFNGLTGT | 1.57 | 1.57 | 0.15 | 0.06 | 0.01 | 0.57 | 0.57 |
| SPIKE | 147 | FGGFNFSQILPDPSKPSKRS | 1.00 | 0.46 | 0.88 | 0.52 | 0.13 | 1.47 | 0.38 |
| SPIKE | 220 | TDEMIAQYTSALLA | 1.40 | 0.92 | 0.21 | 0.29 | 0.23 | 1.00 | 0.89 |
| SPIKE | 221 | QIPFAMQMAYRFNGIGV | 1.25 | 0.92 | 0.00 | 0.14 | 0.24 | 0.13 | 0.00 |
| SPIKE | 176 | QKLIANQFNSAIGKI | 1.00 | 0.79 | 0.14 | 0.58 | 0.57 | 0.29 | 0.71 |
| SPIKE | 181 | ANQFNSAIGKIQDSL | 1.00 | 0.92 | 0.14 | 0.26 | 0.00 | 1.30 | 0.57 |
| SPIKE | 191 | TGRLQSLQTYVTQQL | 1.14 | 0.92 | 1.28 | 0.14 | 1.29 | 0.86 | 0.71 |
| SPIKE | 196 | GKGYHLMSFPQSAPH | 1.00 | 0.76 | 0.00 | 0.30 | 0.67 | 0.01 | 0.70 |
| SPIKE | 223 | LDKYFKNHTSPDVDL | 1.33 | 1.02 | 0.00 | 0.33 | 0.12 | 0.45 | 0.00 |
| SPIKE | 224 | ISGINASVVNIQKEI | 1.11 | 1.02 | 0.22 | 0.43 | 0.22 | 0.53 | 0.67 |

SARS-CoV-2 Epitope Conservation with Related Coronaviruses at the TCR-Face

Also investigated was the conservation of the selected peptides with related coronaviruses that infect humans including highly pathogenic SARS-CoV and MERS-CoV and low pathogenicity common cold coronaviruses (CCCs) 0C43, HKU1, NL63, and 229E. Prior exposure to these viruses may have established T-cell memory that can be recalled upon SARS-CoV-2 infection and vaccination. As well, SARS-CoV-2 infection and vaccination establishes T-cell memory that can influence responses in future infections to these viruses or yet-to-emerge coronaviruses. To identify potentially cross-reactive sequences, the TCR-face of SARS-CoV-2 epitopes was screened for homology with coronaviruses that infect humans, using the JanusMatrix algorithm (Table 3A and Table 3B). All of the membrane and envelope sequences shared identical TCR-face patterns with SARS-CoV. Half the spike clusters were unique to SARS-CoV-2 and the other half are conserved with SARS-CoV. Only three clusters were cross-conserved outside SARS viruses. Given reports of pre-existing T cell immunity in people with no SARS-CoV-2 experience, we relaxed the requirement for 100% identity at every TCR-face position. Fixing two positions shown to be extensively involved in TCR interactions (positions 5 and 8), JanusMatrix predicted an expanded cross-conservation landscape for SARS-CoV-2 spike and membrane clusters. Most spike clusters were conserved, by these criteria, in the subset of coronaviruses that infect humans. The remainder of the sequences with cross-reactivity potential are cross-conserved among highly pathogenic beta-coronaviruses or among high and low pathogenicity beta-coronaviruses. Only three clusters were unique to SARS-CoV-2 by the criteria described above, and none are solely conserved with SARS-CoV. The single membrane clusters was cross-conserved in the highly pathogenic beta-coronaviruses and coronaviruses that infect humans subsets.

As the vast majority of people were not exposed to SARS-CoV and MERS-CoV, the cross-conservation between SARS-CoV-2 and CCCs only was explored. Of the 32 peptides, only 2 are SARS-CoV-2-specific. Eighteen clusters (56.6%) are cross-conserved across 0C43, HKU1, NL63, and 229E and 12 (37.5%) are cross-conserved in at least one of these four CCCs.

### Clinical Cohort

Persons with and without SARS-CoV-2 infection history were selected to provide PBMC samples for validation of predicted T cell epitopes. COVID-19 convalescents with PCR-confirmed SARS-CoV-2 infection (N=15) over March-June 2020 were recruited between 30 and 180 days after their most recent positive test and a minimum of 14 days after symptoms resolved (Table 59, Table 60). Donors exhibited a wide range of COVID-19 symptoms and experienced either mild or moderate disease according to WHO criteria. Blood draws from convalescents ranged from approximately one to six months from diagnosis. Healthy individuals (N=10) provided cell samples from February 2016 up to November 2019 and had no opportunity for SARS-CoV-2 exposure. Both cohorts contain a balanced proportion of females and males and similar average age and age range. 60% of convalescent donors are from racial and ethnic minorities. No ethnicity information is available for the healthy cohort.

TABLE 4
COVID-19 convalescent and healthy donor cohort characteristics
Donor Characteristics

| | |
|---|---|
| Naïve | Convalescent |
| (N = 10) | (N = 15) |

Age (years)

| | | |
|---|---|---|
| Average | 48.5 | 44.8 |
| Range | 26-79 | 23-71 |

Gender

| | | |
|---|---|---|
| Female | 40% (4/10) | 53.33% (8/15) |
| Male | 60% (6/10) | 46.67% (7/15) |

Ethnicity

| | | |
|---|---|---|
| Caucasian | N/A | 40% (6/15) |
| Hispanic/Latino | N/A | 33.33% (5/15) |
| African American | N/A | 6.67% (1/15) |
| Asian/Pacific Islander | N/A | 20% (3/15) |

Disease Severity

| | | |
|---|---|---|
| Mild | N/A | 73.33% (11/15) |
| Moderate | N/A | 26.67% (4/15) |
| Severe | N/A | 0 |
| Critical | N/A | 0 |

Recruitment

| | | |
|---|---|---|
| Sample Collection Date | February 2016- | May-October 2020 |
| | November 2019 | |
| Date Tested Positive | N/A | March-June 2020 |
| Time Since Last | N/A | 33-158 |
| Positive Test (Days) | | |
| Time Since Symptoms | N/A | 26-116 |
| Resolved (Days) | | |
| Date Last Tested Positive | N/A | March-June 2020 |
| Date Symptoms Resolved | N/A | March-June 2020 |
| PCR Positivity | N/A | 100% (15/15) |

TABLE 5
Additional COVID-19 convalescent and healthy donor cohort characteristics
Donor Characteristics

| | |
|---|---|
| Naïve | Convalescent |
| (N = 10) | (N = 15) |

BMI

| | | |
|---|---|---|
| Range | N/A | 21.1-43.4 |
| Obese | N/A | 13.33% (2/15) |
| Overweight | N/A | 46.67% (7/15) |
| Normal | N/A | 40% (6/15) |
| Underweight | N/A | 0 |

Symptoms

| | | |
|---|---|---|
| Fever | N/A | 73.33% (11/15) |
| Fever Duration (days) | N/A | 1.5-14 |
| Cough | N/A | 80% (12/15) |
| Shortness of breath | N/A | 46.67% (7/15) |
| Muscle pain | N/A | 80% (12/15) |
| Headache | N/A | 93.33% (14/15) |
| Sore throat | N/A | 53.33% (8/15) |
| Loss of taste or smell | N/A | 73.33% (11/15) |
| Nausea | N/A | 26.67% (4/15) |
| Weight Loss | N/A | 6.67% (1/15) |
| Loss of appetite | N/A | 20% (3/15) |
| Diarrhea | N/A | 40% (6/15) |
| Urinary Incontinence | N/A | 6.67% (1/15) |
| Fatigue | N/A | 20% (3/15) |
| Malaise | N/A | 6.67% (1/15) |
| Sweats | N/A | 20% (3/15) |
| Chills | N/A | 20% (3/15) |
| Dizziness | N/A | 13.33% (2/15) |
| Pneumonia | N/A | 13.33% (2/15) |
| Anemia | N/A | 6.67% (1/15) |
| Sensitivity to light | N/A | 6.67% (1/15) |
| Peeling Skin | N/A | 6.67% (1/15) |
| Chest Pain | N/A | 6.67% (1/15) |
| Back Pain | N/A | 6.67% (1/15) |
| Numbing of foot | N/A | 6.67% (1/15) |

Disease Severity

| | | |
|---|---|---|
| Mild | N/A | 73.33% (11/15) |
| Moderate | N/A | 26.67% (4/15) |
| Severe | N/A | 0 |
| Critical | N/A | 0 |

Past Medical History

| | |
|---|---|
| Asthma | 20% (3/15) |
| Fibroids | 6.67% (1/15) |
| Myopia | 20% (3/15) |
| Hypertension | 13.33% (2/15) |
| GERD | 20% (3/15) |
| Hyperopia | 13.33% (2/15) |
| Arthritis | 6.67% (1/15) |
| Hypothyroidism | 13.33% (2/15) |
| Cataract | 6.67% (1/15) |
| Osteopenia | 6.67% (1/15) |
| Breast cancer (in remission) | 6.67% (1/15) |
| Osteoarthritis | 6.67% (1/15) |

Allergies

| | |
|---|---|
| Seasonal | 6.67% (1/15) |
| Dust | 6.67% (1/15) |
| Sulfa | 13.33% (2/15) |
| Ceclor | 6.67% (1/15) |
| Dairy | 6.67% (1/15) |
| Unknown medications | 6.67% (1/15) |

Current Medications

| | |
|---|---|
| Metoprolol | 6.67% (1/15) |
| Inhaler | 6.67% (1/15) |
| Natural D-Hist | 6.67% (1/15) |
| Vitamin C | 6.67% (1/15) |
| Aspirin | 6.67% (1/15) |
| Losartin | 6.67% (1/15) |
| Protonix | 6.67% (1/15) |
| Amlodipine | 6.67% (1/15) |
| Meprazole | 6.67% (1/15) |
| Naprosyn | 6.67% (1/15) |
| Vitamin B | 6.67% (1/15) |
| Multivitamin | 6.67% (1/15) |
| Fish Oil | 6.67% (1/15) |
| Zinc | 6.67% (1/15) |
| Magnesium | 6.67% (1/15) |
| Tumeric | 6.67% (1/15) |
| Atenolol | 6.67% (1/15) |
| Levothryoxine | 6.67% (1/15) |
| Pantoprazole | 6.67% (1/15) |
| Lisinopril | 6.67% (1/15) |
| Vitamin D3 | 6.67% (1/15) |

Lifestyle Habits

| | |
|---|---|
| Tobacco Use (used to smoke) | 40% (6/15) |
| Light Caffeine Use | 6.67% (1/15) |
| Heavy Caffeine Use | 66.67% (10/15) |
| Light Alcohol Use | 26.67% (4/15) |
| Moderate Alcohol Use | 26.67% (4/15) |
| Recreational Drug Use | 6.67% (1/15) |
| Marijuana Use | 33.33% (5/15) |

### Predicted Epitopes are Recognized in Natural SARS-CoV-2 Infection

To determine what predicted effector CD4⁺ T cell epitope clusters are recognized by T cells raised in SARS-CoV-2 infection, ex vivo IFNγ Fluorospot assays were conducted using whole PBMC preparations from convalescent donors. Immune recall in the Fluorospot assay was stimulated using either individual or pooled peptides. Responses that were both ≥25 spot forming cells over background and >5-fold over background were considered positive. In preliminary studies, a small cohort of donors were stimulated with lower concentrations of peptide (0.313 m/mL per pooled peptide, 10 μg/mL for individual peptide stimulations). These concentrations were increased to potentially heighten assay sensitivity, however, it was found that there was no significant difference in the frequency of epitope-specific clones nor in the number of peptides detected per donor between the assay conditions, thus both data sets are combined here.

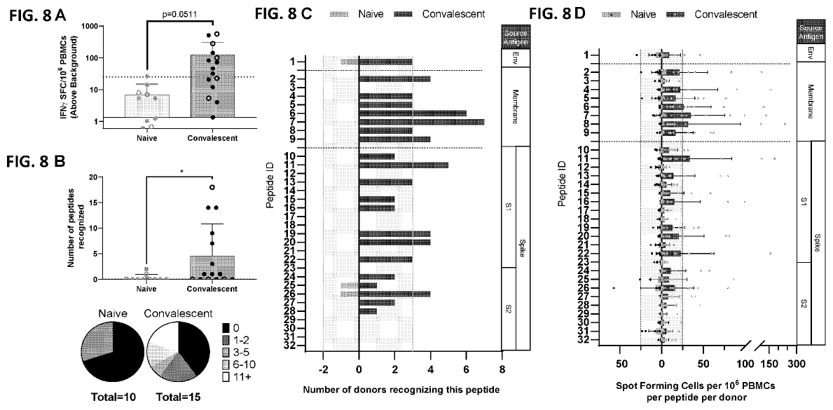

Overall, it was found that the majority of COVID-19 convalescent donors (9/15; 60%) responded to a pool of the 32 peptides (FIG. 8A). In contrast, only a single healthy control donor out of ten demonstrated immune recall, just above the threshold, to this pool. Individual peptide stimulations showed that COVID-19 convalescent donors had positive responses to envelope, membrane, and spike peptides, including responses in the S1 and S2 domains and RBD (FIG. 8D).

For 21/32 (66%) peptides, a recall response was observed in at least one convalescent donor, (corresponding to 1/1 envelope-derived epitope, 7/8 (87.5%) membrane-derived epitopes, 13/23 (56.5%) spike-derived epitopes); 14/32 (44%) peptides were confirmed in at least 20% of convalescent donors (FIG. 8C).

The number of peptides confirmed per donor ranged from 0 to 18 with an average response of 4-5 peptides per convalescent donor (FIG. 8B). Of the healthy donors, only two recognized any of the peptides ex vivo: the aforementioned donor responding to the total peptide pool recalled the sole envelope epitope (peptide 1) and an S2-derived sequence (peptide 25), while another donor mounted a response to an S2 domain sequence (peptide 26). Although rare, these findings suggest that there may be a memory response that cross-reacts with prior common cold coronavirus infection.

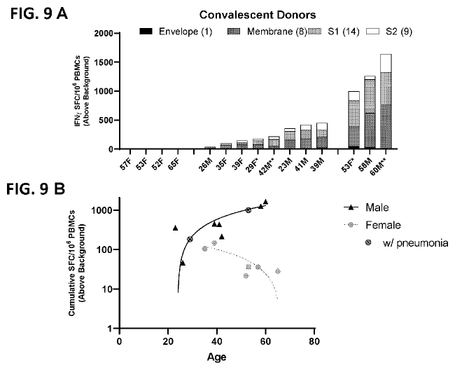

Given the variable patterns of epitope-specific responses, the cumulative response to specific antigens by batching the response to individual peptides was evaluated (FIG. 9A). As expected from the individual donor peptide analysis, T cells specific for membrane-derived epitopes were most frequently recalled with 11/15 donors demonstrating significant anti-membrane responses and membrane epitope-specific T cells making up >40% of the total recall response in 10/11 donors. We also found that despite greater variability in responses to individual S1-derived epitopes, the magnitude of the response to these peptides was similar to the magnitude of membrane-specific T cell responses in individual donors.

Interestingly, the quality of each donor's cumulative response could be used to distinguish three distinct immunophenotypes within the cohort. These cohorts were defined by whether individuals mounted a (1) robust, (2) weak, or (3) no T cell response. Furthermore, divergent trends in terms of responses could be identified for males and females (FIG. 9B).

While similar T cell response levels were observed in the younger individuals evaluated in this study, regardless of sex, they diverge with age with higher effector T cell responses in males only (R=0.757, p=0.011, n=7); conversely, older women (>50 years) primarily exhibit decreased T cell activity. Correlation analysis in the complete cohort of women did not achieve significance (R<0.0001, p=0.993, n=8) due to two outliers (both moderate cases; pneumonia without requiring supplemental oxygen or hospitalization) and possibly because of the small size of the study. However, in the absence of these outliers, the cohort of women with mild cases shows a significant correlation between effector T cell responses and age (R=0.704, p=0.037, n=6)

Collectively, these findings highlight the high prevalence of membrane-targeted T cell responses in most COVID-19 cases and demonstrates the importance of targeting spike in SARS-CoV-2 vaccination to hasten and focus T cell responses to this antigen upon infection.

### Pre-Existing SARS-CoV-2 Immunity in Healthy Donors is Stimulated by Predicted Epitopes Following Antigen-Specific Cell Expansion

While convalescent donors as a group recognized a majority of predicted epitopes ex vivo, as individuals, each recognized only a smaller subset. We hypothesized that SARS-CoV-2-induced T cell clones underwent variable expansion and contraction over the course of disease resulting in memory T cell populations resulting in frequencies of T cell responses that were both detectable and undetectable ex vivo. To uncover low frequency epitope-specific T cells, PBMCs were stimulated with SARS-CoV-2 peptides and expanded in short-term culture and then re-stimulated with individual or pools of peptides in an IFNγ Fluorospot assay. Although changes to cellular phenotypes over the course of in vitro expansion do not represent the natural immune response to infection, expansion of epitope-specific T cells present ex vivo and their detection by cultured Fluorospot assay may augment the repertoire of immunogenic SARS-CoV-2 T cell epitopes and suggest greater T cell memory is generated than thought from ex vivo recall alone.

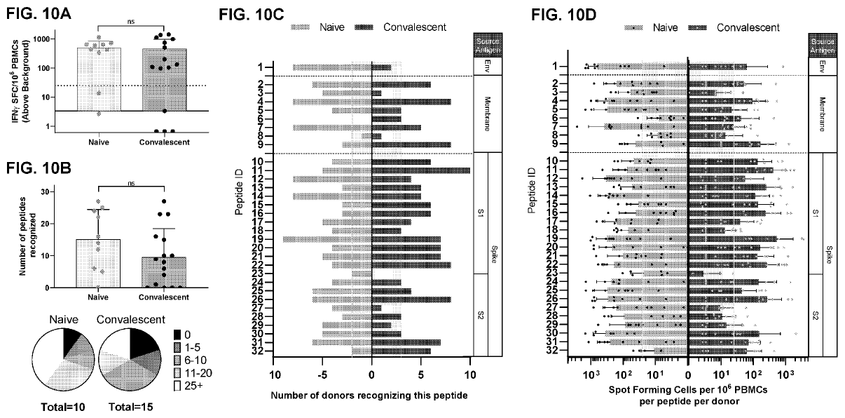

Using the same criteria defining an IFNγ response as above, we found that 11/15 (73.3%) convalescent donors mounted a response to the pool of 32 peptides (FIG. 10A). Like the ex vivo assay, individual peptide stimulations showed positive responses to envelope and membrane peptides, as well as spike peptides covering the S1 and S2 domains and RBD (FIG. 10D). Only a single S2-derived epitope was not recalled. Convalescent donors recognized between 0 and 27 peptides with an average of 9-10 peptide responses per donor. (FIG. 10B). At least one convalescent donor recognized 31/32 (96.9%) peptides, with the majority of peptides (26/32; including 0/1 envelope epitope, 6/8 membrane epitopes, and 20/23 spike epitopes) being confirmed in >20% of our cohort. (FIG. 10C). Changes in IFNγ responses between ex vivo and cultured assay measurements identify broader patterns of peptide recognition associated with source antigen (Data not shown). For example, responses identified in convalescent donors to envelope and membrane-derived antigens were rarely replicated following culture in donors that exhibited positive responses ex vivo. In contrast, responses to spike-derived peptides were primarily identified following culture, however those that did response ex vivo were much more likely to maintain positive responses following culture as well. These differences may reflect variable memory phenotypes (effector vs. central), proliferative capacities, and/or activation/exhaustion profiles of epitope-specific T cell populations following natural infection.

Healthy donors mounted strong recall responses to SARS-CoV-2 T cell epitopes following expansion culture in sharp contrast to the ex vivo response. To the pool of 32 peptides, 8/10 (80%) healthy donors elicited detectable IFNγ responses (FIG. 10A). To individual peptide restimulations, cross-reactive T cell responses were found to envelope, membrane, and spike S1, S2, and RBD-derived peptides (FIG. 10D). On average individual control donors responded to 15 peptides, ranging from 0-27 recognized peptides per donor (FIG. 10B). Following culture, 31/32 (96.9%) peptides stimulated responses in at least one control donor. Of these, only a single peptide was not confirmed in ≥20% of the healthy cohort (FIG. 10C). The 30 peptides correspond to 1/1 envelope peptide, 6/8 membrane peptides, and 23/23 spike peptides. Overall, the magnitude and prevalence of cross-reactive T cell responses in donors with no SARS-CoV-2 history suggests T cell memory to common cold coronavirus infection may contribute to immune response to SARS-CoV-2 infection. SARS-CoV-2 peptide vaccination stimulates type 1 immunity Confirmation that these predicted SARS-CoV-2 peptides are recognized by T cells raised in infection suggested that they may elicit de novo immune responses by vaccination. Peptide vaccination may prime CD4 and CD8 T cells and generate immune memory that is recalled upon infection to support protective humoral and cellular mechanisms of immunity. Of the 32 peptides, we selected the highest EpiMatrix scoring peptides (above 20) for vaccination. (One peptide was removed to due to poor solubility in the pool and was replaced with the 21^{st} ranked peptide.) Twenty peptides is in range with what is used in peptide vaccine clinical trials. Peptides were formulated with poly-ICLC (Hiltonol), an adjuvant composed of carboxymethylcellulose, poly-inosinic-poly-cytidylic acid, and poly-L-lysine double-stranded RNA, that stimulates the TLR-3 and MDA-5 innate immune pathways and Th1-skewed CD4 T cell responses.

HLA-DR3 transgenic mice were primed and boosted with the peptide vaccine (EPV-CoV-19) to assess in vivo vaccine immunogenicity in the context of human MHC restriction, which is not feasible using wild-type mice. MHC Class II-mediated cellular immunity in the mouse MHC-II knockout/HLA-DR3 knock-in transgenic strain is completely restricted by human MHC, not by its murine ortholog, and presents peptides that are also recognized by human T cells. Thus, vaccine immunogenicity in the HLA-DR3 mouse model may support human application of SARS-CoV-2 peptides. Mice were immunized by the intradermal route. A published peptide vaccine clinical trial utilizing poly-ICLC adjuvant and intradermal route of immunization reported minimal injection site reactions and no toxicity associated with the immunizations.

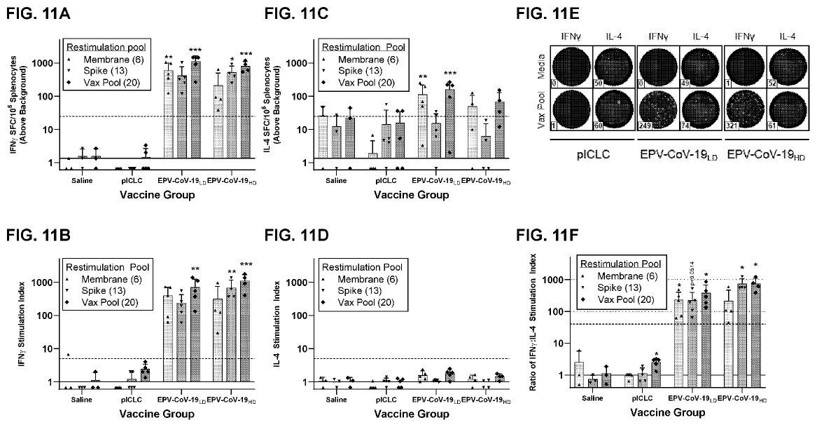

The type 1/type 2 T cell balance stimulated by vaccination was assessed by measuring cytokine production. IFNγ production was measured as a marker of type 1 responses and IL-4 and IL-5 as markers of type 2 responses. In an IFNγ/IL-4 dual cytokine Fluorospot assay (FIG. 11A), splenocytes from individual mice were stimulated with pools of all vaccine peptides, as well as membrane-derived peptides or spike-derived peptides in the vaccine. All low- and high-dose vaccinated mice mounted an IFNγ response to each pool, as defined by SFC (FIG. 11B) and SI (FIG. 11C) criteria. Although the magnitude of response to each pool was statistically no different between the low- and high-dose groups, the ratio of spike-specific:membrane-specific IFNγ responses was significantly higher in animals that received the high-dose vaccine. Control mice that received either poly-ICLC only or saline did not respond. IL-4 secretion meeting both positivity criteria was not detected in either immunized or mock-immunized mice (FIG. 11D-E). A ratio of the IFNγ and IL-4 responses was used as an indicator of the type 1/type 2 T cell balance. For both the low-dose and high-dose vaccine groups, the IFNγ/IL-4 ratio skews strongly toward type 1 (FIG. 11F).

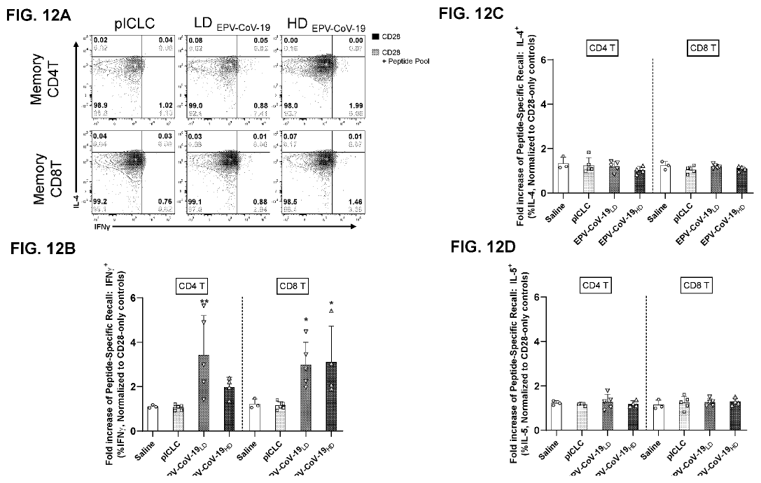

To assess cytokine production in T cell subpopulations, we used intracellular cytokine staining and flow cytometry to measure frequency, mean fluorescence intensity, and differentiation status of splenic CD4⁺ and CD8⁺ T cells responding to EPV-CoV-19 peptides. Representative flow cytometry data, including gating, is shown in FIG. 12A. EPV-CoV-19 vaccination at both low and high doses stimulated statistically significant increases in numbers of IFNγ-producing memory T cells that were recalled specifically by vaccine peptides, as well as increased average amounts of IFNγ produced per cell compared to control mice (FIG. 12B and data not shown). In contrast, neither IL-4 nor IL-5 production from memory CD4⁺ and CD8⁺ T cells could be restimulated by vaccine peptides in vitro, above controls (FIG. 12C-D). Although we do note a minimal increase in the frequency of IL-5-producing CD4⁺ T cells from vaccinated animals (Data not shown) there is no concurrent increase in IL-5 production per cell, nor is the magnitude of this response comparable to the induction of Th1 subsets, thus minimizing the functional significance of this observation.

Overall, both the low-dose and high-dose vaccine groups exhibit IFNγ/(IL-4+IL-5) ratios that sharply skew toward Th1/Tc1 (FIG. 12E).

Taken altogether, these results show EPV-CoV-19 stimulates a strong T cell response that can be recalled to elicit a potent type 1-skewed response in a dose-independent manner while also critically avoiding significant type 2 induction that is associated with enhanced respiratory disease. Using these same experiments as outlined in Example 7, it is expected that the polypeptides of the instant disclosure (e.g., but not limited to, a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 4-224 and 454-456, as well as Ii-key constructs of the present disclosure, including those of Table 2) will stimulate a strong T cell response that can be recalled to elicit a potent type 1-skewed response in a dose-independent manner while also critically avoiding significant type 2 induction that is associated with enhanced respiratory disease

### Example 8

### Overall Design:

A total of 28 healthy participants who meet the eligibility criteria will be enrolled in this study. The participants will be divided into 4 sequential cohorts of 7 participants each, with randomization of treatment within cohorts so that 5 participants receive active vaccine and 2 receive saline placebo. The first 3 cohorts will consist of participants seronegative for severe acute respiratory syndrome coronavirus 2 (SARS-CoV-2) antibody who will receive either placebo or vaccine with 10 μg/peptide (e.g., but not limited to, a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 4-224 and 454-456, as well as Ii-key constructs of the present disclosure, including those of Table 2) if in the first cohort, 50 μg/peptide if in the second cohort and 150 μg/peptide if in the third. The fourth cohort will consist of participants seropositive for SARS-CoV-2 antibody who will receive either saline placebo or a vaccine dose of 10 μg/peptide.

The dose levels for dose escalation are planned as 10 μg, 50 μg, and 150 μg per peptide, administered intradermally. The starting dose for vaccine containing a polypeptide as disclosed herein (e.g., but not limited to, a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 4-224 and 454-456, as well as Ii-key constructs of the present disclosure, including those of Table 2) will be 10 μg per peptide (for up to 20 peptides). The maximum dose, 150 μg per peptide, is lower than the subcutaneous or intramuscular doses (300 μg per peptide) used in Phase 2/3 studies reported by Ott et al for their neoantigen peptide vaccine clinical study. The total peptide dose proposed for the FIH study will be well below those administered safely in previous clinical studies using synthetic peptides.

A staggered dosing will be used for each cohort:

On Day 1, 1 participant will receive a vaccine comprising polypeptides of the instant disclosure and 1 participant will receive saline placebo.

The remaining participants will be dosed at least 48 hours (h) later provided satisfactory safety and tolerability is demonstrated for the participants dose on Day 1.

All participants in the dose ranging part of the study (Cohort 1 to Cohort 3) will go through a Screening Period of up to 14 days (Day −14 to Day −1), while those with positive serum antibodies for SARS-CoV-2 (Cohort 4) will have a Screening Period of up to 30 days (Day −30 to Day −1). Study intervention (including saline placebo and therapeutic vaccine as disclosed herein) will be given to all participants at the study site on Day 1 and Day 15. Participants will be released at the discretion of the Investigator following completion of the assessment at 60 minutes post each vaccination provided there are no safety concerns identified from the review of the clinical safety data. All participants will return to the study site on Day 3 (2 days after the first vaccination), Day 8 (one week after the first vaccination), Day 22 (one week after the second vaccination), Day 43 (28 days after the second vaccination), and at 2, 6, and 12 months after the second vaccination for safety, tolerability, and immunogenicity follow-up. Participants will receive monthly phone calls from 3 months after the second vaccination until the end of study (EOS) for safety and efficacy follow-up, except for the months (M6 and M12) where site visit is required. An unscheduled visit for safety and coronavirus disease 2019 (COVID-19) symptom follow-up may be needed as judged by the Investigator.

Safety, tolerability, and immunogenicity data for each cohort will be reviewed by the Safety Monitoring Committee (SMC) before the dose escalation to the next cohort and before involving SARS-CoV-2 antibody positive participants. Dose escalation will only take place after one week of safety follow-up for the second vaccination of the last participant in the previous cohort is completed and provided the vaccine as disclosed herein is well tolerated following review of all safety, tolerability, and immunogenicity data from the previous cohort by the SMC. Similarly, the cohort of SARS-CoV-2 antibody positive participants will only receive investigational product after one week of safety follow-up for the second vaccination of the last participant from the dose ranging groups is completed and provided the vaccine as disclosed herein is well tolerated following review of all safety, tolerability, and immunogenicity data from the previous cohort by the SMC. Due to the logistics of the bioanalytical immunogenicity analysis, immunogenicity data for the most recently enrolled cohort will not be included in the safety review for the cohort, but will be included when available in subsequent reviews. Screening procedures may continue between dose escalations to facilitate enrollment of remaining participants.

### Brief Summary:

The purpose of this study is to assess the safety, tolerability, and immunogenicity of a T cell epitope-driven vaccine comprising one or more of the instantly-disclosed polypeptide (e.g., but not limited to, a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 4-224 and 454-456, as well as Ii-key constructs of the present disclosure, including those of Table 2) compared with saline placebo in healthy participants. Study details include:

The study duration will be up to: 13 months for Cohort 1 to Cohort 3 and 13.5 months for Cohort 4

The treatment duration will be up to: 15 days

The visit frequency will be: Day 1, Day 3, weekly from Day 8 to Day 22, followed by Day 43, and at 2, 6, and 12 months after the second vaccination; follow-up by phone calls will be performed monthly from 3 months after the second vaccination till the EOS, except for the months (M6 and M12) where site visit is required.

### Number of Participants:

A maximum of 28 healthy participants will be randomly assigned to study intervention.

Note: “Enrolled” means a participant's, or their legally acceptable representative's, agreement to participate in a clinical study following completion of the informed consent process and screening. Potential participants who are screened for the purpose of determining eligibility for the study, but do not participate in the study, are not considered enrolled, unless otherwise specified by the protocol. A participant will be considered enrolled if the informed consent is not withdrawn prior to participating in any study activity after screening.

### Intervention Groups and Duration:

Total duration of study participation for participants in the dose ranging part of the study (Cohort 1 to Cohort 3) will be up to 13 months and that for participants with positive serum antibodies for SARS-CoV-2 (Cohort 4) will be up to 13.5 months: a Screening Period of up to 14 days, a Treatment and Follow-up Period of 75 days (from first dosing to 60 days after the second dosing), and a Long-term Follow-up Period up to 12 months after the second vaccination.

Participants will receive study intervention (including saline placebo and a vaccine as disclosed herein [e.g., 10 μg, 50 μg, and 150 μg per peptide for up to 20 peptides, which may be, e.g., but not limited to, a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 4-224 and 454-456, as well as Ii-key constructs of the present disclosure, including those of Table 2)) administered by the Investigator/authorized study personnel at the study site on Day 1 and Day 15.

On each dosing day, the study intervention will be given as four separate i.d. injections (0.5 mL per injection) to each of the four extremities by Mantoux technique. As a vaccine may be composed of four peptide pools (five peptides (which may be, e.g., but not limited to, a peptide or polypeptide comprising, consisting, or consisting essentially of an amino acid sequence of SEQ ID NOS: 4-224 and 454-456 (and/or fragments or variants thereof), and optionally 1 to 12 additional amino acids distributed in any ratio on the N terminus and/or C-terminus of the polypeptide of SEQ ID NOS: 4-224 and 454-456, as well as Ii-key constructs of the present disclosure, including those of Table 2)) per pool), each pool will be injected at one injection site. The injection sites at each time will be schematically documented in the case report form (CRF). Participants will be released at the discretion of the Investigator following completion of the assessment at 60 minutes post each vaccination provided there are no safety concerns identified from the review of the clinical safety data.

### Stopping/Halting Rules:

For each participant, the booster (second dosing) will be halted if one of the following occurs: a Grade 2 adverse event (AE) lasting for more than two weeks or, a Grade 3 AE, a hypersensitivity reaction of any grade, or positive pregnancy test.

For each dosing cohort, study intervention will be halted if one of the following occurs: two Grade 2 AEs lasting for more than two weeks, two Grade 3AEs, one Grade 2 AEs lasting for more than two weeks combined with one Grade 3 AE, or one Grade 4 AE. The study/study enrollment will be halted at the same time if study intervention is halted for a dosing cohort.

Adverse events will be graded using FDA Guidance for Industry: Toxicity Grading Scale for Healthy Adult and Adolescent Volunteers Enrolled in Preventive Vaccine Clinical Trials (September 2007).

### Safety Monitoring Committee:

A SMC has been appointed for this study. The SMC includes Principal Investigator (PI), medical monitor, and Sponsor medical representative and is appointed to monitor the safety and scientific integrity of a human research intervention, and to make recommendations to the Sponsor regarding the stopping of a study for efficacy, for harms, or for futility. The composition of the committee is dependent upon the scientific skills and knowledge required for monitoring the study.

### Study Population:

### Inclusion Criteria

Participants are eligible to be included in the study only if all of the following criteria apply:
1. Ability to provide written informed consent prior to initiation of any study procedures.
2. Be able to understand and agrees to comply with planned study procedures and be available for all study visits.
3. Agrees to the collection of venous blood per protocol.
4. Male or non-pregnant female, 18 to 55 years of age, inclusive, at time of enrollment.
5. Body Mass Index 18-35 kg/m², inclusive, at Screening.
6. Women of childbearing potential must have a negative urine or serum pregnancy test within 24 hours prior to each vaccination.
7. Oral temperature is less than 100.4 degrees Fahrenheit (38.0 degrees Celsius).
8. Pulse no greater than 100 beats per minute.
9. Systolic blood pressure is 85 to 150 mmHg, inclusive.
10. Clinical screening laboratory evaluations (white blood cell [WBC], hemoglobin [Hgb], platelets [PLTs], alanine transaminase [ALT], aspartate transaminase [AST], creatinine [Cr], alkaline phosphatase [ALP], total bilirubin [TBL], Lipase, prothrombin time [PT], and partial thromboplastin time [PTT]) are within acceptable normal reference ranges at the clinical laboratory being used.
11. Must agree to have samples stored for secondary research.
12. The participant must agree to refrain from donating blood or plasma during the study (outside of this study).
13. Negative human immunodeficiency virus (HIV) diagnostic test.
14. Contraceptive use by men and women must be consistent with local regulations regarding the methods of contraception for those participating in clinical studies.

NOTE: The reliability of sexual abstinence for male and/or female enrollment eligibility needs to be evaluated in relation to the duration of the clinical study and the preferred and usual lifestyle of the participant. Periodic abstinence (e.g., calendar, ovulation, symptothermal, or post ovulation methods) and withdrawal are not acceptable methods of contraception.

a. Male Participants:

A male participant must agree to use a highly effective contraception as detailed in Appendix 4 of this protocol during the intervention period and an additional 90 days (a spermatogenesis cycle) after the last dose of study intervention and refrain from donating sperm during this period.

b. Female Participants:

A female participant is eligible to participate if she is not pregnant (see Appendix 4), not breastfeeding, and at least one of the following conditions applies: Not a woman of childbearing potential (WOCBP) as defined in Appendix 4.

OR

A WOCBP who agrees to follow the contraceptive guidance in Appendix 4 during the intervention period and an additional 90 days after the last dose of study intervention.

Additional Criteria Applicable Only to Cohorts 1-3:
15. Negative test for SARS-CoV-2 in nasopharyngeal swabs/sputum swabs through real-time reverse transcriptase polymerase chain reaction (RT-PCR) and BinaxNow™ COVID-19 test on Day −1.
16. Negative test for serum IgG antibodies to SARS-CoV-2 (titer<1:80) measured by antibody assay (per standardized assay at central laboratory, Mt. Sinai School of Medicine).

Additional Criteria Applicable Only to Cohort 4:
17. Negative test for SARS-CoV-2 in nasopharyngeal swabs/sputum swabs through RT-PCR and BinaxNow™ COVID-19 test on Day −30 through Day −1.
18. Positive test for serum IgG antibodies to SARS-CoV-2 (titer≥1:80) measured by antibody assay (per standardized assay at central laboratory, Mt. Sinai School of Medicine).
19. Fulfills either one of the following criteria with regard to prior COVID-19 symptoms:
   a. Never had symptoms of COVID-19.
OR
   b. Experienced mild to moderate symptoms of COVID-19 that resolved at least 30 days prior to vaccination and never required hospitalization.

### Exclusion Criteria

Participants are excluded from the study if any of the following criteria apply:
1. Positive pregnancy test either at Screening or just prior to each vaccine administration.
2. Female participant who is breastfeeding or plans to breastfeed from the time of the first vaccination through 60 days after the last vaccination.
3. Has any medical disease or condition that, in the opinion of the site PI or appropriate sub-investigator, precludes study participation.

Note: including acute, subacute, intermittent or chronic medical disease or condition that would place the participant at an unacceptable risk of injury, render the participant unable to meet the requirements of the protocol, or may interfere with the evaluation of responses or the participant's successful completion of this study.
4. Presence of self-reported or medically documented significant medical or psychiatric condition(s).

Note: significant medical or psychiatric conditions include but are not limited to:
(1) respiratory disease (e.g., chronic obstructive pulmonary disease [COPD], asthma) requiring daily medications currently or any treatment of respiratory disease exacerbations (e.g., asthma exacerbation) in the last 5 years. Asthma medications: inhaled, oral, or intravenous corticosteroids, leukotriene modifiers, long and short acting beta agonists, theophylline, ipratropium, biologics.
(2) significant cardiovascular disease (e.g., congestive heart failure, cardiomyopathy, ischemic heart disease) or history of myocarditis or pericarditis as an adult.
(3) neurological or neurodevelopmental conditions (e.g., history of migraines in the past 5 years, epilepsy, stroke, seizures in the last 3 years, encephalopathy, focal neurologic deficits, Guillain-Barré syndrome, encephalomyelitis or transverse myelitis).
(4) ongoing malignancy or recent diagnosis of malignancy in the last five years excluding basal cell and squamous cell carcinoma of the skin, which are allowed.
(5) an autoimmune disease, including hypothyroidism without a defined non-autoimmune cause, localized, or history of psoriasis.
(6) An immunodeficiency of any cause.
5. Has an acute illness, as determined by the site PI or appropriate sub-investigator, with or without fever (oral temperature≥38.0 degrees Celsius [100.4 degrees Fahrenheit]) within 72 h prior to each vaccination.

Note: an acute illness which is nearly resolved with only minor residual symptoms remaining is allowable if, in the opinion of the site PI or appropriate sub-investigator, the residual symptoms will not interfere with the ability to assess safety parameters as required by the protocol.
6. Has a positive test result for hepatitis B surface antigen, hepatitis C virus antibody, or HIV types 1 or 2 antibodies at Screening.
7. Has participated in another investigational study involving any investigational product (including study drug, biologic, or device) within 60 days, or 5 half-lives, whichever is longer, before the first vaccine administration.
8. Currently enrolled in or plans to participate in another clinical trial with an investigational agent (including licensed or unlicensed vaccine, drug, biologic, device, blood product, or medication) that will be received during the study reporting period (or 13 months after the first vaccination).
9. Has a history of hypersensitivity or severe allergic reaction (e.g., anaphylaxis, generalized urticaria, angioedema, other significant reaction) to any previous licensed or unlicensed vaccines.
10. Chronic use (more than 14 continuous days) of any medications that may be associated with impaired immune responsiveness. This includes, but not limited to, systemic corticosteroids exceeding 10 mg/day of prednisone equivalent, allergy injections, immunoglobulin, interferon, immunomodulators, cytotoxic drugs, or other similar or toxic drugs during the preceding 6-month period prior to the first vaccine administration. The use of low dose topical, ophthalmic, inhaled and intranasal steroid preparations will be permitted.
11. Received immunoglobulins and/or any blood or blood products within the 4 months before the first vaccine administration or at any time during the study.
12. Has any blood dyscrasias or significant disorder of coagulation.
13. Has a history of alcohol abuse or other recreational drug (excluding cannabis) use within 6 months before the first vaccine administration.
14. Has any abnormality or permanent body art (e.g., tattoo) that will interfere with the ability to observe local reactions at the injection site (deltoid/thigh region).
15. Received or plans to receive a licensed, live vaccine within 4 weeks before or after each vaccination.
16. Received or plans to receive a licensed, inactivated vaccine within 2 weeks before or after each vaccination.
17. Receipt of any other SARS-CoV-2 or other experimental coronavirus vaccine at any time prior to or during the study.
18. On current treatment with investigational agents for prophylaxis of COVID-19.
19. Current use of any prescription or over-the-counter medications within 7 days prior to vaccination, unless approved by the Investigator.
20. Plan to travel outside the US (continental US, Hawaii, and Alaska) from enrollment through 28 days after the second vaccination.
21. Participant allergic to any component of the investigational vaccine, or a more severe allergic reaction and history of allergies in the past.
22. Direct contact with a person who has tested positive for SARS-CoV-2 within 30 days of enrollment.

Additional Criteria Applicable Only to Cohorts 1-3:
23. A history of laboratory-confirmed SARS-CoV-2 infection.
24. Participant has had clinical signs and symptoms consistent with SARS-CoV-2 infection (including stuffy or runny nose, score throat, shortness of breath, cough, low energy or tiredness, muscle or body aches, headache, chills or shivering, feeling hot or feverish, nausea, vomit, diarrhea, reduced sense of smell and taste, as specified in the FDA guidance) within 2 weeks prior to the first vaccination.

Additional Criterion Applicable Only to Cohort 4:

Participant has had clinical signs and symptoms consistent with SARS-CoV-2 infection (including stuffy or runny nose, score throat, shortness of breath, cough, low energy or tiredness, muscle or body aches, headache, chills or shivering, feeling hot or feverish, nausea, vomit, diarrhea, reduced sense of smell and taste, as specified in the FDA guidance) and a positive SARS-CoV-2 test result within 30 days prior to the first vaccination.

1.-97. (canceled)

98. A composition comprising:
a polypeptide comprising an amino acid sequence selected from the group consisting of SEQ ID NOS: 224, 454-456, and/or fragments and variants thereof;
a polypeptide comprising an amino acid sequence selected from the group consisting of SEQ ID NOS: 4-224, 454-456 further comprising 1 to 12 additional amino acids distributed in any ratio on the N terminus and/or C-terminus of the polypeptide;
a nucleic acid encoding said polypeptide;
a plasmid encoding said polypeptide;
a vector encoding said polypeptide; or
a pharmaceutical composition comprising said polypeptide, said nucleic acid, said plasmid, or said vector, said pharmaceutical composition further comprising a pharmaceutically-acceptable carrier and/or excipient.

99. The composition according to claim 98, wherein said variant or fragment of an amino acid sequence selected from the group consisting of SEQ ID NOS: 4-224, 454-456 retains WIC binding propensity and TCR specificity, and/or retains anti-SARS-CoV-2 activity.

100. The composition of claim 98 comprising an amino acid sequence having at least 80% homology to any one of SEQ ID NOS: 4-224, 454-456, and fragments thereof, wherein said polypeptide retains MHC binding propensity and the same TCR specificity, and/or retains anti-SARS-CoV-2 activity.

101. The composition of claim 98, wherein said nucleic acid encoding said polypeptide comprises a sequence selected from the group consisting of SEQ ID NOS: 232-452 and 457-459 and fragments or variants thereof.

102. The composition of claim 98, wherein said polypeptide is a chimeric or fusion polypeptide, wherein said polypeptide is joined, linked, or inserted into a heterologous polypeptide.

103. A vaccine comprising the polypeptide, the nucleic acid, the plasmid or the vector according to claim 98 and a pharmaceutically-acceptable excipient, carrier, and/or adjuvant.

104. A method for inducing immunity against a coronavirus infection, optionally a SARS-CoV-2 infection, Severe Acute Respiratory Syndrome (SARS-CoV) or Middle East respiratory syndrome coronavirus (MERS-CoV)) and/or diseases caused by said coronavirus in a subject in need thereof, the method comprising administering to the subject a therapeutically effective amount of the composition according to claim 98, or
a vaccine comprising the polypeptide, the nucleic acid, the plasmid or the vector according to claim 98 and a pharmaceutically-acceptable excipient, carrier, and/or adjuvant.

105. The method according to claim 104, wherein the step of administration additionally includes administration of an SARS-CoV-2 virus, wherein the virus is a live attenuated virus or inactivated virus.

106. The method according to claim 104, the method comprising administering to the subject a therapeutically effective amount of one or more of the polypeptides or the nucleic acids.

107. The method according to claim 106, wherein the step of administration additionally includes administration of a SARS-CoV-2 virus, wherein the virus is a live attenuated virus or inactivated virus.

108. A method for measuring a CMI response against a coronavirus in a subject, said method comprising; collecting a whole blood sample from said subject wherein said whole blood sample comprises cells of the immune system which are capable of producing immune effector molecules following stimulation by an antigen, incubating a mixture comprising the whole blood sample, at least one polypeptide according to claim 98, or the polypeptide of claim 98 with an amount of an isolated simple sugar effective to enhance the stimulation by the antigen and/or heparin, and measuring the presence of, or elevation in, a level of an immune effector molecule wherein a presence or level of said immune effector molecule is indicative of the capacity of said subject to mount a cell-mediated immune response.

109. The method of claim 108 wherein the subject is a human.

110. The method of claim 108 wherein the whole blood is collected in a tube comprising at least one of said polypeptide.

111. The method of claim 108 wherein the whole blood is collected in a tube comprising heparin.

112. The method of claim 108 wherein the whole blood sample is incubated with the at least one of said polypeptide for from 5 to 50 hours.

113. The method of claim 108 wherein the immune effector molecule is a cytokine.

114. The method of claim 113 wherein the cytokine is IFN-γ, GM-CSF, an interleukin or a TNF-α.

115. The method of claim 108, wherein the subject is infected by a coronavirus.

116. The method of claim 108, wherein the immune cells are NK cells, T-cells, B-cells, dendritic cells, macrophages, or monocytes.

117. An assay for identifying SARS-CoV-2 or a related coronavirus-specific immediate effector T cells in a subject, comprising: (a) providing a sample from said subject containing T cells; (b) exposing said T cells to an immunogenic amount of at least one polypeptide according to claim 98; and (c) prior to the generation of new immediate effector T cells in the sample, determining whether said T cells are activated by said polypeptide by measuring secretion of a cytokine from said T cells; wherein activation of said T cells identifies the presence of SARS-CoV-2 or a related coronavirus-specific immediate effector T cells that were present in the original sample, in said subject.

118. An assay for identifying coronavirus-specific immediate effector T cells in a subject, comprising: (a) providing a sample from said subject containing T cells; (b) exposing said T cells to an immunogenic amount of at least one polypeptide according to claim 98; (c) incubating said T cells for a period of time which is not sufficient to effect differentiation of quiescent T cells to immediate effector T cells; and (d) determining whether said T cells are activated by said polypeptide by measuring secretion of a cytokine from said T cells, wherein activation of said T cells identifies the presence of SARS-CoV-2 or a related coronavirus-specific immediate effector T cells in said subject.

119. A method of diagnosing infection in a human host by, or exposure of a human host to, a SARS-CoV-2 or a related coronavirus, which method comprises the steps of: (i) contacting a population of T cells from the host with one or more polypeptides according to claim 98; and (ii) determining in vitro whether the T cells of said T cell population show a recognition response to said polypeptide.

120. A method of preventing, treating, or ameliorating a disease by SARS-CoV-2 infection in a subject in need thereof, the method comprising administering to the subject a therapeutically effective amount of one or more of a composition according to claim 98.


SEQUENCE LISTING (459 sequences)

SEQ ID NO: 1 - protein, 75 aa, Severe acute respiratory syndrome coronavirus 2
MYSFVSEETGTLIVNSVLLFLAFVVFLLVTLAILTALRLCAYCCNIVNVSLVKPSFYVYSRVKNLNSSRVPDLLV

SEQ ID NO: 2 - protein, 222 aa, Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 3 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 16 aa, Severe acute respiratory syndrome coronavirus 2
PSFYVYSRVKNLNSSR

SEQ ID NO: 5 - protein, 15 aa, Severe acute respiratory syndrome coronavirus 2
SFYVYSRVKNLNSSR

SEQ ID NO: 6 - protein, 14 aa, Severe acute respiratory syndrome coronavirus 2
FYVYSRVKNLNSSR

SEQ ID NO: 7 - protein, 13 aa, Severe acute respiratory syndrome coronavirus 2
YVYSRVKNLNSSR

SEQ ID NO: 8 - protein, 11 aa, Severe acute respiratory syndrome coronavirus 2
YVYSRVKNLNS

SEQ ID NO: 9 - protein, 15 aa, Severe acute respiratory syndrome coronavirus 2
LKKLLEQWNLVIGFL

SEQ ID NO: 10 - protein, 14 aa, Severe acute respiratory syndrome coronavirus 2
KKLLEQWNLVIGFL

SEQ ID NO: 11 - protein, 13 aa, Severe acute respiratory syndrome coronavirus 2
KLLEQWNLVIGFL

SEQ ID NO: 12 - protein, 12 aa, Severe acute respiratory syndrome coronavirus 2
LLEQWNLVIGFL

SEQ ID NO: 13 - protein, 9 aa, Severe acute respiratory syndrome coronavirus 2
LLEQWNLVI

SEQ ID NO: 14 - protein, 15 aa, Severe acute respiratory syndrome coronavirus 2
LEQWNLVIGFLFLTW

SEQ ID NO: 15 - protein, 14 aa, Severe acute respiratory syndrome coronavirus 2
EQWNLVIGFLFLTW

SEQ ID NO: 16 - protein, 13 aa, Severe acute respiratory syndrome coronavirus 2
QWNLVIGFLFLTW

SEQ ID NO: 17 - protein, 12 aa, Severe acute respiratory syndrome coronavirus 2
WNLVIGFLFLTW

SEQ ID NO: 18 - protein, 9 aa, Severe acute respiratory syndrome coronavirus 2
WNLVIGFLF

SEQ ID NO: 19 - protein, 17 aa, Severe acute respiratory syndrome coronavirus 2
ICLLQFAYANRNRFLYI

SEQ ID NO: 20 - protein, 16 aa, Severe acute respiratory syndrome coronavirus 2
CLLQFAYANRNRFLYI

SEQ ID NO: 21 - protein, 15 aa, Severe acute respiratory syndrome coronavirus 2
LLQFAYANRNRFLYI

SEQ ID NO: 22 - protein, 14 aa, Severe acute respiratory syndrome coronavirus 2
LQFAYANRNRFLYI

SEQ ID NO: 23 - protein, 11 aa, Severe acute respiratory syndrome coronavirus 2
LQFAYANRNRF

SEQ ID NO: 24 - protein, 19 aa, Severe acute respiratory syndrome coronavirus 2
RNRFLYIIKLIFLWLLWPV

SEQ ID NO: 25 - protein, 18 aa, Severe acute respiratory syndrome coronavirus 2
NRFLYIIKLIFLWLLWPV

SEQ ID NO: 26 - protein, 17 aa, Severe acute respiratory syndrome coronavirus 2
RFLYIIKLIFLWLLWPV

SEQ ID NO: 27 - protein, 16 aa, Severe acute respiratory syndrome coronavirus 2
FLYIIKLIFLWLLWPV

SEQ ID NO: 28 - protein, 13 aa, Severe acute respiratory syndrome coronavirus 2
FLYIIKLIFLWLL

SEQ ID NO: 29 - protein, 15 aa, Severe acute respiratory syndrome coronavirus 2
LACFVLAAVYRINWI

SEQ ID NO: 30 - protein, 14 aa, Severe acute respiratory syndrome coronavirus 2
ACFVLAAVYRINWI

SEQ ID NO: 31 - protein, 13 aa, Severe acute respiratory syndrome coronavirus 2
CFVLAAVYRINWI

SEQ ID NO: 32 - protein, 12 aa, Severe acute respiratory syndrome coronavirus 2
FVLAAVYRINWI

SEQ ID NO: 33 - protein, 9 aa, Severe acute respiratory syndrome coronavirus 2
FVLAAVYRI

SEQ ID NO: 34 - protein, 15 aa, Severe acute respiratory syndrome coronavirus 2
LSYFIASFRLFARTR

SEQ ID NO: 35 - protein, 14 aa, Severe acute respiratory syndrome coronavirus 2
SYFIASFRLFARTR

SEQ ID NO: 36 - protein, 13 aa, Severe acute respiratory syndrome coronavirus 2
YFIASFRLFARTR

SEQ ID NO: 37 - protein, 12 aa, Severe acute respiratory syndrome coronavirus 2
FIASFRLFARTR

SEQ ID NO: 38 - protein, 9 aa, Severe acute respiratory syndrome coronavirus 2
FIASFRLFA

SEQ ID NO: 39 - protein, 18 aa, Severe acute respiratory syndrome coronavirus 2
IASFRLFARTRSMWSFNP

SEQ ID NO: 40 - protein, 17 aa, Severe acute respiratory syndrome coronavirus 2
ASFRLFARTRSMWSFNP

SEQ ID NO: 41 - protein, 16 aa, Severe acute respiratory syndrome coronavirus 2
SFRLFARTRSMWSFNP

SEQ ID NO: 42 - protein, 15 aa, Severe acute respiratory syndrome coronavirus 2
FRLFARTRSMWSFNP

SEQ ID NO: 43 - protein, 12 aa, Severe acute respiratory syndrome coronavirus 2
FRLFARTRSMWS

SEQ ID NO: 44 - protein, 15 aa, Severe acute respiratory syndrome coronavirus 2
RGHLRIAGHHLGRCD

SEQ ID NO: 45 - protein, 14 aa, Severe acute respiratory syndrome coronavirus 2
GHLRIAGHHLGRCD

SEQ ID NO: 46 - protein, 13 aa, Severe acute respiratory syndrome coronavirus 2
HLRIAGHHLGRCD

SEQ ID NO: 47 - protein, 12 aa, Severe acute respiratory syndrome coronavirus 2
LRIAGHHLGRCD

SEQ ID NO: 48 - protein, 9 aa, Severe acute respiratory syndrome coronavirus 2
LRIAGHHLG

SEQ ID NO: 49 - protein, 15 aa, Severe acute respiratory syndrome coronavirus 2
PKEITVATSRTLSYY

SEQ ID NO: 50 - protein, 14 aa, Severe acute respiratory syndrome coronavirus 2
KEITVATSRTLSYY

SEQ ID NO: 51 - protein, 13 aa, Severe acute respiratory syndrome coronavirus 2
EITVATSRTLSYY

SEQ ID NO: 52 - protein, 12 aa, Severe acute respiratory syndrome coronavirus 2
ITVATSRTLSYY

SEQ ID NO: 53 - protein, 9 aa, Severe acute respiratory syndrome coronavirus 2
ITVATSRTL

SEQ ID NO: 54 - protein, 16 aa, Severe acute respiratory syndrome coronavirus 2
TLSYYKLGASQRVAGD

SEQ ID NO: 55 - protein, 15 aa, Severe acute respiratory syndrome coronavirus 2
LSYYKLGASQRVAGD

SEQ ID NO: 56 - protein, 14 aa, Severe acute respiratory syndrome coronavirus 2
SYYKLGASQRVAGD

SEQ ID NO: 57 - protein, 13 aa, Severe acute respiratory syndrome coronavirus 2
YYKLGASQRVAGD

SEQ ID NO: 58 - protein, 10 aa, Severe acute respiratory syndrome coronavirus 2
YYKLGASQRV

SEQ ID NO: 59 - protein, 17 aa, Severe acute respiratory syndrome coronavirus 2
IGNYKLNTDHSSSSDNI

SEQ ID NO: 60 - protein, 16 aa, Severe acute respiratory syndrome coronavirus 2
GNYKLNTDHSSSSDNI

SEQ ID NO: 61 - protein, 15 aa, Severe acute respiratory syndrome coronavirus 2
NYKLNTDHSSSSDNI

SEQ ID NO: 62 - protein, 14 aa, Severe acute respiratory syndrome coronavirus 2
YKLNTDHSSSSDNI

SEQ ID NO: 63 - protein, 11 aa, Severe acute respiratory syndrome coronavirus 2
YKLNTDHSSSS

SEQ ID NO: 64 - protein, 15 aa, Severe acute respiratory syndrome coronavirus 2
PPAYTNSFTRGVYYP

SEQ ID NO: 65 - protein, 14 aa, Severe acute respiratory syndrome coronavirus 2
PAYTNSFTRGVYYP

SEQ ID NO: 66 - protein, 13 aa, Severe acute respiratory syndrome coronavirus 2
AYTNSFTRGVYYP

SEQ ID NO: 67 - protein, 12 aa, Severe acute respiratory syndrome coronavirus 2
YTNSFTRGVYYP

SEQ ID NO: 68 - protein, 9 aa, Severe acute respiratory syndrome coronavirus 2
YTNSFTRGV

SEQ ID NO: 69 - protein, 15 aa, Severe acute respiratory syndrome coronavirus 2
QDLFLPFFSNVTWFH

SEQ ID NO: 70 - protein, 14 aa, Severe acute respiratory syndrome coronavirus 2
DLFLPFFSNVTWFH

SEQ ID NO: 71 - protein, 13 aa, Severe acute respiratory syndrome coronavirus 2
LFLPFFSNVTWFH

SEQ ID NO: 72 - protein, 12 aa, Severe acute respiratory syndrome coronavirus 2
FLPFFSNVTWFH

SEQ ID NO: 73 - protein, 9 aa, Severe acute respiratory syndrome coronavirus 2
FLPFFSNVT

SEQ ID NO: 74 - protein, 16 aa, Severe acute respiratory syndrome coronavirus 2
NVTWFHAIHVSGTNGT

SEQ ID NO: 75 - protein, 15 aa, Severe acute respiratory syndrome coronavirus 2
VTWFHAIHVSGTNGT

SEQ ID NO: 76 - protein, 14 aa, Severe acute respiratory syndrome coronavirus 2
TWFHAIHVSGTNGT

SEQ ID NO: 77 - protein, 13 aa, Severe acute respiratory syndrome coronavirus 2
WFHAIHVSGTNGT

SEQ ID NO: 78 - protein, 10 aa, Severe acute respiratory syndrome coronavirus 2
WFHAIHVSGT

SEQ ID NO: 79 - protein, 17 aa, Severe acute respiratory syndrome coronavirus 2
NDGVYFASTEKSNIIRG

SEQ ID NO: 80 - protein, 16 aa, Severe acute respiratory syndrome coronavirus 2
DGVYFASTEKSNIIRG

SEQ ID NO: 81 - protein, 15 aa, Severe acute respiratory syndrome coronavirus 2
GVYFASTEKSNIIRG

SEQ ID NO: 82 - protein, 14 aa, Severe acute respiratory syndrome coronavirus 2
VYFASTEKSNIIRG

SEQ ID NO: 83 - protein, 11 aa, Severe acute respiratory syndrome coronavirus 2
VYFASTEKSNI

SEQ ID NO: 84 - protein, 17 aa, Severe acute respiratory syndrome coronavirus 2
TQSLLIVNNATNVVIKV

SEQ ID NO: 85 - protein, 16 aa, Severe acute respiratory syndrome coronavirus 2
QSLLIVNNATNVVIKV

SEQ ID NO: 86 - protein, 15 aa, Severe acute respiratory syndrome coronavirus 2
SLLIVNNATNVVIKV

SEQ ID NO: 87 - protein, 14 aa, Severe acute respiratory syndrome coronavirus 2
LLIVNNATNVVIKV

SEQ ID NO: 88 - protein, 12 aa, Severe acute respiratory syndrome coronavirus 2
LLIVNNATNVVI

SEQ ID NO: 89 - protein, 17 aa, Severe acute respiratory syndrome coronavirus 2
FLGVYYHKNNKSWMESE

SEQ ID NO: 90 - protein, 16 aa, Severe acute respiratory syndrome coronavirus 2
LGVYYHKNNKSWMESE

SEQ ID NO: 91 - protein, 15 aa, Severe acute respiratory syndrome coronavirus 2
GVYYHKNNKSWMESE

SEQ ID NO: 92 - protein, 14 aa, Severe acute respiratory syndrome coronavirus 2
VYYHKNNKSWMESE

SEQ ID NO: 93 - protein, 11 aa, Severe acute respiratory syndrome coronavirus 2
VYYHKNNKSWM

SEQ ID NO: 94 - protein, 18 aa, Severe acute respiratory syndrome coronavirus 2
ESEFRVYSSANNCTFEYV

SEQ ID NO: 95 - protein, 17 aa, Severe acute respiratory syndrome coronavirus 2
SEFRVYSSANNCTFEYV

SEQ ID NO: 96 - protein, 16 aa, Severe acute respiratory syndrome coronavirus 2
EFRVYSSANNCTFEYV

SEQ ID NO: 97 - protein, 15 aa, Severe acute respiratory syndrome coronavirus 2
FRVYSSANNCTFEYV

SEQ ID NO: 98 - protein, 12 aa, Severe acute respiratory syndrome coronavirus 2
FRVYSSANNCTF

SEQ ID NO: 99 - protein, 15 aa, Severe acute respiratory syndrome coronavirus 2
DGYFKIYSKHTPINL

SEQ ID NO: 100 - protein, 14 aa, Severe acute respiratory syndrome coronavirus 2
GYFKIYSKHTPINL

SEQ ID NO: 101 - protein, 13 aa, Severe acute respiratory syndrome coronavirus 2
YFKIYSKHTPINL

SEQ ID NO: 102 - protein, 12 aa, Severe acute respiratory syndrome coronavirus 2
FKIYSKHTPINL

SEQ ID NO: 103 - protein, 9 aa, Severe acute respiratory syndrome coronavirus 2
FKIYSKHTP

SEQ ID NO: 104 - protein, 17 aa, Severe acute respiratory syndrome coronavirus 2
PRTFLLKYNENGTITDA

SEQ ID NO: 105 - protein, 16 aa, Severe acute respiratory syndrome coronavirus 2
RTFLLKYNENGTITDA

SEQ ID NO: 106 - protein, 15 aa, Severe acute respiratory syndrome coronavirus 2
TFLLKYNENGTITDA

SEQ ID NO: 107 - protein, 14 aa, Severe acute respiratory syndrome coronavirus 2
FLLKYNENGTITDA

SEQ ID NO: 108 - protein, 11 aa, Severe acute respiratory syndrome coronavirus 2
FLLKYNENGTI

SEQ ID NO: 109 - protein, 15 aa, Severe acute respiratory syndrome coronavirus 2
TSNFRVQPTESIVRF

SEQ ID NO: 110 - protein, 14 aa, Severe acute respiratory syndrome coronavirus 2
SNFRVQPTESIVRF

SEQ ID NO: 111 - protein, 13 aa, Severe acute respiratory syndrome coronavirus 2
NFRVQPTESIVRF

SEQ ID NO: 112 - protein, 12 aa, Severe acute respiratory syndrome coronavirus 2
FRVQPTESIVRF

SEQ ID NO: 113 - protein, 9 aa, Severe acute respiratory syndrome coronavirus 2
FRVQPTESI

SEQ ID NO: 114 - protein, 15 aa, Severe acute respiratory syndrome coronavirus 2
FGEVFNATRFASVYA

SEQ ID NO: 115 - protein, 14 aa, Severe acute respiratory syndrome coronavirus 2
GEVFNATRFASVYA

SEQ ID NO: 116 - protein, 13 aa, Severe acute respiratory syndrome coronavirus 2
EVFNATRFASVYA

SEQ ID NO: 117 - protein, 12 aa, Severe acute respiratory syndrome coronavirus 2
VFNATRFASVYA

SEQ ID NO: 118 - protein, 9 aa, Severe acute respiratory syndrome coronavirus 2
VFNATRFAS

SEQ ID NO: 119 - protein, 14 aa, Severe acute respiratory syndrome coronavirus 2
FASVYAWNRKRISN

SEQ ID NO: 120 - protein, 13 aa, Severe acute respiratory syndrome coronavirus 2
ASVYAWNRKRISN

SEQ ID NO: 121 - protein, 12 aa, Severe acute respiratory syndrome coronavirus 2
SVYAWNRKRISN

SEQ ID NO: 122 - protein, 11 aa, Severe acute respiratory syndrome coronavirus 2
VYAWNRKRISN

SEQ ID NO: 123 - protein, 10 aa, Severe acute respiratory syndrome coronavirus 2
VYAWNRKRIS

SEQ ID NO: 124 - protein, 15 aa, Severe acute respiratory syndrome coronavirus 2
VADYSVLYNSASFST

SEQ ID NO: 125 - protein, 14 aa, Severe acute respiratory syndrome coronavirus 2
ADYSVLYNSASFST

SEQ ID NO: 126 - protein, 13 aa, Severe acute respiratory syndrome coronavirus 2
DYSVLYNSASFST

SEQ ID NO: 127 - protein, 12 aa, Severe acute respiratory syndrome coronavirus 2
YSVLYNSASFST

SEQ ID NO: 128 - protein, 15 aa, Severe acute respiratory syndrome coronavirus 2
GGNYNYLYRLFRKSN

SEQ ID NO: 129 - protein, 14 aa, Severe acute respiratory syndrome coronavirus 2
GNYNYLYRLFRKSN

SEQ ID NO: 130 - protein, 13 aa, Severe acute respiratory syndrome coronavirus 2
NYNYLYRLFRKSN

SEQ ID NO: 131 - protein, 12 aa, Severe acute respiratory syndrome coronavirus 2
YNYLYRLFRKSN

SEQ ID NO: 132 - protein, 9 aa, Severe acute respiratory syndrome coronavirus 2
YNYLYRLFR

SEQ ID NO: 133 - protein, 15 aa, Severe acute respiratory syndrome coronavirus 2
NYLYRLFRKSNLKPF

SEQ ID NO: 134 - protein, 14 aa, Severe acute respiratory syndrome coronavirus 2
YLYRLFRKSNLKPF

SEQ ID NO: 135 - protein, 13 aa, Severe acute respiratory syndrome coronavirus 2
LYRLFRKSNLKPF

SEQ ID NO: 136 - protein, 12 aa, Severe acute respiratory syndrome coronavirus 2
YRLFRKSNLKPF

SEQ ID NO: 137 - protein, 9 aa, Severe acute respiratory syndrome coronavirus 2
YRLFRKSNL

SEQ ID NO: 138 - protein, 14 aa, Severe acute respiratory syndrome coronavirus 2
RLFRKSNLKPFERD

SEQ ID NO: 139 - protein, 13 aa, Severe acute respiratory syndrome coronavirus 2
LFRKSNLKPFERD

SEQ ID NO: 140 - protein, 12 aa, Severe acute respiratory syndrome coronavirus 2
FRKSNLKPFERD

SEQ ID NO: 141 - protein, 9 aa, Severe acute respiratory syndrome coronavirus 2
FRKSNLKPF

SEQ ID NO: 142 - protein, 18 aa, Severe acute respiratory syndrome coronavirus 2
VEGFNCYFPLQSYGFQPT

SEQ ID NO: 143 - protein, 17 aa, Severe acute respiratory syndrome coronavirus 2
EGFNCYFPLQSYGFQPT

SEQ ID NO: 144 - protein, 16 aa, Severe acute respiratory syndrome coronavirus 2
GFNCYFPLQSYGFQPT

SEQ ID NO: 145 - protein, 15 aa, Severe acute respiratory syndrome coronavirus 2
FNCYFPLQSYGFQPT

SEQ ID NO: 146 - protein, 12 aa, Severe acute respiratory syndrome coronavirus 2
FNCYFPLQSYGF

SEQ ID NO: 147 - protein, 17 aa, Severe acute respiratory syndrome coronavirus 2
LQSYGFQPTNGVGYQPY

SEQ ID NO: 148 - protein, 16 aa, Severe acute respiratory syndrome coronavirus 2
QSYGFQPTNGVGYQPY

SEQ ID NO: 149 - protein, 15 aa, Severe acute respiratory syndrome coronavirus 2
SYGFQPTNGVGYQPY

SEQ ID NO: 150 - protein, 14 aa, Severe acute respiratory syndrome coronavirus 2
YGFQPTNGVGYQPY

SEQ ID NO: 151 - protein, 11 aa, Severe acute respiratory syndrome coronavirus 2
YGFQPTNGVGY

SEQ ID NO: 152 - protein, 15 aa, Severe acute respiratory syndrome coronavirus 2
VLSFELLHAPATVCG

SEQ ID NO: 153 - protein, 14 aa, Severe acute respiratory syndrome coronavirus 2
LSFELLHAPATVCG

SEQ ID NO: 154 - protein, 13 aa, Severe acute respiratory syndrome coronavirus 2
SFELLHAPATVCG

SEQ ID NO: 155 - protein, 12 aa, Severe acute respiratory syndrome coronavirus 2
FELLHAPATVCG

SEQ ID NO: 156 - protein, 9 aa, Severe acute respiratory syndrome coronavirus 2
FELLHAPAT

SEQ ID NO: 157 - protein, 15 aa, Severe acute respiratory syndrome coronavirus 2
NKCVNFNFNGLTGTG

SEQ ID NO: 158 - protein, 14 aa, Severe acute respiratory syndrome coronavirus 2
KCVNFNFNGLTGTG

SEQ ID NO: 159 - protein, 13 aa, Severe acute respiratory syndrome coronavirus 2
CVNFNFNGLTGTG

SEQ ID NO: 160 - protein, 12 aa, Severe acute respiratory syndrome coronavirus 2
VNFNFNGLTGTG

SEQ ID NO: 161 - protein, 9 aa, Severe acute respiratory syndrome coronavirus 2
VNFNFNGLT

SEQ ID NO: 162 - protein, 15 aa, Severe acute respiratory syndrome coronavirus 2
NFNFNGLTGTGVLTE

SEQ ID NO: 163 - protein, 14 aa, Severe acute respiratory syndrome coronavirus 2
FNFNGLTGTGVLTE

SEQ ID NO: 164 - protein, 13 aa, Severe acute respiratory syndrome coronavirus 2
NFNGLTGTGVLTE

SEQ ID NO: 165 - protein, 12 aa, Severe acute respiratory syndrome coronavirus 2
FNGLTGTGVLTE

SEQ ID NO: 166 - protein, 9 aa, Severe acute respiratory syndrome coronavirus 2
FNGLTGTGV

SEQ ID NO: 167 - protein, 14 aa, Severe acute respiratory syndrome coronavirus 2
TQLNRALTGIAVEQ

SEQ ID NO: 168 - protein, 13 aa, Severe acute respiratory syndrome coronavirus 2
QLNRALTGIAVEQ

SEQ ID NO: 169 - protein, 12 aa, Severe acute respiratory syndrome coronavirus 2
LNRALTGIAVEQ

SEQ ID NO: 170 - protein, 9 aa, Severe acute respiratory syndrome coronavirus 2
LNRALTGIA

SEQ ID NO: 171 - protein, 15 aa, Severe acute respiratory syndrome coronavirus 2
TDEMIAQYTSALLAG

SEQ ID NO: 172 - protein, 14 aa, Severe acute respiratory syndrome coronavirus 2
DEMIAQYTSALLAG

SEQ ID NO: 173 - protein, 13 aa, Severe acute respiratory syndrome coronavirus 2
EMIAQYTSALLAG

SEQ ID NO: 174 - protein, 12 aa, Severe acute respiratory syndrome coronavirus 2
MIAQYTSALLAG

SEQ ID NO: 175 - protein, 10 aa, Severe acute respiratory syndrome coronavirus 2
MIAQYTSALL

SEQ ID NO: 176 - protein, 17 aa, Severe acute respiratory syndrome coronavirus 2
QIPFAMQMAYRFNGIGV

SEQ ID NO: 177 - protein, 16 aa, Severe acute respiratory syndrome coronavirus 2
IPFAMQMAYRFNGIGV

SEQ ID NO: 178 - protein, 15 aa, Severe acute respiratory syndrome coronavirus 2
PFAMQMAYRFNGIGV

SEQ ID NO: 179 - protein, 14 aa, Severe acute respiratory syndrome coronavirus 2
FAMQMAYRFNGIGV

SEQ ID NO: 180 - protein, 11 aa, Severe acute respiratory syndrome coronavirus 2
FAMQMAYRFNG

SEQ ID NO: 181 - protein, 15 aa, Severe acute respiratory syndrome coronavirus 2
QKLIANQFNSAIGKI

SEQ ID NO: 182 - protein, 14 aa, Severe acute respiratory syndrome coronavirus 2
KLIANQFNSAIGKI

SEQ ID NO: 183 - protein, 13 aa, Severe acute respiratory syndrome coronavirus 2
LIANQFNSAIGKI

SEQ ID NO: 184 - protein, 12 aa, Severe acute respiratory syndrome coronavirus 2
IANQFNSAIGKI

SEQ ID NO: 185 - protein, 9 aa, Severe acute respiratory syndrome coronavirus 2
IANQFNSAI

SEQ ID NO: 186 - protein, 15 aa, Severe acute respiratory syndrome coronavirus 2
ANQFNSAIGKIQDSL

SEQ ID NO: 187 - protein, 14 aa, Severe acute respiratory syndrome coronavirus 2
NQFNSAIGKIQDSL

SEQ ID NO: 188 - protein, 13 aa, Severe acute respiratory syndrome coronavirus 2
QFNSAIGKIQDSL

SEQ ID NO: 189 - protein, 12 aa, Severe acute respiratory syndrome coronavirus 2
FNSAIGKIQDSL

SEQ ID NO: 190 - protein, 9 aa, Severe acute respiratory syndrome coronavirus 2
FNSAIGKIQ

SEQ ID NO: 191 - protein, 15 aa, Severe acute respiratory syndrome coronavirus 2
TGRLQSLQTYVTQQL

SEQ ID NO: 192 - protein, 14 aa, Severe acute respiratory syndrome coronavirus 2
GRLQSLQTYVTQQL

SEQ ID NO: 193 - protein, 13 aa, Severe acute respiratory syndrome coronavirus 2
RLQSLQTYVTQQL

SEQ ID NO: 194 - protein, 12 aa, Severe acute respiratory syndrome coronavirus 2
LQSLQTYVTQQL

SEQ ID NO: 195 - protein, 9 aa, Severe acute respiratory syndrome coronavirus 2
LQSLQTYVT

SEQ ID NO: 196 - protein, 15 aa, Severe acute respiratory syndrome coronavirus 2
GKGYHLMSFPQSAPH

SEQ ID NO: 197 - protein, 14 aa, Severe acute respiratory syndrome coronavirus 2
KGYHLMSFPQSAPH

SEQ ID NO: 198 - protein, 13 aa, Severe acute respiratory syndrome coronavirus 2
GYHLMSFPQSAPH

SEQ ID NO: 199 - protein, 12 aa, Severe acute respiratory syndrome coronavirus 2
YHLMSFPQSAPH

SEQ ID NO: 200 - protein, 9 aa, Severe acute respiratory syndrome coronavirus 2
YHLMSFPQS

SEQ ID NO: 201 - protein, 15 aa, Severe acute respiratory syndrome coronavirus 2
DVVIGIVNNTVYDPL

SEQ ID NO: 202 - protein, 14 aa, Severe acute respiratory syndrome coronavirus 2
VVIGIVNNTVYDPL

SEQ ID NO: 203 - protein, 13 aa, Severe acute respiratory syndrome coronavirus 2
VIGIVNNTVYDPL

SEQ ID NO: 204 - protein, 10 aa, Severe acute respiratory syndrome coronavirus 2
VIGIVNNTVY

SEQ ID NO: 205 - protein, 16 aa, Severe acute respiratory syndrome coronavirus 2
LDKYFKNHTSPDVDLG

SEQ ID NO: 206 - protein, 15 aa, Severe acute respiratory syndrome coronavirus 2
DKYFKNHTSPDVDLG

SEQ ID NO: 207 - protein, 14 aa, Severe acute respiratory syndrome coronavirus 2
KYFKNHTSPDVDLG

SEQ ID NO: 208 - protein, 13 aa, Severe acute respiratory syndrome coronavirus 2
YFKNHTSPDVDLG

SEQ ID NO: 209 - protein, 10 aa, Severe acute respiratory syndrome coronavirus 2
YFKNHTSPDV

SEQ ID NO: 210 - protein, 15 aa, Severe acute respiratory syndrome coronavirus 2
MYSFVSEETGTLIVN

SEQ ID NO: 211 - protein, 17 aa, Artificial Sequence
  Description of Artificial Sequence Synthetic peptide
ISLLQFAYANRNRFLYI

SEQ ID NO: 212 - protein, 17 aa, Severe acute respiratory syndrome coronavirus 2
IASFRLFARTRSMWSFN

SEQ ID NO: 213 - protein, 15 aa, Artificial Sequence
  Description of Artificial Sequence Synthetic peptide
RGHLRIAGHHLGRSD

SEQ ID NO: 214 - protein, 14 aa, Severe acute respiratory syndrome coronavirus 2
PPAYTNSFTRGVYY

SEQ ID NO: 215 - protein, 16 aa, Artificial Sequence
  Description of Artificial Sequence Synthetic peptide
NEGVYFASTEKSNIIR

SEQ ID NO: 216 - protein, 17 aa, Severe acute respiratory syndrome coronavirus 2
QTLLALHRSYLTPGDSS

SEQ ID NO: 217 - protein, 18 aa, Artificial Sequence
  Description of Artificial Sequence Synthetic peptide
FASVYAWNRKRISNSVAD

SEQ ID NO: 218 - protein, 23 aa, Severe acute respiratory syndrome coronavirus 2
GGNYNYLYRLFRKSNLKPFERDI

SEQ ID NO: 219 - protein, 14 aa, Artificial Sequence
  Description of Artificial Sequence Synthetic peptide
NKSVNFNFNGLTGT

SEQ ID NO: 220 - protein, 20 aa, Severe acute respiratory syndrome coronavirus 2
FGGFNFSQILPDPSKPSKRS

SEQ ID NO: 221 - protein, 14 aa, Severe acute respiratory syndrome coronavirus 2
TDEMIAQYTSALLA

SEQ ID NO: 222 - protein, 18 aa, Artificial Sequence
  Description of Artificial Sequence Synthetic peptide
LQIPFAMQMAYRFNGIGV

SEQ ID NO: 223 - protein, 15 aa, Severe acute respiratory syndrome coronavirus 2
LDKYFKNHTSPDVDL

SEQ ID NO: 224 - protein, 15 aa, Severe acute respiratory syndrome coronavirus 2
ISGINASVVNIQKEI

SEQ ID NO: 225 - protein, 16 aa, Artificial Sequence
  Description of Artificial Sequence Synthetic peptide
LRMKLPKPPKPVSKMR

SEQ ID NO: 226 - protein, 4 aa, Artificial Sequence
  Description of Artificial Sequence Synthetic peptide
LRMK

SEQ ID NO: 227 - protein, 16 aa, Artificial Sequence
  Description of Artificial Sequence Synthetic peptide
YRMKLPKPPKPVSKMR

SEQ ID NO: 228 - protein, 7 aa, Artificial Sequence
  Description of Artificial Sequence Synthetic peptide
LRMKLPK

SEQ ID NO: 229 - protein, 8 aa, Artificial Sequence
  Description of Artificial Sequence Synthetic peptide
LRMKLPKS

SEQ ID NO: 230 - protein, 11 aa, Artificial Sequence
  Description of Artificial Sequence Synthetic peptide
LRMKLPKSAKP

SEQ ID NO: 231 - protein, 14 aa, Artificial Sequence
  Description of Artificial Sequence Synthetic peptide
LRMKLPKSAKPVSK

SEQ ID NO: 232 - protein, 21 aa, Artificial Sequence
  Description of Artificial Sequence Synthetic peptide
  MOD_RES (5)..(5): 5-aminopentanoic acid
LRMKXPSFYVYSRVKNLNSSR

SEQ ID NO: 233 - protein, 20 aa, Artificial Sequence
  Description of Artificial Sequence Synthetic peptide
  MOD_RES (5)..(5): 5-aminopentanoic acid
LRMKXSFYVYSRVKNLNSSR

SEQ ID NO: 234 - protein, 19 aa, Artificial Sequence
  Description of Artificial Sequence Synthetic peptide
  MOD_RES (5)..(5): 5-aminopentanoic acid
LRMKXFYVYSRVKNLNSSR

SEQ ID NO: 235 - protein, 18 aa, Artificial Sequence
  Description of Artificial Sequence Synthetic peptide
  MOD_RES (5)..(5): 5-aminopentanoic acid
LRMKXYVYSRVKNLNSSR

SEQ ID NO: 236 - protein, 16 aa, Artificial Sequence
  Description of Artificial Sequence Synthetic peptide
  MOD_RES (5)..(5): 5-aminopentanoic acid
LRMKXYVYSRVKNLNS

SEQ ID NO: 237 - protein, 20 aa, Artificial Sequence
  Description of Artificial Sequence Synthetic peptide
  MOD_RES (5)..(5): 5-aminopentanoic acid
LRMKXLKKLLEQWNLVIGFL

SEQ ID NO: 238 - protein, 19 aa, Artificial Sequence
  Description of Artificial Sequence Synthetic peptide
  MOD_RES (5)..(5): 5-aminopentanoic acid
LRMKXKKLLEQWNLVIGFL

SEQ ID NO: 239 - protein, 18 aa, Artificial Sequence
  Description of Artificial Sequence Synthetic peptide
  MOD_RES (5)..(5): 5-aminopentanoic acid
LRMKXKLLEQWNLVIGFL

SEQ ID NO: 240 - protein, 17 aa, Artificial Sequence
  Description of Artificial Sequence Synthetic peptide
  MOD_RES (5)..(5): 5-aminopentanoic acid
LRMKXLLEQWNLVIGFL

SEQ ID NO: 241 - protein, 14 aa, Artificial Sequence
  Description of Artificial Sequence Synthetic peptide
  MOD_RES (5)..(5): 5-aminopentanoic acid
LRMKXLLEQWNLVI

SEQ ID NO: 242 - protein, 20 aa, Artificial Sequence
  Description of Artificial Sequence Synthetic peptide
  MOD_RES (5)..(5): 5-aminopentanoic acid
LRMKXLEQWNLVIGFLFLTW

SEQ ID NO: 243 - protein, 19 aa, Artificial Sequence
  Description of Artificial Sequence Synthetic peptide
  MOD_RES (5)..(5): 5-aminopentanoic acid
LRMKXEQWNLVIGFLFLTW

SEQ ID NO: 244 - protein, 18 aa, Artificial Sequence
  Description of Artificial Sequence Synthetic peptide
  MOD_RES (5)..(5): 5-aminopentanoic acid
LRMKXQWNLVIGFLFLTW

SEQ ID NO: 245 - protein, 17 aa, Artificial Sequence
  Description of Artificial Sequence Synthetic peptide
  MOD_RES (5)..(5): 5-aminopentanoic acid
LRMKXWNLVIGFLFLTW

SEQ ID NO: 246 - protein, 14 aa, Artificial Sequence
  Description of Artificial Sequence Synthetic peptide
  MOD_RES (5)..(5): 5-aminopentanoic acid
LRMKXWNLVIGFLF

SEQ ID NO: 247 - protein, 22 aa, Artificial Sequence
  Description of Artificial Sequence Synthetic peptide
  MOD_RES (5)..(5): 5-aminopentanoic acid
LRMKXICLLQFAYANRNRFLYI

SEQ ID NO: 248 - protein, 21 aa, Artificial Sequence
  Description of Artificial Sequence Synthetic peptide
  MOD_RES (5)..(5): 5-aminopentanoic acid
LRMKXCLLQFAYANRNRFLYI

SEQ ID NO: 249 - protein, 20 aa, Artificial Sequence
  Description of Artificial Sequence Synthetic peptide
  MOD_RES (5)..(5): 5-aminopentanoic acid
LRMKXLLQFAYANRNRFLYI

SEQ ID NO: 250 - protein, 19 aa, Artificial Sequence
  Description of Artificial Sequence Synthetic peptide
  MOD_RES (5)..(5): 5-aminopentanoic acid
LRMKXLQFAYANRNRFLYI

SEQ ID NO: 251 - protein, 16 aa, Artificial Sequence
  Description of Artificial Sequence Synthetic peptide
  MOD_RES (5)..(5): 5-aminopentanoic acid
LRMKXLQFAYANRNRF

SEQ ID NO: 252 - protein, 24 aa, Artificial Sequence
  Description of Artificial Sequence Synthetic peptide
  MOD_RES (5)..(5): 5-aminopentanoic acid
LRMKXRNRFLYIIKLIFLWLLWPV

SEQ ID NO: 253 - protein, 23 aa, Artificial Sequence
  Description of Artificial Sequence Synthetic peptide
  MOD_RES (5)..(5): 5-aminopentanoic acid
LRMKXNRFLYIIKLIFLWLLWPV

SEQ ID NO: 254 - protein, 22 aa, Artificial Sequence
  Description of Artificial Sequence Synthetic peptide
  MOD_RES (5)..(5): 5-aminopentanoic acid
LRMKXRFLYIIKLIFLWLLWPV

SEQ ID NO: 255 - protein, 21 aa, Artificial Sequence
  Description of Artificial Sequence Synthetic peptide
  MOD_RES (5)..(5): 5-aminopentanoic acid
LRMKXFLYIIKLIFLWLLWPV

SEQ ID NO: 256 - protein, 18 aa, Artificial Sequence
  Description of Artificial Sequence Synthetic peptide
  MOD_RES (5)..(5): 5-aminopentanoic acid
LRMKXFLYIIKLIFLWLL

SEQ ID NO: 257 - protein, 20 aa, Artificial Sequence
  Description of Artificial Sequence Synthetic peptide
  MOD_RES (5)..(5): 5-aminopentanoic acid
LRMKXLACFVLAAVYRINWI

SEQ ID NO: 258 - protein, 19 aa, Artificial Sequence
  Description of Artificial Sequence Synthetic peptide
  MOD_RES (5)..(5): 5-aminopentanoic acid
LRMKXACFVLAAVYRINWI

SEQ ID NO: 259 - protein, 18 aa, Artificial Sequence
  Description of Artificial Sequence Synthetic peptide
  MOD_RES (5)..(5): 5-aminopentanoic acid
LRMKXCFVLAAVYRINWI

SEQ ID NO: 260 - protein, 17 aa, Artificial Sequence
  Description of Artificial Sequence Synthetic peptide
  MOD_RES (5)..(5): 5-aminopentanoic acid
LRMKXFVLAAVYRINWI

SEQ ID NO: 261 - protein, 14 aa, Artificial Sequence
  Description of Artificial Sequence Synthetic peptide
  MOD_RES (5)..(5): 5-aminopentanoic acid
LRMKXFVLAAVYRI

SEQ ID NO: 262 - protein, 20 aa, Artificial Sequence
  Description of Artificial Sequence Synthetic peptide
  MOD_RES (5)..(5): 5-aminopentanoic acid
LRMKXLSYFIASFRLFARTR

SEQ ID NO: 263 - protein, 19 aa, Artificial Sequence
  Description of Artificial Sequence Synthetic peptide
  MOD_RES (5)..(5): 5-aminopentanoic acid
LRMKXSYFIASFRLFARTR

SEQ ID NO: 264 - protein, 18 aa, Artificial Sequence
  Description of Artificial Sequence Synthetic peptide
  MOD_RES (5)..(5): 5-aminopentanoic acid
LRMKXYFIASFRLFARTR

SEQ ID NO: 265 - protein, 17 aa, Artificial Sequence
  Description of Artificial Sequence Synthetic peptide
  MOD_RES (5)..(5): 5-aminopentanoic acid
LRMKXFIASFRLFARTR

SEQ ID NO: 266 - protein, 14 aa, Artificial Sequence
  Description of Artificial Sequence Synthetic peptide
  MOD_RES (5)..(5): 5-aminopentanoic acid
LRMKXFIASFRLFA

SEQ ID NO: 267 - protein, 23 aa, Artificial Sequence
  Description of Artificial Sequence Synthetic peptide
  MOD_RES (5)..(5): 5-aminopentanoic acid
LRMKXIASFRLFARTRSMWSFNP

SEQ ID NO: 268 - protein, 22 aa, Artificial Sequence
  Description of Artificial Sequence Synthetic peptide
  MOD_RES (5)..(5): 5-aminopentanoic acid
LRMKXASFRLFARTRSMWSFNP

SEQ ID NO: 269 - protein, 21 aa, Artificial Sequence
  Description of Artificial Sequence Synthetic peptide
  MOD_RES (5)..(5): 5-aminopentanoic acid
LRMKXSFRLFARTRSMWSFNP

SEQ ID NO: 270 - protein, 20 aa, Artificial Sequence
  Description of Artificial Sequence Synthetic peptide
  MOD_RES (5)..(5): 5-aminopentanoic acid
LRMKXFRLFARTRSMWSFNP

SEQ ID NO: 271 - protein, 17 aa, Artificial Sequence
  Description of Artificial Sequence Synthetic peptide
  MOD_RES (5)..(5): 5-aminopentanoic acid
LRMKXFRLFARTRSMWS

SEQ ID NO: 272 - protein, 20 aa, Artificial Sequence
  Description of Artificial Sequence Synthetic peptide
  MOD_RES (5)..(5): 5-aminopentanoic acid
LRMKXRGHLRIAGHHLGRCD

SEQ ID NO: 273 - protein, 19 aa, Artificial Sequence
  Description of Artificial Sequence Synthetic peptide
  MOD_RES (5)..(5): 5-aminopentanoic acid
LRMKXGHLRIAGHHLGRCD

SEQ ID NO: 274 - protein, 18 aa, Artificial Sequence
  Description of Artificial Sequence Synthetic peptide
  MOD_RES (5)..(5): 5-aminopentanoic acid
LRMKXHLRIAGHHLGRCD

SEQ ID NO: 275 - protein, 17 aa, Artificial Sequence
  Description of Artificial Sequence Synthetic peptide
  MOD_RES (5)..(5): 5-aminopentanoic acid
LRMKXLRIAGHHLGRCD

SEQ ID NO: 276 - protein, 14 aa, Artificial Sequence
  Description of Artificial Sequence Synthetic peptide
  MOD_RES (5)..(5): 5-aminopentanoic acid
LRMKXLRIAGHHLG

SEQ ID NO: 277 - protein, 20 aa, Artificial Sequence
  Description of Artificial Sequence Synthetic peptide
  MOD_RES (5)..(5): 5-aminopentanoic acid
LRMKXPKEITVATSRTLSYY

SEQ ID NO: 278 - protein, 19 aa, Artificial Sequence
  Description of Artificial Sequence Synthetic peptide
  MOD_RES (5)..(5): 5-aminopentanoic acid
LRMKXKEITVATSRTLSYY

SEQ ID NO: 279 - protein, 18 aa, Artificial Sequence
  Description of Artificial Sequence Synthetic peptide
  MOD_RES (5)..(5): 5-aminopentanoic acid
LRMKXEITVATSRTLSYY

SEQ ID NO: 280 - protein, 17 aa, Artificial Sequence
  Description of Artificial Sequence Synthetic peptide
  MOD_RES (5)..(5): 5-aminopentanoic acid
LRMKXITVATSRTLSYY

SEQ ID NO: 281 - protein, 14 aa, Artificial Sequence
  Description of Artificial Sequence Synthetic peptide
  MOD_RES (5)..(5): 5-aminopentanoic acid
LRMKXITVATSRTL

SEQ ID NO: 282 - protein, 21 aa, Artificial Sequence
  Description of Artificial Sequence Synthetic peptide
  MOD_RES (5)..(5): 5-aminopentanoic acid
LRMKXTLSYYKLGASQRVAGD

SEQ ID NO: 283 - protein, 20 aa, Artificial Sequence
  Description of Artificial Sequence Synthetic peptide
  MOD_RES (5)..(5): 5-aminopentanoic acid
LRMKXLSYYKLGASQRVAGD

SEQ ID NO: 284 - protein, 19 aa, Artificial Sequence
  Description of Artificial Sequence Synthetic peptide
  MOD_RES (5)..(5): 5-aminopentanoic acid
LRMKXSYYKLGASQRVAGD

SEQ ID NO: 285 - protein, 18 aa, Artificial Sequence
  Description of Artificial Sequence Synthetic peptide
  MOD_RES (5)..(5): 5-aminopentanoic acid
LRMKXYYKLGASQRVAGD

SEQ ID NO: 286 - protein, 15 aa, Artificial Sequence
  Description of Artificial Sequence Synthetic peptide
  MOD_RES (5)..(5): 5-aminopentanoic acid
LRMKXYYKLGASQRV

SEQ ID NO: 287 - protein, 22 aa, Artificial Sequence
  Description of Artificial Sequence Synthetic peptide
  MOD_RES (5)..(5): 5-aminopentanoic acid
LRMKXIGNYKLNTDHSSSSDNI

SEQ ID NO: 288 - protein, 21 aa, Artificial Sequence
  Description of Artificial Sequence Synthetic peptide
  MOD_RES (5)..(5): 5-aminopentanoic acid
LRMKXGNYKLNTDHSSSSDNI

SEQ ID NO: 289 - protein, 20 aa, Artificial Sequence
  Description of Artificial Sequence Synthetic peptide
  MOD_RES (5)..(5): 5-aminopentanoic acid
LRMKXNYKLNTDHSSSSDNI

SEQ ID NO: 290 - protein, 19 aa, Artificial Sequence
  Description of Artificial Sequence Synthetic peptide
  MOD_RES (5)..(5): 5-aminopentanoic acid
LRMKXYKLNTDHSSSSDNI

SEQ ID NO: 291 - protein, 16 aa, Artificial Sequence
  Description of Artificial Sequence Synthetic peptide
  MOD_RES (5)..(5): 5-aminopentanoic acid
LRMKXYKLNTDHSSSS

SEQ ID NO: 292 - protein, 20 aa, Artificial Sequence
  Description of Artificial Sequence Synthetic peptide
  MOD_RES (5)..(5): 5-aminopentanoic acid
LRMKXPPAYTNSFTRGVYYP

SEQ ID NO: 293 - protein, 19 aa, Artificial Sequence
  Description of Artificial Sequence Synthetic peptide
  MOD_RES (5)..(5): 5-aminopentanoic acid
LRMKXPAYTNSFTRGVYYP

SEQ ID NO: 294 - protein, 18 aa, Artificial Sequence
  Description of Artificial Sequence Synthetic peptide
  MOD_RES (5)..(5): 5-aminopentanoic acid
LRMKXAYTNSFTRGVYYP

SEQ ID NO: 295 - protein, 17 aa, Artificial Sequence
  Description of Artificial Sequence Synthetic peptide
  MOD_RES (5)..(5): 5-aminopentanoic acid
LRMKXYTNSFTRGVYYP

SEQ ID NO: 296 - protein, 14 aa, Artificial Sequence
  Description of Artificial Sequence Synthetic peptide
  MOD_RES (5)..(5): 5-aminopentanoic acid
LRMKXYTNSFTRGV

SEQ ID NO: 297 - protein, 20 aa, Artificial Sequence
  Description of Artificial Sequence Synthetic peptide
  MOD_RES (5)..(5): 5-aminopentanoic acid
LRMKXQDLFLPFFSNVTWFH

SEQ ID NO: 298 - protein, 19 aa, Artificial Sequence
  Description of Artificial Sequence Synthetic peptide
  MOD_RES (5)..(5): 5-aminopentanoic acid
LRMKXDLFLPFFSNVTWFH

SEQ ID NO: 299 - protein, 18 aa, Artificial Sequence
  Description of Artificial Sequence Synthetic peptide
  MOD_RES (5)..(5): 5-aminopentanoic acid
LRMKXLFLPFFSNVTWFH

SEQ ID NO: 300 - protein, 17 aa, Artificial Sequence
  Description of Artificial Sequence Synthetic peptide
  MOD_RES (5)..(5): 5-aminopentanoic acid
LRMKXFLPFFSNVTWFH

SEQ ID NO: 301 - protein, 14 aa, Artificial Sequence
  Description of Artificial Sequence Synthetic peptide
  MOD_RES (5)..(5): 5-aminopentanoic acid
LRMKXFLPFFSNVT

SEQ ID NO: 302 - protein, 16 aa, Artificial Sequence
  Description of Artificial Sequence Synthetic peptide
NVTWFHAIHVSGTNGT

SEQ ID NO: 303 - protein, 20 aa, Artificial Sequence
  Description of Artificial Sequence Synthetic peptide
  MOD_RES (5)..(5): 5-aminopentanoic acid
LRMKXVTWFHAIHVSGTNGT

SEQ ID NO: 304 - protein, 19 aa, Artificial Sequence
  Description of Artificial Sequence Synthetic peptide
  MOD_RES (5)..(5): 5-aminopentanoic acid
LRMKXTWFHAIHVSGTNGT

SEQ ID NO: 305 - protein, 18 aa, Artificial Sequence
  Description of Artificial Sequence Synthetic peptide
  MOD_RES (5)..(5): 5-aminopentanoic acid
LRMKXWFHAIHVSGTNGT

SEQ ID NO: 306 - protein, 15 aa, Artificial Sequence
  Description of Artificial Sequence Synthetic peptide
  MOD_RES (5)..(5): 5-aminopentanoic acid
LRMKXWFHAIHVSGT

SEQ ID NO: 307 - protein, 22 aa, Artificial Sequence
  Description of Artificial Sequence Synthetic peptide
  MOD_RES (5)..(5): 5-aminopentanoic acid
LRMKXNDGVYFASTEKSNIIRG

SEQ ID NO: 308 - protein, 21 aa, Artificial Sequence
  Description of Artificial Sequence Synthetic peptide
  MOD_RES (5)..(5): 5-aminopentanoic acid
LRMKXDGVYFASTEKSNIIRG

SEQ ID NO: 309 - protein, 20 aa, Artificial Sequence
  Description of Artificial Sequence Synthetic peptide
  MOD_RES (5)..(5): 5-aminopentanoic acid
LRMKXGVYFASTEKSNIIRG

SEQ ID NO: 310 - protein, 19 aa, Artificial Sequence
  Description of Artificial Sequence Synthetic peptide
  MOD_RES (5)..(5): 5-aminopentanoic acid
LRMKXVYFASTEKSNIIRG

SEQ ID NO: 311 - protein, 16 aa, Artificial Sequence
  Description of Artificial Sequence Synthetic peptide
  MOD_RES (5)..(5): 5-aminopentanoic acid
LRMKXVYFASTEKSNI

SEQ ID NO: 312 - protein, 22 aa, Artificial Sequence
  Description of Artificial Sequence Synthetic peptide
  MOD_RES (5)..(5): 5-aminopentanoic acid
LRMKXTQSLLIVNNATNVVIKV

SEQ ID NO: 313 - protein, 21 aa, Artificial Sequence
  Description of Artificial Sequence Synthetic peptide
  MOD_RES (5)..(5): 5-aminopentanoic acid
LRMKXQSLLIVNNATNVVIKV

SEQ ID NO: 314 - protein, 20 aa, Artificial Sequence
  Description of Artificial Sequence Synthetic peptide
  MOD_RES (5)..(5): 5-aminopentanoic acid
LRMKXSLLIVNNATNVVIKV

SEQ ID NO: 315 - protein, 19 aa, Artificial Sequence
  Description of Artificial Sequence Synthetic peptide
  MOD_RES (5)..(5): 5-aminopentanoic acid
LRMKXLLIVNNATNVVIKV

SEQ ID NO: 316 - protein, 17 aa, Artificial Sequence
  Description of Artificial Sequence Synthetic peptide
  MOD_RES (5)..(5): 5-aminopentanoic acid
LRMKXLLIVNNATNVVI

SEQ ID NO: 317 - protein, 22 aa, Artificial Sequence
  Description of Artificial Sequence Synthetic peptide
  MOD_RES (5)..(5): 5-aminopentanoic acid
LRMKXFLGVYYHKNNKSWMESE

SEQ ID NO: 318 - protein, 21 aa, Artificial Sequence
  Description of Artificial Sequence Synthetic peptide
  MOD_RES (5)..(5): 5-aminopentanoic acid
LRMKXLGVYYHKNNKSWMESE

SEQ ID NO: 319 - protein, 20 aa, Artificial Sequence
  Description of Artificial Sequence Synthetic peptide
  MOD_RES (5)..(5): 5-aminopentanoic acid
LRMKXGVYYHKNNKSWMESE

SEQ ID NO: 320 - protein, 19 aa, Artificial Sequence
  Description of Artificial Sequence Synthetic peptide
  MOD_RES (5)..(5): 5-aminopentanoic acid
LRMKXVYYHKNNKSWMESE

SEQ ID NO: 321 - protein, 16 aa, Artificial Sequence
  Description of Artificial Sequence Synthetic peptide
  MOD_RES (5)..(5): 5-aminopentanoic acid
LRMKXVYYHKNNKSWM

SEQ ID NO: 322 - protein, 23 aa, Artificial Sequence
  Description of Artificial Sequence Synthetic peptide
  MOD_RES (5)..(5): 5-aminopentanoic acid
LRMKXESEFRVYSSANNCTFEYV

SEQ ID NO: 323 - protein, 22 aa, Artificial Sequence
  Description of Artificial Sequence Synthetic peptide
  MOD_RES (5)..(5): 5-aminopentanoic acid
LRMKXSEFRVYSSANNCTFEYV

SEQ ID NO: 324 - protein, 21 aa, Artificial Sequence
  Description of Artificial Sequence Synthetic peptide
  MOD_RES (5)..(5): 5-aminopentanoic acid
LRMKXEFRVYSSANNCTFEYV

SEQ ID NO: 325 - protein, 20 aa, Artificial Sequence
  Description of Artificial Sequence Synthetic peptide
  MOD_RES (5)..(5): 5-aminopentanoic acid
LRMKXFRVYSSANNCTFEYV

SEQ ID NO: 326 - protein, 17 aa, Artificial Sequence
  Description of Artificial Sequence Synthetic peptide
  MOD_RES (5)..(5): 5-aminopentanoic acid
LRMKXFRVYSSANNCTF

SEQ ID NO: 327 - protein, 20 aa, Artificial Sequence
  Description of Artificial Sequence Synthetic peptide
  MOD_RES (5)..(5): 5-aminopentanoic acid
LRMKXDGYFKIYSKHTPINL

SEQ ID NO: 328 - protein, 19 aa, Artificial Sequence
  Description of Artificial Sequence Synthetic peptide
  MOD_RES (5)..(5): 5-aminopentanoic acid
LRMKXGYFKIYSKHTPINL

SEQ ID NO: 329 - protein, 18 aa, Artificial Sequence
  Description of Artificial Sequence Synthetic peptide
  MOD_RES (5)..(5): 5-aminopentanoic acid
LRMKXYFKIYSKHTPINL

SEQ ID NO: 330 - protein, 17 aa, Artificial Sequence
  Description of Artificial Sequence Synthetic peptide
  MOD_RES (5)..(5): 5-aminopentanoic acid
LRMKXFKIYSKHTPINL

SEQ ID NO: 331 - protein, 14 aa, Artificial Sequence
  Description of Artificial Sequence Synthetic peptide
  MOD_RES (5)..(5): 5-aminopentanoic acid
LRMKXFKIYSKHTP

SEQ ID NO: 332 - protein, 22 aa, Artificial Sequence
  Description of Artificial Sequence Synthetic peptide
  MOD_RES (5)..(5): 5-aminopentanoic acid
LRMKXPRTFLLKYNENGTITDA

SEQ ID NO: 333 - protein, 21 aa, Artificial Sequence
  Description of Artificial Sequence Synthetic peptide
  MOD_RES (5)..(5): 5-aminopentanoic acid
LRMKXRTFLLKYNENGTITDA

SEQ ID NO: 334 - protein, 20 aa, Artificial Sequence
  Description of Artificial Sequence Synthetic peptide
  MOD_RES (5)..(5): 5-aminopentanoic acid
LRMKXTFLLKYNENGTITDA

SEQ ID NO: 335 - protein, 19 aa, Artificial Sequence
  Description of Artificial Sequence Synthetic peptide
  MOD_RES (5)..(5): 5-aminopentanoic acid
LRMKXFLLKYNENGTITDA

SEQ ID NO: 336 - protein, 16 aa, Artificial Sequence
  Description of Artificial Sequence Synthetic peptide
  MOD_RES (5)..(5): 5-aminopentanoic acid
LRMKXFLLKYNENGTI

SEQ ID NO: 337 - protein, 20 aa, Artificial Sequence
  Description of Artificial Sequence Synthetic peptide
  MOD_RES (5)..(5): 5-aminopentanoic acid
LRMKXTSNFRVQPTESIVRF

SEQ ID NO: 338 - protein, 19 aa, Artificial Sequence
  Description of Artificial Sequence Synthetic peptide
  MOD_RES (5)..(5): 5-aminopentanoic acid
LRMKXSNFRVQPTESIVRF

SEQ ID NO: 339 - protein, 18 aa, Artificial Sequence
  Description of Artificial Sequence Synthetic peptide
  MOD_RES (5)..(5): 5-aminopentanoic acid
LRMKXNFRVQPTESIVRF

SEQ ID NO: 340 - protein, 17 aa, Artificial Sequence
  Description of Artificial Sequence Synthetic peptide
  MOD_RES (5)..(5): 5-aminopentanoic acid
LRMKXFRVQPTESIVRF

SEQ ID NO: 341 - protein, 14 aa, Artificial Sequence
  Description of Artificial Sequence Synthetic peptide
  MOD_RES (5)..(5): 5-aminopentanoic acid
LRMKXFRVQPTESI

SEQ ID NO: 342 - protein, 20 aa, Artificial Sequence
  Description of Artificial Sequence Synthetic peptide
  MOD_RES (5)..(5): 5-aminopentanoic acid
LRMKXFGEVFNATRFASVYA

SEQ ID NO: 343 - protein, 19 aa, Artificial Sequence
  Description of Artificial Sequence Synthetic peptide
  MOD_RES (5)..(5): 5-aminopentanoic acid
LRMKXGEVFNATRFASVYA

SEQ ID NO: 344 - protein, 18 aa, Artificial Sequence
  Description of Artificial Sequence Synthetic peptide
  MOD_RES (5)..(5): 5-aminopentanoic acid
LRMKXEVFNATRFASVYA

SEQ ID NO: 345 - protein, 17 aa, Artificial Sequence
  Description of Artificial Sequence Synthetic peptide
  MOD_RES (5)..(5): 5-aminopentanoic acid
LRMKXVFNATRFASVYA

SEQ ID NO: 346 - protein, 14 aa, Artificial Sequence
  Description of Artificial Sequence Synthetic peptide
  MOD_RES (5)..(5): 5-aminopentanoic acid
LRMKXVFNATRFAS

SEQ ID NO: 347 - protein, 19 aa, Artificial Sequence
  Description of Artificial Sequence Synthetic peptide
  MOD_RES (5)..(5): 5-aminopentanoic acid
LRMKXFASVYAWNRKRISN

SEQ ID NO: 348 - protein, 18 aa, Artificial Sequence
  Description of Artificial Sequence Synthetic peptide
  MOD_RES (5)..(5): 5-aminopentanoic acid
LRMKXASVYAWNRKRISN

SEQ ID NO: 349 - protein, 17 aa, Artificial Sequence
  Description of Artificial Sequence Synthetic peptide
  MOD_RES (5)..(5): 5-aminopentanoic acid
LRMKXSVYAWNRKRISN

SEQ ID NO: 350 - protein, 16 aa, Artificial Sequence
  Description of Artificial Sequence Synthetic peptide
  MOD_RES (5)..(5): 5-aminopentanoic acid
LRMKXVYAWNRKRISN

SEQ ID NO: 351 - protein, 15 aa, Artificial Sequence
  Description of Artificial Sequence Synthetic peptide
  MOD_RES (5)..(5): 5-aminopentanoic acid
LRMKXVYAWNRKRIS

SEQ ID NO: 352 - protein, 20 aa, Artificial Sequence
  Description of Artificial Sequence Synthetic peptide
  MOD_RES (5)..(5): 5-aminopentanoic acid
LRMKXVADYSVLYNSASFST

SEQ ID NO: 353 - protein, 19 aa, Artificial Sequence
  Description of Artificial Sequence Synthetic peptide
  MOD_RES (5)..(5): 5-aminopentanoic acid
LRMKXADYSVLYNSASFST

SEQ ID NO: 354 - protein, 18 aa, Artificial Sequence
  Description of Artificial Sequence Synthetic peptide
  MOD_RES (5)..(5): 5-aminopentanoic acid
LRMKXDYSVLYNSASFST

SEQ ID NO: 355 - protein, 17 aa, Artificial Sequence
  Description of Artificial Sequence Synthetic peptide
  MOD_RES (5)..(5): 5-aminopentanoic acid
LRMKXYSVLYNSASFST

SEQ ID NO: 356 - protein, 20 aa, Artificial Sequence
  Description of Artificial Sequence Synthetic peptide
  MOD_RES (5)..(5): 5-aminopentanoic acid
LRMKXGGNYNYLYRLFRKSN

SEQ ID NO: 357 - protein, 19 aa, Artificial Sequence
  Description of Artificial Sequence Synthetic peptide
  MOD_RES (5)..(5): 5-aminopentanoic acid
LRMKXGNYNYLYRLFRKSN

SEQ ID NO: 358 - protein, 18 aa, Artificial Sequence
  Description of Artificial Sequence Synthetic peptide
  MOD_RES (5)..(5): 5-aminopentanoic acid
LRMKXNYNYLYRLFRKSN

SEQ ID NO: 359 - protein, 17 aa, Artificial Sequence
  Description of Artificial Sequence Synthetic peptide
  MOD_RES (5)..(5): 5-aminopentanoic acid
LRMKXYNYLYRLFRKSN

SEQ ID NO: 360 - protein, 14 aa, Artificial Sequence
  Description of Artificial Sequence Synthetic peptide
  MOD_RES (5)..(5): 5-aminopentanoic acid
LRMKXYNYLYRLFR

SEQ ID NO: 361 - protein, 20 aa, Artificial Sequence
  Description of Artificial Sequence Synthetic peptide
  MOD_RES (5)..(5): 5-aminopentanoic acid
LRMKXNYLYRLFRKSNLKPF

SEQ ID NO: 362 - protein, 19 aa, Artificial Sequence
  Description of Artificial Sequence Synthetic peptide
  MOD_RES (5)..(5): 5-aminopentanoic acid
LRMKXYLYRLFRKSNLKPF

SEQ ID NO: 363 - protein, 18 aa, Artificial Sequence
  Description of Artificial Sequence Synthetic peptide
  MOD_RES (5)..(5): 5-aminopentanoic acid
LRMKXLYRLFRKSNLKPF

SEQ ID NO: 364 - protein, 17 aa, Artificial Sequence
  Description of Artificial Sequence Synthetic peptide
  MOD_RES (5)..(5): 5-aminopentanoic acid
LRMKXYRLFRKSNLKPF

SEQ ID NO: 365 - protein, 14 aa, Artificial Sequence
  Description of Artificial Sequence Synthetic peptide
  MOD_RES (5)..(5): 5-aminopentanoic acid
LRMKXYRLFRKSNL

SEQ ID NO: 366 - protein, 19 aa, Artificial Sequence
  Description of Artificial Sequence Synthetic peptide
  MOD_RES (5)..(5): 5-aminopentanoic acid
LRMKXRLFRKSNLKPFERD

SEQ ID NO: 367 - protein, 18 aa, Artificial Sequence
  Description of Artificial Sequence Synthetic peptide
  MOD_RES (5)..(5): 5-aminopentanoic acid
LRMKXLFRKSNLKPFERD

SEQ ID NO: 368 - protein, 17 aa, Artificial Sequence
  Description of Artificial Sequence Synthetic peptide
  MOD_RES (5)..(5): 5-aminopentanoic acid
LRMKXFRKSNLKPFERD

SEQ ID NO: 369 - protein, 14 aa, Artificial Sequence
  Description of Artificial Sequence Synthetic peptide
  MOD_RES (5)..(5): 5-aminopentanoic acid
LRMKXFRKSNLKPF

SEQ ID NO: 370 - protein, 23 aa, Artificial Sequence
  Description of Artificial Sequence Synthetic peptide
  MOD_RES (5)..(5): 5-aminopentanoic acid
LRMKXVEGFNCYFPLQSYGFQPT

SEQ ID NO: 371 - protein, 22 aa, Artificial Sequence
  Description of Artificial Sequence Synthetic peptide
  MOD_RES (5)..(5): 5-aminopentanoic acid
LRMKXEGFNCYFPLQSYGFQPT

SEQ ID NO: 372 - protein, 21 aa, Artificial Sequence
  Description of Artificial Sequence Synthetic peptide
  MOD_RES (5)..(5): 5-aminopentanoic acid
LRMKXGFNCYFPLQSYGFQPT

SEQ ID NO: 373 - protein, 20 aa, Artificial Sequence
  Description of Artificial Sequence Synthetic peptide
  MOD_RES (5)..(5): 5-aminopentanoic acid
LRMKXFNCYFPLQSYGFQPT

SEQ ID NO: 374 - protein, 17 aa, Artificial Sequence
  Description of Artificial Sequence Synthetic peptide
  MOD_RES (5)..(5): 5-aminopentanoic acid
LRMKXFNCYFPLQSYGF

SEQ ID NO: 375 - protein, 22 aa, Artificial Sequence
  Description of Artificial Sequence Synthetic peptide
  MOD_RES (5)..(5): 5-aminopentanoic acid
LRMKXLQSYGFQPTNGVGYQPY

SEQ ID NO: 376 - protein, 21 aa, Artificial Sequence
  Description of Artificial Sequence Synthetic peptide
  MOD_RES (5)..(5): 5-aminopentanoic acid
LRMKXQSYGFQPTNGVGYQPY

SEQ ID NO: 377 - protein, 20 aa, Artificial Sequence
  Description of Artificial Sequence Synthetic peptide
  MOD_RES (5)..(5): 5-aminopentanoic acid
LRMKXSYGFQPTNGVGYQPY

SEQ ID NO: 378 - protein, 19 aa, Artificial Sequence
  Description of Artificial Sequence Synthetic peptide
  MOD_RES (5)..(5): 5-aminopentanoic acid
LRMKXYGFQPTNGVGYQPY

SEQ ID NO: 379 - protein, 16 aa, Artificial Sequence
  Description of Artificial Sequence Synthetic peptide
  MOD_RES (5)..(5): 5-aminopentanoic acid
LRMKXYGFQPTNGVGY

SEQ ID NO: 380 - protein, 20 aa, Artificial Sequence
  Description of Artificial Sequence Synthetic peptide
  MOD_RES (5)..(5): 5-aminopentanoic acid
LRMKXVLSFELLHAPATVCG

SEQ ID NO: 381 - protein, 19 aa, Artificial Sequence
  Description of Artificial Sequence Synthetic peptide
  MOD_RES (5)..(5): 5-aminopentanoic acid
LRMKXLSFELLHAPATVCG

SEQ ID NO: 382 - protein, 18 aa, Artificial Sequence
  Description of Artificial Sequence Synthetic peptide
  MOD_RES (5)..(5): 5-aminopentanoic acid
LRMKXSFELLHAPATVCG

SEQ ID NO: 383 - protein, 17 aa, Artificial Sequence
  Description of Artificial Sequence Synthetic peptide
  MOD_RES (5)..(5): 5-aminopentanoic acid
LRMKXFELLHAPATVCG

SEQ ID NO: 384 - protein, 14 aa, Artificial Sequence
  Description of Artificial Sequence Synthetic peptide
  MOD_RES (5)..(5): 5-aminopentanoic acid
LRMKXFELLHAPAT

SEQ ID NO: 385 - protein, 20 aa, Artificial Sequence
  Description of Artificial Sequence Synthetic peptide
  MOD_RES (5)..(5): 5-aminopentanoic acid
LRMKXNKCVNFNFNGLTGTG

SEQ ID NO: 386 - protein, 19 aa, Artificial Sequence
  Description of Artificial Sequence Synthetic peptide
  MOD_RES (5)..(5): 5-aminopentanoic acid
LRMKXKCVNFNFNGLTGTG

SEQ ID NO: 387 - protein, 18 aa, Artificial Sequence
  Description of Artificial Sequence Synthetic peptide
  MOD_RES (5)..(5): 5-aminopentanoic acid
LRMKXCVNFNFNGLTGTG

SEQ ID NO: 388 - protein, 17 aa, Artificial Sequence
  Description of Artificial Sequence Synthetic peptide
  MOD_RES (5)..(5): 5-aminopentanoic acid
LRMKXVNFNFNGLTGTG

SEQ ID NO: 389 - protein, 14 aa, Artificial Sequence
  Description of Artificial Sequence Synthetic peptide
  MOD_RES (5)..(5): 5-aminopentanoic acid
LRMKXVNFNFNGLT

SEQ ID NO: 390 - protein, 20 aa, Artificial Sequence
  Description of Artificial Sequence Synthetic peptide
  MOD_RES (5)..(5): 5-aminopentanoic acid
LRMKXNFNFNGLTGTGVLTE

SEQ ID NO: 391 - protein, 19 aa, Artificial Sequence
  Description of Artificial Sequence Synthetic peptide
  MOD_RES (5)..(5): 5-aminopentanoic acid
LRMKXFNFNGLTGTGVLTE

SEQ ID NO: 392 - protein, 18 aa, Artificial Sequence
  Description of Artificial Sequence Synthetic peptide
  MOD_RES (5)..(5): 5-aminopentanoic acid
LRMKXNFNGLTGTGVLTE

SEQ ID NO: 393 - protein, 17 aa, Artificial Sequence
  Description of Artificial Sequence Synthetic peptide
  MOD_RES (5)..(5): 5-aminopentanoic acid
LRMKXFNGLTGTGVLTE

SEQ ID NO: 394 - protein, 14 aa, Artificial Sequence
  Description of Artificial Sequence Synthetic peptide
  MOD_RES (5)..(5): 5-aminopentanoic acid
LRMKXFNGLTGTGV

SEQ ID NO: 395 - protein, 19 aa, Artificial Sequence
  Description of Artificial Sequence Synthetic peptide
  MOD_RES (5)..(5): 5-aminopentanoic acid
LRMKXTQLNRALTGIAVEQ

SEQ ID NO: 396 - protein, 18 aa, Artificial Sequence
  Description of Artificial Sequence Synthetic peptide
  MOD_RES (5)..(5): 5-aminopentanoic acid
LRMKXQLNRALTGIAVEQ

SEQ ID NO: 397 - protein, 17 aa, Artificial Sequence
  Description of Artificial Sequence Synthetic peptide
  MOD_RES (5)..(5): 5-aminopentanoic acid
LRMKXLNRALTGIAVEQ

SEQ ID NO: 398 - protein, 14 aa, Artificial Sequence
  Description of Artificial Sequence Synthetic peptide
  MOD_RES (5)..(5): 5-aminopentanoic acid
LRMKXLNRALTGIA

SEQ ID NO: 399 - protein, 20 aa, Artificial Sequence
  Description of Artificial Sequence Synthetic peptide
  MOD_RES (5)..(5): 5-aminopentanoic acid
LRMKXTDEMIAQYTSALLAG

SEQ ID NO: 400 - protein, 19 aa, Artificial Sequence
  Description of Artificial Sequence Synthetic peptide
  MOD_RES (5)..(5): 5-aminopentanoic acid
LRMKXDEMIAQYTSALLAG

SEQ ID NO: 401 - protein, 18 aa, Artificial Sequence
  Description of Artificial Sequence Synthetic peptide
  MOD_RES (5)..(5): 5-aminopentanoic acid
LRMKXEMIAQYTSALLAG

SEQ ID NO: 402 - protein, 17 aa, Artificial Sequence
  Description of Artificial Sequence Synthetic peptide
  MOD_RES (5)..(5): 5-aminopentanoic acid
LRMKXMIAQYTSALLAG

SEQ ID NO: 403 - protein, 15 aa, Artificial Sequence
  Description of Artificial Sequence Synthetic peptide
  MOD_RES (5)..(5): 5-aminopentanoic acid
LRMKXMIAQYTSALL

SEQ ID NO: 404 - protein, 22 aa, Artificial Sequence
  Description of Artificial Sequence Synthetic peptide
  MOD_RES (5)..(5): 5-aminopentanoic acid
LRMKXQIPFAMQMAYRFNGIGV

SEQ ID NO: 405 - protein, 21 aa, Artificial Sequence
  Description of Artificial Sequence Synthetic peptide
  MOD_RES (5)..(5): 5-aminopentanoic acid
LRMKXIPFAMQMAYRFNGIGV

SEQ ID NO: 406 - protein, 20 aa, Artificial Sequence
  Description of Artificial Sequence Synthetic peptide
  MOD_RES (5)..(5): 5-aminopentanoic acid
LRMKXPFAMQMAYRFNGIGV

SEQ ID NO: 407 - protein, 19 aa, Artificial Sequence
  Description of Artificial Sequence Synthetic peptide
  MOD_RES (5)..(5): 5-aminopentanoic acid
LRMKXFAMQMAYRFNGIGV

SEQ ID NO: 408 - protein, 16 aa, Artificial Sequence
  Description of Artificial Sequence Synthetic peptide
  MOD_RES (5)..(5): 5-aminopentanoic acid
LRMKXFAMQMAYRFNG

SEQ ID NO: 409 - protein, 20 aa, Artificial Sequence
  Description of Artificial Sequence Synthetic peptide
  MOD_RES (5)..(5): 5-aminopentanoic acid
LRMKXQKLIANQFNSAIGKI

SEQ ID NO: 410 - protein, 19 aa, Artificial Sequence
  Description of Artificial Sequence Synthetic peptide
  MOD_RES (5)..(5): 5-aminopentanoic acid
LRMKXKLIANQFNSAIGKI

SEQ ID NO: 411 - protein, 18 aa, Artificial Sequence
  Description of Artificial Sequence Synthetic peptide
  MOD_RES (5)..(5): 5-aminopentanoic acid
LRMKXLIANQFNSAIGKI

SEQ ID NO: 412 - protein, 17 aa, Artificial Sequence
  Description of Artificial Sequence Synthetic peptide
  MOD_RES (5)..(5): 5-aminopentanoic acid
LRMKXIANQFNSAIGKI

SEQ ID NO: 413 - protein, 14 aa, Artificial Sequence
  Description of Artificial Sequence Synthetic peptide
  MOD_RES (5)..(5): 5-aminopentanoic acid
LRMKXIANQFNSAI

SEQ ID NO: 414 - protein, 20 aa, Artificial Sequence
  Description of Artificial Sequence Synthetic peptide
  MOD_RES (5)..(5): 5-aminopentanoic acid
LRMKXANQFNSAIGKIQDSL

SEQ ID NO: 415 - protein, 19 aa, Artificial Sequence
  Description of Artificial Sequence Synthetic peptide
  MOD_RES (5)..(5): 5-aminopentanoic acid
LRMKXNQFNSAIGKIQDSL

SEQ ID NO: 416 - protein, 18 aa, Artificial Sequence
  Description of Artificial Sequence Synthetic peptide
  MOD_RES (5)..(5): 5-aminopentanoic acid
LRMKXQFNSAIGKIQDSL

SEQ ID NO: 417 - protein, 17 aa, Artificial Sequence
  Description of Artificial Sequence Synthetic peptide
  MOD_RES (5)..(5): 5-aminopentanoic acid
LRMKXFNSAIGKIQDSL

SEQ ID NO: 418 - protein, 14 aa, Artificial Sequence
  Description of Artificial Sequence Synthetic peptide
  MOD_RES (5)..(5): 5-aminopentanoic acid
LRMKXFNSAIGKIQ

SEQ ID NO: 419 - protein, 20 aa, Artificial Sequence
  Description of Artificial Sequence Synthetic peptide
  MOD_RES (5)..(5): 5-aminopentanoic acid
LRMKXTGRLQSLQTYVTQQL

SEQ ID NO: 420 - protein, 19 aa, Artificial Sequence
  Description of Artificial Sequence Synthetic peptide
  MOD_RES (5)..(5): 5-aminopentanoic acid
LRMKXGRLQSLQTYVTQQL

SEQ ID NO: 421 - protein, 18 aa, Artificial Sequence
  Description of Artificial Sequence Synthetic peptide
  MOD_RES (5)..(5): 5-aminopentanoic acid
LRMKXRLQSLQTYVTQQL

SEQ ID NO: 422 - protein, 17 aa, Artificial Sequence
  Description of Artificial Sequence Synthetic peptide
  MOD_RES (5)..(5): 5-aminopentanoic acid
LRMKXLQSLQTYVTQQL

SEQ ID NO: 423 - protein, 14 aa, Artificial Sequence
  Description of Artificial Sequence Synthetic peptide
  MOD_RES (5)..(5): 5-aminopentanoic acid
LRMKXLQSLQTYVT

SEQ ID NO: 424 - protein, 20 aa, Artificial Sequence
  Description of Artificial Sequence Synthetic peptide
  MOD_RES (5)..(5): 5-aminopentanoic acid
LRMKXGKGYHLMSFPQSAPH

SEQ ID NO: 425 - protein, 19 aa, Artificial Sequence
  Description of Artificial Sequence Synthetic peptide
  MOD_RES (5)..(5): 5-aminopentanoic acid
LRMKXKGYHLMSFPQSAPH

SEQ ID NO: 426 - protein, 18 aa, Artificial Sequence
  Description of Artificial Sequence Synthetic peptide
  MOD_RES (5)..(5): 5-aminopentanoic acid
LRMKXGYHLMSFPQSAPH

SEQ ID NO: 427 - protein, 17 aa, Artificial Sequence
  Description of Artificial Sequence Synthetic peptide
  MOD_RES (5)..(5): 5-aminopentanoic acid
LRMKXYHLMSFPQSAPH

SEQ ID NO: 428 - protein, 14 aa, Artificial Sequence
  Description of Artificial Sequence Synthetic peptide
  MOD_RES (5)..(5): 5-aminopentanoic acid
LRMKXYHLMSFPQS

SEQ ID NO: 429 - protein, 20 aa, Artificial Sequence
  Description of Artificial Sequence Synthetic peptide
  MOD_RES (5)..(5): 5-aminopentanoic acid
LRMKXDVVIGIVNNTVYDPL

SEQ ID NO: 430 - protein, 19 aa, Artificial Sequence
  Description of Artificial Sequence Synthetic peptide
  MOD_RES (5)..(5): 5-aminopentanoic acid
LRMKXVVIGIVNNTVYDPL

SEQ ID NO: 431 - protein, 18 aa, Artificial Sequence
  Description of Artificial Sequence Synthetic peptide
  MOD_RES (5)..(5): 5-aminopentanoic acid
LRMKXVIGIVNNTVYDPL

SEQ ID NO: 432 - protein, 15 aa, Artificial Sequence
  Description of Artificial Sequence Synthetic peptide
  MOD_RES (5)..(5): 5-aminopentanoic acid
LRMKXVIGIVNNTVY

SEQ ID NO: 433 - protein, 21 aa, Artificial Sequence
  Description of Artificial Sequence Synthetic peptide
  MOD_RES (5)..(5): 5-aminopentanoic acid
LRMKXLDKYFKNHTSPDVDLG

SEQ ID NO: 434 - protein, 20 aa, Artificial Sequence
  Description of Artificial Sequence Synthetic peptide
  MOD_RES (5)..(5): 5-aminopentanoic acid
LRMKXDKYFKNHTSPDVDLG

SEQ ID NO: 435 - protein, 19 aa, Artificial Sequence
  Description of Artificial Sequence Synthetic peptide
  MOD_RES (5)..(5): 5-aminopentanoic acid
LRMKXKYFKNHTSPDVDLG

SEQ ID NO: 436 - protein, 18 aa, Artificial Sequence
  Description of Artificial Sequence Synthetic peptide
  MOD_RES (5)..(5): 5-aminopentanoic acid
LRMKXYFKNHTSPDVDLG

SEQ ID NO: 437 - protein, 15 aa, Artificial Sequence
  Description of Artificial Sequence Synthetic peptide
  MOD_RES (5)..(5): 5-aminopentanoic acid
LRMKXYFKNHTSPDV

SEQ ID NO: 438 - protein, 20 aa, Artificial Sequence
  Description of Artificial Sequence Synthetic peptide
  MOD_RES (5)..(5): 5-aminopentanoic acid
LRMKXMYSFVSEETGTLIVN

SEQ ID NO: 439 - protein, 22 aa, Artificial Sequence
  Description of Artificial Sequence Synthetic peptide
  MOD_RES (5)..(5): 5-aminopentanoic acid
LRMKXISLLQFAYANRNRFLYI

SEQ ID NO: 440 - protein, 22 aa, Artificial Sequence
  Description of Artificial Sequence Synthetic peptide
  MOD_RES (5)..(5): 5-aminopentanoic acid
LRMKXIASFRLFARTRSMWSFN

SEQ ID NO: 441 - protein, 20 aa, Artificial Sequence
  Description of Artificial Sequence Synthetic peptide
  MOD_RES (5)..(5): 5-aminopentanoic acid
LRMKXRGHLRIAGHHLGRSD

SEQ ID NO: 442 - protein, 19 aa, Artificial Sequence
  Description of Artificial Sequence Synthetic peptide
  MOD_RES (5)..(5): 5-aminopentanoic acid
LRMKXPPAYTNSFTRGVYY

SEQ ID NO: 443 - protein, 21 aa, Artificial Sequence
  Description of Artificial Sequence Synthetic peptide
  MOD_RES (5)..(5): 5-aminopentanoic acid
LRMKXNEGVYFASTEKSNIIR

SEQ ID NO: 444 - protein, 22 aa, Artificial Sequence
  Description of Artificial Sequence Synthetic peptide
  MOD_RES (5)..(5): 5-aminopentanoic acid
LRMKXQTLLALHRSYLTPGDSS

SEQ ID NO: 445 - protein, 23 aa, Artificial Sequence
  Description of Artificial Sequence Synthetic peptide
  MOD_RES (5)..(5): 5-aminopentanoic acid
LRMKXFASVYAWNRKRISNSVAD

SEQ ID NO: 446 - protein, 28 aa, Artificial Sequence
  Description of Artificial Sequence Synthetic peptide
  MOD_RES (5)..(5): 5-aminopentanoic acid
LRMKXGGNYNYLYRLFRKSNLKPFERDI

SEQ ID NO: 447 - protein, 19 aa, Artificial Sequence
  Description of Artificial Sequence Synthetic peptide
  MOD_RES (5)..(5): 5-aminopentanoic acid
LRMKXNKSVNFNFNGLTGT

SEQ ID NO: 448 - protein, 25 aa, Artificial Sequence
  Description of Artificial Sequence Synthetic peptide
  MOD_RES (5)..(5): 5-aminopentanoic acid
LRMKXFGGFNFSQILPDPSKPSKRS

SEQ ID NO: 449 - protein, 19 aa, Artificial Sequence
  Description of Artificial Sequence Synthetic peptide
  MOD_RES (5)..(5): 5-aminopentanoic acid
LRMKXTDEMIAQYTSALLA

SEQ ID NO: 450 - protein, 23 aa, Artificial Sequence
  Description of Artificial Sequence Synthetic peptide
  MOD_RES (5)..(5): 5-aminopentanoic acid
LRMKXLQIPFAMQMAYRFNGIGV

SEQ ID NO: 451 - protein, 20 aa, Artificial Sequence
  Description of Artificial Sequence Synthetic peptide
  MOD_RES (5)..(5): 5-aminopentanoic acid
LRMKXLDKYFKNHTSPDVDL

SEQ ID NO: 452 - protein, 20 aa, Artificial Sequence
  Description of Artificial Sequence Synthetic peptide
  MOD_RES (5)..(5): 5-aminopentanoic acid
LRMKXISGINASVVNIQKEI

SEQ ID NO: 453 - protein, 5 aa, Artificial Sequence
  Description of Artificial Sequence Synthetic peptide
  MOD_RES (5)..(5): 5-aminopentanoic acid
LRMKX

SEQ ID NO: 454 - protein, 16 aa, Severe acute respiratory syndrome coronavirus 2
NDGVYFASTEKSNIIR

SEQ ID NO: 455 - protein, 18 aa, Severe acute respiratory syndrome coronavirus 2
FASVYAWNRKRISNCVAD

SEQ ID NO: 456 - protein, 14 aa, Severe acute respiratory syndrome coronavirus 2
NKCVNFNFNGLTGT

SEQ ID NO: 457 - protein, 21 aa, Artificial Sequence
  Description of Artificial Sequence Synthetic peptide
  MOD_RES (5)..(5): 5-aminopentanoic acid
LRMKXNDGVYFASTEKSNIIR

SEQ ID NO: 458 - protein, 23 aa, Artificial Sequence
  Description of Artificial Sequence Synthetic peptide
  MOD_RES (5)..(5): 5-aminopentanoic acid
LRMKXFASVYAWNRKRISNCVAD

SEQ ID NO: 459 - protein, 19 aa, Artificial Sequence
  Description of Artificial Sequence Synthetic peptide
  MOD_RES (5)..(5): 5-aminopentanoic acid
LRMKXNKCVNFNFNGLTGT